(19) United States
(12) Patent Application Publication
(10) Pub. No.: US 2026/0262643 A1
(43) Pub. Date: Sep. 10, 2026

(54) HERBICIDAL BENZOXAZINES

(71) Applicant: FMC CORPORATION, Philadelphia, PA (US)

(72) Inventors: Alison Mary LEVENS, Newark, DE (US); Stephen Frederick MCCANN, Newark, DE (US); Saptarshi DE, Hyderabad (IN); Michael HOLMES, Newark, DE (US); Yu TANG, Newark, DE (US)

(21) Appl. No.: 18/993,243

(22) PCT Filed: Jul. 12, 2023
(86) PCT No.: PCT/US2023/027467

§ 371 Date: Jan. 10, 2025

Related U.S. Application Data
(60) Provisional application No. 63/389,027, filed on Jul. 14, 2022.

(51) Int. Cl. A01N 43/90; A01P 13/02; C07D 413/10; C07D 413/14

(52) U.S. Cl. CPC A01N 43/90; A01P 13/02; C07D 413/10; C07D 413/14

(57) ABSTRACT

Disclosed are compounds of Formula 1, including all stereoisomers, N-oxides, and salts thereof, agricultural compositions containing them and their use as herbicides

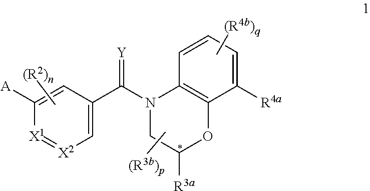

A is a 5- or 6-membered heterocyclic ring, containing ring members selected from carbon atoms and up to 4 heteroatoms independently selected from up to 2 O, up to 2 S and up to 4 N atoms, one or two carbon or sulfur ring members of the heterocycle can optionally be in the oxidized form of a carbonyl, sulfonyl, sulfinyl moiety, said ring bound to the remainder of Formula 1 through a carbon atom or a heteroatom, and optionally substituted with 1 to 4 R¹;
and R^{1a}, R^{1b}, R², R^{3a}, R^{3b}, R^{4a}, R^{4b}, X¹, X², n, p and q are as defined in the disclosure.

### FIELD OF THE INVENTION

This invention relates to certain benzoxazine herbicides, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

### SUMMARY OF THE INVENTION

This invention is directed to compounds of Formula 1, all stereoisomers, N-oxides, and salts thereof, agricultural compositions containing them and their use as herbicides:

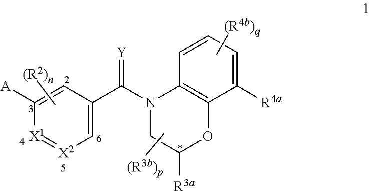

wherein
A is a 5- or 6-membered heterocyclic ring, containing ring members selected from carbon atoms and up to 4 heteroatoms independently selected from up to 2 O, up to 2 S and up to 4 N atoms, one or two carbon or sulfur ring members of the heterocycle can optionally be in the oxidized form of a carbonyl, thiocarbonyl, sulfonyl, sulfinyl moiety, said ring bound to the remainder of Formula 1 through a carbon atom or a heteroatom, and optionally substituted with 1 to 4 R¹;
R¹ is independently R^{1a}, (R^{1b})ₘ, R^{1c} or any combination thereof;
R^{1a} is H, halogen, cyano, nitro, amino, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ alkyloxacycloalkyl, C₂-C₆ thiacycloalkyl, C₃-C₇ thiacycloalkylalkyl, C₃-C₇ alkylthiacycloalkyl, C₂-C₆ (O-thia)cycloalkyl, C₃-C₇ (O-thia)cycloalkylalkyl, C₃-C₇ alkyl(O-thia)cycloalkyl, C₂-C₆ (O₂thia)cycloalkyl, C₃-C₇ (O₂thia)cycloalkylalkyl, C₃-C₇ alkyl(O₂thia)cycloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl, C₁-C₇ haloalkoxy, C₁-C₇ alkylthio, C₂-C₇ alkylthioalkyl, C₁-C₅ alkylsulfinyl, C₁-C₅ alkylsulfonyl, C₁-C₄ alkylsulfonate, C₁-C₅ haloalkylthio, C₁-C₅ haloalkylsulfinyl, C₁-C₅ haloalkylsulfonyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₄-C₇ alkylthiocycloalkyl, C₄-C₇ alkylsulfinylcycloalkyl, C₄-C₇ alkylsulfonylcycloalkyl, C₄-C₇ haloalkylthiocycloalkyl, C₂-C₇ haloalkylsulfinylcycloalkyl, C₂-C₇ haloalkylsulfonylcycloalkyl, C₂-C₇ alkylsulfoximinoalkyl, C₂-C₅ cyanoalkyl, C₄-C₇ cyanocycloalkyl, C₁-C₄ nitroalkyl, C₁-C₇ alkylamino, C₂-C₇ dialkylamino, C₃-C₅ alkylcarbonyl(alkyl)amino, C₃-C₅ alkoxycarbonyl(alkyl)amino, C₂-C₄ alkylsulfonyl(alkyl)amino, C₂-C₆ alkylcarbonyl, C₃-C₆ alkylcarbonylalkyl, C₂-C₆ alkoxycarbonyl, C₃-C₆ alkoxycarbonylalkyl, C₃-C₆ trialkylsilyl or C₅-C₈ trialkylsilylalkynyl; or phenyl optionally substituted with up to 3 substituents independently selected from the group consisting of halogen, cyano, C₁-C₂ alkyl, C₁-C₂ haloalkyl, C₁-C₂ alkoxy and C₁-C₂ haloalkoxy;
R^{1b} is H, halogen, cyano, nitro, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy, C₁-C₄ alkylthio or C₂-C₄ alkoxycarbonyl;
m is 0, 1 or 2;
R^{1c} is H, C₁-C₇ alkyl, C₃-C₇ cycloalkyl or C₁-C₇ haloalkyl;
X¹ and X² are independently N or CR²;
n is 0, 1, 2or 3;
each R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₃-C₅ cycloalkylsulfonate, C₁-C₄ haloalkylsulfonate, C₁-C₄ haloalkylthio, C₁-C₄ haloalkylsulfinyl, C₁-C₄ haloalkylsulfonyl C₂-C₅ cyanoalkyl, C₄-C₆ cyanocycloalkyl or C₂-C₅ alkoxycarbonyl; or
   two adjacent R² may be taken together to form a saturated or unsaturated 5- to 8-membered ring, containing carbon atoms and optionally 1 to 3 oxygen, sulfur or nitrogen atoms as ring members, one or two carbon or sulfur ring members of the heterocycle can optionally be in the oxidized form of a carbonyl, sulfonyl, sulfinyl moiety, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy;
Y is O or S;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₂-C₅ cyanoalkyl, C₄-C₆ cyanocycloalkyl, C₁-C₄ nitroalkyl, C₃-C₆ alkylcarbonylalkyl, C₂-C₆ oxacycloalkyl, C₂-C₆ oxacycloalkylalkyl, C₃-C₇ cycloalkoxy, C₄-C₇ cycloalkoxyalkyl or C₃-C₆ alkoxycarbonylalkyl;
each R^{3b} is independently H, halogen or C₁-C₃ alkyl; or
R^{3a} and R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂; or
two R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂;
p is 0, 1, 2 or 3;
R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl, C₅-C₈ trialkylsilylalkynyl, C₁-C₅ alkylthio, C₁-C₅ haloalkylthio or C₂-C₅ alkoxycarbonyl;
q is 0, 1 or 2;
each R^{4b} is independently H, halogen, cyano, nitro, C₁-C₄ alkyl, C₁-C₄ haloalkyl, C₁-C₄ alkoxy or C₁-C₄ alkylthio;
provided that the compounds of Formula 1 are other than:
[3-(5-cyclopropyl-1,2,4-oxadiazol-3-yl)phenyl](2-ethyl-2,3-dihydro-4H-1,4-benzoxazin-4-yl)-methanone (CAS Registry No. 1798020-19-5);
(8-chloro-2-ethyl-2,3-dihydro-4H-1,4-benzoxazin-4-yl)[3-(1H-tetrazol-1-yl)phenyl]methanone (CAS Registry No. 2093742-48-2);
(2-ethyl-2,3-dihydro-4H-1,4-benzoxazin-4-yl)[2-(1H-1,2,4-triazol-1-yl)-4-pyridinyl]-methanone (CAS Registry No. 1808378-56-4);
(8-chloro-2-ethyl-2,3-dihydro-4H-1,4-benzoxazin-4-yl)[2-(1H-1,2,4-triazol-1-yl)-4-pyridinyl]-methanone (CAS Registry No. 1808849-41-3);
(2,3-dihydro-2,7-dimethyl-4H-1,4-benzoxazin-4-yl)[2-(1H-1,2,4-triazol-1-yl)-4-pyridinyl]-methanone (CAS Registry No. 1436224-65-5);
(8-chloro-2,3-dihydro-2-methyl-4H-1,4-benzoxazin-4-yl)[2-(1H-pyrazol-1-yl)-4-pyridinyl]-methanone (CAS Registry No. 2224006-86-2);
(2,3-Dihydro-2-methyl-4H-1,4-benzoxazin-4-yl)[3-(1-pyrrolidinyl)phenyl]methanone (CAS Registry No. 2733463-68-6);
Methanone, (3,4-dihydrospiro[2H-1,4-benzoxazine-2,1′-cyclopropan]-4-yl)[3-(1-pyrrolidinyl)phenyl](CAS Registry No. 2733410-16-5);
(2,3-Dihydro-2-methyl-4H-1,4-benzoxazin-4-yl)[3-(1H-pyrrol-1-yl)phenyl]methanone (CAS Registry No. 2305402-15-5);
(3,4-Dihydrospiro[2H-1,4-benzoxazine-2,1′-cyclopropan]-4-yl)[3-(1H-pyrrol-1-yl)phenyl]methanone (CAS Registry No. 2305290-36-0);
(8-Chloro-2,3-dihydro-2-methyl-4H-1,4-benzoxazin-4-yl)[3-[5-(methoxymethyl)-1,3,4-oxadiazol-2-yl]phenyl]methanone (CAS Registry No. 2223792-20-7);
(7-Fluoro-2,3-dihydro-2-methyl-4H-1,4-benzoxazin-4-yl)[2-(4-methyl-1-piperazinyl)-4-pyridinyl]methanone (CAS Registry No. 2212440-53-2);
(7-Fluoro-2,3-dihydro-2-methyl-4H-1,4-benzoxazin-4-yl)[3-(1H-1,2,4-triazol-5-yl)phenyl]methanone (CAS Registry No. 2094921-82-9);
(2-Ethyl-2,3-dihydro-4H-1,4-benzoxazin-4-yl)[5-(4-morpholinyl)-3-pyridinyl]methanone (CAS Registry No. 1957585-10-2);
(8-Chloro-2,3-dihydro-2-methyl-4H-1,4-benzoxazin-4-yl)[5-(4-morpholinyl)-3-pyridinyl]methanone (CAS Registry No. 1957541-06-8);
(8-Chloro-2-ethyl-2,3-dihydro-4H-1,4-benzoxazin-4-yl)[5-(4-morpholinyl)-3-pyridinyl]methanone (CAS Registry No. 1956163-57-7);
(2,3-Dihydro-2,6-dimethyl-4H-1,4-benzoxazin-4-yl)[5-(4-morpholinyl)-3-pyridinyl]methanone (CAS Registry No. 1955383-94-4);
(2,3-Dihydro-2,2-dimethyl-4H-1,4-benzoxazin-4-yl)[5-(4-morpholinyl)-3-pyridinyl]methanone (CAS Registry No. 1955104-90-1);
(6-Chloro-2,3-dihydro-2-methyl-4H-1,4-benzoxazin-4-yl)[5-(4-morpholinyl)-3-pyridinyl]methanone (CAS Registry No. 1954398-26-5);
(2,3-Dihydro-6-methoxy-2-methyl-4H-1,4-benzoxazin-4-yl)[5-(4-morpholinyl)-3-pyridinyl]methanone (CAS Registry No. 1947266-43-4); and
(2,3-Dihydro-6-methoxy-2-methyl-4H-1,4-benzoxazin-4-yl)[3-(1H-imidazol-1-yl)phenyl]methanone (CAS Registry No. 1384688-76-9).

More particularly, this invention pertains to a compound of Formula 1 (including all stereoisomers), an N-oxide or a salt thereof. This invention also relates to a herbicidal composition comprising a compound of the invention (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the invention (e.g., as a composition described herein).

This invention also includes a herbicidal mixture comprising (a) a compound selected from Formula 1, N-oxides, and salts thereof, and (b) at least one additional active ingredient selected from (b1) through (b16), and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention.

The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of” Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating” refers reaction in which nucleophile displaces a leaving group such as halide or sulfonate from a carbon-containing radical. Unless otherwise indicated, the term “alkylating” does not limit the carbon-containing radical to alkyl.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Hydroxyalkyl” denotes a hydroxy substitution on alkyl. “Hydroxycycloalkyl” denotes a hydroxy substitution on cycloalkyl. “Hydroxyhaloalkyl” denotes a hydroxy substitution on haloalkyl. “Alkoxycycloalkyl” denotes an alkoxy substitution on cycloalkyl. “Alkoxyhaloalkyl” denotes an alkoxy substitution on haloalkyl. Examples of “hydroxyalkyl”, “hydroxycycloalkyl”, “hydroxyhaloalkyl”, “alkoxycycloalkyl”, “alkoxyhaloalkyl” include the following structures

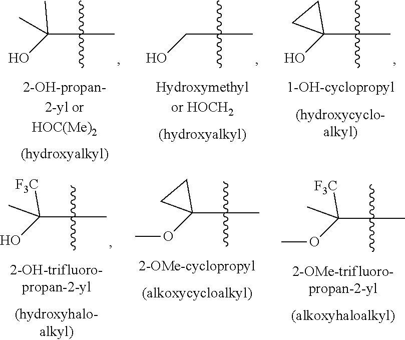

“Alkoxyalkoxy” denotes alkoxy substitution on alkoxy. “Alkylthio” includes branched or straight-chain alkylthio moieties such as methylthio, ethylthio, and the different propylthio, butylthio, pentylthio and hexylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂, CH₃CH₂SCH₂CH₂ and their different isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. Examples of “alkylsulfonate” include CH₃S(O)₂O—, CH₃CH₂S(O)₂O—, CH₃CH₂CH₂S(O)₂O—, (CH₃)₂CHS(O)₂O—, and the different butylsulfonate, pentylsulfonate and hexylsulfonate isomers. “Cyanoalkyl” denotes an alkyl group substituted with one cyano group. Examples of “cyanoalkyl” include NCCH₂ and NCCH₂CH₂ (alternatively identified as CH₂CH₂CN). “Nitroalkyl” denotes an alkyl group substituted with one nitro group. Examples of “nitroalkyl” include NO₂CH₂ and NO₂CH₂CH₂ (alternatively identified as CH₂CH₂NO₂). “Cyano” means NC—, and “formyl” means HC(═O)—. “Alkylamino” includes an NH radical substituted with straight-chain or branched alkyl. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N. “Alkylsily” includes a silyl radical substituted with straight-chain or branched alkyl. “trialkylsily” includes a silyl radical substituted with three straight-chain or branched alkyl. Examples of “trialkylsily” include (CH₃)₃Si—, and (CH₃CH₂)₃Si—. “trialkylsilyalkynyl” denotes trialkylsily substitution on alkynyl. Examples of “trialkylsilyalkynyl” include (CH₃)₃SiC≡C—, and (CH₃CH₂)₃SiC≡C—.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “alkylcycloalkyl” denotes an alkyl group bonded to a cycloalkyl moiety. The term “cycloalkoxy” denotes cycloalkyl group bonded through oxygen. Examples of “cycloalkoxy” include cyclopropoxy, cyclobutoxy, and cyclopentoxy. The term “cycloalkoxyalkyl” denotes cycloalkoxy substitution on an alkyl moiety. Examples of “cycloalkoxyalkyl” include cyclopropoxymethyl, cyclobutoxyethyl, and cyclopentoxymethyl, and other cycloalkoxy moieties bonded to straight-chain or branched alkyl groups. The term “oxacycloalkyl” denotes a cycloalkyl with one carbon ring member replaced with an oxygen atom. Examples of “oxacycloalkyl” include oxacyclopropyl, oxacyclobutyl and oxacyclopentyl. The term “thiacycloalkyl” denotes a cycloalkyl with one carbon ring member replaced with a sulfur atom. Examples of “thiacycloalkyl” include thiacyclopropyl, thiacyclobutyl and thiacyclopentyl. The term “(O-thia)cycloalkyl” denotes a cycloalkyl with one carbon ring member replaced with a —SO group. Examples of “(O-thia)cycloalkyl” include (O-thia)cyclopropy, (O-thia)cyclobutyl and (O-thia)cyclopentyl. The term “(O₂thia)cycloalkyl” denotes a cycloalkyl with one carbon ring member replaced with a —SO₂ group. Examples of “(O₂thia)cycloalkyl” include (O₂thia)cyclopropy, (O₂thia)cyclobutyl and (O₂thia)cyclopentyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkoxyalkyl”, “haloalkylthio”, “haloalkenyl”, “haloalkynyl”, “halocycloalkyl”, “haloalkylcycloalkyl”, “haloalkylsulfinyl”, “haloalkylsulfonyl” and the like, are as defined analogously to the term “haloalkyl”. Examples of “haloalkoxy” include CF₃O—, CCl₃CH₂O—, HCF₂CH₂CH₂O— and CF₃CH₂O—. Examples of “haloalkoxyalkyl” include CF₃OCH₂—, CCl₃CH₂OCH₂—, HCF₂CH₂CH₂OCH₂— and CF₃CH₂OCH₂—. Examples of “haloalkylthio” include CCl₃S—, CF₃S—, CCl₃CH₂S— and ClCH₂CH₂CH₂S—. Examples of “haloalkenyl” include (Cl)₂C═CH— (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “halocycloalkyl” include 1-chlorocyclopropyl, 2-chlorocyclopropyl, 2-fluorocyclopropyl, 1-chlorocyclobutyl, 1-fluorocyclobutyl and 2-fluorocyclobutyl. Examples of “haloalkylcycloalkyl” include 1-(chloromethyl)cyclopropyl, 2-(chloromethyl)cyclopropyl, 2-(fluoromethyl)cyclopropyl, 1-(chloromethyl)cyclobutyl, 2-(fluoroethyl)cyclobutyl and 2-(fluoromethyl)cyclobutyl.

“Alkylcarbonyl” denotes a straight-chain or branched alkyl moiety bonded to a C(═O) moiety. Examples of “alkylcarbonyl” include CH₃C(═O)—, CH₃CH₂C(═O)—, CH₃CH₂CH₂C(═O)—, (CH₃)₂CHC(═O)— and the different butyl- or pentylcarbonyl isomers. “Alkoxycarbonyl” denotes a straight-chain or branched alkoxy moieties bonded to a C(═O) moiety. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers. C(═O) or C(O) designates carbonyl. The term “alkoxycarbonylalkyl” denotes a straight-chain or branched alkoxycarbonyl moiety bonded through an alkyl moiety. The term “alkylcarbonylalkyl” denotes a straight or branched alkylcarbonyl moiety bonded through an alkyl moiety. The term “alkylcarbonyloxy” denotes an alkylcarbony moiety bonded through oxygen. Examples of alkylcarbonyloxy include CH₃C(═O)O—, CH₃CH₂C(═O)O—, CH₃CH₂CH₂C(═O)O— and (CH₃)₂CHC(═O)—. The term “alkenyloxy” denotes an alkenyl moiety bonded through oxygen. Examples of “alkenyloxy” include CH₂═CHCH₂O—, 1-propenyloxy or CH₃CH═CHO—, 2-butenyloxy or CH₃CH═CHCH₂O—, and the different butenyloxy, pentenyloxy and hexenyloxy isomers. Examples of “alkenyloxy” may also contain more than one double bond. The term “alkynyloxy” denotes an alkynyl moiety bonded through oxygen. Examples of “alkynyloxy” include CHCCH₂O—, 1-propynyloxy or CH₃CCO—, 2-butynyloxy or CH₃CCCH₂O—, and the different butynyloxy, pentynyloxy and hexynyloxy isomers. Examples of “alkynyloxy” may also contain more than one triple bond. The term alkanediyl or alkenediyl refers to a linear or branched alkane or alkene linking chain respectively. Examples of alkanediyl include —CH₂—, —CH₂CH(CH₃)— or —CH₂CH₂CH₂—. Examples of alkenediyl include —CH═CH—, —CH₂C═CH— or —CH═C(CH₃)—. The term “adjacent” in the context of locating a substituent means “next to” or “immediately next to”.

“Alkylsulfoximinoalkyl” denotes an alkylsulfoximine or cycloalkylsulfoximine substitution on alkyl or cycloalkyl. Examples of “alkylsulfoximinoalkyl” include the following structures

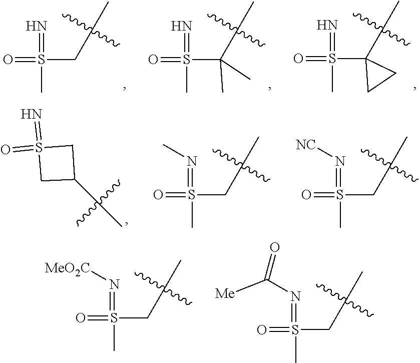

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₃-C₈ alkylcarbonylalkyl can be, for example, CH₃COCH₂—, CH₃COCH₂CH₂— or CH₃CH₂CH₂COCH₂CH₂CH₂CH₂—; C₄-C₇ alkylcycloalkyl can be, for example, methylcyclopropyl, methylcyclobutyl, ethylcyclopropyl, or propylcyclobutyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a group contains a substituent which can be hydrogen, for example R² or R⁵, then when this substituent is taken as hydrogen, it is recognized that this is equivalent to said group being unsubstituted. When one or more positions on a group are said to be “not substituted” or “unsubstituted”, then hydrogen atoms are attached to take up any free valency. Unless otherwise indicated as being optionally substituted, the term “phenyl” means unsubstituted phenyl. Unless otherwise indicated as being optionally substituted, the term “benzyl” means unsubstituted benzyl.

When a compound is substituted with a substituent bearing a subscript that indicates the number of said substituents can exceed 1, said substituents (when they exceed 1) are independently selected from the group of defined substituents, e.g., (R^{3b})ₚ, wherein n is 0, 1, 2 or 3. When p is 0, then hydrogen may be at the position even if not recited in the substituent definition. When a functional group or a compound is shown to be optionally substituted with a substituent, the said functional group or compound may be unsubstituted or substituted. When one or more positions on a group are said to be “not substituted” or “unsubstituted”, then hydrogen atoms are attached to take up any free valency.

The attachment point of (R^{3b})ₚ is illustrated as floating. Each R^{3b} can be attached to any of the 3 available aromaticcarbons by replacement of a hydrogen atom.

The term “ring system” denotes two or more fused rings. The term “bicyclic ring system” denotes a ring system consisting of two fused rings.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds of Formula 1 typically exist in more than one form, and Formula 1 thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

As noted above, two adjacent R² may be taken together to form a 5- to 8-membered ring, the 5- to 8-membered ring can be (among others) saturated or unsaturated, optionally substituted with one or more substituents selected from a group of substituents as defined in the Summary of the Invention. Examples of a 5- to 8-membered unsaturated aromatic ring optionally substituted with from one or more substituents include the rings U-1 through U-60 illustrated in Exhibit 1 wherein R^{v} is independently H, halogen, cyano, nitro, C₁-C₄ alkyl, C₃-C₆ cycloalkyl, C₁-C₄ haloalkyl, C₁-C₄ alkoxy and C₁-C₄ haloalkoxy and r is an integer from 0 to 2, limited by the number of available positions on each U group. The U group can share any two available neighboring atoms with the connecting ring.

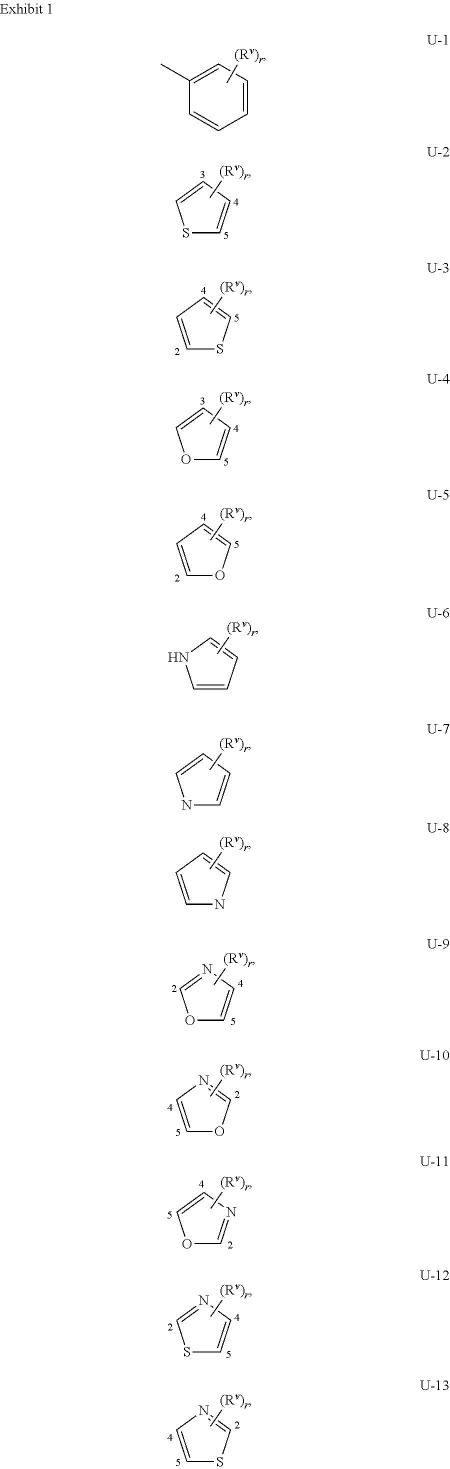

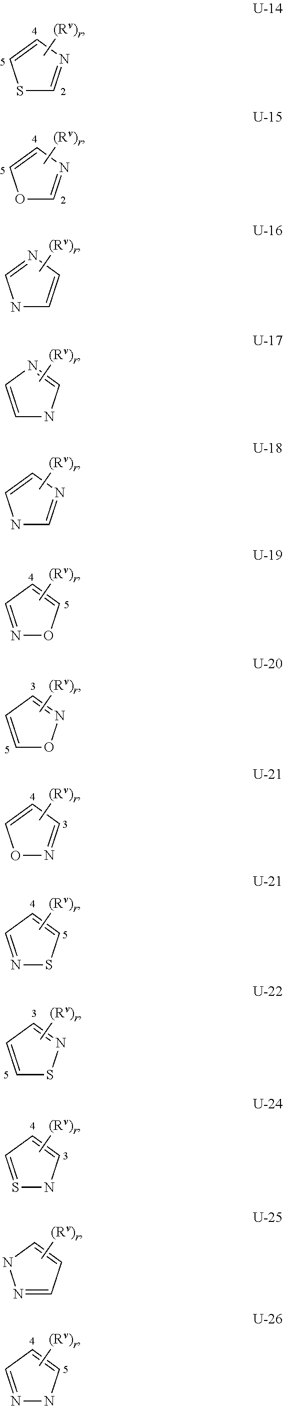

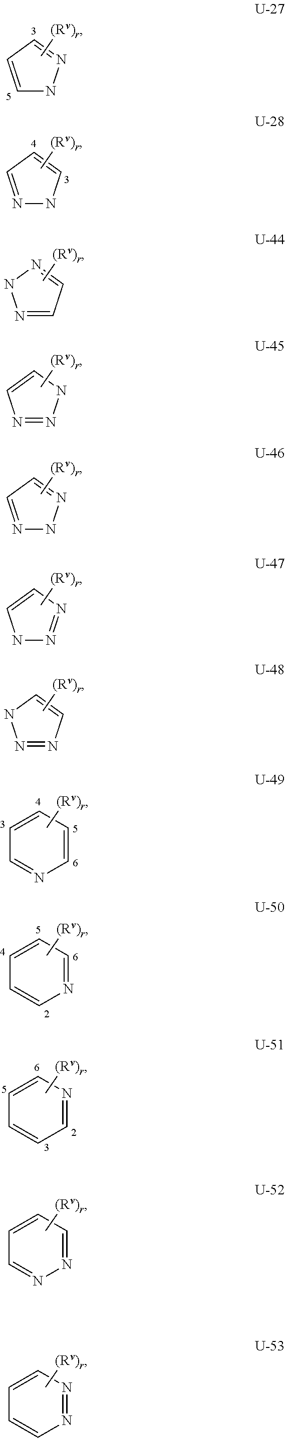

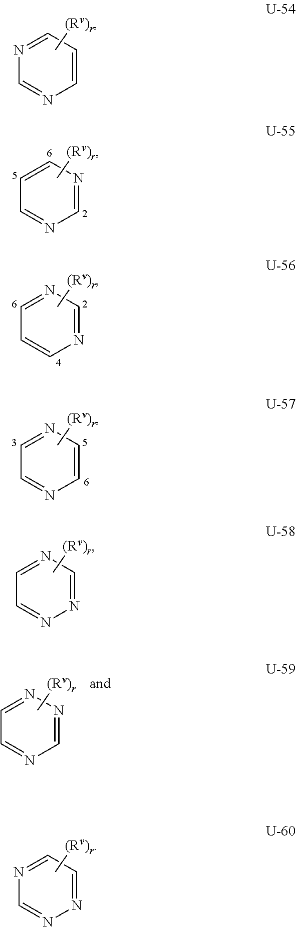

Note that when the 5- to 8-membered saturated or unsaturated non-aromatic heterocyclic ring is optionally substituted with one or more substituents selected from the group of substituents as defined in the Summary of the Invention for R², one or two carbon ring members of the heterocycle can optionally be in the oxidized form of a carbonyl moiety.

Examples of a 5- to 8-membered heterocyclic ring that is saturated or non-aromatic unsaturated heterocyclic ring containing ring members selected from up to two O atoms and up to two S atoms, and optionally substituted on carbon atom ring members with up to four R^{v} includes the rings T-1 through T-35 as illustrated in Exhibit 2. Note that the T group can share any two available neighboring atoms with the connecting ring. The optional substituents corresponding to R^{v} can be attached to any available carbon or nitrogen by replacing a hydrogen atom. For these T rings, r is typically an integer from 0 to 4, limited by the number of available positions on each T group. The term “optionally substituted” means “substituted or unsubstituted”. Note that when T² is N, the nitrogen atom can complete its valence by substitution with either H or the substituents corresponding to R^{v} as defined in the Summary of the Invention.

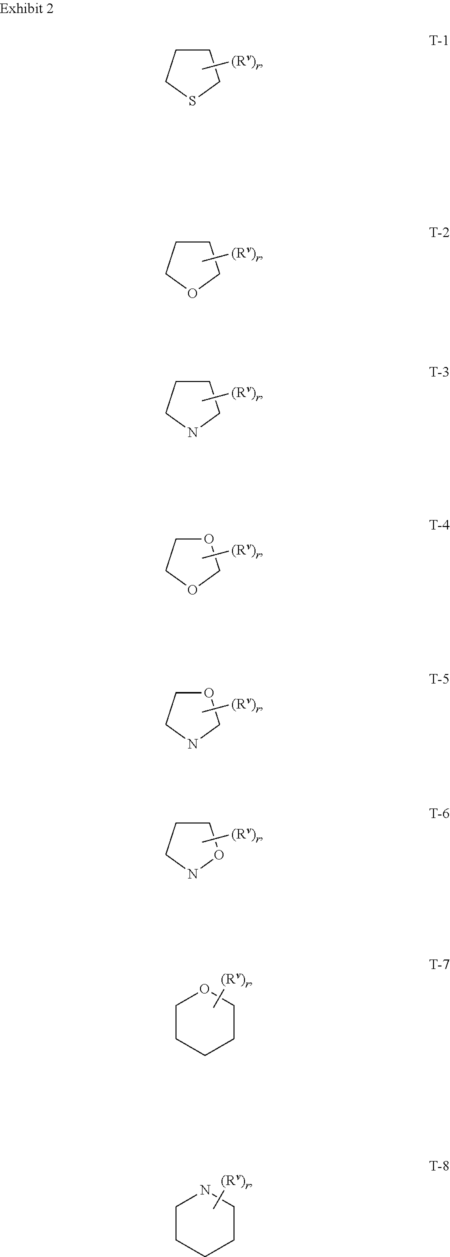

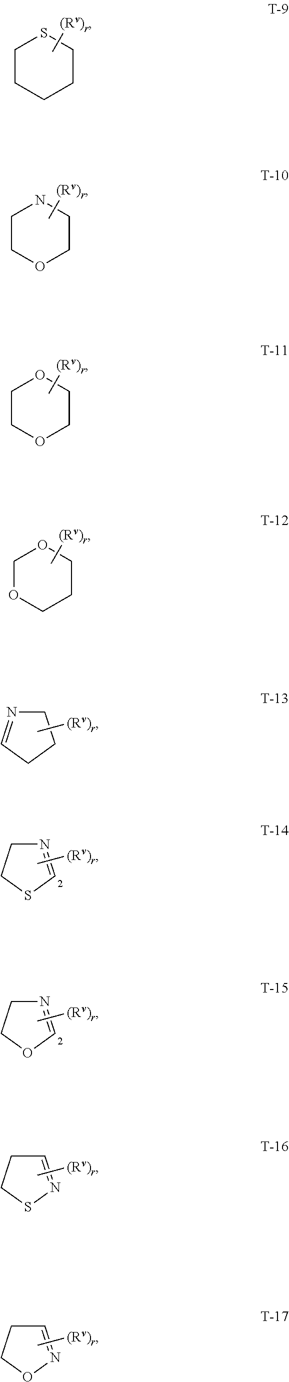

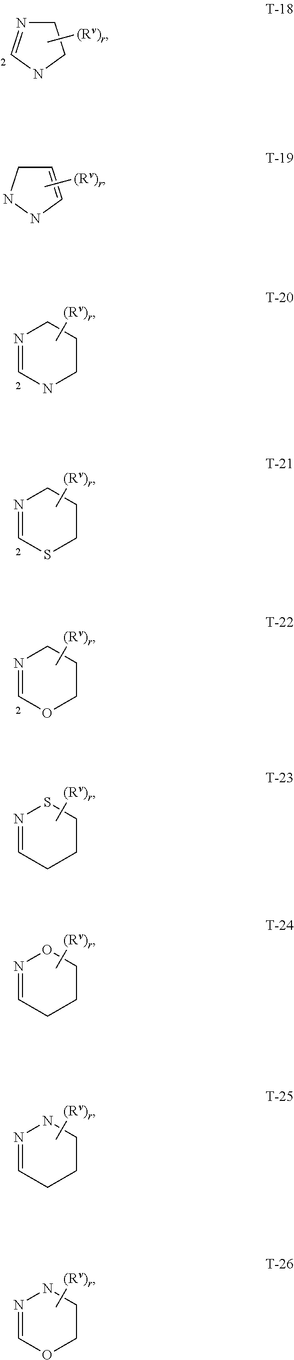

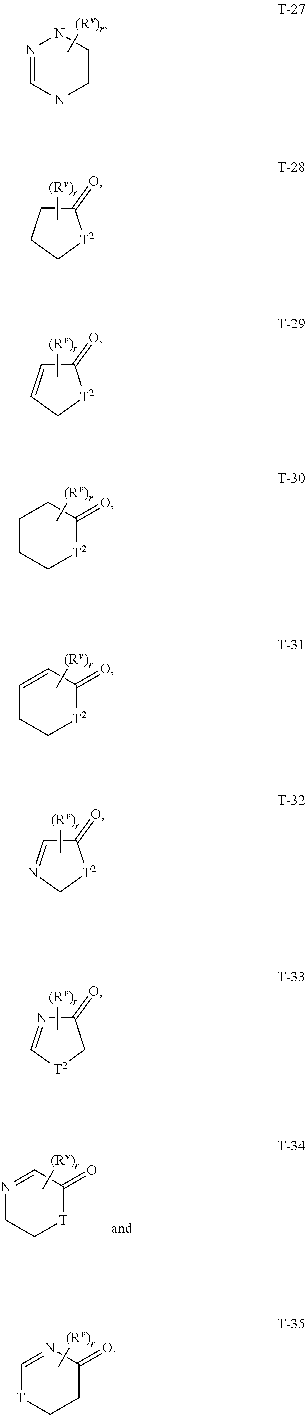

Although R^{v} groups are shown in the structures U-1 through U-60, and T1-T35, it is noted that they do not need to be present since they are optional substituents. Note that when R^{v} is H when attached to an atom, this is the same as if said atom is unsubstituted. The nitrogen atoms that require substitution to fill their valence are substituted with H or R^{v}. Note that when the attachment point between (R^{v})ᵣ and the U (or T) group is illustrated as floating, (R^{v})ᵣ can be attached to any available carbon atom or nitrogen atom of the U group.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Embodiments of the present invention as described in the Summary of the Invention include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.

Embodiment 1. A compound of Formula 1, stereoisomers, N-oxides, and salts thereof, agricultural compositions containing them and their use as herbicides as described in the Summary of the Invention.

### A

Embodiment 2X. A compound of Formula 1 or Embodiment 1 wherein A is selected from

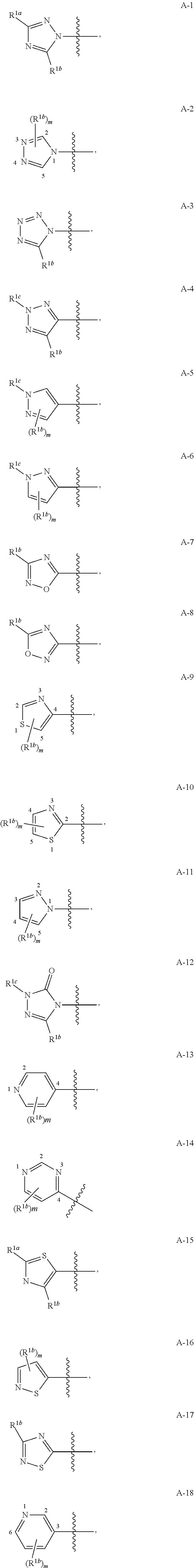

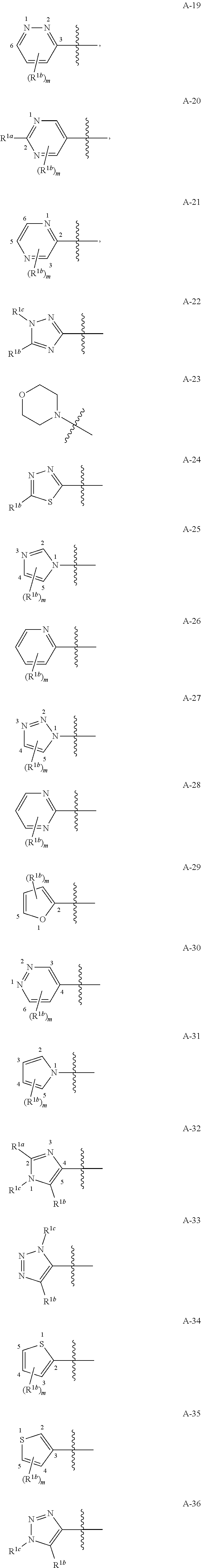

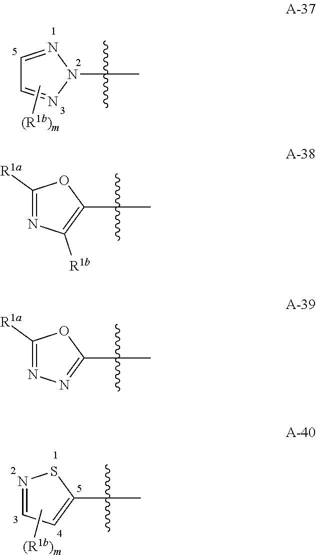

Embodiment 2. A compound of Embodiment 2X wherein A is A-1, A-2, A-3, A-4, A-5, A-6, A-7, A-8, A-9, A-10, A-11, A-12 or A-13.

Embodiment 2a. A compound of Embodiment 2 wherein A is A-1, A-2, A-3, A-4, A-5, A-7, A-8, A-9, A-10, A-11, A-12 or A-13.

Embodiment 2aa. A compound of Embodiment 2a wherein A is A-1, A-3, A-4, A-5, A-12 or A-13.

Embodiment 2aaa. A compound of Embodiment 2aa wherein A is A-1, A-4 or A-5.

Embodiment 2b. A compound of Embodiment 2a wherein A is A-1.

Embodiment 2c. A compound of Embodiment 2a wherein A is A-2.

Embodiment 2d. A compound of Embodiment 2a wherein A is A-3.

Embodiment 2e. A compound of Embodiment 2a wherein A is A-4.

Embodiment 2f. A compound of Embodiment 2a wherein A is A-5.

Embodiment 2g. A compound of Embodiment 2a wherein A is A-7.

Embodiment 2h. A compound of Embodiment 2a wherein A is A-8.

Embodiment 2i. A compound of Embodiment 2a wherein A is A-9.

Embodiment 2j. A compound of Embodiment 2a wherein A is A-10.

Embodiment 2k. A compound of Embodiment 2a wherein A is A-11.

Embodiment 2l. A compound of Embodiment 2a wherein A is A-12.

Embodiment 2m. A compound of Embodiment 2a wherein A is A-13.

Embodiment 2n. A compound of Embodiment 2X wherein A is A-1, A-2, A-3, A-4, A-5, A-6, A-7, A-8, A-9, A-10, A-11, A-12, A-13, A-14, A-15, A-16 or A-17.

Embodiment 2o. A compound of Embodiment 2n wherein A is A-1, A-3, A-4, A-5, A-11, A-12, A-13, A-14, A-15, A-16 or A-17.

Embodiment 2p. A compound of Embodiment 20 wherein A is A-1, A-4, A-5, A-12, A-14, A-15 or A-17.

Embodiment 2q. A compound of Embodiment 2p wherein A is A-1, A-4 or A-15.

X¹ and X²

Embodiment 3. A compound of Formula 1 or Embodiment 1 wherein X¹ and X² are independently N or CR²;

Embodiment 3a. A compound of Embodiment 3 wherein both X¹ and X² are CR².

Embodiment 3b. A compound of Embodiment 3 wherein X¹ is N and X² is CR².

Embodiment 3c. A compound of Embodiment 3 wherein X¹ is CR² and X² is N.

Embodiment 3d. A compound of Embodiment 3 wherein both X¹ and X² are N.

### R^{1a}

Embodiment 4. A compound of Formula 1 or Embodiment 1 wherein R^{1a} is H, halogen, cyano, nitro, amino, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl, C₁-C₇ haloalkoxy, C₁-C₇ alkylthio, C₂-C₇ alkylthioalkyl, C₁-C₅ alkylsulfinyl, C₁-C₅ alkylsulfonyl or C₁-C₅ haloalkylthio.

Embodiment 4a. A compound of Embodiment 4 wherein R^{1a} is H, halogen, cyano, nitro, amino, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl or C₁-C₇ haloalkoxy.

Embodiment 4b. A compound of Embodiment 4a wherein R^{1a} is H, halogen, cyano, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl or C₁-C₇ haloalkoxy.

Embodiment 4c. A compound of Embodiment 4b wherein R^{1a} is H, halogen, cyano, C₁-C₇ alkyl, C₃-C₇ cycloalkyl, C₄-C₇ cycloalkylalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₃-C₇ cycloalkoxy, C₄-C₇ cycloalkoxyalkyl or C₁-C₇ haloalkoxy.

Embodiment 4d. A compound of Embodiment 4c wherein R^{1a} is H, halogen, cyano, C₁-C₇ alkyl, C₃-C₇ cycloalkyl, C₄-C₇ cycloalkylalkyl, C₁-C₄ hydroxyalkyl, C₃-C₅ hydroxycycloalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl.

Embodiment 4e. A compound of Embodiment 4d wherein R^{1a} is H, Me, Et, i-Pro, i-Bu, Bu, t-Bu, Br, cyano, c-Bu, c-Pen, c-Hex, HOCH₂, HOC(Me)₂, CH₂OMe, CH₂O-i-Pro, CH₂CH₂OMe, CH₂-c-Hex or 3-oxetanyl.

Embodiment 4f. A compound of Embodiment 4d wherein R^{1a} is H.

Embodiment 4g. A compound of Embodiment 4d wherein R^{1a} is C₁-C₇ alkyl.

Embodiment 4h. A compound of Embodiment 4g wherein R^{1a} is Et, i-Pro or t-Bu.

Embodiment 4i. A compound of Embodiment 4d wherein R^{1a} is C₃-C₇ cycloalkyl.

Embodiment 4j. A compound of Embodiment 4i wherein R^{1a} is c-Bu.

Embodiment 4k. A compound of Embodiment 4g wherein R^{1a} is Me.

Embodiment 4l. A compound of Formula 1 or Embodiment 1 wherein R^{1a} is H, halogen, cyano, nitro, amino, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ alkyloxacycloalkyl, C₂-C₆ thiacycloalkyl, C₃-C₇ thiacycloalkylalkyl, C₃-C₇ alkylthiacycloalkyl, C₂-C₆ (O-thia)cycloalkyl, C₃-C₇ (O-thia)cycloalkylalkyl, C₃-C₇ alkyl(O-thia)cycloalkyl, C₂-C₆ (O₂thia)cycloalkyl, C₃-C₇ (O₂thia)cycloalkylalkyl, C₃-C₇ alkyl(O₂thia)cycloalkyl, C₂-C₆ haloalkenyl, C₂-C₆ haloalkynyl, C₃-C₇ halocycloalkyl, C4-C₇ haloalkylcycloalkyl, C₂-C₇ alkoxyalkyl, C₂-C₇ haloalkoxyalkyl, C1-C₇ alkoxy, C₂-C₅ alkenyloxy, C₂-C₅ alkynyloxy, C₃-C₇ cycloalkoxy, C4-C₇ cycloalkoxyalkyl, C1-C₇ haloalkoxy, C₁-C₇ alkylthio, C₂-C₇ alkylthioalkyl, C₁-C₅ alkylsulfinyl, C₁-C₅ alkylsulfonyl, C₁-C₄ alkylsulfonate, C₁-C₅ haloalkylthio, C₁-C₅ haloalkylsulfinyl, C₁-C₅ haloalkylsulfonyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₄-C₇ alkylthiocycloalkyl, C₄-C₇ alkylsulfinylcycloalkyl, C₄-C₇ alkylsulfonylcycloalkyl, C₄-C₇ haloalkylthiocycloalkyl, C₂-C₇ haloalkylsulfinylcycloalkyl, C₂-C₇ haloalkylsulfonylcycloalkyl, C₂-C₇ alkylsulfoximinoalkyl, C₂-C5 cyanoalkyl, C₄-C₇ cyanocycloalkyl, C₁-C4 nitroalkyl, C₁-C₇ alkylamino, C₂-C₇ dialkylamino, C₃-C₅ alkylcarbonyl(alkyl)amino, C₃-C₅ alkoxycarbonyl(alkyl)amino, C₂-C4 alkylsulfonyl(alkyl)amino, C₂-C6 alkylcarbonyl, C₃-C6 alkylcarbonylalkyl, C₂-C6 alkoxycarbonyl, C₃-C6 alkoxycarbonylalkyl, C₃-C6 trialkylsilyl or C5-C8 trialkylsilylalkynyl; or phenyl optionally substituted with up to 3 substituents independently selected from the group consisting of halogen, cyano, C₁-C₂ alkyl, C₁-C₂ haloalkyl, C₁-C₂ alkoxy and C₁-C₂ haloalkoxy;

### R^{1b}

Embodiment 5. A compound of Formula 1 or Embodiment 1 wherein R^{1b} is H, halogen, cyano, nitro, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy, C₁-C₄ alkylthio or C₂-C₄ alkoxycarbonyl.

Embodiment 5a. A compound of Embodiment 5 wherein R^{1b} is H, halogen, cyano, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy or C₂-C₄ alkoxycarbonyl.

Embodiment 5b. A compound of Embodiment 5a wherein R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl.

Embodiment 5c. A compound of Embodiment 5b wherein R^{1b} is H, Me, i-Pro, CN, CF₃, F or Cl.

Embodiment 5d. A compound of Embodiment 5c wherein R^{1b} is H.

m

Embodiment 6. A compound of Formula 1 or Embodiment 1 wherein m is 0, 1, or 2.

Embodiment 6a. A compound of Embodiment 6 wherein m is 0.

Embodiment 6b. A compound of Embodiment 6 wherein m is 1.

Embodiment 6c. A compound of Embodiment 6 wherein m is 2.

### R^{1c}

Embodiment 7. A compound of Formula 1 or Embodiment 1 wherein R^{1c} is H, C₁-C₇ alkyl, C₃-C₇ cycloalkyl or C₁-C₇ haloalkyl.

Embodiment 7a. A compound of Embodiment 7 wherein R^{1c} is H or C₁-C₇ alkyl,

Embodiment 7b. A compound of Embodiment 7a wherein R^{1c} is H, Me or i-Pro.

Embodiment 7c. A compound of Embodiment 7b wherein R^{1c} is H.

Embodiment 7d. A compound of Embodiment 7b wherein R^{1c} is Me.

Embodiment 7e. A compound of Embodiment 7b wherein R^{1c} is i-Pro.

n

Embodiment 8. A compound of Formula 1 or Embodiment 1 wherein n is 0, 1, 2 or 3.

Embodiment 8a. A compound of Embodiment 8 wherein n is 0.

Embodiment 8b. A compound of Embodiment 8 wherein n is 1.

Embodiment 8c. A compound of Embodiment 8 wherein n is 2.

Embodiment 8d. A compound of Embodiment 8 wherein n is 3.

### R²

Embodiment 9. A compound of Formula 1 or Embodiment 1 wherein R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl; or two adjacent R² may be taken together to form a 5- or 6-membered ring, containing carbon atoms and optionally 1 to 2 oxygen, sulfur or nitrogen atoms as ring members, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy.

Embodiment 9a. A compound of Embodiment 9 wherein R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ haloalkylthio, C₁-C₄ haloalkylsulfinyl, C₁-C₄ haloalkylsulfonyl, C₁-C₄ alkylsulfonate or C₂-C₅ alkoxycarbonyl.

Embodiment 9a. A compound of Embodiment 9 wherein R² is independently H, halogen, cyano, OH, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy.

Embodiment 9b. A compound of Embodiment 9a wherein R² is independently H, halogen, cyano, OH, C₁-C₅ alkyl, C₂-C₅ alkenyl, C₂-C₅ alkynyl, C₃-C₆ cycloalkyl, C₄-C₇ cycloalkylalkyl, C₁-C₅ haloalkyl, C₂-C₅ alkoxyalkyl, C₁-C₅ alkoxy, C₁-C₅ haloalkoxy.

Embodiment 9c. A compound of Embodiment 9a wherein R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy.

Embodiment 9d. A compound of Embodiment 9c wherein R² is independently H, C₁-C₅ alkyl or C₁-C₅ alkoxy.

Embodiment 9d. A compound of Embodiment 9c wherein R² is independently H, OH, CN, OEt, propargyl, allyl, c-Pro, F, Cl, Br, CN, Me, Et, OMe, CF₃, OCF₃ or CH₂CF₃.

Embodiment 9e. A compound of Embodiment 9d wherein R² is independently H, Me or Et.

Embodiment 9f. A compound of Embodiment 9e wherein R² is independently H.

Embodiment 9g. A compound of Embodiment 9f wherein R² is independently Me.

Embodiment 9h. A compound of Embodiment 9g wherein R² is independently Et.

Embodiment 9i. A compound of Embodiment 9 wherein two adjacent R² may be taken together to form a 5- or 6-membered ring, containing carbon atoms and optionally 1 to 2 oxygen, sulfur or nitrogen atoms as ring members, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy.

Embodiment 9j. A compound of Formula 1 or Embodiment 1 wherein each R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₃-C₅ cycloalkylsulfonate, C₁-C₄ haloalkylsulfonate, C₁-C₄ haloalkylthio, C₁-C₄ haloalkylsulfinyl, C₁-C₄ haloalkylsulfonyl C₂-C₅ cyanoalkyl, C₄-C₆ cyanocycloalkyl or C₂-C₅ alkoxycarbonyl; or

two adjacent R² may be taken together to form a saturated or unsaturated 5- to 8-membered ring, containing carbon atoms and optionally 1 to 3 oxygen, sulfur or nitrogen atoms as ring members, one or two carbon or sulfur ring members of the heterocycle can optionally be in the oxidized form of a carbonyl, sulfonyl, sulfinyl moiety, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy.

Embodiment 9k. A compound of Embodiment 9j wherein R² may be taken together to form a saturated or unsaturated 5- to 8-membered ring, containing carbon atoms and optionally 1 to 3 oxygen, sulfur or nitrogen atoms as ring members, one or two carbon or sulfur ring members of the heterocycle can optionally be in the oxidized form of a carbonyl, sulfonyl, sulfinyl moiety, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy.

Embodiment 9l. A compound of Embodiment 9k wherein R² may be taken together to form a 5- or 6-membered ring, containing up to 2 oxygen atoms as the ring members.

### Y

Embodiment 10. A compound of Formula 1 or Embodiment 1 wherein Y is O or S.

Embodiment 10a. A compound of Embodiment 10 wherein Y is O.

Embodiment 10b. A compound of Embodiment 10 wherein Y is S.

### R^{3a}

Embodiment 11. A compound of Formula 1 or Embodiment 1 wherein R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ haloalkylthioalkyl, C₂-C₅ cyanoalkyl, C₄-C₇ cyanocycloalkyl, C₁-C₄ nitroalkyl, C₃-C₆ alkylcarbonylalkyl, C₂-C₆ oxacycloalkyl, C₃-C₇ cycloalkoxy or C₃-C₆ alkoxycarbonylalkyl.

Embodiment 11a. A compound of Embodiment 11 wherein R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl or C₂-C₇ haloalkoxyalkyl.

Embodiment 11b. A compound of Embodiment 11a wherein R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl.

Embodiment 11c. A compound of Embodiment 11b wherein R^{3a} is C₁-C₇ alkyl, C₃-C₇ cycloalkyl, C₁-C₇ haloalkyl or C₂-C₇ alkoxyalkyl.

Embodiment 11d. A compound of Embodiment 11c wherein R^{3a} is Me, Et, Pro, i-Pro, CF₃, CH₂F or CH₂OMe.

Embodiment 11e. A compound of Embodiment 11d wherein R^{3a} is Me.

Embodiment 11f. A compound of Formula 1 or Embodiment 1 wherein R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₂-C₅ cyanoalkyl, C₄-C₆ cyanocycloalkyl, C₁-C₄ nitroalkyl, C₃-C₆ alkylcarbonylalkyl, C₂-C₆ oxacycloalkyl, C₂-C₆ oxacycloalkylalkyl, C₃-C₇ cycloalkoxy, C₄-C₇ cycloalkoxyalkyl or C₃-C₆ alkoxycarbonylalkyl.

Embodiment 11g. A compound of Embodiment 11f wherein R^{3a} is halogen or C₁-C₇ alkyl.

Embodiment 11h. A compound of Embodiment 11g wherein R^{3a} is F, Cl or Me.

### R^{3b}

Embodiment 12. A compound of Formula 1 or Embodiment 1 wherein R^{3b} is H or halogen.

Embodiment 12a. A compound of Embodiment 12 wherein R^{3b} is H.

Embodiment 12b. A compound of Embodiment 12 wherein R^{3b} is halogen.

Embodiment 12c. A compound of Formula 1 or Embodiment 1 wherein R^{3b} is independently H, halogen or C₁-C₃ alkyl; or
R^{3a} and R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂; or
two R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂.
   p

Embodiment 13. A compound of Formula 1 or Embodiment 1 wherein p is 0, 1, 2 or 3.

Embodiment 13a. A compound of Embodiment 13 wherein p is 0.

Embodiment 13b. A compound of Embodiment 13 wherein p is 1.

Embodiment 13c. A compound of Embodiment 13 wherein p is 2.

Embodiment 13d. A compound of Embodiment 13 wherein p is 3.

### R^{4a}

Embodiment 14. A compound of Formula 1 or Embodiment 1 wherein R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl, C₅-C₈ trialkylsilylalkynyl, C₁-C₅ alkylthio, C₁-C₅ haloalkylthio or C₂-C₅ alkoxycarbonyl.

Embodiment 14a. A compound of Embodiment 14 wherein R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl or C₅-C₈ trialkylsilylalkynyl.

Embodiment 14b. A compound of Embodiment 14a wherein R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl or C₅-C₈ trialkylsilylalkynyl.

Embodiment 14c. A compound of Embodiment 14b wherein R^{4a} is H, halogen, cyano, NO₂, C₁-C₅ alkyl, C₂-C₅ alkenyl, C₂-C₅ alkynyl, C₃-C₅ cycloalkyl, C₁-C₅ haloalkyl, C₂-C₅ alkoxyalkyl, C₁-C₅ alkoxy, C₁-C₅ haloalkoxy, C₃-C₆ trialkylsilyl or C₅-C₈ trialkylsilylalkynyl.

Embodiment 14d. A compound of Embodiment 14c wherein R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl.

Embodiment 14e. A compound of Embodiment 14d wherein R^{4a} is H, CN, NO₂, F, Cl, Br, Me, Et, CF₃, CH₂F, OCF₃, OMe, CH₂OMe, CH═CH₂, C≡CSiMe₃, C≡CH or c-Pro.

Embodiment 14f. A compound of Embodiment 14e wherein R^{4a} is H, F, Cl, Br, Me, Et, CF₃, OCF₃, OMe, CH₂OMe, CH═CH₂, C≡CH or c-Pro.

Embodiment 14g. A compound of Embodiment 14e wherein R^{4a} is Cl or Me.

### R^{4b}

Embodiment 15. A compound of Formula 1 or Embodiment 1 wherein R^{4b} is H, halogen, cyano, nitro, C₁-C₄ alkyl, C₁-C₄ haloalkyl, C₁-C₄ alkoxy or C₁-C₄ alkylthio.

Embodiment 15a. A compound of Embodiment 15 wherein R^{4b} is H, halogen, cyano, C₁-C₄ alkyl, C₁-C₄ haloalkyl or C₁-C₄ alkoxy.

Embodiment 15b. A compound of Embodiment 15a wherein R^{4b} is H or halogen.

Embodiment 15c. A compound of Embodiment 15b wherein R^{4b} is H, F or Cl.

Embodiment 15d. A compound of Embodiment 15c wherein R^{4b} is H.

Embodiment 15e. A compound of Embodiment 15c wherein R^{4b} is F.

Embodiment 15f. A compound of Embodiment 15c wherein R^{4b} is Cl.

q

Embodiment 16. A compound of Formula 1 or Embodiment 1 wherein q is q is 0, 1 or 2.

Embodiment 16a. A compound of Embodiment 16 wherein q is 0.

Embodiment 16b. A compound of Embodiment 16 wherein q is 1.

Embodiment 16c. A compound of Embodiment 16 wherein q is 2.

Embodiment 17. A compound of Formula 1 or Embodiment 1 wherein the stereochemistry of the carbon atom with * is (1′) depicted as Formula 1′ below.

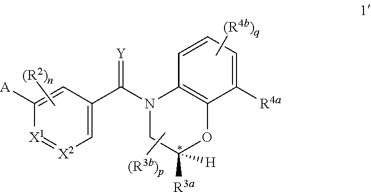

Embodiment 18. A compound of Formula 1 or Embodiment 1 wherein the stereochemistry of the carbon atom with * is (1″), depicted as Formula 1″ below.

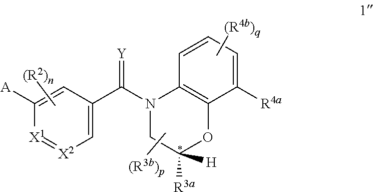

Embodiment 19. A compound of Formula 1 or Embodiment 1 that is other than the compound of Formula 1 wherein A is A-1, X¹ is CH, X² is CH, R^{1a} is H, R^{1b} is H, (R²)ₙ is 2-Me, R^{3a} is (1′)-Me, (R^{3b})ₚ is H, R^{4a} is Cl and (R^{4b})_{q} is H (i.e. Compound 40).

Embodiment 20. A compound of Formula 1 or Embodiment 1 that is other than the compound of Formula 1 wherein A is A-1, X¹ is CH, X² is CH, R^{1a} is H, R^{1b} is i-Pro, (R²)ₙ is H, R^{3a} is (1′)-Me, (R^{3b})ₚ is H, R^{4a} is Me and (R^{4b})_{q} is H (i.e. Compound 56).

Embodiment 21. A compound of Formula 1 or Embodiment 1 that is other than the compound of Formula 1 wherein A is A-1, X¹ is CH, X² is CH, R^{1a} is CH₂c-Hex, R^{1b} is H, (R²)ₙ is H, R^{3a} is (1′)-Me, (R^{3b})ₚ is H, R^{4a} is Cl and (R^{4b})_{q} is H (i.e. Compound 90).

Embodiments of this invention, including Embodiments 1-21 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-12a above as well as any other embodiments described herein, and any combination thereof, pertain to the compositions and methods of the present invention.

Combinations of Embodiments 1-21 are illustrated by:

Embodiment X. A compound of Formula 1 as described in the Summary of the Invention wherein
A is A-1, A-2, A-3, A-4, A-5, A-7, A-8, A-9, A-10, A-11, A-12 or A-13; and X¹ and X² are independently N or CR².

Embodiment XX. A compound of Embodiment X wherein
A is A-1.

Embodiment A. A compound of Embodiment XX wherein
both X¹ and X² are CR²;
R^{1a} is H, halogen, cyano, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy or C₂-C₄ alkoxycarbonyl;
R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ haloalkylthioalkyl, C₂-C₅ cyanoalkyl, C₁-C₄ nitroalkyl, C₃-C₆ alkylcarbonylalkyl, C₂-C₆ oxacycloalkyl, C₃-C₇ cycloalkoxy or C₃-C₆ alkoxycarbonylalkyl;
R^{3b} is H or halogen;
R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl or C₅-C₈ trialkylsilylalkynyl;
R^{4b} is H, halogen, cyano, nitro, C₁-C₄ alkyl, C₁-C₄ haloalkyl, C₁-C₄ alkoxy or C₁-C₄ alkylthio.

Embodiment A1. A compound of Embodiment A wherein
R^{1a} is H, halogen, cyano, C₁-C₇ alkyl, C₃-C₇ cycloalkyl, C₄-C₇ cycloalkylalkyl, C₁-C₇ haloalkyl, C₂-C₆ oxacycloalkyl, C₃-C₇ oxacycloalkylalkyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl or C₂-C₇ haloalkoxyalkyl;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H, halogen, cyano, C₁-C₄ alkyl, C₁-C₄ haloalkyl or C₁-C₄ alkoxy.

Embodiment A2. A compound of Embodiment A1 wherein
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl; and
R^{4b} is H or halogen.

Embodiment A3. A compound of Embodiment A2 wherein
R^{1a} is H, Me, Et, i-Pro, i-Bu, Bu, t-Bu, Br, cyano, c-Bu, c-Pen, c-Hex, CH₂OMe, CH₂O-i-Pro, CH₂CH₂OMe, CH₂-c-Hex or 3-oxetanyl;
R^{1b} is H, Me, i-Pro, CN, CF₃, F or Cl.
R² is independently H, OH, CN, OEt, propargyl, allyl, c-Pro, F, Cl, Br, CN, Me, Et, OMe, CF₃, OCF₃ or CH₂CF₃; R^{3a} is Me, Et, Pro, i-Pro, CF₃, CH₂F or CH₂OMe.
R^{4a} is H, CN, NO₂, F, Cl, Br, Me, Et, CF₃, CH₂F, OCF₃, OMe, CH₂OMe, CH═CH₂, C≡CSiMe₃, C≡CH or c-Pro.

Embodiment B. A compound of Embodiment XX wherein
X¹ is N and X² is CR².
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

Embodiment B1. A compound of Embodiment B wherein
R^{1a} is H;
R^{1b} is H;
R² is independently H, C₁-C₅ alkyl or C₁-C₅ alkoxy;
R^{3a} is C₁-C₇ alkyl, C₃-C₇ cycloalkyl, C₁-C₇ haloalkyl or C₂-C₇ alkoxyalkyl.
R^{3b} is H or halogen.
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl.
R^{4b} is H, halogen, cyano, C₁-C₄ alkyl, C₁-C₄ haloalkyl or C₁-C₄ alkoxy.

Embodiment C. A compound of Embodiment XX wherein
X¹ is CR² and X² is N;
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

Specific embodiments include compounds of Formula 1 selected from the group consisting of:
[(2S)-8-Chloro-2,3-dihydro-2-methyl-4H-1,4-benzoxazin-4-yl][2-methyl-5-[3-(1-methylethyl)-1H-1,2,4-triazol-1-yl]phenyl]methanone (Compound 73);
[(2S)-8-Chloro-2,3-dihydro-2-methyl-4H-1,4-benzoxazin-4-yl][5-[3-(1,1-dimethylethyl)-1H-1,2,4-triazol-1-yl]-2-methylphenyl]methanone (Compound 76);
[(2S)-8-Chloro-2,3-dihydro-2-methyl-4H-1,4-benzoxazin-4-yl][2-methoxy-5-[3-(1-methylethyl)-1H-1,2,4-triazol-1-yl]phenyl]methanone (Compound 84);
[(2S)-8-Chloro-2,3-dihydro-2-methyl-4H-1,4-benzoxazin-4-yl][5-(3-ethyl-1H-1,2,4-triazol-1-yl)-2-methoxyphenyl]methanone (Compound 88);
[(2S)-8-Chloro-2,3-dihydro-2-methyl-4H-1,4-benzoxazin-4-yl][2-ethyl-5-[3-(1-methylethyl)-1H-1,2,4-triazol-1-yl]phenyl]methanone (Compound 94);
[(2S)-8-Chloro-2,3-dihydro-2-methyl-4H-1,4-benzoxazin-4-yl][5-(3-cyclobutyl-1H-1,2,4-triazol-1-yl)-2-methylphenyl]methanone (Compound 96);
[(2S)-8-Chloro-2,3-dihydro-2-methyl-4H-1,4-benzoxazin-4-yl][3-(3-cyclobutyl-1H-1,2,4-triazol-1-yl)phenyl]methanone (Compound 93);
[(2S)-2,3-Dihydro-2,8-dimethyl-4H-1,4-benzoxazin-4-yl][2-methoxy-5-(1H-1,2,4-triazol-1-yl)phenyl]methanone (Compound 63);

Embodiment 5. A compound of Formula 1 as described in the Summary of the Disclosure wherein
A is selected from

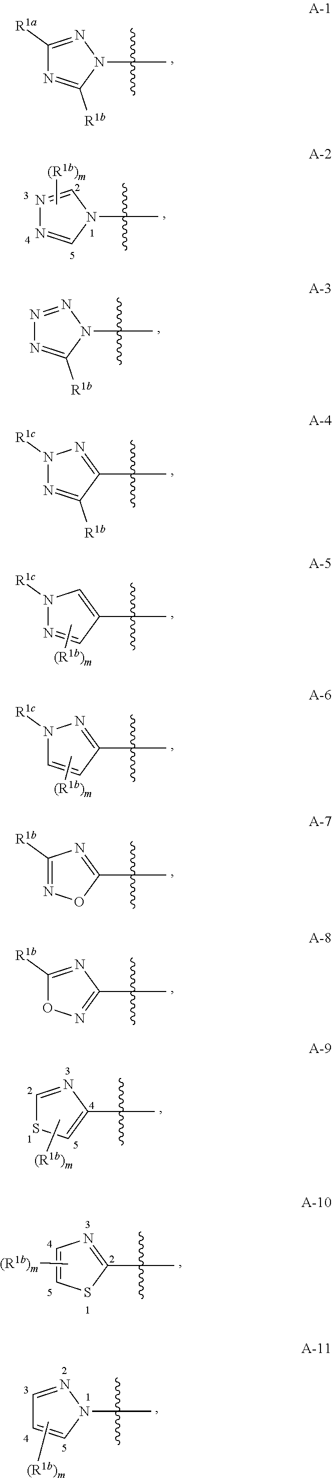

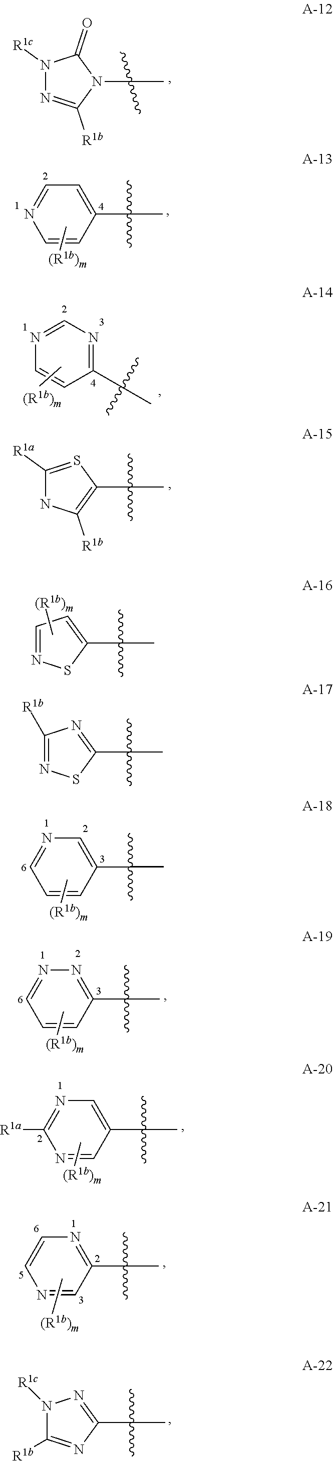

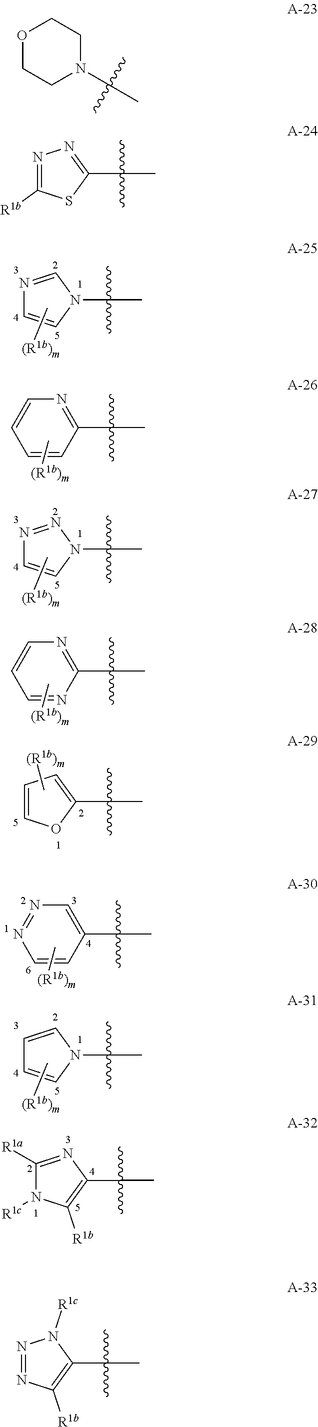

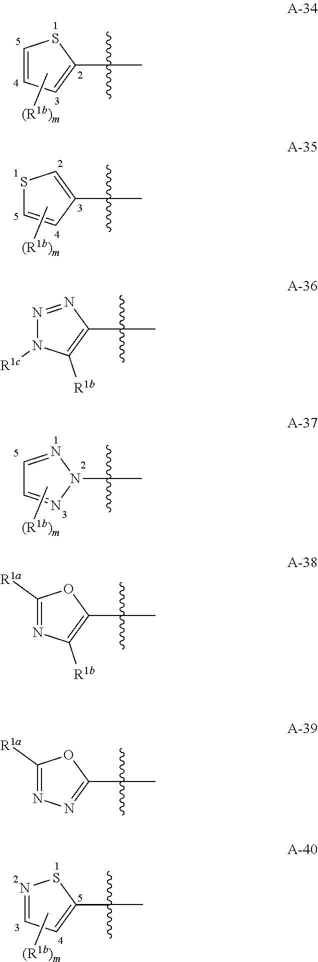

Embodiment S1. The compound of Embodiment S wherein
A is A-1, A-4, A-5, A-12, A-14, A-15 or A-17.

Embodiment S2. The compound of Embodiment S wherein
A is A-1.

Embodiment S3. The compound of Embodiment S2 wherein
both X¹ and X² are CR²;
R^{1a} is H, halogen, cyano, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy or C₂-C₄ alkoxycarbonyl;
R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ haloalkylthioalkyl, C₂-C₅ cyanoalkyl, C₁-C₄ nitroalkyl, C₃-C₆ alkylcarbonylalkyl, C₂-C₆ oxacycloalkyl, C₃-C₇ cycloalkoxy or C₃-C₆ alkoxycarbonylalkyl;
R^{3b} is H or halogen;
R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl or C₅-C₈ trialkylsilylalkynyl;
R^{4b} is H, halogen, cyano, nitro, C₁-C₄ alkyl, C₁-C₄ haloalkyl, C₁-C₄ alkoxy or C₁-C₄ alkylthio.

Embodiment S4. The compound of Embodiment S3 wherein
R^{1a} is H, halogen, cyano, C₁-C₇ alkyl, C₃-C₇ cycloalkyl, C₄-C₇ cycloalkylalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl or C₂-C₇ haloalkoxyalkyl;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H, halogen, cyano, C₁-C₄ alkyl, C₁-C₄ haloalkyl or C₁-C₄ alkoxy.

Embodiment S5. The compound of Embodiment S4 wherein
R^{1a} is H, halogen, cyano, C₁-C₇ alkyl, C₃-C₇ cycloalkyl, C₄-C₇ cycloalkylalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl; and
R^{4b} is H or halogen.

Embodiment S6. The compound of Embodiment S5 wherein
R^{1a} is H, Me, Et, i-Pro, i-Bu, Bu, t-Bu, Br, cyano, c-Bu, c-Pen, c-Hex, HOCH₂, HOC(Me)₂, CH₂OMe, CH₂O-i-Pro, CH₂CH₂OMe, CH₂-c-Hex or 3-oxetanyl;
R^{1b} is H, Me, i-Pro, CN, CF₃, F or Cl;
R² is independently H, OH, CN, OEt, propargyl, allyl, c-Pro, F, Cl, Br, CN, Me, Et, OMe, CF₃, OCF₃ or CH₂CF₃;
R^{3a} is Me, Et, Pro, i-Pro, CF₃, CH₂F or CH₂OMe; and
R^{4a} is H, CN, NO₂, F, Cl, Br, Me, Et, CF₃, CH₂F, OCF₃, OMe, CH₂OMe, CH═CH₂, C≡CSiMe₃, C≡CH or c-Pro.

Embodiment S7. The compound of Embodiment S2 wherein
X¹ is N and X² is CR².
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

Embodiment S8. The compound of Embodiment S7 wherein
R^{1a} is H;
R^{1b} is H;
R² is independently H, C₁-C₅ alkyl or C₁-C₅ alkoxy;
R^{3a} is C₁-C₇ alkyl, C₃-C₇ cycloalkyl, C₁-C₇ haloalkyl or C₂-C₇ alkoxyalkyl.
R^{3b} is H or halogen.
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl.
R^{4b} is H, halogen, cyano, C₁-C₄ alkyl, C₁-C₄ haloalkyl or C₁-C₄ alkoxy.

Embodiment S9. The compound of Embodiment S2 wherein
X¹ is CR² and X² is N;
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

Embodiment S10. The compound of Embodiment S2 wherein
R² may be taken together to form a 5- or 6-membered ring, containing up to 2 oxygen atoms as the ring members.

Embodiment S11. Specific embodiments include compounds of Formula 1 selected from the group consisting of:
[(2S)-8-Chloro-2,3-dihydro-2-methyl-4H-1,4-benzoxazin-4-yl][2-methyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-[3-(1,1-dimethylethyl)-1H-1,2,4-triazol-1-yl]-2-methylphenyl]methanone;
[(2S)-8-Chloro-2,3-dihydro-2-methyl-4H-1,4-benzoxazin-4-yl][2-methoxy-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-ethyl-1H-1,2,4-triazol-1-yl)-2-methoxyphenyl]methanone;
[(2S)-8-Chloro-2,3-dihydro-2-methyl-4H-1,4-benzoxazin-4-yl][2-ethyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-cyclobutyl-1H-1,2,4-triazol-1-yl)-2-methylphenyl]methanone;
[(2S)-8-Chloro-2,3-dihydro-2-methyl-4H-1,4-benzoxazin-4-yl][3-(3-cyclobutyl-1H-1,2,4-triazol-1-yl)phenyl]methanone;
[(2S)-2,3-Dihydro-2,8-dimethyl-4H-1,4-benzoxazin-4-yl][2-methoxy-5-(1H-1,2,4-triazol-1-yl)phenyl]methanone;

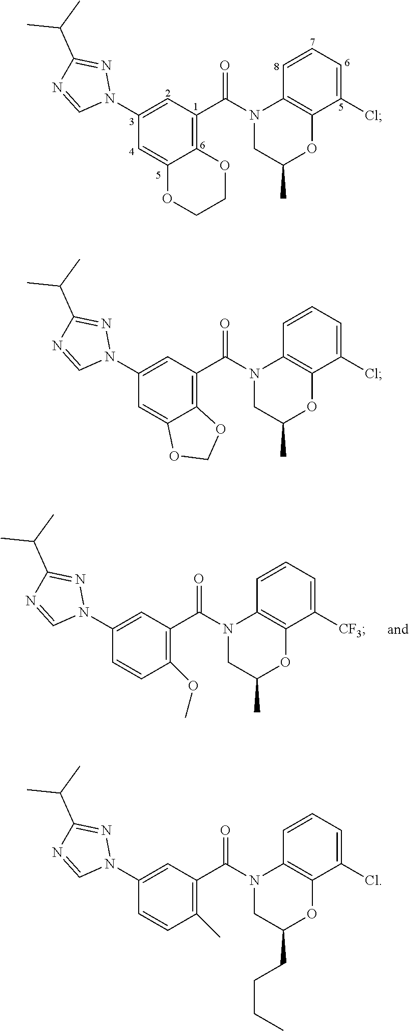 [SMILES: CC(C)c1ncn(-c2cc3c(c(C(=O)N4C[C@H](C)Oc5c(Cl)cccc54)c2)OCCO3)n1 ; CC(C)c1ncn(-c2cc3c(c(C(=O)N4C[C@H](C)Oc5c(Cl)cccc54)c2)OCO3)n1 ; COc1ccc(-n2cnc(C(C)C)n2)cc1C(=O)N1C[C@H](C)Oc2c1cccc2C(F)(F)F ; CCCC[C@H]1CN(C(=O)c2cc(-n3cnc(C(C)C)n3)ccc2C)c2cccc(Cl)c2O1]

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) dehydrooritate dehydrogenase (DHODH) inhibitors, (b16) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b17) herbicide safeners, and salts of compounds of (b1) through (b17).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-TH-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-TH-indol-6-yl)-2-Pyridinecarboxylic cyanomethyl ester (CAS No. 2251111-18-7), aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat, paraquat and 1-(2-carboxyethyl)-4-(2-pyrimidinyl)pyridazinium (CAS No. 2285384-11-2).

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-Isoxazolecarboxylic ethyl ester (CAS No. 1949837-17-5), cinidon-ethyl, fluazolate, flufenoximacil, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), flusulfinam, iptriazopyrid, isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST (homogentisate solanesyltransferase) inhibitors” (b13) disrupt a plant's ability to convert homogentisate to 2-methyl-6-solanyl-1,4-benzoquinone, thereby disrupting carotenoid biosynthesis. Examples of HST inhibitors include cyclopyrimorate (6-chloro-3-(2-cyclopropyl-6-methylphenoxy)-4-pyridazinyl 4-morpholinecarboxylate), haloxydine, pyriclor, 3-(2-chloro-3,6-difluorophenyl)-4-hydroxy-1-methyl-1,5-naphthyridin-2(1H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

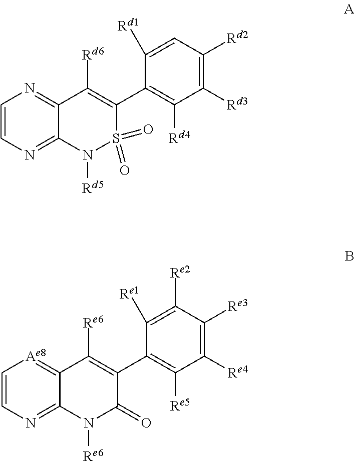

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C≡CH; R^{e7} is OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-TH-inden-1-yl]-6-(1-fluoroethyl)-1,3,5-triazine-2,4-diamine), isoxaben and triaziflam.

“DHODH (dihydroorotate dehydrogenase) inhibitors” (b15) act through inhibiting catalysis of the fourth step of pyrimidine biosynthesis in plant systems. Inhibition of pyrimidine biosynthesis leads to the cessation of plant growth. Examples of DOHDH inhibitors include tetflupyrolimet ((3S,4S)—N-(2-fluorophenyl)-1-methyl-2-oxo-4-[3-(trifluoromethyl)phenyl]-3-pyrrolidinecarboxamide) and (3S,4R)—N-(2,3-difluorophenyl)-1-methyl-4-[1-methyl-5-(trifluoromethyl)-1H-pyrazol-3-yl]-2-oxo-3-pyrrolidinecaboxamide.

“Other herbicides” (b16) include herbicides that act through a variety of different modes of action such as mitotic disruptors (e.g., flamprop-M-methyl and flamprop-M-isopropyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bixlozone, broclozone, bromobutide, cinmethylin, clomazone, cumyluron, daimuron, difenzoquat, dimesulfazet, epyrifenacil, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb, 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6) and 5-[[(2,6-difluorophenyl)methoxy]methyl]-4,5-dihydro-5-methyl-3-(3-methyl-2-thienyl)isoxazole. “Other herbicides” (b16) also include a compound of Formula (b16A)

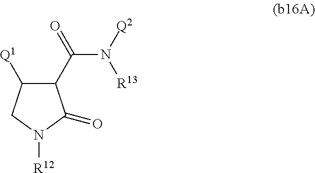

wherein
R¹² is H, C₁-C₆ alkyl, C₁-C₆ haloalkyl or C₄-C₈ cycloalkyl;
R¹³ is H, C₁-C₆ alkyl or C₁-C₆ alkoxy;
Q¹ is an optionally substituted ring system selected from the group consisting of phenyl, thienyl, pyridinyl, benzodioxolyl, naphthyl, naphthalenyl, benzofuranyl, furanyl, benzothiophenyl and pyrazolyl, wherein when substituted said ring system is substituted by 1 to 3 R¹⁴;
Q² is an optionally substituted ring system selected from the group consisting of phenyl, pyridinyl, benzodioxolyl, pyridinonyl, thiadiazolyl, thiazolyl, and oxazolyl, wherein when substituted said ring system is substituted by 1 to 3 R¹⁵;
each R¹⁴ is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R¹⁵ is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R¹⁶ is independently halogen, C₁-C₆ alkyl or C₁-C₆ haloalkyl;
R¹⁷ is C₁-C₄ alkoxycarbonyl.

In one Embodiment wherein “other herbicides” (b16) also include a compound of Formula (b16A), it is preferred that R¹² is H or C₁-C₆ alkyl; more preferably R¹² is H or methyl. Preferrably R¹³ is H. Preferably Q¹ is either a phenyl ring or a pyridinyl ring, each ring substituted by 1 to 3 R¹⁴; more preferably Q¹ is a phenyl ring substituted by 1 to 2 R¹⁴. Preferably Q² is a phenyl ring substituted by 1 to 3 R¹⁵; more preferably Q² is a phenyl ring substituted by 1 to 2 R¹⁵. Preferably each R¹⁴ is independently halogen, C₁-C₄ alkyl, C₁-C₃ haloalkyl, C₁-C₃ alkoxy or C₁-C₃ haloalkoxy; more preferably each R¹⁴ is independently chloro, fluoro, bromo, C₁-C₂ haloalkyl, C₁-C₂ haloalkoxy or C₁-C₂ alkoxy. Preferrably each R¹⁵ is independently halogen, C₁-C₄ alkyl, C₁-C₃ haloalkoxy; more preferably each R¹⁵ is independently chloro, fluoro, bromo, C₁-C₂ haloalkyl, C₁-C₂ haloalkoxy or C₁-C₂ alkoxy. Specifically preferred as “other herbicides” (b16) include any one of the following (b16A-1) through (b16A-15):

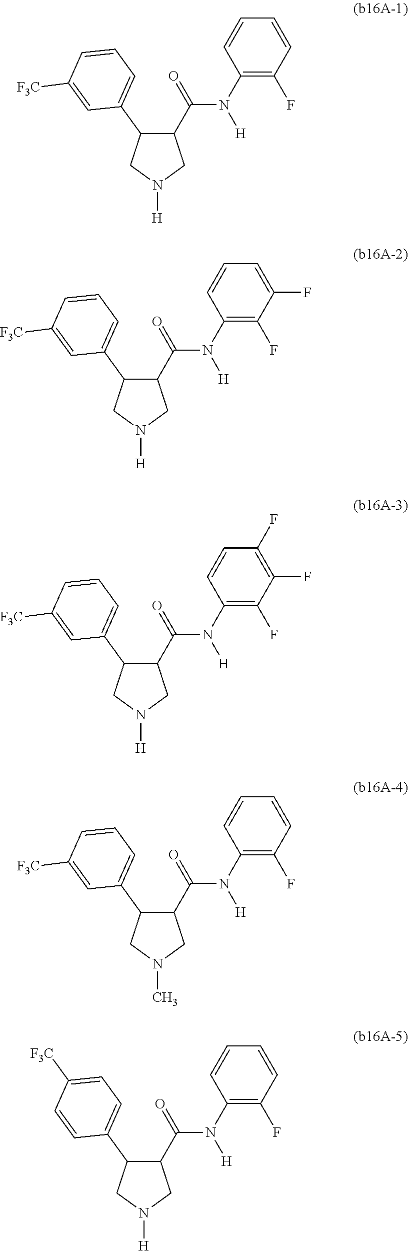 [SMILES: O=C(Nc1ccccc1F)C1CNCC1c1cccc(C(F)(F)F)c1 ; O=C(Nc1cccc(F)c1F)C1CNCC1c1cccc(C(F)(F)F)c1 ; O=C(Nc1ccc(F)c(F)c1F)C1CNCC1c1cccc(C(F)(F)F)c1 ; CN1CC(C(=O)Nc2ccccc2F)C(c2cccc(C(F)(F)F)c2)C1 ; O=C(Nc1ccccc1F)C1CNCC1c1ccc(C(F)(F)F)cc1]

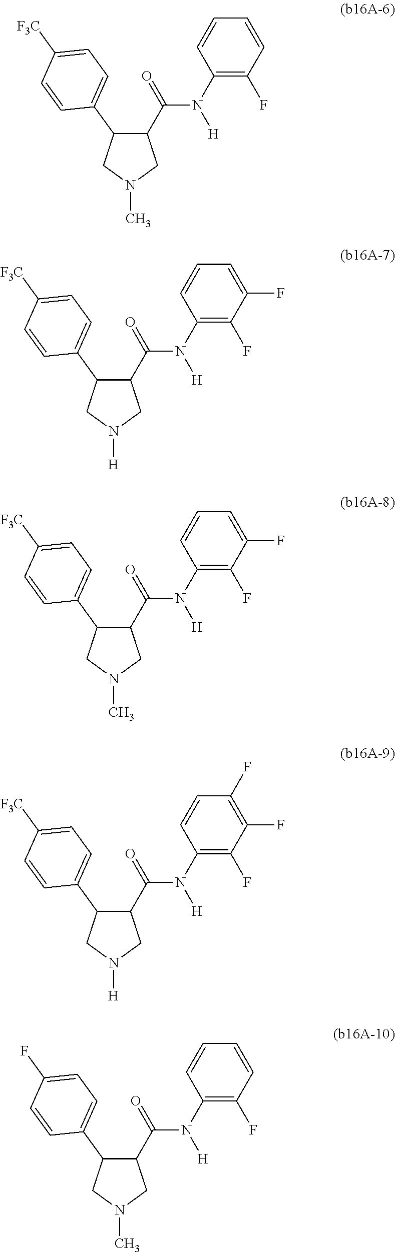 [SMILES: CN1CC(C(=O)Nc2ccccc2F)C(c2ccc(C(F)(F)F)cc2)C1 ; O=C(Nc1cccc(F)c1F)C1CNCC1c1ccc(C(F)(F)F)cc1 ; CN1CC(C(=O)Nc2cccc(F)c2F)C(c2ccc(C(F)(F)F)cc2)C1 ; O=C(Nc1ccc(F)c(F)c1F)C1CNCC1c1ccc(C(F)(F)F)cc1 ; CN1CC(C(=O)Nc2ccccc2F)C(c2ccc(F)cc2)C1]

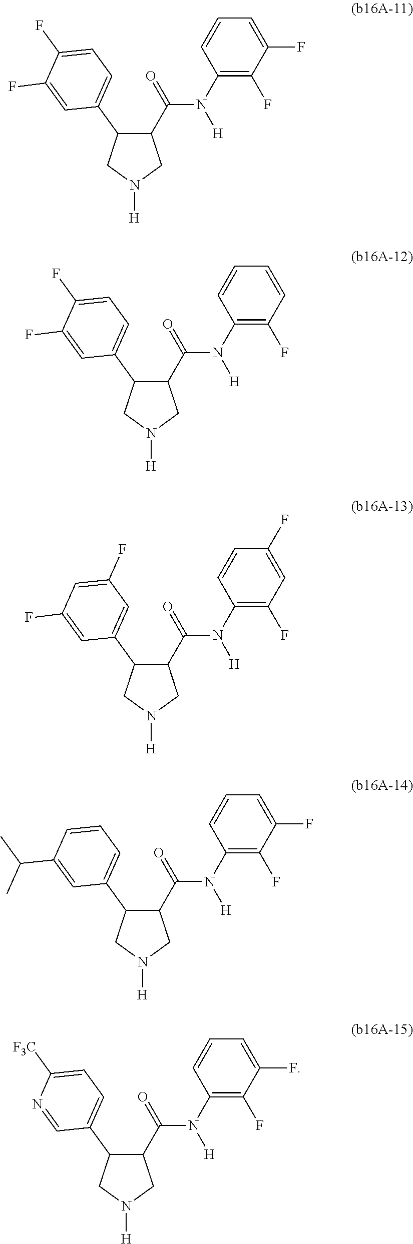 [SMILES: O=C(Nc1cccc(F)c1F)C1CNCC1c1ccc(F)c(F)c1 ; O=C(Nc1ccccc1F)C1CNCC1c1ccc(F)c(F)c1 ; O=C(Nc1ccc(F)cc1F)C1CNCC1c1cc(F)cc(F)c1 ; CC(C)c1cccc(C2CNCC2C(=O)Nc2cccc(F)c2F)c1 ; O=C(Nc1cccc(F)c1F)C1CNCC1c1ccc(C(F)(F)F)nc1]

“Other herbicides” (b16) also include a compound of Formula (b16B)

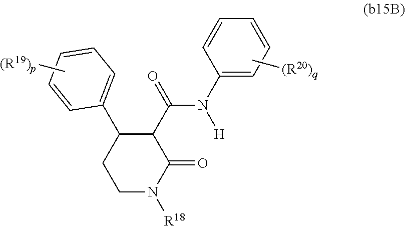

wherein
R¹⁸ is H, C₁-C₆ alkyl, C₁-C₆ haloalkyl or C₄-C₈ cycloalkyl;
each R¹⁹ is independently halogen, C₁-C₆ haloalkyl or C₁-C₆ haloalkoxy; p is an integer of 0, 1, 2 or 3;
each R²⁰ is independently halogen, C₁-C₆ haloalkyl or C₁-C₆ haloalkoxy; and q is an integer of 0, 1, 2 or 3.

In one Embodiment wherein “other herbicides” (b16) also include a compound of Formula (b16B), it is preferred that R¹⁸ is H, methyl, ethyl or propyl; more preferably R¹⁸ is H or methyl; most preferably R¹⁸ is H. Preferrably each R¹⁹ is independently chloro, fluoro, C₁-C₃ haloalkyl or C₁-C₃ haloalkoxy; more preferably each R¹⁹ is independently chloro, fluoro, C₁ fluoroalkyl (i.e. fluoromethyl, difluoromethyl or trifluoromethyl) or C₁ fluoroalkoxy (i.e. trifluoromethoxy, difluoromethoxy or fluoromethoxy). Preferably each R²⁰ is independently chloro, fluoro, C₁ haloalkyl or C₁ haloalkoxy; more preferably each R²⁰ is independently chloro, fluoro, C₁ fluoroalkyl (i.e. fluoromethyl, difluorormethyl or trifluromethyl) or C₁ fluoroalkoxy (i.e. trifluoromethoxy, difluoromethoxy or fluoromethoxy). Specifically preferred as “other herbicides” (b16) include any one of the following (b16B-1) through (b16B-19):

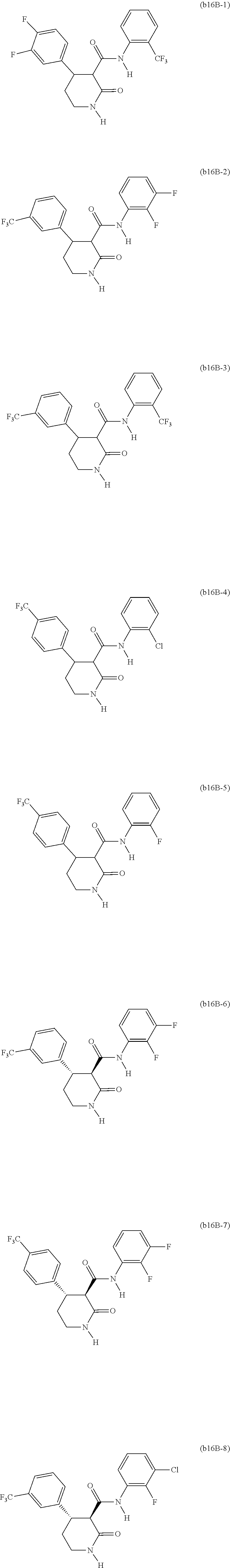

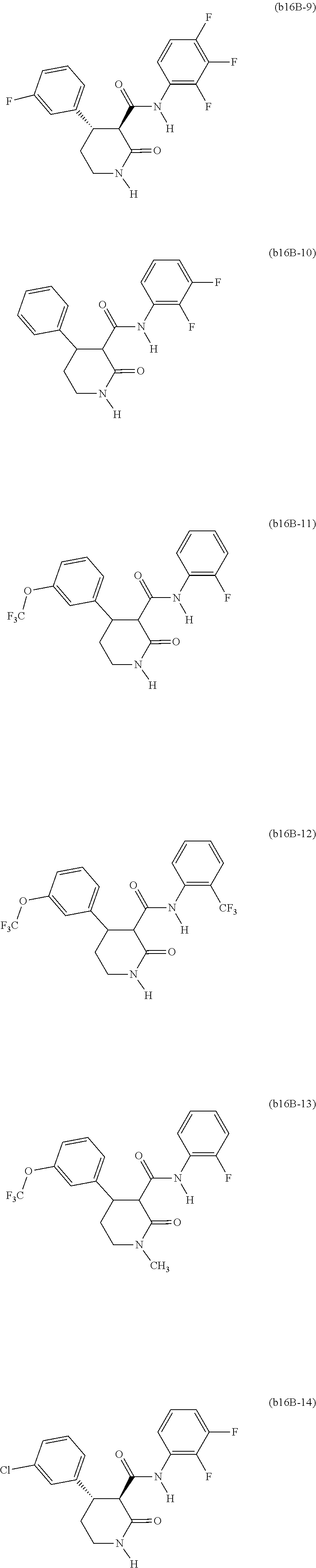 [SMILES: O=C1NCC[C@H](c2cccc(F)c2)[C@H]1C(=O)Nc1ccc(F)c(F)c1F ; O=C1NCCC(c2ccccc2)C1C(=O)Nc1cccc(F)c1F ; O=C1NCCC(c2cccc(OC(F)(F)F)c2)C1C(=O)Nc1ccccc1F ; O=C1NCCC(c2cccc(OC(F)(F)F)c2)C1C(=O)Nc1ccccc1C(F)(F)F ; CN1CCC(c2cccc(OC(F)(F)F)c2)C(C(=O)Nc2ccccc2F)C1=O ; O=C1NCC[C@H](c2cccc(Cl)c2)[C@H]1C(=O)Nc1cccc(F)c1F]

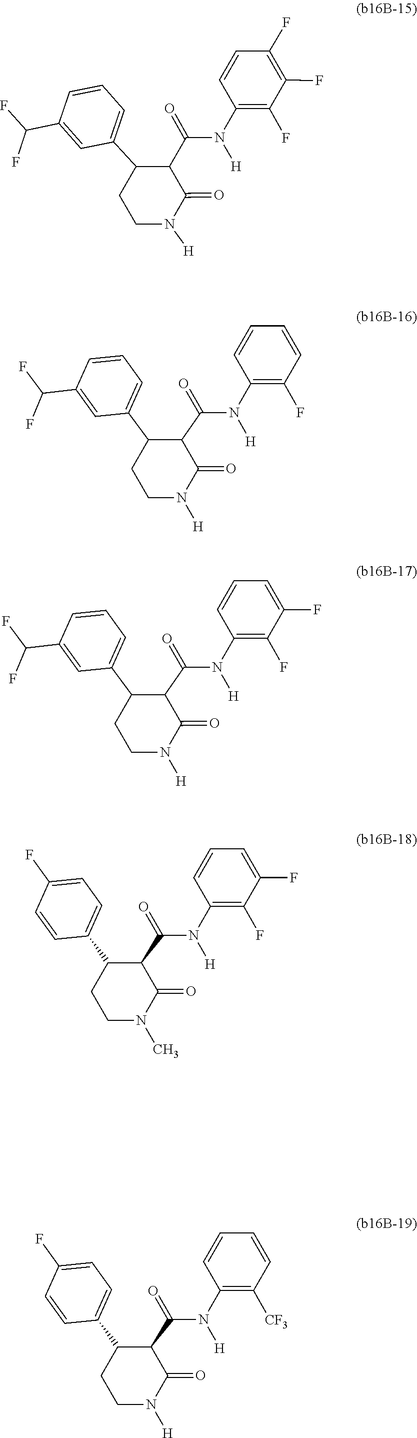

Another Embodiment wherein “other herbicides” (b16) also include a compound of Formula (b16C),

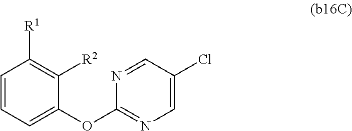

wherein R¹ is Cl, Br or CN; and R² is C(═O)CH₂CH₂CF₃, CH₂CH₂CH₂CH₂CF₃ or 3-CHF₂-isoxazol-5-yl.

“Herbicide safeners” (b17) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl)sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

Preferred for better control of undesired vegetation (e.g., lower use rate such as from greater-than-additive effects, broader spectrum of weeds controlled, or enhanced crop safety) or for preventing the development of resistant weeds are mixtures of a compound of this invention with a herbicide selected from the group consisting of 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-1H-indol-6-yl)-2-Pyridinecarboxylic cyanomethyl ester (CAS No. 2251111-18-7), 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6), atrazine, azimsulfuron, beflubutamid, beflubutamid-M, bixlozone, broclozone, benzisothiazolinone, 1-(2-carboxyethyl)-4-(2-pyrimidinyl)pyridazinium (CAS No. 2285384-11-2) and salts thereof, carfentrazone-ethyl, chlorimuron-ethyl, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylic ethyl ester (CAS No. 1949837-17-5), chlorsulfuron-methyl, clomazone, clopyralid potassium, cloransulam-methyl, 2-[(2,4-dichlorophenyl)methyl]-4,4-dimethyl-isoxazolidinone, 2-[(2,5-dichlorophenyl)methyl]-4,4-dimethyl-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl.

One or more of the following methods and variations as described in Schemes 1-16 can be used to prepare the compounds of Formula 1. The definitions of A, R^{1a}, R^{1b}, R², R^{3a}, R^{3b}, R^{4a}, R^{4b}, X¹, X², n, p and q in the compounds of Formulae 1-26 below are as defined above in the Summary of the Invention unless otherwise noted. Compounds of Formulae 1a, 1b, 1c, 1d, 8a, 8b, 10a, 16a and 16b are various subsets of the compounds of Formulae 1, 8, 10 and 16; and all substituents for Formulae 1a, 1b, 1c, 1d, 8a, 8b, 10a, 16a and 16b are as defined above for Formula 1 unless otherwise noted in the disclosure including the schemes.

As shown in Scheme 1, compounds of Formula 1a (i.e. compounds of Formula 1 wherein Y is S) can be prepared by treatment of compounds of Formula 1b (i.e. Formula 1 wherein Y is O) with a thionation reagent such as Lawesson's reagent, tetraphosphorus decasulfide or diphosphorus pentasulfide in an appropriate solvent (e.g. tetrahydrofuran or toluene) at temperatures typically ranging from 0° C. to the reflux temperature of the solvent.

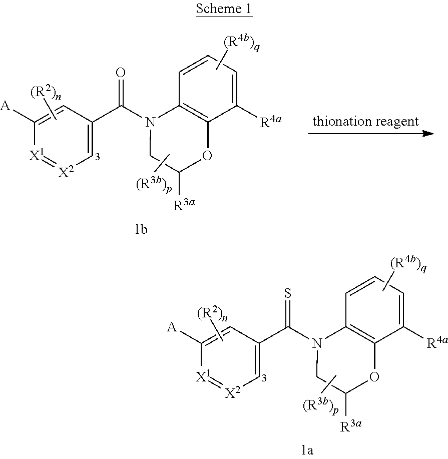

Compounds of Formula 1b (i.e. compounds of Formula 1 wherein Y is O) can be prepared using standard cross-coupling reactions, such as those described in Science of Synthesis: Cross Coupling and Heck-Type Reactions 1, Molander, G. A. (volume editor), Thieme (2013) and Science of Synthesis: Cross Coupling and Heck-Type Reactions 2, Wolfe, J. P. (volume editor), Thieme (2013); and references cited therein. For example, compounds of Formula 1c (i.e. Formula 1b wherein A is bonded through a nitrogen ring member) can be prepared from compounds of Formula 2 (wherein Z is a halogen or pseudohalogen such as Cl, Br, I or OTf) via palladium- or copper-mediated coupling with heterocycles of Formula 3 (wherein H is connected to a nitrogen ring member of A), as shown in Scheme 2. Ullmann-type coupling conditions, such as those described in J. Org. Chem. 2004, 69, 5578-5587, are often suitable and are typically catalyzed by a copper salt such as copper(I) iodide or copper(I) oxide and a ligand, such as trans-N,N′-dimethylcyclohexane-1,2-diamine, trans-1,2-diaminocyclohexane, N,N′-dimethylethylenediamine, 1,10-phenanthroline, 8-quinolinol, (S)-proline or 2-picolinic acid, in the presence of a base (e.g. potassium carbonate, cesium carbonate or potassium phosphate) in an appropriate solvent (e.g. N,N-dimethylformamide, dimethyl sulfoxide, N,N-dimethylacetamide, toluene, 1,4-dioxane or acetonitrile). Temperatures between ambient temperature and 150° C. are generally appropriate for the reaction. Palladium-catalyzed coupling conditions may also be suitable for some heterocycles. Appropriate palladium catalysts include but are not limited to tetrakis(triphenylphosphine)palladium(0), [1,1′-bis(diphenylphosphino)ferrocene]dichloropalladium(II), bis(triphenylphosphine)palladium(II) dichloride, palladium(II) acetate or tris(dibenzylideneacetone)dipalladium(0). In some cases, the addition of a ligand is beneficial, including but not limited to, 2,2′-bis(diphenylphosphino)-1,1′-binaphthalene (BINAP), 2-dicyclohexylphosphino-2′,6′-dimethoxybiphenyl (SPhos), 2-dicyclohexylphosphino-2′,4′,6′-triisopropylbiphenyl (XPhos), 4,5-bis(diphenylphosphino)-9,9-dimethylxanthene (Xantphos), 2-(di-tert-butylphosphino)biphenyl (JohnPhos), 2-dicyclohexylphosphino-2′,6′-diisopropoxy-1,1′-biphenyl (RuPhos) or 2-di-tert-butylphosphino-3,4,5,6-tetramethyl-2′,4′,6′-triisopropyl-1,1′-biphenyl (Me₄t-ButylXphos). These reactions are generally run with a base (e.g. sodium tert-butoxide, lithium bis(trimethylsilyl)amide, cesium carbonate, potassium carbonate or potassium phosphate tribasic) and a solvent (e.g. tetrahydrofuran, 1,4-dioxane, toluene or tert-butanol) at temperatures generally ranging from ambient temperature to 150° C. For reviews of these methods, see: Chem. Rev. 2008, 108, 3054-3131; Chem. Sci. 2010, 1, 13-31; Beilstein J. Org. Chem. 2011, 7, 59-74; Chem. Rev. 2016, 116, 12564-12649; and Angew. Chem. Int. Ed. 2017, 56, 16136-16179 and Tetrahedron 2019, 75, 4199-4211. One skilled in the art will recognize that when more than one nitrogen atom is present in the heterocycle of Formula 3, formation of regioisomers is possible and standard purification methods such as chromatography, can generally be used to separate the mixture. Heterocycles of Formula 3 are generally commercially available or known in the literature.

Alternatively, when the aryl ring is sufficiently activated, for example when X¹ is N or when R² is a suitably positioned electron-withdrawing group, compounds of Formula 1c can generally be prepared without the addition of a metal catalyst. This is achieved by reaction of compounds of Formula 2 (wherein Z is a halogen such as F or Cl) with heterocycles of Formula 3 in the presence of a base (e.g. potassium carbonate, sodium carbonate, cesium carbonate, potassium phosphate, sodium hydroxide, potassium tert-butoxide, sodium hydride or potassium hydride) in an appropriate solvent (e.g. N,N-dimethylformamide, dimethyl sulfoxide, N-methyl-2-pyrrolidone, tetrahydrofuran, 1,4-dioxane, 1,2-dimethoxyethane, diglyme, acetonitrile or toluene). Temperatures ranging from 0° C. to 200° C. are generally suitable for the reaction. For examples of this reaction in the literature, see: WO 2012/054510; WO 2020/207941 and J. Med. Chem. 2014, 57, 10013-10030.

In some instances, compounds of Formula 1c may be more readily accessed using standard heterocyclic synthesis procedures known to those skilled in the art. For appropriate methods, see Science of Synthesis, Volumes 12-13, Neier, R., Storr, R. C. and Gilchrist, T. L. (volume editors), Thieme (2002-2003); Comprehensive Heterocyclic Chemistry IV, Volumes 4-6, Black, D. S., Cossy, J. and Stevens, C. V. (editors-in-chief), Elsevier (2022); WO 2015/160636 and WO 2017/205709; and references cited therein.

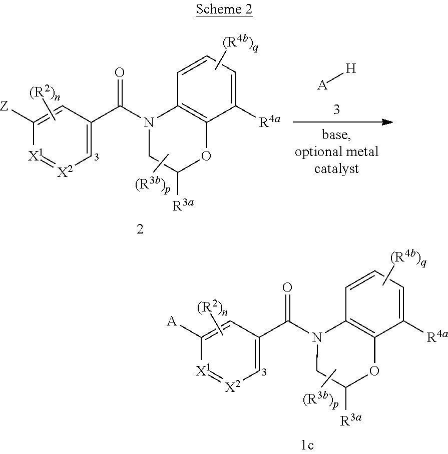

As shown in Scheme 3, compounds of Formula 1d (i.e. compounds of Formula 1b wherein A is bonded through a carbon ring member), can be prepared by well-known metal-catalyzed cross-coupling reactions between a heterocycle of Formula 4 (wherein Z is a halogen or pseudohalogen such as Cl, Br, I or OTf and is connected to a carbon ring member) and an organometallic compound of Formula 5 (wherein M is a transmetalating group), such as, but not limited to, a boronic acid (e.g. M is B(OH)₂), boronate ester (e.g. M is B(—O(CMe₂)₂O—) or organotin reagent (e.g. M is Sn(n-Bu)₃, SnMe₃). The metal catalysts used in these couplings include, but are not limited to, tetrakis(triphenylphosphine)palladium(0), [1,1′-bis(diphenylphosphino)ferrocene]dichloropalladium(II), bis(triphenylphosphine)palladium(II) dichloride, palladium(II) acetate or tris(dibenzylideneacetone)dipalladium(0). In some cases, the addition of a ligand is beneficial, including but not limited to, 2-dicyclohexylphosphino-2′,4′,6′-triisopropylbiphenyl (XPhos), 2-dicyclohexylphosphino-2′,6′-dimethoxybiphenyl (SPhos), tricyclohexylphosphine or tri(2-furyl)phosphine. Generally, these reactions are run in solvents such as N,N-dimethylformamide, dimethyl sulfoxide, N-methyl-2-pyrrolidone, toluene, 1,2-dimethoxyethane, 1,4-dioxane, tetrahydrofuran, acetonitrile or ethanol and temperatures generally range from ambient temperature to 150° C. One skilled in the art will recognize that the reaction conditions will depend on the organometallic species used in the reaction, for example, when the compound of Formula 5 is a boron reagent, a base, often an aqueous base, is required. Suitable bases include potassium carbonate, sodium carbonate, cesium carbonate, sodium bicarbonate or potassium phosphate. When the compound of Formula 5 is a tin reagent, a base is not required. For reviews of transition metal-catalyzed cross-coupling reactions, see: E. Negishi, Handbook of Organopalladium Chemistry for Organic Synthesis, John Wiley and Sons, Inc., New York, 2002; N. Miyaura, Cross-Coupling Reactions: A Practical Guide, Springer, New York, 2002; H. C. Brown et al., Organic Synthesis via Boranes, Vol. 3, Aldrich Chemical Co., Milwaukee, WI, 2002; Suzuki et al., Chem. Rev. 1995, 95, 2457-2483 and Molander et al., Acc. Chem. Res. 2007, 40, 275-286. and Chem. Soc. Rev. 2013, 42, 5270. For relevant examples of reactions in the literature using boron reagents, see: WO 2012/137982, WO 2015/017610, WO 2016/040223 and WO 2020/182990. For relevant examples of reactions in the literature using tin reagents, see: WO 2019/195810 and WO 2021/242677. Heterocycles of Formula 4 are generally commercially available or known in the literature.

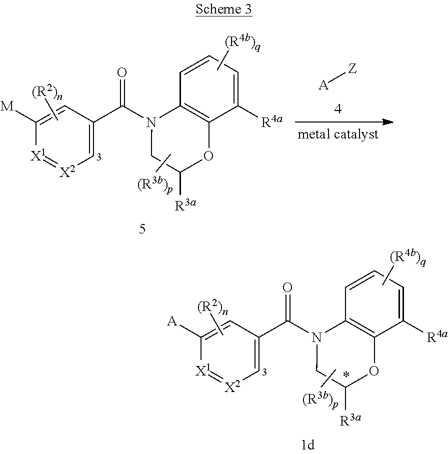

One skilled in the art will recognize that in some instances, due to the availability or stability of the required reagents, it may be beneficial to reverse the polarity of the coupling partners, as shown in Scheme 4. In this scenario, heterocycles of Formula 6 (wherein M is a transmetalating group such as, but not limited to, B(OH)₂, B(—O(CMe₂)₂O—), BF₃K, Sn(n-Bu)₃ or SnMe₃ or ZnBr and is connected to a carbon ring member) are coupled with compounds of Formula 2 (wherein Z is a halogen or pseudohalogen such as Cl, Br, I or OTf). For relevant examples in the literature, see: WO 2012/063207 and WO 2019/162323. For examples of cross-couplings of (hetero)aryl halides with alkyl coupling partners, see: Chem. Rev. 2011, 111, 1417-1492; ACS Med. Chem. Lett. 2020, 11, 597-604 and J. Org. Chem. 2021, 86, 10380-10396; and references cited therein. Heterocycles of Formula 6 are generally commercially available or known in the literature.

In some instances, compounds of Formula 1d may be more readily accessed using standard heterocyclic synthesis procedures known to those skilled in the art. For appropriate methods, see Science of Synthesis, Volumes 11-13 and 15, Schaumann, E., Neier, R., Storr, R. C.; Gilchrist, T. L. and Black, D. S. (volume editors), Thieme (2001-2004) and Comprehensive Heterocyclic Chemistry IV, Volumes 4-7, Black, D. S., Cossy, J. and Stevens, C. V. (editors-in-chief), Elsevier (2022); and references cited therein.

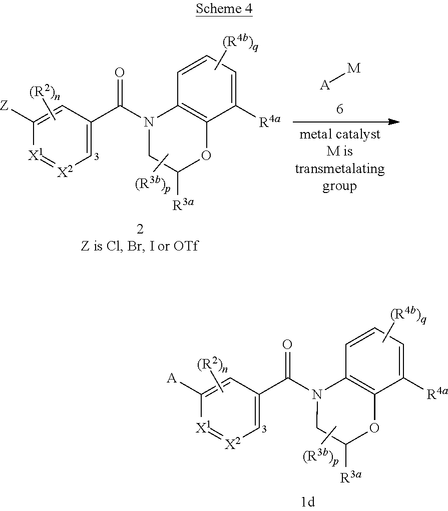

As shown in Scheme 5, organometallic compounds of Formula 5 (wherein M is a transmetalating group such as, but not limited to, B(—O(CMe₂)₂O—), Sn(n-Bu)₃ or SnMe₃) can be prepared from compounds of Formula 2 (wherein Z is a halogen or pseudohalogen such as Cl, Br, I or OTf) using well-known metal-catalyzed cross-coupling reactions. For example, pinacol boronic esters (wherein M=B(—O(CMe₂)₂O—), can generally be prepared using palladium-catalyzed borylation conditions, such as those described in J. Org. Chem. 1995, 60, 7508-7510 and J. Org. Chem. 2021, 86, 103-109; and references cited therein. A compound of Formula 2 is treated with bis(pinacolato)diboron in the presence of a palladium catalyst like [1,1′-bis(diphenylphosphino)ferrocene]dichloropalladium(II) and a base like potassium acetate in a solvent like dioxane or dimethyl sulfoxide, at temperatures ranging from ambient temperature to the reflux temperature of the solvent. Similarly, trialkyl tin reagents (e.g. wherein M=SnMe₃, SnBu₃) can generally be prepared from a compound of Formula 2 by treatment with a stannane like hexamethylditin or hexabutylditin in the presence of a palladium catalyst like tetrakis(triphenylphosphine)palladium(0) in a solvent like dioxane or toluene, at temperatures ranging from ambient temperature to the reflux temperature of the solvent. For examples of this reaction in the literature, see: WO 03/077918 and WO 2021/050964. For additional details on preparation and use of organoboron and organotin reagents, see Science of Synthesis, Volumes 5-6, Moloney, M. G. and Kaufmann, D. E. (volume editors), Thieme (2002-2004); and references cited therein.

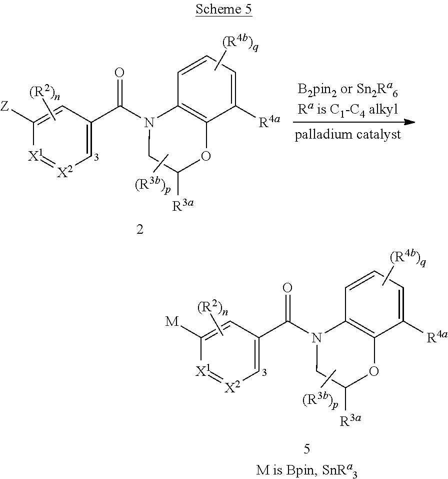

As shown in Scheme 6, compounds of Formulae 1 or 2 can be prepared by reacting a carboxylic acid of Formula 7 with a benzoxazine of Formula 8. The reaction proceeds via activation of the carboxylic acid of Formula 7 followed by reaction with the benzoxazine of Formula 8. The carboxylic acid can be activated with a coupling reagent or by conversion of the carboxylic acid to an acid halide, such as an acid chloride. For example, compounds of Formulae 7 and 8 can be reacted in the presence of a coupling reagent such as propylphosphonic anhydride (T3P), N-(3-dimethylaminopropyl)-N′-ethylcarbodiimide hydrochloride (EDC), 1,1′-carbonyldiimidazole (CDI) or2-chloro-1-methylpyridinium iodide (Mukaiyama's reagent). Polymer supported reagents, such as polymer-supported cyclohexylcarbodiimide, are also suitable. These reactions are typically run in an appropriate solvent (e.g. dichloromethane, 1,2-dichloroethane, ethyl acetate, acetonitrile or N,N-dimethylformamide) in the presence of a base (e.g. triethylamine, N,N-diisopropylethylamine or pyridine), optionally with a catalytic amount of 4-(dimethylamino)pyridine, at temperatures ranging from 0° C. to the reflux temperature of the solvent. Alternatively, a carboxylic acid of Formula 7 can be converted to an acid chloride by treatment with a reagent such as thionyl chloride, oxalyl chloride, phosphoryl chloride, phosphorus trichloride or phosphorus pentachloride, either neat or in an appropriate solvent (e.g. dichloromethane, 1,2-dichloroethane or toluene) and optionally with a catalytic amount of N,N-dimethylformamide, at temperatures ranging from 0° C. to the reflux temperature of the solvent. Subsequent reaction of the acid chloride with benzoxazine of Formula 8 generally occurs in the presence of a base (e.g. triethylamine, N,N-diisopropylethylamine or pyridine), optionally with a catalytic amount of 4-(dimethylamino)pyridine, in an appropriate solvent (e.g. tetrahydrofuran, dioxane, toluene, dichloromethane, chloroform, 1,2-dichloroethane or ethyl acetate) at temperatures ranging from 0° C. to the reflux temperature of the solvent. For relevant examples of this reaction in the literature, see: J. Med. Chem. 2012, 55, 10475-10489; WO2010/049302 and WO 2016/001631. Carboxylic acids of formula 7 are generally commercially available or known in the literature. In some cases, the corresponding acid chlorides are also commercially available.

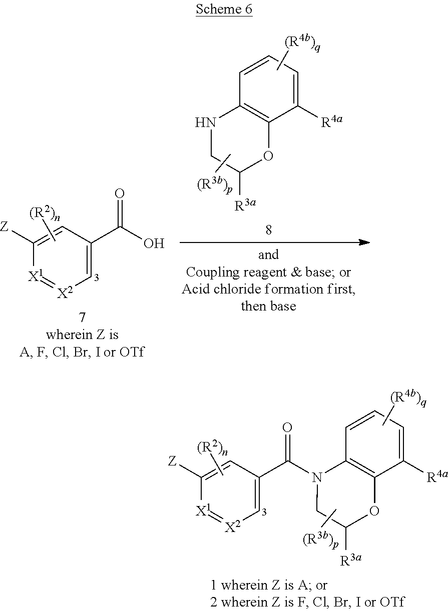

Several methods useful for preparing benzoxazines of Formula 8 are well-known in the literature. In addition, some benzoxazines of Formula 8 are commercially available. One method to prepare benzoxazines of Formulae 8a (i.e. the compounds of Formula 8 wherein R^{b}is H and p is 0 or 1) or 9 (wherein R^{b} is an appropriate protecting group such as Bn or p-methoxybenzyl and p is 0 or 1) is illustrated in Scheme 7 below, by treatment of benzoxazinones of Formula 10 (wherein R^{b} is H or an appropriate protecting group such as Bn or p-methoxybenzyl and p is 0 or 1) with a reducing agent, such as borane or lithium aluminum hydride in a solvent like tetrahydrofuran at temperatures ranging from 0° C. to the reflux temperature of the solvent. For a relevant example of this reaction in the literature, see WO 2015/095795. Some benzoxazinones of Formula 10 are commercially available or known in the literature.

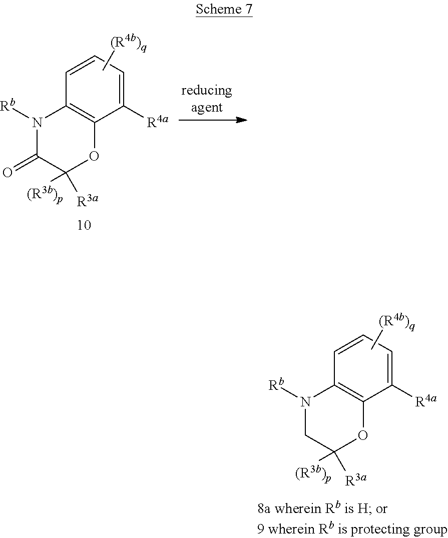

As shown in Scheme 8, benzoxazines of Formulae 8b (i.e., compounds of Formula 8 wherein R^{b} is H, and R^{3b} at the α-position to the N atom are F and p is 2 or 3) or 11 (wherein R^{b} is an appropriate protecting group such as Bn or p-methoxybenzyl and R^{3b} at the α-position to the N atom are F and p is 2 or 3) can be prepared by fluorination of benzoxazinones of Formula 10 (wherein R^{b} is H or an appropriate protecting group such as Bn or p-methoxybenzyl and p is 0 or 1). For example, α,α-difluoroamines may be prepared by first chlorinating the amide with a reagent like oxalyl chloride in an appropriate solvent (e.g. dichloromethane, carbon tetrachloride, tert-butyl methyl ether or cyclopentyl methyl ether) at temperatures generally between 0° C. and the reflux temperature of the solvent. This is followed by fluorination with a reagent like sodium fluoride, potassium fluoride or triethylamine trihydrofluoride in an appropriate solvent (e.g acetonitrile, dichloromethane or 1,3-dimethyl-2-imidazolidinone) at temperatures generally between 0° C. and the reflux temperature of the solvent. Alternatively, the amide may be first converted to a thioamide by treatment with a thionation reagent such as Lawesson's reagent or diphosphorus pentasulfide in an appropriate solvent (e.g. toluene, xylene, dioxane or tetrahydrofuran) at temperatures between ambient temperature and the reflux temperature of the solvent. In a second step, fluorination can be achieved using a reagent like bis(2-methoxyethyl)aminosulfur trifluoride (Deoxo-Fluor®) in an appropriate solvent (e.g. dichloromethane), optionally in the presence of a Lewis acid catalyst (e.g. antimony(III) chloride), at temperatures generally between 0° C. and ambient temperature. For relevant examples of this reaction in the literature, see: EP 1437342; WO 2006/049014; Tetrahedron, 2013, 69, 8943-8951 and ChemMedChem, 2013, 8, 779-799; and references cited therein.

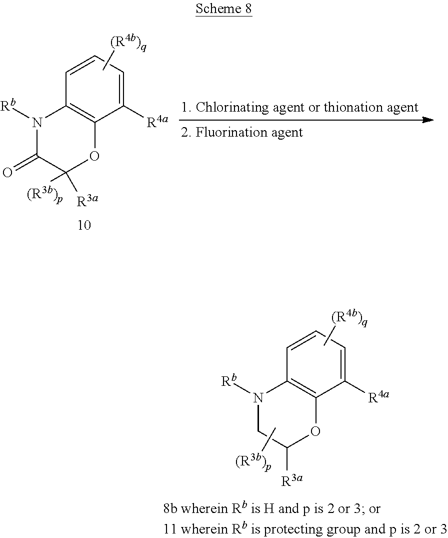

As shown in Scheme 9, benzoxazinones of Formula 10a (i.e., compounds of Formula 10 wherein R^{b} is H) can be prepared from compounds of Formula 12 (wherein R^{a} is C₁-C₄ alkyl, typically methyl or ethyl) by nitro reduction followed by cyclization which occurs during reduction or upon heating. This reaction is readily achieved under a range of conditions, such as iron metal in the presence of an acid like acetic acid, hydrochloric acid or aqueous ammonium chloride, optionally with a solvent such as methanol, ethanol, ethyl acetate or N,N-dimethylformamide at temperatures ranging from ambient temperature to the reflux temperature of the solvent. Other suitable conditions include zinc metal with acetic acid or aqueous ammonium chloride, and stannous chloride in aqueous hydrochloric acid or ethanol. Alternatively, the reaction can be achieved using a transition metal catalyst, such as palladium on carbon, platinum oxide or Raney nickel under an atmosphere of hydrogen, in an appropriate solvent (e.g. methanol, ethanol, ethyl acetate or tetrahydrofuran). Temperatures typically range from ambient temperature to 80° C. This reaction can generally be done in a Parr hydrogenator. For relevant examples of this reaction in the literature, see: WO 2015/095795 and Angew. Chem. Int. Ed. 2014, 53, 6126-6130.

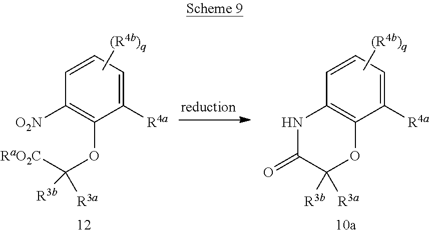

As shown in Scheme 10, compounds of Formula 12 can be prepared by Mitsunobu reaction of nitrophenols of Formula 13 with α-hydroxy esters of Formula 14 (wherein R^{a} is C₁-C₄ alkyl, typically methyl or ethyl). Typical reaction conditions include triphenylphosphine and an azodicarboxylate such as diethyl azodicarboxylate or diisopropyl azodicarboxylate, in a solvent like tetrahydrofuran or dichloromethane at temperatures ranging from about −10° C. to the reflux temperature of the solvent. Alternatively, compounds of Formula 12 can be prepared by reaction of nitrophenols of Formula 13 with α-halo esters of Formula 15 (wherein LG is a leaving group such as Cl or Br and R^{a} is C₁-C₄ alkyl, typically methyl or ethyl) in the presence of a base, such as potassium carbonate or cesium carbonate, in a solvent like N,N-dimethylformamide, acetone or 1,4-dioxane, at temperatures typically ranging from 0° C. to the reflux temperature of the solvent. For relevant examples of these reactions in the literature, see: Bioorg. Med. Chem. 2013, 23, 4501-4505; Bioorg. Med. Chem. 2007, 15, 5912-5949 and WO 2017/205536. Nitrophenols, α-hydroxy esters and α-halo esters of Formulae 13, 14 and 15 are generally commercially available or known in the literature.

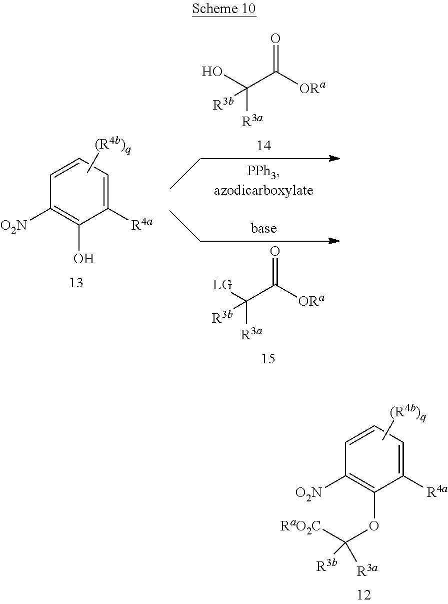

Alternatively, benzoxazines of Formula 8 can be prepared according to the following sequence. As shown in Scheme 11, β-aminoalcohols of Formula 16 (wherein R^{b} is H or an appropriate protecting group like Ts or Bn, and X is a halogen or pseudohalogen such as F, Cl, Br, I or OTs) can be prepared by ring opening of epoxides of Formula 18 by nucleophilic attack of anilines of Formula 17. This reaction can generally be achieved by heating the aniline and epoxide either neat or in an appropriate solvent (e.g. ethanol or N,N-dimethylformamide) at temperatures generally ranging from 40° C. to 180° C. Optionally, a base (e.g. potassium carbonate or sodium hydride), Lewis acid (e.g. ytterbium(III) trifluoromethanesulfonate or lithium bromide) or phase transfer catalyst (e.g. benzyltriethylammonium chloride or tetrabutylammonium bromide) can be added to facilitate the reaction. One skilled in the art will recognize that the epoxide substituents and reaction conditions impact the regioselectivity of the reaction, for example acidic conditions may reverse the regioselectivity of epoxide ring-opening. For relevant examples of these reactions in the literature, see: Ind. Eng. Chem. Res. 2003, 42, 680-686; Eur. J. Org. Chem. 2004, 3597-3600; J. Het Chem. 2010, 47, 1406-1410 and WO 2015/095792; and references cited therein. Anilines and epoxides of Formulae 17 and 18 are generally commercially available or known in the literature.

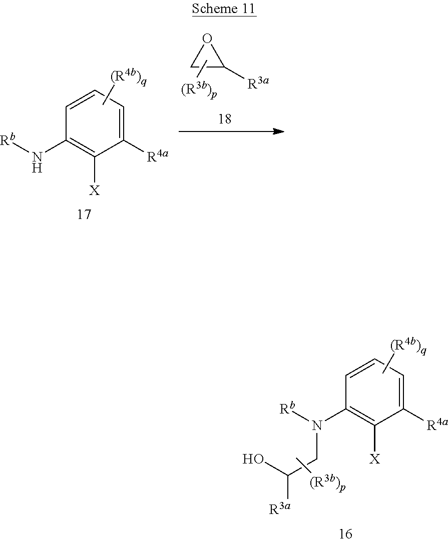

As shown in Scheme 12, N-tosyl protected benzoxazines of Formula 20 can be prepared from β-aminoalcohols of Formula 16a (i.e. compounds of Formula 16 wherein R^{b} is Ts and X is typically F but in some instances is OTs) by treatment with a base such as, but not limited to, sodium hydroxide, potassium tert-butoxide or sodium hydride, optionally in the presence of a phase transfer catalyst like tetrabutylammonium bromide, typically in a solvent such as tetrahydrofuran at temperatures ranging from ambient temperature to the reflux temperature of the solvent. For examples of this reaction in the literature, see: Ind. Eng. Chem. Res. 2003, 42, 680-686; WO 2015/095792 and J. Mol. Catal. A Chem. 2008, 288, 28-32. Benzoxazines of Formula 8 can be prepared by removal of the N-tosyl protecting group. This can be achieved under reducing conditions, for example, by treatment with magnesium metal in methanol at temperatures ranging from 0° C. to the reflux temperature of the solvent, optionally with sonication. Alternatively, the reaction can be achieved using acidic hydrolysis conditions, for example, treatment with sulfuric acid either neat or in a solvent like dichloromethane, at temperatures generally ranging from 0° C. to the reflux temperature of the solvent. For relevant examples of these reactions in the literature, see: Chem. Commun. 1999, 2095-2096; WO 2015/095792 and J. Org. Chem. 2021, 86, 16573-16581; and references cited therein.

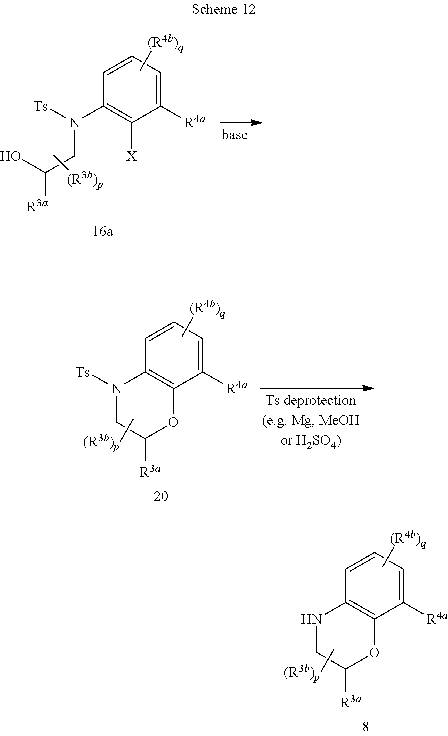

Alternatively, as shown in Scheme 13, benzoxazines of Formulae 8 (wherein R^{b} is H) or 21 (wherein R^{b} is a suitable protecting group like Ts or Bn) can be prepared from (3-aminoalcohols of Formula 16b (i.e. compounds of Formula 16 wherein R^{b} is H or a suitable protecting group and X is a halogen such as Cl, Br or I) by transition metal-catalyzed O-arylation. This reaction can be catalyzed by a copper salt such as copper(I) iodide and a ligand such as 1,10-phenanthroline, or by a palladium salt or complex such as palladium(II) acetate or tris(dibenzylideneacetone)dipalladium(0) and a phosphine ligand such as 2-di-tert-butylphosphino-2′-(N,N-dimethylamino)biphenyl (t-BuDavePhos) or rac-2-(Di-tert-butylphosphino)-1,1′-binaphthyl (TrixiePhos). These reactions are run in a solvent like dioxane or toluene, in the presence of a base such as sodium tert-butoxide, cesium carbonate or potassium phosphate, at temperatures generally ranging from ambient temperature to the reflux temperature of the solvent. For examples of this reaction in the literature, see: J. Am. Chem. Soc. 2000, 122, 12907-12908; J. Am. Chem. Soc. 2001, 123, 12202-12206; J. Mol. Catal. A Chem. 2008, 288, 28-32 and Tetrahedron Lett. 2009, 50, 3790-3793. Where protecting groups are utilized, removal using standard procedures known to those skilled in the art provides benzoxazines of Formula 8.

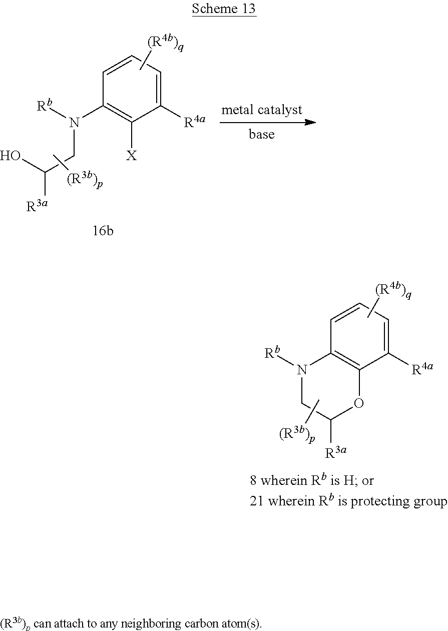

In some instances, benzoxazines of Formula 8 may be more readily accessed according to the sequence shown in Scheme 14. Compounds of Formula 22 can be prepared by ring opening of epoxides of Formula 18 by nucleophilic attack of phenols of Formula 13. This reaction can generally be achieved in the presence of a base (e.g. monosodium phosphate, sodium phosphate, potassium carbonate, sodium hydride, sodium hydroxide, cesium fluoride or 1,8-diazabicyclo[5.4.0]undec-7-ene) in an appropriate solvent (e.g. acetonitrile, N,N-dimethylformamide, dimethyl sulfoxide, dichloromethane, toluene, methanol, iso-propanol and/or water), at temperatures generally ranging from ambient temperature to 180° C. Lewis acids (e.g. zinc chloride or boron trifluoride diethyl etherate) may also be used to facilitate the reaction. One skilled in the art will recognize that the epoxide substituents and reaction conditions impact the regioselectivity of the reaction, for example acidic conditions may reverse the regioselectivity of epoxide ring-opening. For relevant examples of this reaction in the literature, see: WO 2000/010994; US 2007/0185097 A1; WO 2009/009501; J. Med. Chem. 2023, 66, 1583-1600 and Angew. Chem. Int. Ed. 2023, 62, e202217064. Subsequently, compounds of Formula 23 can be prepared by nitro reduction, which can be achieved using standard conditions as described previously (see Scheme 9), for example palladium on carbon under an atmosphere of hydrogen in an appropriate solvent like methanol. For relevant examples of this reaction in the literature, see: WO 2009/009501, WO 2010/047956 and WO 2019/162323. Benzoxazines of Formula 8 can be prepared from compounds of Formula 23 by treatment with an acid (e.g. phosphoric acid or p-toluenesulfonic acid) in an appropriate solvent (e.g. xylene or toluene) at temperatures generally ranging from ambient temperature to the reflux temperature of the solvent. For relevant examples of this reaction in the literature, see: WO 2017/108723 and Synth. Commun. 1998, 28, 4105-4121. In some instances, it may be beneficial to convert the alcohol to a leaving group (e.g. Cl or OMs) to facilitate the reaction, which may then proceed in the presence of a base (e.g. potassium carbonate) in an appropriate solvent (e.g. N,N-dimethylformamide) at temperatures ranging from ambient temperature to the reflux temperature of the solvent. For a relevant example of this reaction in the literature, see: WO 2002/070726. Alternatively, in some instances, use of an appropriate aniline protecting group (e.g. tosyl) enables ring closure using Mitsunobu conditions as described previously (see Scheme 10), for example triphenyl phosphine and diethyl azodicarboxylate in an appropriate solvent like tetrahydrofuran. For relevant examples of this reaction in the literature, see: Org. Biomol. Chem. 2010, 8, 2823-2828 and J. Org. Chem. 2015, 80, 3815-3824.

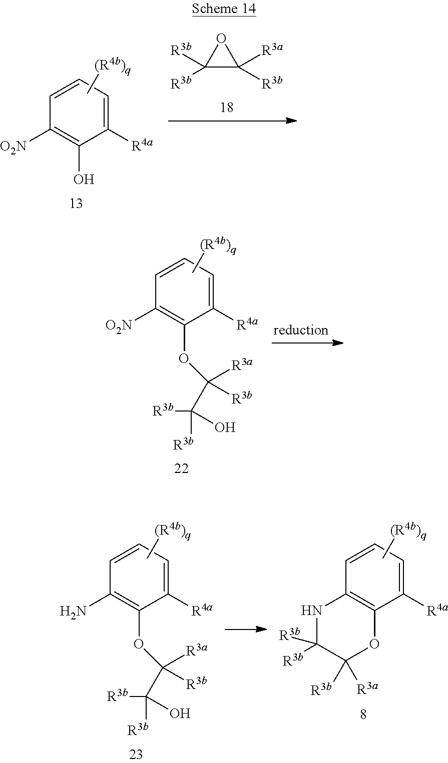

As shown in Scheme 15, benzoxazines of Formula 8c (i.e., compounds of Formula 8 wherein one R^{3b} at the α-position to the NH (i.e. α-amino R^{3b}) is R^{c} and the remaining α-amino R^{3b} is H; R^{c} is H or C₁-C₃ alkyl; or R^{c} can be taken together with R^{3a} or the α-oxo R^{3b} to form a ring) can be prepared from compounds of Formula 24 (wherein R^{c} is H or C₁-C₃ alkyl; or R^{c} can be taken together with R^{3a} or R^{3b} to form a ring) by nitro reduction followed by reductive amination. This reaction can be achieved using a transition metal catalyst, such as but not limited to palladium on carbon, platinum on carbon or Raney nickel under an atmosphere of hydrogen, in an appropriate solvent (e.g. methanol, ethanol, iso-propanol, ethyl acetate, toluene or tetrahydrofuran). Temperatures typically range from ambient temperature to 80° C. This reaction can generally be done in a Parr hydrogenator, optionally above atmospheric pressure. Alternatively, the reaction can be achieved in a stepwise manner. Nitro reduction can be achieved under a range of conditions, such as iron metal in the presence of an acid like acetic acid, hydrochloric acid or aqueous ammonium chloride, optionally with a solvent such as methanol, ethanol, ethyl acetate, tetrahydrofuran or N,N-dimethylformamide at temperatures ranging from ambient temperature to the reflux temperature of the solvent. Other suitable conditions include zinc metal with acetic acid or aqueous ammonium chloride, and stannous chloride in aqueous hydrochloric acid or ethanol. A second step involves imine reduction which can be achieved using a reducing agent like sodium borohydride, sodium cyanoborohydride or lithium aluminum hydride in an appropriate solvent (e.g. ethanol, methanol, tetrahydrofuran or dichloromethane, optionally with a co-solvent or additive like water or acetic acid) at temperatures generally between −78° C. and the reflux temperature of the solvent. Other suitable reducing conditions for this step include but are not limited to hydrogen and palladium on carbon in a solvent such as methanol, or triethylsilane and trifluoroacetic acid in a solvent such as dichloromethane. For relevant examples of this reaction in the literature, see: J. Org. Chem. 2002, 67, 6097-6103; WO2014/171527; J. Org. Chem. 2015, 80, 3815-3824; WO2015/124868; WO 2017/172505 and WO 2022/035799.

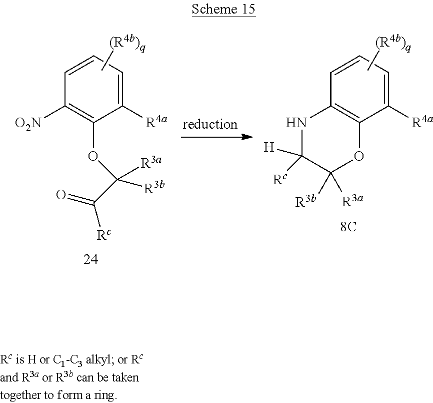

As shown in Scheme 16, compounds of Formula 24 can be prepared by reaction of nitrophenols of Formula 13 with compounds of Formula 26 (wherein LG is a leaving group such as Cl or Br and R^{c} is H or C₁-C₃ alkyl; or R^{c} can be taken together with R^{3a} or R^{3b} to form a ring) in the presence of a base, such as potassium carbonate, cesium carbonate, sodium bicarbonate or sodium hydride, optionally with additives such as sodium iodide, in a solvent like N,N-dimethylformamide, acetone or 1,4-dioxane, at temperatures typically ranging from 0° C. to the reflux temperature of the solvent. Alternatively, Mitsunobu reaction conditions may be appropriate in some cases, by reaction of nitrophenols of Formula 13 with Compounds of Formula 25 (wherein Re is H or C₁-C₃ alkyl; or R^{c} can be taken together with R^{3a} or R^{3b} to form a ring). Typical reaction conditions include triphenylphosphine and an azodicarboxylate such as diethyl azodicarboxylate or diisopropyl azodicarboxylate, in a solvent like tetrahydrofuran or dichloromethane at temperatures ranging from about −10° C. to the reflux temperature of the solvent. For relevant examples of these reactions in the literature, see: J. Med. Chem. 1988, 31, 1548-1558; WO 2001/090088; WO 2004/080973 and WO 2018/013770. Nitrophenols, α-hydroxy carbonyls and α-halo carbonyls of Formulae 13, 25 and 26 are generally commercially available or known in the literature. In some instances, compounds of Formula 24 may be more readily accessed using protecting groups to mask the carbonyl group, such as an acetal group to mask an aldehyde, or via standard functional group interconversions of one carbonyl functional group to another.

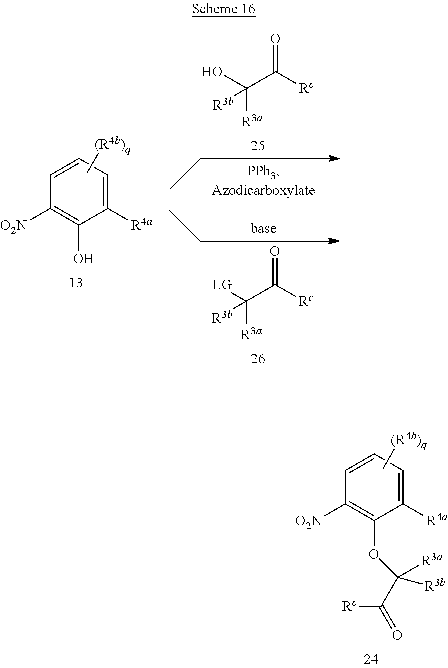

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. All NMR spectra are reported in CDCl₃ downfield from tetramethylsilane at 500 MHz unless otherwise indicated where s means singlet, brs means broad singlet, d means doublet, t means triplet, q means quarte, p means pentet and m means multiplet.

### Synthesis Example 1

### Preparation of [(2S)-8-chloro-2-methyl-2,3-dihydro-1,4-benzoxazin-4-yl]-[5-(3-isopropyl-1,2,4-triazol-1-yl)-2-methoxy-phenyl]methanone (Compound 84)

### Step A: Preparation of methyl (2S)-2-(2-chloro-6-nitro-phenoxy)propanoate

To a stirred mixture of 2-chloro-6-nitro-phenol (15 g, 86 mmol) and triphenylphosphine (29 g, 112 mmol) in anhydrous tetrahydrofuran (245 mL) was added methyl (R)-(+)-lactate (12.4 mL, 130 mmol). The mixture was cooled to 0° C. then a solution of diisopropyl azodicarboxylate (22 mL, 112 mmol) in anhydrous tetrahydrofuran (25 mL) was added over 20 min. The reaction mixture was stirred at room temperature overnight then was concentrated. A mixture of hexanes and diethyl ether (1:1, 700 mL) was added and the mixture was stirred for 2 h, resulting in formation of a precipitate. The mixture was filtered through a pad of Celite, rinsing with hexanes/diethyl ether (1:1, 200 mL) and the filtrate was concentrated. The crude material was purified by column chromatography on silica gel (gradient of 10-25% ethyl acetate in hexanes) to afford the title compound as a yellow oil (23 g).

¹H NMR (CDCl₃) δ 7.71 (dd, 1H), 7.60 (dd, 1H), 7.19-7.16 (m, 1H), 4.93 (q, 1H), 3.71 (s, 3H), 1.69 (d, 3H).

### Step B: Preparation of (2S)-8-chloro-2-methyl-4H-1,4-benzoxazin-3-one

To a stirred solution of methyl (2S)-2-(2-chloro-6-nitro-phenoxy)propanoate (i.e. the product of Step A) (22 g, 86 mmol) in ethanol (310 mL) at 50° C. was added a solution of ammonium chloride (9.2 g, 173 mmol) in water (35 mL). Iron powder (14.5 g, 259 mmol) was then added portionwise over 12 min as the reaction mixture was heated from 50° C. to 70° C. After stirring at 70° C. for 48 h, the mixture was cooled to room temperature and filtered through a pad of Celite. The filtrate was concentrated then ethyl acetate and water were added. The layers were separated and the aqueous phase was extracted with ethyl acetate. The combined organic extracts were washed with saturated aqueous ammonium chloride solution, dried over anhydrous magnesium sulfate, filtered through a pad of Celite and concentrated to afford the title compound as a white solid (16.6 g), which was used without further purification.

¹H NMR (CDCl₃) δ 8.47 (br s, 1H), 7.06 (dd, 1H), 6.91-6.88 (m, 1H), 6.72 (dd, 1H), 4.77 (q, 1H), 1.63 (d, 3H).

### Step C: Preparation of (2S)-8-chloro-2-methyl-3,4-dihydro-2H-1,4-benzoxazine

To a stirred solution of (2S)-8-chloro-2-methyl-4H-1,4-benzoxazin-3-one (i.e. the product of Step B) (9.1 g, 46 mmol) in anhydrous tetrahydrofuran (100 mL) at 0° C. was added borane tetrahydrofuran complex (1 M in tetrahydrofuran, 92 mL, 92 mmol) over 30 min. The reaction mixture was stirred at room temperature overnight, then was cooled to 0° C. and methanol (70 mL) was slowly added. After stirring at room temperature for 1 h, the mixture was concentrated and partitioned between ethyl acetate and water. The layers were separated and the organic phase was washed with water, brine, dried over anhydrous magnesium sulfate and concentrated to afford the title compound as a white solid (8.3 g), which was used without further purification.

¹H NMR (CDCl₃) δ 6.73 (dd, 1H), 6.67-6.64 (m, 1H), 6.49 (dd, 1H), 4.34-4.28 (m, 1H), 3.82 (br s, 1H), 3.37 (dd, 1H), 3.13 (dd, 1H), 1.44 (d, 3H).

### Step D: Preparation of [(2S)-8-chloro-2-methyl-2,3-dihydro-1,4-benzoxazin-4-yl]-(5-iodo-2-methoxy-phenyl)methanone

To a stirred solution of (2S)-8-chloro-2-methyl-3,4-dihydro-2H-1,4-benzoxazine (i.e. the product of Step C) (0.7 g, 3.8 mmol), 5-iodo-2-methoxy-benzoic acid (1.06 g, 3.8 mmol) and triethylamine (1.6 mL, 11.4 mmol) in 1,2-dichloroethane (13 mL) was added propylphosphonic anhydride (50 wt. % in ethyl acetate, 4.9 mL, 8.2 mmol). The mixture was stirred at 70° C. for 24 h then was cooled to room temperature and concentrated. The crude material was purified by column chromatography on silica gel (gradient of 0-30% ethyl acetate in hexanes). The isolated material was dissolved in ethyl acetate, washed with 1 N aqueous hydrochloric acid solution, 1 N aqueous sodium hydroxide solution, brine, dried over anhydrous magnesium sulfate, concentrated then further purified by column chromatography on silica gel (gradient of 0-20% ethyl acetate in hexanes) to afford the title compound as a white foam (1.3 g).

MS (ES⁺) m/z Found: (M+H)⁺, C₁₇H₁₅ClINO₃, 444.2, requires 444.0.

### Step E: Preparation of [(2S)-8-chloro-2-methyl-2,3-dihydro-1,4-benzoxazin-4-yl]-[5-(3-isopropyl-1,2,4-triazol-1-yl)-2-methoxy-phenyl]methanone

A dry vial was charged with [(2S)-8-chloro-2-methyl-2,3-dihydro-1,4-benzoxazin-4-yl]-(5-iodo-2-methoxy-phenyl)methanone (i.e. the product of Step D) (1.03 g, 2.32 mmol), 3-isopropyl-1H-1,2,4-triazole (0.31 g, 2.79 mmol), potassium carbonate (0.64 g, 4.64 mmol) and copper(I) iodide (88 mg, 0.46 mmol) then purged with nitrogen gas for 10 min. Anhydrous N,N-dimethylformamide (8 mL) was added, the mixture was sparged with nitrogen gas for 5 min then trans-N,N′-dimethylcyclohexane-1,2-diamine (0.18 mL, 1.16 mmol) was added. The mixture was stirred at 105° C. overnight then cooled to room temperature and filtered through a pad of Celite. The filtrate was washed with water (×3), filtered through a plug of silica gel and concentrated. The crude material was purified by reverse phase chromatography with a C18 column (gradient of water/acetonitrile) to afford the title compound as a white solid (630 mg).

MS (ES⁺) m/z Found: (M+H)⁺, C₂₂H₂₃ClN₄O₃, 427.4, requires 427.2.

### Synthesis Example 2

### Preparation of [(2S)-8-Chloro-2,3-dihydro-2-methyl-4H-1,4-benzoxazin-4-yl][2-methyl-5-(3-methyl-1H-1,2,4-triazol-1-yl)phenyl]methanone (Compound 74) and [(2S)-8-Chloro-2,3-dihydro-2-methyl-4H-1,4-benzoxazin-4-yl][2-methyl-5-(5-methyl-TH-1,2,4-triazol-1-yl)phenyl]methanone (Compound 126)

### Step A: Preparation of [(2S)-8-chloro-2-methyl-2,3-dihydro-1,4-benzoxazin-4-yl]-(5-iodo-2-methyl-phenyl)methanone

To a stirred solution of (2S)-8-chloro-2-methyl-3,4-dihydro-2H-1,4-benzoxazine (i.e. the product of Step C in Synthesis Example 1) (1.0 g, 5.4 mmol), 5-iodo-2-methyl-benzoic acid (1.4 g, 5.4 mmol) and N,N-diisopropylethylamine (2.4 mL, 13.6 mmol) in ethyl acetate (15 mL) at 0° C. was added propylphosphonic anhydride (50 wt. % in ethyl acetate, 5.2 mL, 8.7 mmol). The mixture was stirred at room temperature overnight then heated at 70° C. for 24 h. The mixture was cooled to room temperature and additional N,N-diisopropylethylamine (2.4 mL, 13.6 mmol) and propylphosphonic anhydride (50 wt. % in ethyl acetate, 5.2 mL, 8.7 mmol) were added then the mixture was stirred at 70° C. for 22 h. The mixture was cooled to room temperature and water was added. The layers were separated, the aqueous phase was extracted with ethyl acetate and the combined organic extracts were washed with 1 N aqueous hydrochloric acid solution, brine, dried over anhydrous sodium sulfate and concentrated. The crude material was purified by column chromatography on silica gel (gradient of 0-20% ethyl acetate in hexanes) to afford the title compound containing 5-iodo-2-methyl-benzoic acid.The material was dissolved in ethyl acetate, washed with 1 N aqueous sodium hydroxide solution (×2), brine (×1), dried over anhydrous sodium sulfate and concentrated to afford the title compound as a white foam (1.6 g).

MS (ES⁺) m/z Found: (M+H)⁺, C₁₇H₁₅ClINO₂, 428.1, requires 428.0.

### Step B: Preparation of [(2S)-8-Chloro-2,3-dihydro-2-methyl-4H-1,4-benzoxazin-4-yl][2-methyl-5-(3-methyl-1H-1,2,4-triazol-1-yl)phenyl]methanone (isomer 1) (Compound 74) and [(2S)-8-Chloro-2,3-dihydro-2-methyl-4H-1,4-benzoxazin-4-yl][2-methyl-5-(5-methyl-1H-1,2,4-triazol-1-yl)phenyl]methanone (isomer 2) (Compound 126)

A dry vial was charged with [(2S)-8-chloro-2-methyl-2,3-dihydro-1,4-benzoxazin-4-yl]-(5-iodo-2-methyl-phenyl)methanone (i.e. the product of Step A) (0.18 g, 0.42 mmol), 3-methyl-1H-1,2,4-triazole (42 mg, 0.50 mmol), potassium carbonate (0.12 g, 0.88 mmol) and copper(I) iodide (8.0 mg, 0.04 mmol) then purged with nitrogen gas for 10 min. Anhydrous N,N-dimethylformamide (3 mL) and trans-N,N′-dimethylcyclohexane-1,2-diamine (0.02 mL, 0.13 mmol) were added and the mixture was stirred at 110° C. for 19 h. The mixture was cooled to room temperature, diluted with ethyl acetate and saturated aqueous sodium bicarbonate solution and stirred for 10 min. The layers were separated, the aqueous layer was extracted with ethyl acetate (×1) and the combined organic extracts were washed with brine (×2), dried over anhydrous sodium sulfate and concentrated. The crude material was purified by column chromatography on silica gel (gradient of 0-100% ethyl acetate in hexanes) to afford the title compounds of isomer 1 (90 mg) as a white solid and isomer 2 (24 mg) as a yellow oil.

Isomer 1: MS (ES⁺) m/z Found: (M+H)⁺, C₂₀H₁₉ClN₄O₂, 383.4, requires 383.1.

Isomer 2: MS (ES⁺) m/z Found: (M+H)⁺, C₂₀H₁₉ClN₄O₂, 383.4, requires 383.1.

### Synthesis Example 3

### Preparation of [3-(3-Bromo-1H-1,2,4-triazol-1-yl)phenyl][(2S)-2,3-dihydro-2,8-dimethyl-4H-1,4-benzoxazin-4-yl]methanone (Compound 61)

### Step A: Preparation of methyl (2S)-2-(2-methyl-6-nitro-phenoxy)propanoate

To a stirred mixture of 2-methyl-6-nitro-phenol (10 g, 65 mmol) and triphenylphosphine (22 g, 85 mmol) in anhydrous tetrahydrofuran (100 mL) was added methyl (R)-(+)-lactate (9.4 mL, 98 mmol). The mixture was cooled to 0° C. then a solution of diisopropyl azodicarboxylate (17 mL, 85 mmol) in anhydrous tetrahydrofuran (20 mL) was added slowly. The reaction mixture was stirred at room temperature overnight then was concentrated. A mixture of hexanes and diethyl ether (1:1, 350 mL) was added and the mixture was stirred for 1 h, resulting in formation of a precipitate. The mixture was filtered through a pad of Celite, rinsing with hexanes/diethyl ether (1:1, 150 mL) and the filtrate was concentrated. The crude material was purified by column chromatography on silica gel (gradient of 0-30% ethyl acetate in hexanes) to afford the title compound as a yellow oil (14.5 g).

¹H NMR (CDCl₃) δ 7.65-7.63 (m, 1H), 7.41-7.39 (m, 1H), 7.12-7.08 (m, 1H), 4.56 (q, 1H), 3.71 (s, 3H), 2.38 (s, 3H), 1.60 (d, 3H).

### Step B: Preparation of (2S)-2,8-dimethyl-4H-1,4-benzoxazin-3-one

To a stirred solution of methyl (2S)-2-(2-methyl-6-nitro-phenoxy)propanoate (i.e. the product of Step A) (13.9 g, 58.2 mmol) in ethanol (210 mL) at 50° C. was added a solution of ammonium chloride (6.2 g, 116 mmol) in water (23 mL). Iron powder (9.7 g, 175 mmol) was then added portionwise over 15 min as the reaction mixture was heated from 50° C. to 70° C. After stirring at 70° C. for 23 h, the mixture was cooled to room temperature and filtered through a pad of Celite. The filtrate was concentrated then ethyl acetate and water were added. The layers were separated and the aqueous phase was extracted with ethyl acetate (×1). The combined organic extracts were washed with saturated aqueous ammonium chloride solution (×1), dried over anhydrous magnesium sulfate, filtered through a pad of Celite and concentrated to afford the title compound as a white solid (9.2 g), which was used without further purification.

¹H NMR (CDCl₃) δ 7.80 (br s, 1H), 6.87-6.83 (m, 2H), 6.64-6.60 (m, 1H), 4.66 (q, 1H), 2.25 (s, 3H), 1.58 (d, 3H).

### Step C: Preparation of (2S)-2,8-dimethyl-3,4-dihydro-2H-1,4-benzoxazine

To a stirred solution of (2S)-2,8-dimethyl-4H-1,4-benzoxazin-3-one (i.e. the product of Step B) (10.3 g, 58 mmol) in anhydrous tetrahydrofuran (100 mL) at 0° C. was added borane tetrahydrofuran complex (1 M in tetrahydrofuran, 116 mL, 116 mmol) over 30 min. The reaction mixture was stirred at room temperature overnight then was cooled to 0° C. and methanol (70 mL) was slowly added. After stirring at room temperature for 1 h, the mixture was concentrated and partitioned between ethyl acetate and water. The layers were separated and the organic phase was washed with water (×1), brine (×1), dried over anhydrous magnesium sulfate and concentrated to afford the title compound as a light brown oil (9.4 g), which was used without further purification.

¹H NMR (CDCl₃) δ 6.68-6.65 (m, 1H), 6.57-6.55 (m, 1H), 6.48-6.46 (m, 1H), 4.28-4.22 (m, 1H), 3.68 (br s, 1H), 3.34 (dd, 1H), 3.10 (dd, 1H), 2.20 (s, 3H), 1.40 (d, 3H).

### Step D: Preparation of [(2S)-2,8-dimethyl-2,3-dihydro-1,4-benzoxazin-4-yl]-(3-iodophenyl)methanone

To a stirred solution of (2S)-2,8-dimethyl-3,4-dihydro-2H-1,4-benzoxazine (i.e. the product of Step C) (8.5 g, 52 mmol), 3-iodobenzoic acid (12.9 g, 52 mmol) and triethylamine (17 mL, 120 mmol) in 1,2-dichloroethane (125 mL) was slowly added propylphosphonic anhydride (50 wt. % in ethyl acetate, 50 mL, 84 mmol). The mixture was stirred at 60° C. for 15 h then was cooled to room temperature and water was added. The layers were separated, the aqueous phase was extracted with dichloromethane (×1) and the combined organic extracts were washed with 1 N aqueous hydrochloric acid solution (×1) and 1 N aqueous sodium hydroxide solution (×1). The sodium hydroxide layer was extracted with dichloromethane (×1) then the combined organic extracts were washed with brine (×1), dried over anhydrous magnesium sulfate and concentrated. The crude material was purified by column chromatography on silica gel (gradient of 0-20% ethyl acetate in hexanes) to afford the title compound as a white foam (17.6 g).

¹H NMR (CDCl₃) δ 7.88-7.87 (m, 1H), 7.77-7.74 (m, 1H), 7.38-7.37 (m, 1H), 7.07-7.04 (m, 1H), 6.88-6.87 (m, 1H), 6.81-6.47 (m, 2H), 4.50-4.44 (m, 1H), 4.37-4.23 (m, 1H), 3.33 (dd, 1H), 2.22 (s, 3H), 1.43 (d, 3H).

### Step E: Preparation of [3-(3-Bromo-1H-1,2,4-triazol-1-yl)phenyl][(2S)-2,3-dihydro-2,8-dimethyl-4H-1,4-benzoxazin-4-yl]methanone

A dry vial was charged with [(2S)-2,8-dimethyl-2,3-dihydro-1,4-benzoxazin-4-yl]-(3-iodophenyl)methanone (i.e. the product of Step D) (1.14 g, 2.89 mmol), 3-bromo-1H-1,2,4-triazole (0.51 g, 3.47 mmol), potassium carbonate (0.84 g, 6.1 mmol) and copper(I) iodide (55 mg, 0.29 mmol) then purged with nitrogen gas for 10 min. Anhydrous N,N-dimethylformamide (11 mL) and trans-N,N′-dimethylcyclohexane-1,2-diamine (0.14 mL, 0.87 mmol) were added and the mixture was stirred at 110° C. for 17 h. The mixture was cooled to room temperature and was diluted with water and ethyl acetate. The layers were separated, the aqueous phase was extracted with ethyl acetate (×2) and the combined organic extracts were washed with water (×3), brine (×1), dried over anhydrous sodium sulfate and concentrated. The crude material was purified by column chromatography on silica gel (gradient of 0-60% ethyl acetate in hexanes) to afford the title compound as a slightly green solid. The solid was dissolved in ethyl acetate, washed with saturated aqueous ammonium chloride solution (×3), dried over anhydrous sodium sulfate and concentrated to afford the title compound as a pale yellow solid (0.62 g).

¹H NMR (CDCl₃) δ 8.33 (s, 1H), 7.77-7.72 (m, 2H), 7.50-7.47 (m, 2H), 6.89-6.88 (m, 1H), 6.69-6.37 (m, 2H), 4.55-4.48 (m, 1H), 4.46-4.32 (m, 1H), 3.36 (dd, 1H), 2.23 (s, 3H), 1.46 (d, 3H).

### Synthesis Example 4

### Preparation of [(2S)-8-Chloro-2,3-dihydro-2-methyl-4H-1,4-benzoxazin-4-yl][3-(1-methyl-1H-pyrazol-4-yl)phenyl]methanone (Compound 119)

To a stirred solution of (2S)-8-chloro-2-methyl-3,4-dihydro-2H-1,4-benzoxazine (i.e. the product of Step C in Synthesis Example 1) (91 mg, 0.49 mmol), 3-(1-methylpyrazol-4-yl)benzoic acid (90 mg, 0.44 mmol) and triethylamine (0.21 mL, 1.5 mmol) in 1,2-dichloroethane (3 mL) was added propylphosphonic anhydride (50 wt. % in ethyl acetate, 0.63 mL, 1.1 mmol). The mixture was stirred at 70° C. for 24 h then was cooled to room temperature and concentrated. The crude material was purified by column chromatography on silica gel (gradient of 0-50% ethyl acetate in hexanes) to afford the title compound as a white foam (124 mg).

¹H NMR (CDCl₃) δ 7.72 (s, 1H), 7.62-7.61 (m, 1H), 7.58 (s, 1H), 7.55-7.53 (m, 1H), 7.36-7.32 (m, 1H), 7.28-7.26 (m, 1H), 7.11-7.09 (m, 1H), 6.99-6.86 (m, 1H), 6.62-6.59 (m, 1H), 4.59-4.53 (m, 1H), 4.32-4.29 (m, 1H), 3.95 (s, 3H), 3.44 (dd, 1H), 1.48 (d, 3H).

### Synthesis Example 5

### Preparation of [3-(3-Cyclopentyl-1H-1,2,4-triazol-1-yl)phenyl][(2S)-2,3-dihydro-2,8-dimethyl-4H-1,4-benzoxazin-4-yl]methanone (Compound 80)

A stirred solution of [3-(3-Bromo-1H-1,2,4-triazol-1-yl)phenyl][(2S)-2,3-dihydro-2,8-dimethyl-4H-1,4-benzoxazin-4-yl]methanone (i.e. the product of Synthesis Example 3) (136 mg, 0.33 mmol) in anhydrous tetrahydrofuran (1 mL) was briefly sparged with nitrogen gas (~1 min) then [1,1′-bis(diphenylphosphino)ferrocene]dichloropalladium(II) (24 mg, 0.03 mmol) was added. Cyclopentylzinc bromide (0.5 M in tetrahydrofuran, 2.6 mL, 1.3 mmol) was added dropwise at room temperature then the mixture was stirred at 60° C. for 1 h. The mixture was cooled to room temperature and saturated aqueous ammonium chloride solution was added. The mixture was extracted with ethyl acetate (×2) and the combined organic extracts were washed with brine (×1), dried over anhydrous sodium sulfate and concentrated. The crude material was purified by column chromatography on silica gel (gradient of 0-100% ethyl acetate in hexanes) to afford the title compound as a pale orange foam (53 mg).

¹H NMR (CDCl₃) δ 8.33 (s, 1H), 7.80-7.73 (m, 2H), 7.47-7.39 (m, 2H), 6.89-6.87 (m, 1H), 6.76-6.45 (m, 2H), 4.55-4.47 (m, 1H), 4.44-4.32 (m, 1H), 3.36 (dd, 1H), 3.28 (p, 1H), 2.23 (s, 3H), 2.14-2.07 (m, 2H), 1.95-1.88 (m, 2H), 1.87-1.79 (m, 2H), 1.74-1.67 (m, 2H), 1.45 (d, 3H).

### Synthesis Example 6

### Preparation of [(2S)-2,3-Dihydro-2,8-dimethyl-4H-1,4-benzoxazin-4-yl][3-(3-ethynyl-1H-1,2,4-triazol-1-yl)phenyl]methanone (Compound 64)

### Step A: Preparation of [(2S)-2,8-dimethyl-2,3-dihydro-1,4-benzoxazin-4-yl]-[3-[3-(2-trimethylsilylethynyl)-1,2,4-triazol-1-yl]phenyl]methanone

A dry vial was charged with [3-(3-bromo-1H-1,2,4-triazol-1-yl)phenyl][(2S)-2,3-dihydro-2,8-dimethyl-4H-1,4-benzoxazin-4-yl]methanone (i.e. the product of Synthesis Example 3) (134 mg, 0.32 mmol), copper(I) iodide (12 mg, 0.06 mmol), anhydrous N,N-dimethylformamide (3 mL) and triethylamine (0.09 mL, 0.65 mmol) and the mixture was sparged with nitrogen gas for 4 min. Ethynyltrimethylsilane (0.23 mL, 1.6 mmol) was added, the mixture was sparged with nitrogen gas for 2 min, then tetrakis(triphenylphosphine)palladium(0) (70 mg, 0.06 mmol) was added and the mixture was stirred at 50° C. for 13 h. The mixture was cooled to room temperature then was diluted with ethyl acetate and washed with saturated aqueous sodium bicarbonate solution (×2), brine (×1), dried over anhydrous sodium sulfate and concentrated. The crude material was purified by column chromatography on silica gel (gradient of 0-60% ethyl acetate in hexanes) to afford the title compound as a brown foam (98 mg).

¹H NMR (CDCl₃) δ 8.41 (s, 1H), 7.81-7.75 (m, 2H), 7.49-7.45 (m, 2H), 6.89-6.87 (m, 1H), 6.76-6.41 (m, 2H), 4.55-4.47 (m, 1H), 4.44-4.32 (m, 1H), 3.36 (dd, 1H), 2.23 (s, 3H), 1.45 (d, 3H), 0.29 (s, 9H).

### Step B: Preparation of [(2S)-2,3-Dihydro-2,8-dimethyl-4H-1,4-benzoxazin-4-yl][3-(3-ethynyl-1H-1,2,4-triazol-1-yl)phenyl]methanone

To a solution of [(2S)-2,8-dimethyl-2,3-dihydro-1,4-benzoxazin-4-yl]-[3-[3-(2-trimethylsilylethynyl)-1,2,4-triazol-1-yl]phenyl]methanone (i.e. the product of Step A) (81 mg, 0.19 mmol) in methanol (4 mL) was added potassium carbonate (30 mg, 0.22 mmol). The mixture was stirred at room temperature for 4.5 h then was filtered through a pad of Celite, rinsing with ethyl acetate and the filtrate was concentrated. The crude material was purified by column chromatography on silica gel (gradient of 0-50% ethyl acetate in hexanes) to afford the title compound as a pale yellow foam (42 mg).

¹H NMR (CDCl₃) δ 8.42 (s, 1H), 7.81-7.76 (m, 2H), 7.50-7.47 (m, 2H), 6.89-6.88 (m, 1H), 6.76-6.39 (m, 2H), 4.56-4.48 (m, 1H), 4.45-4.33 (m, 1H), 3.36 (dd, 1H), 3.17 (s, 1H), 2.23 (s, 3H), 1.46 (d, 3H).

### Synthesis Example 7

### Preparation of [(2S)-2,3-Dihydro-2,8-dimethyl-4H-1,4-benzoxazin-4-yl][3-[3-(methoxymethyl)-1H-1,2,4-triazol-1-yl]phenyl]methanone (Compound 101)

A dry 40 mL vial was charged with [3-(3-Bromo-1H-1,2,4-triazol-1-yl)phenyl][(2S)-2,3-dihydro-2,8-dimethyl-4H-1,4-benzoxazin-4-yl]methanone (i.e. the product of Synthesis Example 3) (104 mg, 0.25 mmol), potassium trifluoro(methoxymethyl)boranuide (78 mg, 0.52 mmol), [Ir(dF(CF₃)ppy)₂(bpy)]PF₆ (5.3 mg, 0.005 mmol) and [4,4′-bis(1,1-dimethylethyl)-2,2′-bipyridine]nickel (II) dichloride (5.3 mg, 0.013 mmol) then purged with nitrogen gas for 8 min. Anhydrous, degassed 1,4-dioxane (4.8 mL) was added followed by 2,6-lutidine (0.10 mL, 0.88 mmol) then the mixture was stirred and irradiated with two blue Kessil LED lights (one 34 W and one 40 W, approx. 4 cm away, with cooling fan to keep the temperature from significantly increasing) for 18 h. The mixture was partitioned between water and ethyl acetate, the layers were separated and the aqueous phase was extracted with ethyl acetate (×2). The combined organic extracts were washed with brine (×1), dried over anhydrous sodium sulfate and concentrated. The crude material was purified by column chromatography on silica gel (gradient of 0-100% ethyl acetate in hexanes) to afford the title compound as a pale yellow foam (45 mg).

¹H NMR (CDCl₃) δ 8.42 (s, 1H), 7.82-7.76 (m, 2H), 7.48-7.43 (m, 2H), 6.89-6.87 (m, 1H), 6.76-6.41 (m, 2H), 4.63 (s, 2H), 4.55-4.48 (m, 1H), 4.44-4.31 (m, 1H), 3.51 (s, 3H), 3.36 (dd, 1H), 2.23 (s, 3H), 1.45 (d, 3H).

### Synthesis Example 8

### Preparation of [(2S)-2,3-Dihydro-2,8-dimethyl-4H-1,4-benzoxazin-4-yl][3-[3-(3-oxetanyl)-1H-1,2,4-triazol-1-yl]phenyl]methanone (Compound 109)

A dry 40 mL vial was charged with [3-(3-bromo-1H-1,2,4-triazol-1-yl)phenyl][(2S)-2,3-dihydro-2,8-dimethyl-4H-1,4-benzoxazin-4-yl]methanone (i.e. the product of Synthesis Example 3) (101 mg, 0.24 mmol), [Ir(dF(CF₃)ppy)₂(dtbbpy)]PF₆ (3.5 mg, 0.003 mmol) and [4,4′-bis(1,1-dimethylethyl)-2,2′-bipyridine]nickel (II) dichloride (9.1 mg, 0.023 mmol) then purged with nitrogen gas for 7 min. Anhydrous, degassed 1,4-dioxane (3 mL) was added followed by 2,6-lutidine (0.14 mL, 1.2 mmol), tris(trimethylsilyl)silane (0.11 mL, 0.36 mmol) and 3-bromooxetane (0.04 mL, 0.48 mmol) and the mixture was sparged with nitrogen gas for 6 min. The mixture was stirred and irradiated with two blue Kessil LED lights (one 34 W and one 40 W, approx. 6 cm away, with cooling fan to keep the temperature from significantly increasing) for 18 h. The mixture was diluted with saturated aqueous sodium bicarbonate solution (2 mL) and water (1 mL) then extracted with a 4:1 mixture of dichloromethane/isopropanol (×2) and dichloromethane (×1). The combined organic extracts were washed with brine (×1), dried over anhydrous sodium sulfate and concentrated. The crude material was purified by column chromatography on silica gel (gradient of 0-80% ethyl acetate in hexanes) to afford the title compound as a white foam (33 mg).

¹H NMR (CDCl₃) δ 8.42 (s, 1H), 7.82-7.75 (m, 2H), 7.48-7.43 (m, 2H), 6.89-6.88 (m, 1H), 6.78-6.44 (m, 2H), 5.06-5.02 (m, 4H), 4.56-4.34 (m, 3H), 3.37 (dd, 1H), 2.24 (s, 3H), 1.46 (d, 3H).

### Synthesis Example 9

### Preparation of [(2S)-8-Chloro-2,3-dihydro-2-methyl-4H-1,4-benzoxazin-4-yl][3-(2-methyl-2H-1,2,3-triazol-4-yl)phenyl]methanone (Compound 127)

### Step A: Preparation of [(2S)-8-chloro-2-methyl-2,3-dihydro-1,4-benzoxazin-4-yl]-(3-iodophenyl)methanone

To a stirred solution of (2S)-8-chloro-2-methyl-3,4-dihydro-2H-1,4-benzoxazine (i.e. the product of Step C in Synthesis Example 1) (1.5 g, 8.2 mmol) in dichloromethane (45 mL) at 0° C. was added N,N-diisopropylethylamine (2.1 mL, 12 mmol) followed by 3-iodobenzoyl chloride (2.3 g, 8.6 mmol). The mixture was stirred at room temperature overnight then was diluted with dichloromethane, washed with 1 N aqueous hydrochloric acid solution (×1), 1 N aqueous sodium hydroxide solution (×1), brine (×1), dried over anhydrous sodium sulfate and concentrated. The crude material was purified by column chromatography on silica gel (gradient of 0-30% ethyl acetate in hexanes) to afford the title compound as a white foam (3.27 g).

¹H NMR (CDCl₃) δ 7.87 (m, 1H), 7.80-7.78 (m, 1H), 7.40-7.38 (m, 1H), 7.12-7.08 (m, 2H), 6.96-6.61 (m, 2H), 4.57-4.51 (m, 1H), 4.30-4.22 (m, 1H), 3.40 (dd, 1H), 1.47 (d, 3H).

### Step B: Preparation of [(2S)-8-chloro-2-methyl-2,3-dihydro-1,4-benzoxazin-4-yl]-[3-(4,4,5,5-tetramethyl-1,3,2-dioxaborolan-2-yl)phenyl]methanone

A dry vial was charged with [(2S)-8-chloro-2-methyl-2,3-dihydro-1,4-benzoxazin-4-yl]-(3-iodophenyl)methanone (i.e. the product of Step A) (204 mg, 0.49 mmol), bis(pinacolato)diboron (163 mg, 0.64 mmol), potassium acetate (145 mg, 1.48 mmol) and [1,1′-bis(diphenylphosphino)ferrocene]dichloropalladium(II) (18 mg, 0.025 mmol) then purged with nitrogen gas. Anhydrous dimethyl sulfoxide (3 mL) was added and the mixture was stirred at 80° C. overnight. The mixture was cooled to room temperature and was diluted with water and ethyl acetate. The layers were separated, the aqueous phase was extracted with ethyl acetate (×1) and the combined organic extracts were washed with water (×1), brine (×1), dried over anhydrous sodium sulfate and concentrated. The crude material was purified by column chromatography on silica gel (gradient of 0-100% ethyl acetate in hexanes) to afford the title compound as a pale-yellow foam (187 mg).

¹H NMR (CDCl₃) δ 7.98-7.97 (m, 1H), 7.89-7.87 (m, 1H), 7.52-7.49 (m, 1H), 7.37-7.34 (m, 1H), 7.09-6.90 (m, 2H), 6.63-6.59 (m, 1H), 4.55-4.49 (m, 1H), 4.29-4.20 (m, 1H), 3.40 (dd, 1H), 1.45 (d, 3H), 1.35-1.33 (m, 12H).

### Step C: Preparation of [(2S)-8-Chloro-2,3-dihydro-2-methyl-4H-1,4-benzoxazin-4-yl][3-(2-methyl-2H-1,2,3-triazol-4-yl)phenyl]methanone

A microwave vial was charged with [(2S)-8-chloro-2-methyl-2,3-dihydro-1,4-benzoxazin-4-yl]-[3-(4,4,5,5-tetramethyl-1,3,2-dioxaborolan-2-yl)phenyl]methanone (i.e. the product of Step B) (174 mg, 0.42 mmol), 4-bromo-2-methyl-1,2,3-triazole (136 mg, 0.84 mmol), sodium carbonate (147 mg, 1.39 mmol), 1,2-dimethoxyethane (4 mL) and water (1.4 mL) then the mixture was sparged with nitrogen gas for 5 min. Tetrakis(triphenylphosphine)palladium(0) (48 mg, 0.042 mmol) was added then the vial was sealed and stirred for 25 min at 140° C. in a microwave. The mixture was cooled to room temperature, diluted with dichloromethane and washed with saturated aqueous sodium bicarbonate solution (×2), brine (×1), dried over anhydrous sodium sulfate and concentrated. The crude material was purified by column chromatography on silica gel (gradient of 0-100% ethyl acetate in hexanes) to afford the title compound as a pale yellow foam (107 mg).

¹H NMR (CDCl₃) δ 7.94-7.93 (m, 1H), 7.87-7.85 (m, 1H), 7.80 (s, 1H), 7.44-7.39 (m, 2H), 7.11-6.87 (m, 2H), 6.62-6.59 (m, 1H), 4.59-4.53 (m, 1H), 4.34-4.28 (m, 1H), 4.24 (s, 3H), 3.44 (dd, 1H), 1.48 (d, 3H).

### Synthesis Example 10

### Preparation of 4-[3-[[(2S)-2,3-Dihydro-2,8-dimethyl-4H-1,4-benzoxazin-4-yl]carbonyl]phenyl]-2,4-dihydro-3H-1,2,4-triazol-3-one (Compound 122)

### Step A: Preparation of 3-(5-oxo-1H-1,2,4-triazol-4-yl)benzoic acid

To a stirred solution of methyl 3-aminobenzoate (2 g, 13 mmol) in methanol (20 mL) was added trimethyl orthoformate (1.4 mL, 12.6 mmol), methyl carbazate (1.13 g, 12.6 mmol), and p-toluenesulfonic acid monohydrate (50 mg, 0.26 mmol) then the mixture was stirred at 65° C. for 48 h. Sodium methoxide (25 wt. % in methanol, 8.58 g, 39.7 mmol) was added and the mixture was stirred at 50° C. overnight then cooled to room temperature and diluted with water. 1 N aqueous sodium hydroxide solution was added and the mixture was washed with diethyl ether (×3). The aqueous phase was acidified with 1 N aqueous hydrochloric acid solution to pH~2 and extracted with ethyl acetate (×2) then the combined organic extracts were washed with water (×1), brine (×1), dried over anhydrous magnesium sulfate and concentrated to afford the title compound as a pale pink solid (396 mg), which was used without further purification.

¹H NMR (CDCl₃) δ 8.49-8.26 (m, 1H), 8.06 (s, 1H), 7.94 (m, 1H), 7.73-7.70 (m, 1H), 7.54-7.52 (m, 1H), 7.14-7.10 (m, 1H).

### Step B: Preparation of 4-[3-[[(2S)-2,3-Dihydro-2,8-dimethyl-4H-1,4-benzoxazin-4-yl]carbonyl]phenyl]-2,4-dihydro-3H-1,2,4-triazol-3-one

To a stirred solution of (2S)-2,8-dimethyl-3,4-dihydro-2H-1,4-benzoxazine (i.e. the product of Step C in Synthesis Example 3) (315 mg, 1.9 mmol), 3-(5-oxo-1H-1,2,4-triazol-4-yl)benzoic acid (i.e. the product of Step A) (396 mg, 1.9 mmol) and triethylamine (0.81 mL, 5.8 mmol) in ethyl acetate (10 mL) was added propylphosphonic anhydride (50 wt. % in ethyl acetate, 3.07 g, 4.8 mmol). The mixture was stirred at reflux overnight then was cooled to room temperature and diluted with ethyl acetate. The mixture was washed with 1 N aqueous hydrochloric acid solution (×1), 1 N aqueous sodium hydroxide solution (×1), water (×1), brine (×1), dried over anhydrous magnesium sulfate and concentrated. The crude material was purified by column chromatography on silica gel (gradient of 0-40% ethyl acetate in hexanes) to afford the title compound as a white solid (228 mg).

¹H NMR (CDCl₃) δ 9.27 (br s, 1H), 7.75-7.73 (m, 1H), 7.63-7.60 (m, 2H), 7.49-7.45 (m, 2H), 6.90-6.88 (m, 1H), 6.83-6.41 (m, 2H), 4.54-4.46 (m, 1H), 4.42-4.30 (m, 1H), 3.37 (dd, 1H), 2.23 (s, 3H), 1.44 (d, 3H).

### Synthesis Example 11

### Preparation of 4-[3-[[(2S)-2,3-Dihydro-2,8-dimethyl-4H-1,4-benzoxazin-4-yl]carbonyl]phenyl]-2,4-dihydro-2-methyl-3H-1,2,4-triazol-3-one (Compound 117)

To a stirred solution of 4-[3-[[(2S)-2,3-Dihydro-2,8-dimethyl-4H-1,4-benzoxazin-4-yl]carbonyl]phenyl]-2,4-dihydro-3H-1,2,4-triazol-3-one (i.e. the product of Synthesis Example 10) (80 mg, 0.23 mmol) in N,N-dimethylformamide (4 mL) was added potassium carbonate (63 mg, 0.46 mmol) and iodomethane (0.04 mL, 0.69 mmol). The mixture was stirred at room temperature overnight then water was added and the mixture was extracted with diethyl ether (×2). The combined organic extracts were washed with water (×1), brine (×1), dried over anhydrous magnesium sulfate and concentrated to afford the title compound as a light tan solid (52 mg).

¹H NMR (CDCl₃) δ 7.75-7.73 (m, 1H), 7.61-7.59 (m, 2H), 7.46-7.42 (m, 2H), 6.88-6.86 (m, 1H), 6.81-6.36 (m, 2H), 4.55-4.19 (m, 2H), 3.52 (s, 3H), 3.35 (dd, 1H), 2.21 (s, 3H), 1.42 (d, 3H).

### Synthesis Example 12

### Preparation of [(2S)-8-Chloro-2,3-dihydro-2-methyl-4H-1,4-benzoxazin-4-yl][3-(1H-1,2,4-triazol-1-yl)phenyl]methanone (Compound 18)

To a stirred solution of (2S)-8-chloro-2-methyl-3,4-dihydro-2H-1,4-benzoxazine (i.e. the product of Step C in Synthesis Example 1) (1 g, 5.4 mmol), 3-(1,2,4-triazol-1-yl)benzoic acid (i.e. the product of Step E in Synthesis Example 15) (1.03 g, 5.4 mmol) and triethylamine (1.9 mL, 13.6 mmol) in ethyl acetate (10 mL) was added propylphosphonic anhydride (50 wt. % in ethyl acetate, 5.2 g, 8.2 mmol). The mixture was stirred at 70° C. overnight then 60° C. for 2 d. The mixture was cooled to room temperature, diluted with water and extracted with ethyl acetate. The organic extract was washed with 1 N aqueous hydrochloric acid solution (×1), 1 N aqueous sodium hydroxide solution (×1), water (×1), brine (×1), dried over anhydrous magnesium sulfate and concentrated. The crude material was purified by column chromatography on silica gel (gradient of 0-40% ethyl acetate in hexanes) to afford the title compound as a white solid (1.48 g).

¹H NMR (CDCl₃) δ 8.55 (s, 1H), 8.05 (s, 1H), 7.86-7.85 (m, 1H), 7.78-7.75 (m, 1H), 7.47-7.44 (m, 1H), 7.41-7.38 (m, 1H), 7.07-7.05 (m, 1H), 6.97-6.70 (m, 1H), 6.57-6.54 (m, 1H), 4.55-4.50 (m, 1H), 4.32-4.20 (m, 1H), 3.40 (dd, 1H), 1.43 (d, 3H).

### Synthesis Example 13

### Preparation of [(2S)-8-Chloro-2,3-dihydro-2-methyl-4H-1,4-benzoxazin-4-yl][3-(1H-1,2,4-triazol-1-yl)phenyl]methanethione (Compound 139)

To a stirred solution of [(2S)-8-Chloro-2,3-dihydro-2-methyl-4H-1,4-benzoxazin-4-yl][3-(1H-1,2,4-triazol-1-yl)phenyl]methanone (i.e. the product of Synthesis Example 12) (200 mg, 0.56 mmol) in toluene (5 mL) was added Lawesson's reagent (228 mg, 0.56 mmol). The mixture was stirred at 90° C. overnight then cooled to room temperature, diluted with water, extracted with ethyl acetate (×2) and concentrated. The crude material was purified by column chromatography on silica gel (gradient of 0-50% ethyl acetate in hexanes) to afford the title compound as a yellow solid (200 mg).

¹H NMR (CDCl₃) δ 8.50 (br s, 1H), 8.07 (s, 1H), 7.98-7.71 (m, 1H), 7.65-7.63 (m, 1H), 7.38-7.09 (m, 3H), 6.47-6.44 (m, 2H), 5.44-5.08 (m, 1H), 4.76-4.70 (m, 1H), 4.04-3.67 (m, 1H), 1.58 (d, 3H).

### Synthesis Example 14

### Preparation of [8-Chloro-2,3-dihydro-2-(methoxymethyl)-4H-1,4-benzoxazin-4-yl][2-(1H-1,2,4-triazol-1-yl)-4-pyridinyl]methanone (Compound 35)

### Step A: Preparation of N-(3-chloro-2-fluorophenyl)-4-methylbenzenesulfonamide

To a stirred solution of 3-chloro-2-fluoroaniline (50 g, 345 mmol) in pyridine (300 mL) at 0° C. was added p-toluenesulfonyl chloride (72 g, 379 mmol) and the mixture was stirred at room temperature for 16 h. Ice-cooled water (1000 mL) was added and the mixture was extracted with ethyl acetate (1000 ml×2). The combined organic extracts were washed with brine, dried over anhydrous sodium sulfate and concentrated. The crude material was purified by trituration with n-pentane to afford the title compound as an off-white solid (55 g).

MS (ES⁺) m/z Found: (M−H)⁻, C₁₃H₁₁ClFNO₂S, 298, requires 298.0.

### Step B: Preparation of N-(3-chloro-2-fluoro-phenyl)-N-(2,3-dihydroxypropyl)-4-methyl-benzenesulfonamide

To a stirred mixture of N-(3-chloro-2-fluorophenyl)-4-methylbenzenesulfonamide (i.e. the product of Step A) (50 g, 167 mmol) and glycidol (13.6 g, 184 mmol) was added potassium carbonate (2.6 g, 17 mmol) and benzyltriethylammonium chloride (3.7 g, 17 mmol) then the mixture was stirred neat at 90° C. for 16 h. Ice-cooled water (1000 mL) was added and the mixture was extracted with ethyl acetate (1000 mL×2). The combined organic extracts were washed with brine, dried over anhydrous sodium sulfate and concentrated. The crude material was purified by column chromatography on silica gel (20% ethyl acetate in hexanes) to afford the title compound as a colorless liquid (52 g).

MS (ES⁺) m/z Found: (M+H)⁺, C₁₆H₁₇ClFNO₄S, 374, requires 374.1.

### Step C: Preparation of N-[3-[tert-butyl(dimethyl)silyl]oxy-2-hydroxy-propyl]-N-(3-chloro-2-fluoro-phenyl)-4-methyl-benzenesulfonamide

To a stirred solution of N-(3-chloro-2-fluoro-phenyl)-N-(2,3-dihydroxypropyl)-4-methyl-benzenesulfonamide (i.e. the product of Step B) (52 g, 139 mmol) in dichloromethane (500 mL) at 0° C. was added imidazole (10.4 g, 153 mmol) and tert-butyldimethylsilyl chloride (23.0 g, 153 mmol). The mixture was stirred at room temperature for 16 h then ice-cooled water (1000 mL) was added and the mixture was extracted with ethyl acetate (1000 mL×2). The combined organic extracts were washed with brine, dried over anhydrous sodium sulfate and concentrated. The crude material was purified by column chromatography on silica gel (10% ethyl acetate in hexanes) to afford the title compound as a colorless liquid (50 g).

MS (ES⁺) m/z Found: (M+H)⁺, C₂₂H₃₁ClFNO₄SSi, 488, requires 488.1.

### Step D: Preparation of [8-chloro-4-(p-tolylsulfonyl)-2,3-dihydro-1,4-benzoxazin-2-yl]methanol

To a stirred solution of N-[3-[tert-butyl(dimethyl)silyl]oxy-2-hydroxy-propyl]-N-(3-chloro-2-fluoro-phenyl)-4-methyl-benzenesulfonamide (i.e. the product of Step C) (20 g, 41 mmol) in tetrahydrofuran (20 mL) was added sodium hydroxide (6.5 g, 164 mmol) and tetrabutylammonium bromide (1.32 g, 4.10 mmol) then the mixture was stirred at 70° C. for 1 h. Ice-cooled water (500 mL) was added and the mixture was extracted with ethyl acetate (500 mL×2). The combined organic extracts were washed with brine, dried over anhydrous sodium sulfate and concentrated. The crude material was purified by column chromatography on silica gel (10% ethyl acetate in hexanes) to afford the title compound as an off-white solid (4.5 g). In addition, tert-butyl-[[8-chloro-4-(p-tolylsulfonyl)-2,3-dihydro-1,4-benzoxazin-2-yl]methoxy]-dimethyl-silane (5 g) was also isolated.

MS (ES⁺) m/z Found: (M+H)⁺, C₁₆H₁₆ClNO₄S, 354, requires 354.1.

### Step E: Preparation of 8-chloro-2-(methoxymethyl)-4-(p-tolylsulfonyl)-2,3-dihydro-1,4-benzoxazine

To a stirred solution of [8-chloro-4-(p-tolylsulfonyl)-2,3-dihydro-1,4-benzoxazin-2-yl]methanol (i.e. the product of Step D) (2 g, 5.7 mmol) in tetrahydrofuran (20 mL) at 0° C. was added sodium hydride (60% dispersion in mineral oil, 0.26 g, 6.5 mmol). The mixture was stirred at this temperature for 15 min then iodomethane (1.2 g, 8.5 mmol) was added. The mixture was stirred at room temperature for 3 h then ice-cooled water (100 mL) was added. The mixture was extracted with ethyl acetate (100 mL×2) and the combined organic extracts were washed with brine, dried over anhydrous sodium sulfate and concentrated. The crude material was purified by column chromatography on silica gel (10% ethyl acetate in hexanes) to afford the title compound as an off-white solid (1 g).

MS (ES⁺) m/z Found: (M+H)⁺, C₁₇H₁₈ClNO₄S, 368, requires 368.1.

### Step F: Preparation of 8-chloro-2-(methoxymethyl)-3,4-dihydro-2H-1,4-benzoxazine

To a stirred solution of 8-chloro-2-(methoxymethyl)-4-(p-tolylsulfonyl)-2,3-dihydro-1,4-benzoxazine (i.e. the product of Step E) (1 g, 2.7 mmol) in dichloromethane (10 mL) at 0° C. was added sulfuric acid (2.6 mL). The mixture was stirred at room temperature for 16 h then was quenched with solid sodium bicarbonate. Ethyl acetate (50 mL) was added and the mixture was filtered through a pad of Celite, rinsing with ethyl acetate (50 mL). The filtrate was concentrated and the crude material was purified by column chromatography on silica gel (20% ethyl acetate in hexanes) to afford the title compound as a colorless liquid (0.3 g).

MS (ES⁺) m/z Found: (M+H)⁺, C₁₀H₁₂ClNO₂, 214, requires 214.1.

### Step G: Preparation of 2-(1,2,4-triazol-1-yl)pyridine-4-carboxylic acid

To a mixture of ethyl 2-bromoisonicotinate (5 g, 21.7 mmol) and 1,2,4-triazole (1.79 g, 25.9 mmol) in N,N-dimethylformamide (50 mL) was added cesium carbonate (14.1 g, 43.5 mmol) and copper(I) iodide (1.65 g, 8.69 mmol) then the mixture was stirred at 120° C. for 16 h. The mixture was filtered through a pad of Celite, rinsing with N,N-dimethylformamide (50 mL). The filtrate was diluted with water (100 mL) and acidified with 1 N aqueous hydrochloric acid solution. The resulting precipitate was collected by filtration, washed with ice cold water and dried under vacuum to afford the title compound as an off-white solid (3 g).

¹H NMR (DMSO-d₆) δ 14.06 (br s, 1H), 9.44 (s, 1H), 8.74-8.72 (m, 1H), 8.36 (s, 1H), 8.22 (s, 1H), 7.89-7.88 (m, 1H).

### Step H: [8-Chloro-2,3-dihydro-2-(methoxymethyl)-4H-1,4-benzoxazin-4-yl][2-(1H-1,2,4-triazol-1-yl)-4-pyridinyl]methanone

Thionyl chloride (2.4 mL) was added to 2-(1,2,4-triazol-1-yl)pyridine-4-carboxylic acid (i.e. the product of Step G) (0.2 g, 1.05 mmol) and the mixture was stirred at 100° C. for 2 h. The mixture was concentrated then dichloromethane (10 mL) was added and the solution was cooled to 0° C. 8-Chloro-2-(methoxymethyl)-3,4-dihydro-2H-1,4-benzoxazine (i.e. the product of Step F) (0.26 g, 1.22 mmol) and triethylamine (0.31 g 3.06 mmol) were added then the mixture was stirred at room temperature for 16 h. Ice-cooled water (100 mL) was added and the mixture was extracted with dichloromethane (100 mL×2). The combined organic extracts were washed with brine (×1), dried over anhydrous sodium sulfate and concentrated. The crude material was purified by column chromatography on silica gel (50% ethyl acetate in hexanes) to afford the title compound as an off-white solid (80 mg).

¹H NMR (DMSO-d₆, 100° C.) δ 9.28 (s, 1H), 8.61-8.60 (m, 1H), 8.22-8.18 (m, 1H), 7.93 (s, 1H), 7.53-7.52 (m, 1H), 7.30-7.28 (m, 1H), 7.20-7.18 (m, 1H), 6.78-6.74 (m, 1H), 4.69-4.67 (m, 1H), 4.09-4.05 (m, 1H), 3.74-3.58 (m, 3H), 3.32 (s, 3H).

### Synthesis Example 15

### Preparation of (+)-[(2S)-2,3-Dihydro-8-methyl-2-(trifluoromethyl)-4H-1,4-benzoxazin-4-yl][3-(1H-1,2,4-triazol-1-yl)phenyl]methanone (Compound 58) and (−)-[(2R)-2,3-Dihydro-8-methyl-2-(trifluoromethyl)-4H-1,4-benzoxazin-4-yl][3-(1H-1,2,4-triazol-1-yl)phenyl]methanone (Compound 68)

### Step A: Preparation of N-(3-chloro-2-fluoro-phenyl)-4-methyl-N-(3,3,3-trifluoro-2-hydroxy-propyl)benzenesulfonamide

To a stirred mixture of N-(3-chloro-2-fluorophenyl)-4-methylbenzenesulfonamide (i.e. the product of Step A in Synthesis Example 14) (20 g, 67 mmol) and 2-(trifluoromethyl)oxirane (8.2 g, 74 mmol) was added potassium carbonate (0.92 g, 6.7 mmol) and benzyltriethylammonium chloride (1.51 g, 6.7 mmol) then the mixture was stirred neat at 90° C. for 24 h. Ice-cooled water (500 mL) was added and the mixture was extracted with ethyl acetate (500 mL×2). The combined organic extracts were washed with brine, dried over anhydrous sodium sulfate and concentrated. The crude material was purified by column chromatography on silica gel (20% ethyl acetate in hexanes) to afford the title compound as an off-white solid (10 g).

MS (ES⁺) m/z Found: (M+H)⁺, C₁₆H₁₄ClF₄NO₃S, 412, requires 412.0.

### Step B: Preparation of 8-chloro-4-(p-tolylsulfonyl)-2-(trifluoromethyl)-2,3-dihydro-1,4-benzoxazine

To a stirred solution of N-(3-chloro-2-fluoro-phenyl)-4-methyl-N-(3,3,3-trifluoro-2-hydroxy-propyl)benzenesulfonamide (i.e. the product of Step A) (3 g, 7.3 mmol) in tetrahydrofuran (2 mL) was added sodium hydroxide (1.16 g, 29.7 mmol) and tetrabutylammonium bromide (0.22 g, 0.7 mmol) then the mixture was stirred at 70° C. for 1 h. Ice-cooled water (100 mL) was added and the mixture was extracted with ethyl acetate (100 mL×2). The combined organic extracts were washed with brine, dried over anhydrous sodium sulfate and concentrated. The crude material was purified by column chromatography on silica gel (10% ethyl acetate in hexanes) to afford the title compound as an off-white solid (2.2 g).

MS (ES⁺) m/z Found: (M+H)⁺, C₁₆H₁₃ClF₃NO₃S, 392, requires 392.0.

### Step C: Preparation of 8-methyl-4-(p-tolylsulfonyl)-2-(trifluoromethyl)-2,3-dihydro-1,4-benzoxazine

A sealed tube was charged with 8-chloro-4-(p-tolylsulfonyl)-2-(trifluoromethyl)-2,3-dihydro-1,4-benzoxazine (i.e. the product of Step B) (1 g, 2.55 mmol), toluene (5 mL), methylboronic acid (1.5 g, 25 mmol) and potassium phosphate (1.0 g, 4.7 mmol) then the mixture was sparged with argon gas for 10 min. Palladium(II) acetate (57 mg, 0.25 mmol) and SPhos (0.2 g, 0.48 mmol) were added then the tube was sealed and the mixture was stirred at 120° C. for 16 h. This reaction was performed four times then the four reaction mixtures were combined, worked up and purified together. Ice-cooled water was added and the mixture was extracted with ethyl acetate (×2). The combined organic extracts were washed with brine, dried over anhydrous sodium sulfate and concentrated. The crude material was purified by column chromatography on silica gel (5% ethyl acetate in hexanes) to afford the title compound as an off-white solid (1.1 g).

MS (ES⁺) m/z Found: (M+H)⁺, C₁₇H₁₆F₃NO₃S, 372, requires 372.1.

### Step D: Preparation of 8-methyl-2-(trifluoromethyl)-3,4-dihydro-2H-1,4-benzoxazine

To a solution of 8-methyl-4-(p-tolylsulfonyl)-2-(trifluoromethyl)-2,3-dihydro-1,4-benzoxazine (i.e. the product of Step C) (1.5 g, 4.0 mmol) in methanol (10 mL) at 0° C. was added magnesium powder (1 g, 40 mmol) and the mixture was sonicated at room temperature for 2 h. Ethyl acetate (100 mL) was added, the mixture was filtered through a pad of Celite and the filtrate was concentrated. The crude material was purified by column chromatography on silica gel (10% ethyl acetate in hexanes) to afford the title compound as a yellow oil (0.7 g).

MS (ES⁺) m/z Found: (M+H)⁺, C₁₀H₁₀F₃NO, 218, requires 218.1.

### Step E: Preparation of 3-(1,2,4-triazol-1-yl)benzoic acid

To a mixture of ethyl 3-iodobenzoate (20 g, 87 mmol) and 1,2,4-triazole (7.1 g, 103 mmol) in N,N-dimethylformamide (100 mL) was added cesium carbonate (56.5 g, 172 mmol) and copper(I) iodide (6.65 g, 34.8 mmol) then the mixture was stirred at 120° C. for 16 h. The mixture was filtered through a pad of Celite, rinsing with N,N-dimethylformamide (50 mL). The filtrate was diluted with water (100 mL) and acidified with 1 N aqueous hydrochloric acid solution. The resulting precipitate was collected by filtration, washed with ice cold water and dried under vacuum to afford the title compound as an off-white solid (10 g).

MS (ES⁺) m/z Found: (M+H)⁺, C₉H₇N₃O₂, 190, requires 190.1.

### Step F: Preparation of (+)-[(2S)-2,3-Dihydro-8-methyl-2-(trifluoromethyl)-4H-1,4-benzoxazin-4-yl][3-(1H-1,2,4-triazol-1-yl)phenyl]methanone (isomer 1) and (−)-[(2R)-2,3-Dihydro-8-methyl-2-(trifluoromethyl)-4H-1,4-benzoxazin-4-yl][3-(1H-1,2,4-triazol-1-yl)phenyl]methanone (isomer 2)

To a mixture of 8-methyl-2-(trifluoromethyl)-3,4-dihydro-2H-1,4-benzoxazine (i.e. the product of Step D) (0.75 g, 3.45 mmol) and 3-(1,2,4-triazol-1-yl)benzoic acid (i.e. the product of Step E) (0.78 g, 4.14 mmol) in pyridine (20 mL) at 0° C. was added phosphoryl chloride (1.5 g, 10.4 mmol). The mixture was stirred at 0° C. for 2 h then ice-cooled water (200 mL) was added. The mixture was extracted with ethyl acetate (200 mL×2) and the combined organic extracts were washed with brine, dried over anhydrous sodium sulfate and concentrated. The crude material was purified by column chromatography on silica gel (5% ethyl acetate in hexanes) to afford [8-methyl-2-(trifluoromethyl)-2,3-dihydro-1,4-benzoxazin-4-yl]-[3-(1,2,4-triazol-1-yl)phenyl]methanone as a pale yellow solid (0.52 g).

¹H NMR (CDCl₃) δ 8.45 (s, 1H), 8.10 (s, 1H), 7.81-7.79 (m, 2H), 7.48-7.40 (m, 2H), 6.94-6.92 (m, 1H), 6.60-6.56 (m, 1H), 6.44 (br s, 1H), 4.88-4.85 (m, 1H), 4.36-4.32 (m, 1H), 4.20-4.16 (m, 1H), 2.29 (s, 3H).

The enantiomers were separated by chiral supercritical fluid chromatography to afford the title compounds.

Isomer 1: [α]²⁵_{D}=+17.2° (c=0.1% in CHCl₃)

Isomer 2: [α]²⁵_{D}=−13.8° (c=0.1% in CHCl₃)

### Synthesis Example 16

### Preparation of (8-Chloro-2-cyclopropyl-2,3-dihydro-4H-1,4-benzoxazin-4-yl)[3-(1H-1,2,4-triazol-1-yl)phenyl]methanone (Compound 22)

### Step A: Preparation of 8-chloro-2-cyclopropyl-4H-1,4-benzoxazin-3-one

A microwave vial was charged with 2-amino-6-chloro-phenol (0.5 g, 3.5 mmol), ethyl 2-bromo-2-cyclopropyl-acetate (0.6 g, 2.9 mmol), 1,8-diazabicyclo[5.4.0]undec-7-ene (0.49 g, 3.2 mmol) and N-methyl-2-pyrrolidone (8 mL). The vial was sealed and stirred for 4 min at 180° C. in a microwave. The mixture was cooled to room temperature, diluted with ethyl acetate and filtered through a pad of Celite. The filtrate was washed with brine (×2) and concentrated. The crude material was purified by column chromatography on silica gel (gradient of 0-50% ethyl acetate in hexanes) to afford the title compound as an orange solid (0.51 g).

¹H NMR (CDCl₃) δ 8.45 (br s, 1H), 7.06-7.05 (m, 1H), 6.91-6.87 (m, 1H), 6.73-6.71 (m, 1H), 4.20 (d, 1H), 1.33-1.26 (m, 1H), 0.75-0.58 (m, 4H).

### Step B: Preparation of 8-chloro-2-cyclopropyl-3,4-dihydro-2H-1,4-benzoxazine

To a stirred solution of 8-chloro-2-cyclopropyl-4H-1,4-benzoxazin-3-one (i.e. the product of Step A) (0.42 g, 1.9 mmol) in anhydrous tetrahydrofuran (10 mL) at 0° C. was added borane tetrahydrofuran complex (1 M in tetrahydrofuran, 3.2 mL, 3.2 mmol) dropwise. The reaction mixture was stirred at room temperature for 3 h then 50° C. overnight. The mixture was cooled to room temperature and methanol (5 mL) was slowly added. After stirring for 20 min, the mixture was concentrated then dissolved in ethyl acetate and washed with saturated aqueous sodium bicarbonate solution (×1), water (×1), brine (×1), dried over anhydrous magnesium sulfate and concentrated to afford the title compound as a light brown oil (0.37 g), which was used without further purification.

¹H NMR (CDCl₃) δ 6.73-6.71 (m, 1H), 6.66-6.63 (m, 1H), 6.49-6.47 (m, 1H), 3.83 (br s, 1H), 3.52-3.46 (m, 2H), 3.32-3.29 (m, 1H), 1.14-1.08 (m, 1H), 0.72-0.66 (m, 1H), 0.64-0.57 (m, 2H), 0.43-0.37 (m, 1H).

### Step C: Preparation of (8-Chloro-2-cyclopropyl-2,3-dihydro-4H-1,4-benzoxazin-4-yl)[3-(1H-1,2,4-triazol-1-yl)phenyl]methanone

To a stirred solution of 8-chloro-2-cyclopropyl-3,4-dihydro-2H-1,4-benzoxazine (i.e. the product of Step B) (0.11 g, 0.52 mmol), 3-(1,2,4-triazol-1-yl)benzoic acid i.e. the product of Step E in Synthesis Example 15) (0.12 g, 0.63 mmol) and triethylamine (0.22 mL, 1.6 mmol) in dichloromethane (8 mL) was added propylphosphonic anhydride (50 wt. % in ethyl acetate, 0.57 g, 0.90 mmol). The mixture was stirred at 40° C. overnight then was cooled to room temperature and concentrated. The crude material was purified by column chromatography on silica gel (gradient of 0-70% ethyl acetate in hexanes). The isolated material was further purified by column chromatography on silica gel (gradient of 0-20% ethyl acetate in dichloromethane) to afford the title compound as a colorless oil (39 mg).

¹H NMR (CDCl₃) δ 8.53 (s, 1H), 8.10 (s, 1H), 7.87-7.86 (m, 1H), 7.80-7.78 (m, 1H), 7.51-7.48 (m, 1H), 7.44-7.42 (m, 1H), 7.11-7.10 (m, 1H), 6.82 (br s, 1H), 6.60-6.57 (m, 1H), 4.40-4.38 (m, 1H), 3.87-3.83 (m, 1H), 3.60 (dd, 1H), 1.13-1.06 (m, 1H), 0.74-0.68 (m, 1H), 0.66-0.58 (m, 2H), 0.47-0.41 (m, 1H).

### Synthesis Example 17

### Preparation of [(2R)-8-chloro-2-(ethoxymethyl)-2,3-dihydro-1,4-benzoxazin-4-yl]-[5-(3-isopropyl-1,2,4-triazol-1-yl)-2-methyl-phenyl]methanone (Compound 340)

### Step A: Preparation of [(2R)-8-chloro-3,4-dihydro-2H-1,4-benzoxazin-2-yl]methanol

To a stirred solution of 2-amino-6-chloro-phenol (10 g, 70 mmol) in water (100 mL) was added (S)-(+)-epichlorohydrin (7.7 g, 83 mmol) and sodium hydroxide (3.9 g, 98 mmol). The reaction mixture was stirred at room temperature for 20 min then was diluted with water, extracted with ethyl acetate (×2) and the combined organic extracts were dried over anhydrous sodium sulfate and concentrated. The crude material was purified by column chromatography on silica gel (25% ethyl acetate in hexanes) to afford the title compound as a colorless liquid (7 g).

¹H NMR (CDCl₃) δ 6.74 (dd, 1H), 6.70-6.66 (m, 1H), 6.50 (dd, 1H), 4.32-4.28 (m, 1H), 3.91-3.82 (m, 2H), 3.42-3.39 (m, 1H), 3.35-3.31 (m, 1H).

MS (ES⁺) m/z Found: (M+H)⁺, C₉H₁₀ClNO₂, 200, requires 200.0.

### Step B: Preparation of (2R)-8-chloro-2-(ethoxymethyl)-3,4-dihydro-2H-1,4-benzoxazine

To a stirred solution of [(2R)-8-chloro-3,4-dihydro-2H-1,4-benzoxazin-2-yl]methanol (i.e. the product of Step A) (2 g, 10 mmol) in anhydrous tetrahydrofuran (20 mL) at 0° C. was added sodium hydride (60% dispersion in mineral oil, 0.8 g, 20 mmol) and iodoethane (2.3 g, 15 mmol). The reaction mixture was stirred at room temperature for 1 h then was diluted with water, extracted with ethyl acetate (×2) and the combined organic extracts were dried over anhydrous sodium sulfate and concentrated. The crude material was purified by column chromatography on silica gel (15% ethyl acetate in hexanes) to afford the title compound as a colorless liquid (1.5 g).

¹H NMR (CDCl₃) δ 6.72 (dd, 1H), 6.68-6.65 (m, 1H), 6.49 (dd, 1H), 4.39-4.35 (m, 1H), 3.75 (dd, 1H), 3.66-3.55 (m, 3H), 3.48 (dd, 1H), 3.28 (dd, 1H), 1.22 (t, 3H).

MS (ES⁺) m/z Found: (M+H)⁺, C₁₁H₁₄ClNO₂, 228, requires 228.1.

### Step C: Preparation of methyl 5-(3-isopropyl-1,2,4-triazol-1-yl)-2-methyl-benzoate

A microwave vial was charged with methyl 5-bromo-2-methyl-benzoate (2.5 g, 11 mmol), anhydrous N,N-dimethylformamide (15 mL) and 3-isopropyl-1H-1,2,4-triazole (1.83 g, 16.5 mmol), then the mixture was sparged with nitrogen gas for 10 min. Potassium carbonate (4.55 g, 33 mmol), copper(I) iodide (418 mg, 2.2 mmol) and trans-N,N′-dimethylcyclohexane-1,2-diamine (0.86 ml, 5.5 mmol) were added then the mixture was stirred for 3 h at 110° C. in a microwave. The mixture was cooled to room temperature, diluted with water and extracted with ethyl acetate (×2). The combined organic extracts were filtered through a pad of Celite, rinsing with ethyl acetate (25 mL), then the filtrate was washed with brine (×1) and concentrated. The crude material was purified by column chromatography on silica gel (20% ethyl acetate in hexanes) to afford the title compound as an off-white solid (2.4 g).

MS (ES⁺) m/z Found: (M+H)⁺, C₁₄H₁₇N₃O₂, 260, requires 260.1.

### Step D: Preparation of 5-(3-isopropyl-1,2,4-triazol-1-yl)-2-methyl-benzoic acid

To a stirred solution of methyl 5-(3-isopropyl-1,2,4-triazol-1-yl)-2-methyl-benzoate (i.e. the product of Step C) (2.4 g, 9.3 mmol) in tetrahydrofuran (7 mL) and water (3 mL) was added lithium hydroxide monohydrate (583 mg, 13.9 mmol). The reaction mixture was stirred at room temperature for 16 h. The mixture was concentrated then diluted with water (20 mL) and acidified with citric acid. The resulting precipitate was collected by filtration and dried under vacuum to afford the title compound as an off-white solid (1.6 g).

MS (ES⁺) m/z Found: (M+H)⁺, C₁₃H₁₅N₃O₂, 246, requires 246.1.

### Step E: Preparation of [(2R)-8-chloro-2-(ethoxymethyl)-2,3-dihydro-1,4-benzoxazin-4-yl]-[5-(3-isopropyl-1,2,4-triazol-1-yl)-2-methyl-phenyl]methanone

To a stirred solution of 5-(3-isopropyl-1,2,4-triazol-1-yl)-2-methyl-benzoic acid (i.e. the product of Step D) (600 mg, 2.4 mmol) in pyridine (6 mL) at 0° C. was added (2R)-8-chloro-2-(ethoxymethyl)-3,4-dihydro-2H-1,4-benzoxazine (i.e. the product of Step B) (555 mg, 2.4 mmol) and phosphoryl chloride (0.68 mL, 7.3 mmol). The reaction mixture was stirred at room temperature for 16 h then was concentrated. The crude material was purified by column chromatography on silica gel (25% ethyl acetate in hexanes) to afford the title compound as a brown foam (232 mg).

¹H NMR (DMSO-d₆, 90° C.) δ 8.96 (s, 1H), 7.79-7.76 (m, 2H), 7.43-7.41 (m, 2H), 7.18-7.16 (m, 1H), 6.79-6.76 (m, 1H), 4.59-4.57 (m, 1H), 4.09-4.06 (m, 1H), 3.67-3.58 (m, 2H), 3.54-3.47 (m, 3H), 3.07-3.01 (m, 1H), 2.28 (s, 3H), 1.29 (d, 6H), 1.03 (t, 3H).

MS (ES⁺) m/z Found: (M+H)⁺, C₂₄H₂₇ClN₄O₃, 455.3, requires 455.2.

### Synthesis Example 18

### Preparation of [(2S)-8-chloro-2-methyl-2,3-dihydro-1,4-benzoxazin-4-yl]-[5-(3-isopropyl-1,2,4-triazol-1-yl)-2,3-dimethoxy-phenyl]methanone (Compound 194)

### Step A: Preparation of (5-bromo-2,3-dimethoxy-phenyl)-[(2S)-8-chloro-2-methyl-2,3-dihydro-1,4-benzoxazin-4-yl]methanone

To a stirred solution of 5-bromo-2,3-dimethoxy-benzoic acid (1 g, 3.8 mmol) and a few drops of N,N-dimethylformamide in chloroform (10 mL) at 0° C. was added thionyl chloride (1.3 mL, 18 mmol). The reaction mixture was stirred at 60° C. for 3 h then was cooled to room temperature and concentrated. The crude acid chloride was then dissolved in dichloromethane (10 mL) and was added to a solution of (2S)-8-chloro-2-methyl-3,4-dihydro-2H-1,4-benzoxazine (i.e. the product of Step C in Synthesis Example 1) (697 mg, 3.8 mmol) and pyridine (1 mL, 12 mmol) in dichloromethane (10 mL) at 0° C. The mixture was stirred at room temperature for 16 h then was diluted with water, extracted with dichloromethane (×2) and the combined organic extracts were dried over anhydrous sodium sulfate and concentrated. The crude material was purified by column chromatography on silica gel (30% ethyl acetate in hexanes) to afford the title compound as a colorless liquid (1.3 g).

MS (ES⁺) m/z Found: (M+H)⁺, C₁₈H₁₇BrClNO₄, 426.1, requires 426.0.

### Step B: Preparation of [(2S)-8-chloro-2-methyl-2,3-dihydro-1,4-benzoxazin-4-yl]-[5-(3-isopropyl-1,2,4-triazol-1-yl)-2,3-dimethoxy-phenyl]methanone

A solution of (5-bromo-2,3-dimethoxy-phenyl)-[(2S)-8-chloro-2-methyl-2,3-dihydro-1,4-benzoxazin-4-yl]methanone (i.e. the product of Step A) (400 mg, 0.94 mmol) in N,N-dimethylformamide (4 mL) was sparged with nitrogen then potassium carbonate (389 mg, 2.8 mmol), copper(I) iodide (89 mg, 0.47 mmol) and trans-N,N′-dimethylcyclohexane-1,2-diamine (0.074 ml, 0.47 mmol) were added. The reaction mixture was stirred at 120° C. for 16 h in a sealed tube. The mixture was cooled to room temperature, diluted with water, extracted with ethyl acetate (×2) and the combined organic extracts were dried over anhydrous sodium sulfate and concentrated. The crude material was purified by column chromatography on silica gel (35% ethyl acetate in hexanes) to afford the title compound as an off-white semi-solid (270 mg).

¹H NMR (DMSO-d₆, 90° C.) δ 8.99 (s, 1H), 7.56-7.38 (m, 3H), 7.16-7.14 (m, 1H), 6.74 (m, 1H), 4.52 (m, 1H), 4.05 (m, 1H), 3.91 (s, 3H), 3.67 (m, 3H), 3.41-3.37 (m, 1H), 3.07-3.03 (m, 1H), 1.36 (d, 3H), 1.30 (d, 6H).

MS (ES⁺) m/z Found: (M+H)⁺, C₂₃H₂₅ClN₄O₄, 457.27, requires 457.16.

### Synthesis Example 19

### Preparation of [(2S)-8-chloro-2-methyl-2,3-dihydro-1,4-benzoxazin-4-yl]-[2,3-dihydroxy-5-(3-isopropyl-1,2,4-triazol-1-yl)phenyl]methanone (Compound 272)

To a stirred solution of [(2S)-8-chloro-2-methyl-2,3-dihydro-1,4-benzoxazin-4-yl]-[5-(3-isopropyl-1,2,4-triazol-1-yl)-2,3-dimethoxy-phenyl]methanone (i.e. the product of Synthesis Example 18) (3 g, 6.6 mmol) in dichloromethane (60 mL) at −30° C. was added boron tribromide (1 M in dichloromethane, 39.5 ml, 39.5 mmol) dropwise. The mixture was stirred at room temperature for 1 h then was cooled to 0° C. and methanol (100 mL) was added. The mixture was concentrated then diluted with water (25 mL), extracted with ethyl acetate (×2) and the combined organic extracts were dried over anhydrous sodium sulfate and concentrated. The crude material was purified by trituration with n-pentane to afford the title compound as a brown solid (2.55 g).

¹H NMR (DMSO-d₆, 90° C.) δ 8.83 (m, 1H), 7.51-7.49 (m, 1H), 7.30-7.29 (m, 1H), 7.18 (m, 1H), 7.13-7.11 (m, 1H), 6.76-6.71 (m, 1H), 4.55-4.51 (m, 1H), 4.07-4.03 (m, 1H), 3.40-3.35 (m, 1H), 3.05-3.02 (m, 1H), 1.35-1.25 (m, 9H).

MS (ES⁺) m/z Found: (M+H)⁺, C₂₁H₂₁ClN₄O₄, 429.09, requires 429.13.

### Synthesis Example 20

### Preparation of [(2S)-8-chloro-2-methyl-2,3-dihydro-1,4-benzoxazin-4-yl]-[6-(3-isopropyl-1,2,4-triazol-1-yl)-1,3-benzodioxol-4-yl]methanone (Compound 258)

To a stirred solution of [(2S)-8-chloro-2-methyl-2,3-dihydro-1,4-benzoxazin-4-yl]-[2,3-dihydroxy-5-(3-isopropyl-1,2,4-triazol-1-yl)phenyl]methanone (i.e. the product of Synthesis Example 19) (300 mg, 0.7 mmol) in anhydrous N,N-dimethylformamide (5 mL) was added cesium carbonate (1.8 g, 5.5 mmol) and bromochloromethane (0.45 g, 3.5 mmol). The mixture was stirred at 90° C. for 6 h then was cooled to room temperature and ice-cooled water (10 mL) was added. The solid material was collected by filtration and dried to provide the crude product. Purification by column chromatography on silica gel (30% ethyl acetate in hexanes) afforded the title compound as a white solid (100 mg).

¹H NMR (CDCl₃) δ 8.36 (s, 1H), 7.34 (d, 1H), 7.23 (d, 1H), 7.10-7.08 (m, 1H), 7.02-6.40 (m, 2H), 5.92-5.40 (m, 2H), 4.63-4.13 (m, 2H), 3.55-3.30 (m, 1H), 3.16-3.08 (m, 1H), 1.48-1.43 (m, 3H), 1.36 (d, 6H).

MS (ES⁺) m/z Found: (M+H)⁺, C₂₂H₂₁ClN₄O₄, 441.21, requires 441.13.

### Synthesis Example 21

### Preparation of [(2S)-8-chloro-2-methyl-2,3-dihydro-1,4-benzoxazin-4-yl]-[7-(3-isopropyl-1,2,4-triazol-1-yl)-2,3-dihydro-1,4-benzodioxin-5-yl]methanone (Compound 217)

A microwave vial was charged with [(2S)-8-chloro-2-methyl-2,3-dihydro-1,4-benzoxazin-4-yl]-[2,3-dihydroxy-5-(3-isopropyl-1,2,4-triazol-1-yl)phenyl]methanone (i.e. the product of Synthesis Example 19) (400 mg, 0.93 mmol), acetone (8 mL), cesium carbonate (2.4 g, 7.4 mmol) and dibromoethane (0.8 mL, 9.3 mmol). The mixture was stirred for 30 min at 90° C. in a microwave. The mixture was cooled to room temperature and the solids were removed by filtration, rinsing with acetone (10 mL). The filtrate was concentrated and the resulting crude material was purified by preparative HPLC to afford the title compound as a pale brown solid (170 mg).

¹H NMR (DMSO-d₆, 90° C.) δ 8.93 (s, 1H), 7.41-7.14 (m, 4H), 6.74-6.70 (m, 1H), 4.53 (m, 1H), 4.22-3.95 (m, 5H), 3.47-3.40 (m, 1H), 3.07-2.98 (m, 1H), 1.37 (d, 3H), 1.29 (d, 6H).

MS (ES⁺) m/z Found: (M+H)⁺, C₂₃H₂₃ClN₄O₄, 455.27, requires 455.15

By the procedures described herein together with methods known in the art, the following compounds of Tables 1 to 236 can be prepared. The following abbreviations are used in the Tables which follow: t means tertiary, s means secondary, n means normal, i means iso, c means cyclo, Me means methyl, Et means ethyl, Pr means propyl, Bu means butyl, i-Pro means isopropyl, c-Pro means cyclopropyl, t-Bu means tertiary butyl, Ph means phenyl, OMe means methoxy, OEt means ethoxy, SMe means methylthio, —CN means cyano, —NO₂ means nitro, TMS means trimethylsilyl, SOMe means methylsulfinyl, C₂F₅ means CF₂CF₃ and SO₂Me means methylsulfonyl.

TABLE 1

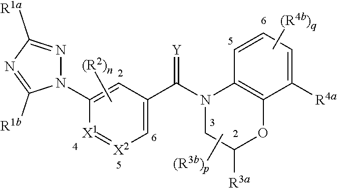

R^{1b} is H, X¹ is CH, X² is CH, (R²)ₙ is 6-OMe, Y is O, (R^{3b})ₚ is H,
R^{3a} is Me, R^{4a} is Cl and (R^{4b})_{q} is H.
R^{1a}
H
CH₂F
OMe
CN
CH₂CHF₂
J-9
i-Pro
CH(Me)CH₂F
J-13
CH₂CH═CH₂
C≡CCF₂H
CH₂SMe
CH(Me)C≡CH
J-7
SO₂Me
CH₂c-Pr
CH(Me)OMe
SO₂CF₃
NHMe
CO₂Me
CH₂OH
CH(Me)OH
C(Me)₂OH
J-22
CH(CF₃)OH
C(Me)(CF₃)OH
J-23
J-24
J-4
CH₂OCF₃
Br
CH₂CH₂F
OCH₂C≡CH
n-Pr
CFMe₂
J-12
C(Me)═CH₂
CH═CF₃
SMe
CH₂C≡CH
J-6
SOMe
c-Bu
CH₂OEt
SOCF₃
J-3
CH₂COMe
COMe
J-25
CF₂OMe
CH(CF₃)OMe
C(Me)(CF₃)OMe
CF₂CH₂OMe
CH₂CF₂OMe
J-26
J-27
CH₂OCHF₂
Cl
CF₃
OCH₂CH═CH₂
Et
CF₃Me
J-11
CH═CH₂
CH(Me)CF₃
OCF₃
C≡CMe
J-5
C(Me)₂SMe
c-Pr
CH₂OMe
SCF₃
J-2
CH₂CH₂OMe
TMS
CH₂CO₂Me
J-28
J-29
CH₂SO₂Me
CH(Me)SO₂Me
C(Me)₂SO₂Me
CH₂CH₂SO₂Me
CH₂SCF₃
CH(Me)SCF₃
F
CHF₂
OEt
Me
CH₂CF₃
J-10
t-Bu
CH(Me)CHF₂
OCHF₂
C≡CH
C≡CCF₃
CH(Me)SMe
C(Me)₂C≡CH
J-8
SCHF₂
J-1
C(Me)₂OMe
CH₂CN
NMe₂
C(Me)₂SCF₃
CH₂SO₂CF₃
CH(Me)SO₂CF₃
C(Me)₂SO₂CF₃
J-30
J-31
J-32
J-33

Some J groups in the tables are defined as below.

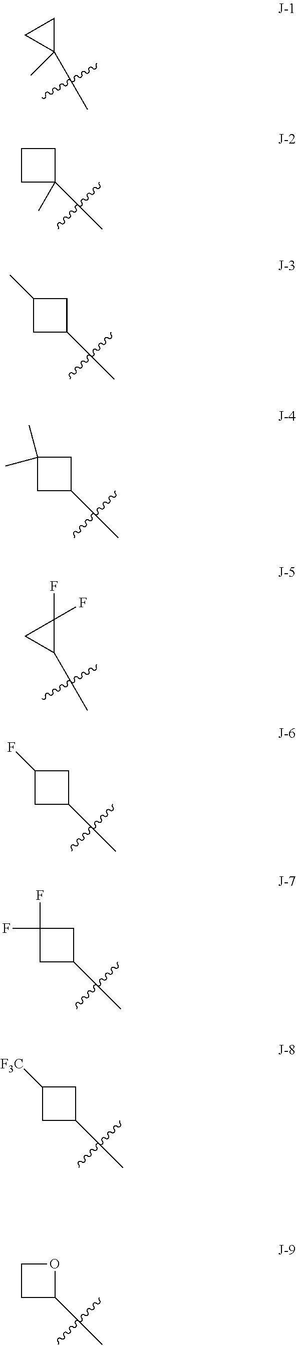

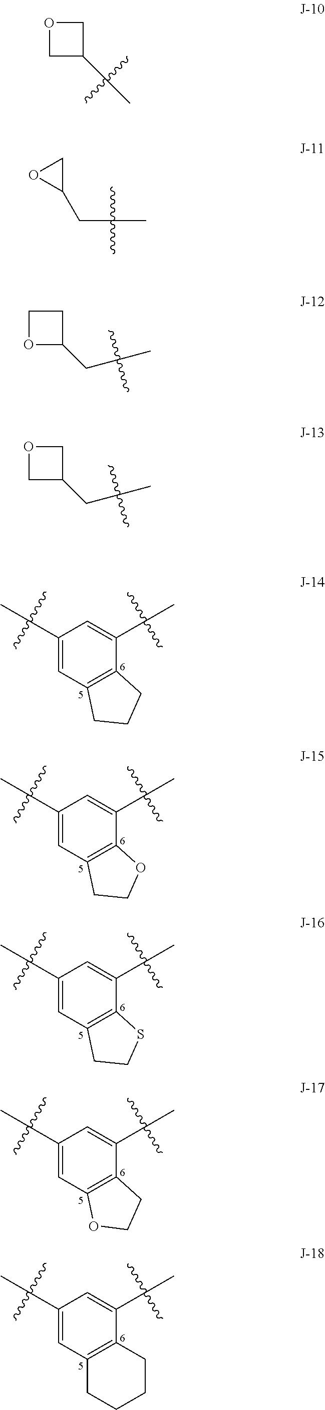

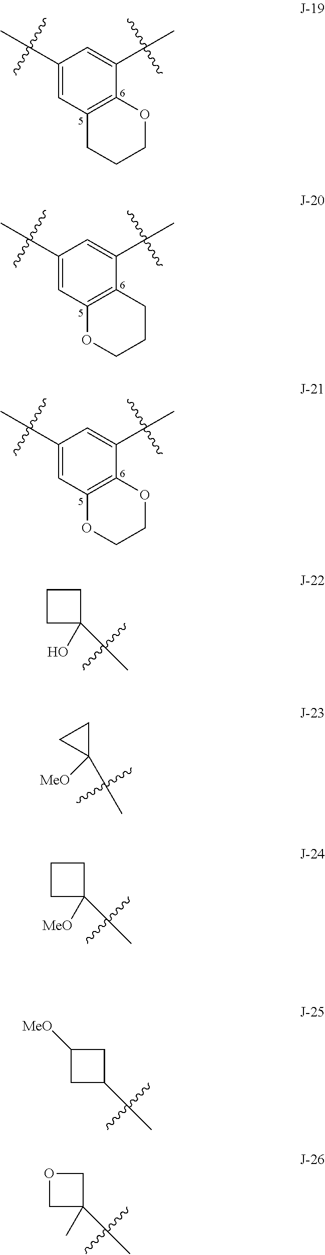

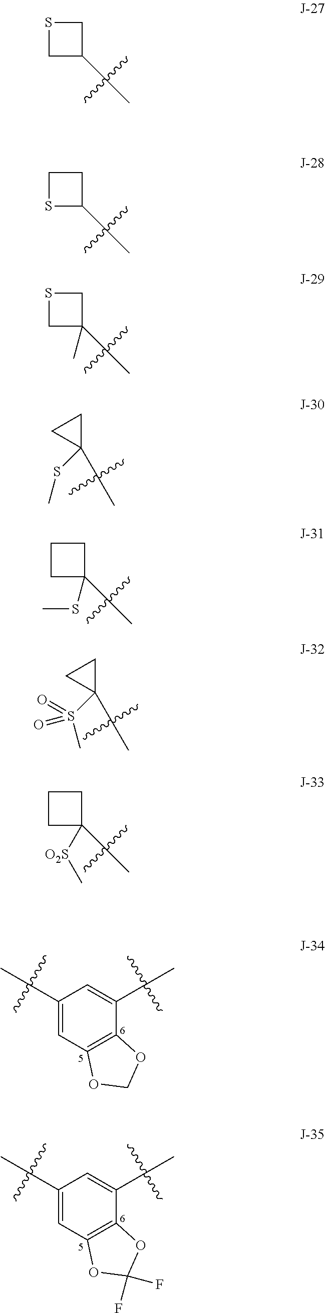

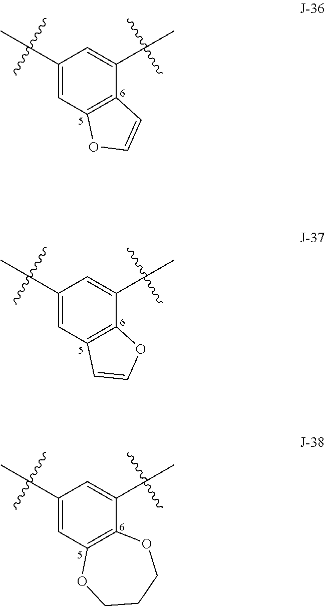 [SMILES: *c1cc(*)c2ccoc2c1 ; *c1cc(*)c2occc2c1 ; *c1cc(*)c2c(c1)OCCCO2]

Table 2 is constructed in the same manner except that the Row Heading “R^{1b} is H, X¹ is CH, X² is CH, (R²)ₙ is 6-OMe, Y is O, (R^{3b})ₚ is H, R^{3a} is Me, R^{4a} is Cl and (R^{4b})_{q} is H.” is replaced with the Row Heading listed for Table 2 below (i.e. “R^{1b} is H, X¹ is CH, X² is CH, (R²)ₙ is H, Y is O, (R^{3b})ₚ is H, R^{3a} is Me, R^{4a} is Cl and (R^{4b})_{q} is H.”). Therefore the first entry in Table 2 is a compound of Formula 1 wherein R^{1b} is H, X¹ is CH, X² is CH, (R²)ₙ is H, Y is O, (R^{3b})ₚ is H, R^{3a} is Me, R^{4a} is Cl, (R^{4b})_{q} is H and R^{1a} is H. Tables 3 through 230 are constructed similarly.

| Table# | Row Heading |
|---|---|
| 2 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | H. |
| 3 | R^{1b} is H, X¹ is CH, X² is CH, (R²)ₙ is 6-F, Y is O, (R^{3b})ₚ is H, R^{3a} is Me, R^{4a} is Cl and (R^{4b})_{q} |
| | is H. |
| 4 | R^{1b} is H, X¹ is CH, X² is CH, (R²)ₙ is 6-Cl, Y is O, (R^{3b})ₚ is H, R^{3a} is Me, R^{4a} is Cl and (R^{4b})_{q} |
| | is H. |
| 5 | R^{1b} is H, X¹ is CH, X² is CH, (R²)ₙ is 6-CN, Y is O, (R^{3b})ₚ is H, R^{3a} is Me, R^{4a} is Cl and (R^{4b})_{q} |
| | is H. |
| 6 | R^{1b} is H, X¹ is CH, X² is CH, (R²)ₙ is 6-NO₂, Y is O, (R^{3b})ₚ is H, R^{3a} is Me, R^{4a} is Cl and (R^{4b})_{q} |
| | is H. |
| 7 | R^{1b} is H, X¹ is CH, X² is CH, (R²)ₙ is 6-OH, Y is O, (R^{3b})ₚ is H, R^{3a} is Me, R^{4a} is Cl and (R^{4b})_{q} |
| | is H. |
| 8 | R^{1b} is H, X¹ is CH, X² is CH, (R²)ₙ is 6-Me, Y is O, (R^{3b})ₚ is H, R^{3a} is Me, R^{4a} is Cl and (R^{4b})_{q} |
| | is H. |
| 9 | R^{1b} is H, X¹ is CH, X² is CH, (R²)ₙ is 6-Et, Y is O, (R^{3b})ₚ is H, R^{3a} is Me, R^{4a} is Cl and (R^{4b})_{q} |
| | is H. |
| 10 | R^{1b} is H, X¹ is CH, X² is CH, (R²)ₙ is 6-n-Pr, Y is O, (R^{3b})ₚ is H, R^{3a} is Me, R^{4a} is Cl and (R^{4b})_{q} |
| | is H. |
| 11 | R^{1b} is H, X¹ is CH, X² is CH, (R²)ₙ is 6-i-Pro, Y is O, (R^{3b})ₚ is H, R^{3a} is Me, R^{4a} is Cl and |
| | (R^{4b})_{q} is H. |
| 12 | R^{1b} is H, X¹ is CH, X² is CH, (R²)ₙ is 6-CH═CH₂, Y is O, (R^{3b})ₚ is H, R^{3a} is Me, R^{4a} is Cl and |
| | (R^{4b})_{q} is H. |
| 13 | R^{1b} is H, X¹ is CH, X² is CH, (R²)ₙ is 6-CH₂CH═CH₂, Y is O, (R^{3b})ₚ is H, R^{3a} is Me, R^{4a} is Cl |
| | and (R^{4b})_{q} is H. |
| 14 | R^{1b} is H, X¹ is CH, X² is CH, (R²)ₙ is 6-C≡CH, Y is O, (R^{3b})ₚ is H, R^{3a} is Me, R^{4a} is Cl and |
| | (R^{4b})_{q} is H. |
| 15 | R^{1b} is H, X¹ is CH, X² is CH, (R²)ₙ is 6-CH₂C≡CH, Y is O, (R^{3b})ₚ is H, R^{3a} is Me, R^{4a} is Cl and |
| | (R^{4b})_{q} is H. |
| 16 | R^{1b} is H, X¹ is CH, X² is CH, (R²)ₙ is 6-OCH₂CH═CH₂, Y is O, (R^{3b})ₚ is H, R^{3a} is Me, R^{4a} is |
| | Cl and (R^{4b})_{q} is H. |
| 17 | R^{1b} is H, X¹ is CH, X² is CH, (R²)ₙ is 6-OCH₂C≡CH, Y is O, (R^{3b})ₚ is H, R^{3a} is Me, R^{4a} is Cl |
| | and (R^{4b})_{q} is H. |
| 18 | R^{1b} is H, X¹ is CH, X² is CH, (R²)ₙ is 6-(J-9), Y is O, (R^{3b})ₚ is H, R^{3a} is Me, R^{4a} is Cl and (R^{4b})_{q} |
| | is H. |
| 19 | R^{1b} is H, X¹ is CH, X² is CH, (R²)ₙ is 6-(J-10), Y is O, (R^{3b})ₚ is H, R^{3a} is Me, R^{4a} is Cl and |
| | (R^{4b})_{q} is H. |
| 20 | R^{1b} is H, X¹ is CH, X² is CH, (R²)ₙ is 6-(J-11), Y is O, (R^{3b})ₚ is H, R^{3a} is Me, R^{4a} is Cl and |
| | (R^{4b})_{q} is H. |
| 21 | R^{1b} is H, X¹ is CH, X² is CH, (R²)ₙ is 6-(J-12), Y is O, (R^{3b})ₚ is H, R^{3a} is Me, R^{4a} is Cl and |
| | (R^{4b})_{q} is H. |
| 22 | R^{1b} is H, X¹ is CH, X² is CH, (R²)ₙ is 6-(J-13), Y is O, (R^{3b})ₚ is H, R^{3a} is Me, R^{4a} is Cl and |
| | (R^{4b})_{q} is H. |
| 23 | R^{1b} is H, X¹ is CH, X² is CH, (R²)ₙ is 6-c-Pr, Y is O, (R^{3b})ₚ is H, R^{3a} is Me, R^{4a} is Cl and (R^{4b})_{q} |
| | is H. |
| 24 | R^{1b} is H, X¹ is CH, X² is CH, (R²)ₙ is 6-c-Bu, Y is O, (R^{3b})ₚ is H, R^{3a} is Me, R^{4a} is Cl and (R^{4b})_{q} |
| | is H. |
| 25 | R^{1b} is H, X¹ is CH, X² is CH, (R²)ₙ is 6-CH₂c-Pr, Y is O, (R^{3b})ₚ is H, R^{3a} is Me, R^{4a} is Cl and |
| | (R^{4b})_{q} is H. |
| 26 | R^{1b} is H, X¹ is CH, X² is CH, (R²)ₙ is 6-CH₂c-Bu, Y is O, (R^{3b})ₚ is H, R^{3a} is Me, R^{4a} is Cl and |
| | (R^{4b})_{q} is H. |
| 27 | R^{1b} is H, X¹ is CH, X² is CH, (R²)ₙ is 6-CH₂F, Y is O, (R^{3b})ₚ is H, R^{3a} is Me, R^{4a} is Cl and |
| | (R^{4b})_{q} is H. |
| 28 | R^{1b} is H, X¹ is CH, X² is CH, (R²)ₙ is 6-CHF₂, Y is O, (R^{3b})ₚ is H, R^{3a} is Me, R^{4a} is Cl and |
| | (R^{4b})_{q} is H. |
| 29 | R^{1b} is H, X¹ is CH, X² is CH, (R²)ₙ is 6-CH₂CH₂F, Y is O, (R^{3b})ₚ is H, R^{3a} is Me, R^{4a} is Cl and |
| | (R^{4b})_{q} is H. |
| 30 | R^{1b} is H, X¹ is CH, X² is CH, (R²)ₙ is 6-CH₂CHF₂, Y is O, (R^{3b})ₚ is H, R^{3a} is Me, R^{4a} is Cl and |
| | (R^{4b})_{q} is H. |
| 31 | R^{1b} is H, X¹ is CH, X² is CH, (R²)ₙ is 6-CH₂CF₃, Y is O, (R^{3b})ₚ is H, R^{3a} is Me, R^{4a} is Cl and |
| | (R^{4b})_{q} is H. |
| 32 | R^{1b} is H, X¹ is CH, X² is CH, (R²)ₙ is 6-CF₂Me, Y is O, (R^{3b})ₚ is H, R^{3a} is Me, R^{4a} is Cl and |
| | (R^{4b})_{q} is H. |
| 33 | R^{1b} is H, X¹ is CH, X² is CH, (R²)ₙ is 6-CH(F)Me, Y is O, (R^{3b})ₚ is H, R^{3a} is Me, R^{4a} is Cl and |
| | (R^{4b})_{q} is H. |
| 34 | R^{1b} is H, X¹ is CH, X² is CH, (R²)ₙ is 6-CH═CF², Y is O, (R^{3b})ₚ is H, R^{3a} is Me, R^{4a} is Cl and |
| | (R^{4b})_{q} is H. |
| 35 | R^{1b} is H, X¹ is CH, X² is CH, (R²)ₙ is 6-C≡CCF₃, Y is O, (R^{3b})ₚ is H, R^{3a} is Me, R^{4a} is Cl and |
| | (R^{4b})_{q} is H. |
| 36 | R^{1b} is H, X¹ is CH, X² is CH, (R²)ₙ is 6-CH₂OMe, Y is O, (R^{3b})ₚ is H, R^{3a} is Me, R^{4a} is Cl and |
| | (R^{4b})_{q} is H. |
| 37 | R^{1b} is H, X¹ is CH, X² is CH, (R²)ₙ is 6-CH₂OCHF₂, Y is O, (R^{3b})ₚ is H, R^{3a} is Me, R^{4a} is Cl |
| | and (R^{4b})_{q} is H. |
| 38 | R^{1b} is H, X¹ is CH, X² is CH, (R²)ₙ is 6-CH₂OCF₃, Y is O, (R^{3b})ₚ is H, R^{3a} is Me, R^{4a} is Cl and |
| | (R^{4b})_{q} is H. |
| 39 | R^{1b} is H, X¹ is CH, X² is CH, (R²)ₙ is 6-OEt, Y is O, (R^{3b})ₚ is H, R^{3a} is Me, R^{4a} is Cl and (R^{4b})_{q} |
| | is H. |
| 40 | R^{1b} is H, X¹ is CH, X² is CH, (R²)ₙ is 6-OCHF₂, Y is O, (R^{3b})ₚ is H, R^{3a} is Me, R^{4a} is Cl and |
| | (R^{4b})_{q} is H. |
| 41 | R^{1b} is H, X¹ is CH, X² is CH, (R²)ₙ is 6-OCF³, Y is O, (R^{3b})ₚ is H, R^{3a} is Me, R^{4a} is Cl and |
| | (R^{4b})_{q} is H. |
| 42 | R^{1b} is H, X¹ is CH, X² is CH, (R²)ₙ is 6-SMe, Y is O, (R^{3b})ₚ is H, R^{3a} is Me, R^{4a} is Cl and (R^{4b})_{q} |
| | is H. |
| 43 | R^{1b} is H, X¹ is CH, X² is CH, (R²)ₙ is 6-SOMe, Y is O, (R^{3b})ₚ is H, R^{3a} is Me, R^{4a} is Cl and |
| | (R^{4b})_{q} is H. |
| 44 | R^{1b} is H, X¹ is CH, X² is CH, (R²)ₙ is 6-SO₂Me, Y is O, (R^{3b})ₚ is H, R^{3a} is Me, R^{4a} is Cl and |
| | (R^{4b})_{q} is H. |
| 45 | R^{1b} is H, X¹ is CH, X² is CH, (R²)ₙ is 6-SCHF₂, Y is O, (R^{3b})ₚ is H, R^{3a} is Me, R^{4a} is Cl and |
| | (R^{4b})_{q} is H. |
| 46 | R^{1b} is H, X¹ is CH, X² is CH, (R²)ₙ is 6-SCF₃, Y is O, (R^{3b})ₚ is H, R^{3a} is Me, R^{4a} is Cl and |
| | (R^{4b})_{q} is H. |
| 47 | R^{1b} is H, X¹ is CH, X² is CH, (R²)ₙ is 6-SOCF₃, Y is O, (R^{3b})ₚ is H, R^{3a} is Me, R^{4a} is Cl and |
| | (R^{4b})_{q} is H. |
| 48 | R^{1b} is H, X¹ is CH, X² is CH, (R²)ₙ is 6-SO₂CF₃, Y is O, (R^{3b})ₚ is H, R^{3a} is Me, R^{4a} is Cl and |
| | (R^{4b})_{q} is H. |
| 49 | R^{1b} is H, X¹ is CH, X² is CR², (R²)ₙ is 5-F, Y is O, (R^{3b})ₚ is H, R^{3a} is Me, R^{4a} is Cl and (R^{4b})_{q} |
| | is H. |
| 50 | R^{1b} is H, X¹ is CH, X² is CR², (R²)ₙ is 5-CN, Y is O, (R^{3b})ₚ is H, R^{3a} is Me, R^{4a} is Cl and (R^{4b})_{q} |
| | is H. |
| 51 | R^{1b} is H, X¹ is CH, X² is CR², (R²)ₙ is 5-Me, Y is O, (R^{3b})ₚ is H, R^{3a} is Me, R^{4a} is Cl and (R^{4b})_{q} |
| | is H. |
| 52 | R^{1b} is H, X¹ is CH, X² is CR², (R²)ₙ is 5-C≡CH, Y is O, (R^{3b})ₚ is H, R^{3a} is Me, R^{4a} is Cl and |
| | (R^{4b})_{q} is H. |
| 53 | R^{1b} is H, X¹ is CH, X² is CR², (R²)ₙ is 5-CH₂C≡CH, Y is O, (R^{3b})ₚ is H, R^{3a} is Me, R^{4a} is Cl |
| | and (R^{4b})_{q} is H. |
| 54 | R^{1b} is H, X¹ is CH, X² is CR², (R²)ₙ is 5-OCH₂C≡CH, Y is O, (R^{3b})ₚ is H, R^{3a} is Me, R^{4a} is Cl |
| | and (R^{4b})_{q} is H. |
| 55 | R^{1b} is H, X¹ is CH, X² is CR², (R²)ₙ is 5-(J-9), Y is O, (R^{3b})ₚ is H, R^{3a} is Me, R^{4a} is Cl and |
| | (R^{4b})_{q} is H. |
| 56 | R^{1b} is H, X¹ is CH, X² is CR², (R²)ₙ is 5-(J-10), Y is O, (R^{3b})ₚ is H, R^{3a} is Me, R^{4a} is Cl and |
| | (R^{4b})_{q} is H. |
| 57 | R^{1b} is H, X¹ is CH, X² is CR², (R²)ₙ is 5-c-Pr, Y is O, (R^{3b})ₚ is H, R^{3a} is Me, R^{4a} is Cl and |
| | (R^{4b})_{q} is H. |
| 58 | R^{1b} is H, X¹ is CH, X² is CR², (R²)ₙ is 5-CH₂F, Y is O, (R^{3b})ₚ is H, R^{3a} is Me, R^{4a} is Cl and |
| | (R^{4b})_{q} is H. |
| 59 | R^{1b} is H, X¹ is CH, X² is CR², (R²)ₙ is 5-CHF₂, Y is O, (R^{3b})ₚ is H, R^{3a} is Me, R^{4a} is Cl and |
| | (R^{4b})_{q} is H. |
| 60 | R^{1b} is H, X¹ is CH, X² is CR², (R²)ₙ is 5-CH₂OMe, Y is O, (R^{3b})ₚ is H, R^{3a} is Me, R^{4a} is Cl and |
| | (R^{4b})_{q} is H. |
| 61 | R^{1b} is H, X¹ is CH, X² is CR², (R²)ₙ is 5-OMe, Y is O, (R^{3b})ₚ is H, R^{3a} is Me, R^{4a} is Cl and |
| | (R^{4b})_{q} is H. |
| 62 | R^{1b} is H, X¹ is CH, X² is CR², (R²)ₙ is 5-OEt, Y is O, (R^{3b})ₚ is H, R^{3a} is Me, R^{4a} is Cl and |
| | (R^{4b})_{q} is H. |
| 63 | R^{1b} is H, X¹ is CH, X² is CR², (R²)ₙ is 5-Oi-Pro, Y is O, (R^{3b})ₚ is H, R^{3a} is Me, R^{4a} is Cl and |
| | (R^{4b})_{q} is H. |
| 64 | R^{1b} is H, X¹ is CH, X² is CR², (R²)ₙ is 5-OCHF₂, Y is O, (R^{3b})ₚ is H, R^{3a} is Me, R^{4a} is Cl and |
| | (R^{4b})_{q} is H. |
| 65 | R^{1b} is H, X¹ is CH, X² is CR², (R²)ₙ is 5-OCF₃, Y is O, (R^{3b})ₚ is H, R^{3a} is Me, R^{4a} is Cl and |
| | (R^{4b})_{q} is H. |
| 66 | R^{1b} is H, X¹ is CH, X² is CR², (R²)ₙ is 5-SMe, Y is O, (R^{3b})ₚ is H, R^{3a} is Me, R^{4a} is Cl and |
| | (R^{4b})_{q} is H. |
| 67 | R^{1b} is H, X¹ is CR², x² is CH, (R²)ₙ is 4-F, Y is O, (R^{3b})ₚ is H, R^{3a} is Me, R^{4a} is Cl and (R^{4b})_{q} |
| | is H. |
| 68 | R^{1b} is H, X¹ is CR², x² is CH, (R²)ₙ is 4-Cl, Y is O, (R^{3b})ₚ is H, R^{3a} is Me, R^{4a} is Cl and (R^{4b})_{q} |
| | is H. |
| 69 | R^{1b} is H, X¹ is CR², x² is CH, (R²)ₙ is 4-CN, X² is CH, Y is O, (R^{3b})ₚ is H, R^{3a} is Me, R^{4a} is |
| | Cl and (R^{4b})_{q} is H. |
| 70 | R^{1b} is H, X¹ is CR², x² is CH, (R²)ₙ is 4-Me, X² is CH, Y is O, (R^{3b})ₚ is H, R^{3a} is Me, R^{4a} is Cl |
| | and (R^{4b})_{q} is H. |
| 71 | R^{1b} is H, X¹ is CR², x² is CH, (R²)ₙ is 4-OMe, X² is CH, Y is O, (R^{3b})ₚ is H, R^{3a} is Me, R^{4a} is |
| | Cl and (R^{4b})_{q} is H. |
| 72 | R^{1b} is H, X¹ is CH, X² is CR², (R²)ₙ is 5-OMe,6-Me, Y is O, (R^{3b})ₚ is H, R^{3a} is Me, R^{4a} is Cl |
| | and (R^{4b})_{q} is H. |
| 73 | R^{1b} is H, X¹ is CH, X² is CR², (R²)ₙ is 5,6-di-Me, Y is O, (R^{3b})ₚ is H, R^{3a} is Me, R^{4a} is Cl and |
| | (R^{4b})_{q} is H. |
| 74 | R^{1b} is H, X¹ is CH, X² is CR², (R²)ₙ is 5-F,6-Me, Y is O, (R^{3b})ₚ is H, R^{3a} is Me, R^{4a} is Cl and |
| | (R^{4b})_{q} is H. |
| 75 | R^{1b} is H, X¹ is CH, X² is CR², (R²)ₙ is 5-OMe,6-Et, Y is O, (R^{3b})ₚ is H, R^{3a} is Me, R^{4a} is Cl and |
| | (R^{4b})_{q} is H. |
| 76 | R^{1b} is H, X¹ is CH, X² is CR², (R²)ₙ is 5-Me,6-Et, Y is O, (R^{3b})ₚ is H, R^{3a} is Me, R^{4a} is Cl and |
| | (R^{4b})_{q} is H. |
| 77 | R^{1b} is H, X¹ is CH, X² is CR², (R²)ₙ is 5-F,6-Et, Y is O, (R^{3b})ₚ is H, R^{3a} is Me, R^{4a} is Cl and |
| | (R^{4b})_{q} is H. |
| 78 | R^{1b} is H, X¹ is CH, X² is CR², (R²)ₙ is 5,6-di-OMe, Y is O, (R^{3b})ₚ is H, R^{3a} is Me, R^{4a} is Cl and |
| | (R^{4b})_{q} is H. |
| 79 | R^{1b} is H, X¹ is CH, X² is CR², (R²)ₙ is 5-Me,6-OMe, Y is O, (R^{3b})ₚ is H, R^{3a} is Me, R^{4a} is Cl |
| | and (R^{4b})_{q} is H. |
| 80 | R^{1b} is H, X¹ is CH, X² is CR², (R²)ₙ is 5-F,6-OMe, Y is O, (R^{3b})ₚ is H, R^{3a} is Me, R^{4a} is Cl and |
| | (R^{4b})_{q} is H. |
| 81 | R^{1b} is H, X¹ is CH, X² is CR², (R²)ₙ is 5-OMe,6-F, Y is O, (R^{3b})ₚ is H, R^{3a} is Me, R^{4a} is Cl and |
| | (R^{4b})_{q} is H. |
| 82 | R^{1b} is H, X¹ is CH, X² is CR², (R²)ₙ is 5-Me,6-F, Y is O, (R^{3b})ₚ is H, R^{3a} is Me, R^{4a} is Cl and |
| | (R^{4b})_{q} is H. |
| 83 | R^{1b} is H, X¹ is CH, X² is CR², (R²)ₙ is 5,6-di-F, Y is O, (R^{3b})ₚ is H, R^{3a} is Me, R^{4a} is Cl and |
| | (R^{4b})_{q} is H. |
| 84 | R^{1b} is H, X¹ is CR², x² is CR², (R²)ₙ is 4,5,6-tri-F, Y is O, (R^{3b})ₚ is H, R^{3a} is Me, R^{4a} is Cl and |
| | (R^{4b})_{q} is H. |
| 85 | R^{1b} is H, X¹ is CR², x² is CR², (R²)ₙ is 4,5-di-F,6-Me, Y is O, (R^{3b})ₚ is H, R^{3a} is Me, R^{4a} is Cl |
| | and (R^{4b})_{q} is H. |
| 86 | R^{1b} is H, X¹ is CR², x² is CR², (R²)ₙ is 4,5-di-F,6-OMe, Y is O, (R^{3b})ₚ is H, R^{3a} is Me, R^{4a} is |
| | Cl and (R^{4b})_{q} is H. |
| 87 | R^{1b} is H, X¹ is N, X² is CH, (R²)ₙ is 6-OMe, Y is O, (R^{3b})ₚ is H, R^{3a} is Me, R^{4a} is Cl and (R^{4b})_{q} |
| | is H. |
| 88 | R^{1b} is H, X¹ is N, X² is CH, (R²)ₙ is 6-Me, Y is O, (R^{3b})ₚ is H, R^{3a} is Me, R^{4a} is Cl and (R^{4b})_{q} |
| | is H. |
| 89 | R^{1b} is H, X¹ is CH, X² is N, (R²)ₙ is 6-OMe, Y is O, (R^{3b})ₚ is H, R^{3a} is Me, R^{4a} is Cl and (R^{4b})_{q} |
| | is H. |
| 90 | R^{1b} is H, X¹ is CH, X² is N, (R²)ₙ is 6-Me, Y is O, (R^{3b})ₚ is H, R^{3a} is Me, R^{4a} is Cl and (R^{4b})_{q} |
| | is H. |
| 91 | R^{1b} is H, X¹ is CH, X² is taken together with C6 to form J-14, Y is O, (R^{3b})ₚ is H, R^{3a} is Me, R^{4a} |
| | is Cl and (R^{4b})_{q} is H. |
| 92 | R^{1b} is H, X¹ is CH, X² is taken together with C6 to form J-15, Y is O, (R^{3b})ₚ is H, R^{3a} is Me, R^{4a} |
| | is Cl and (R^{4b}) is H. |
| 93 | R^{1b} is H, X¹ is CH, X² is taken together with C6 to form J-16, Y is O, (R^{3b})ₚ is H, R^{3a} is Me, R^{4a} |
| | is Cl and (R^{4b})_{q} is H. |
| 94 | R^{1b} is H, X¹ is CH, X² is taken together with C6 to form J-17, Y is O, (R^{3b})ₚ is H, R^{3a} is Me, R^{4a} |
| | is Cl and (R^{4b})_{q} is H. |
| 95 | R^{1b} is H, X¹ is CH, X² is taken together with C6 to form J-18, Y is O, (R^{3b})ₚ is H, R^{3a} is Me, R^{4a} |
| | is Cl and (R^{4b})_{q} is H. |
| 96 | R ^{1b} is H, X¹ is CH, X² is taken together with C6 to form J-19, Y is O, (R^{3b})ₚ is H, R^{3a} is Me, R^{4a} |
| | is Cl and (R^{4b})_{q} is H. |
| 97 | R^{1b} is H, X¹ is CH, X² is taken together with C6 to form J-20, Y is O, (R^{3b})ₚ is H, R^{3a} is Me, R^{4a} |
| | is Cl and (R^{4b})_{q} is H. |
| 98 | R^{1b} is H, X¹ is CH, X² is taken together with C6 to form J-21, Y is O, (R^{3b})ₚ is H, R^{3a} is Me, R^{4a} |
| | is Cl and (R^{4b})_{q} is H. |
| 99 | R^{1b} is F, X¹ is CH, X² is CH, (R²)ₙ is 6-OMe, Y is O, (R^{3b})ₚ is H, R^{3a} is Me, R^{4a} is Cl and (R^{4b})_{q} |
| | ² is H. |
| 100 | R^{1b} is H, X¹ is CH, X² is CH, (R²)ₙ is 6-OMe, Y is O, (R^{3b})ₚ is H, R^{3a} is CH═CH₂, R^{4a} is Cl and |
| | (R^{4b})_{q} is H. |
| 101 | R^{1b} is H, X¹ is CH, X² is CH, (R²)ₙ is 6-OMe, Y is O, (R^{3b})ₚ is H, R^{3a} is C≡CH, R^{4a} is Cl and |
| | (R^{4b})_{q} is H. |
| 102 | R^{1b} is H, X¹ is CH, X² is CH, (R²)ₙ is 6-OMe, Y is O, (R^{3b})ₚ is H, R^{3a} is c-Pr, R^{4a} is Cl and |
| | (R^{4b})_{q} is H. |
| 103 | R^{1b} is H, X¹ is CH, X² is CH, (R²)ₙ is 6-OMe, Y is O, (R^{3b})ₚ is H, R^{3a} is OMe, R^{4a} is Cl and |
| | (R^{4b})_{q} is H. |
| 104 | R^{1b} is H, X¹ is CH, X² is CH, (R²)ₙ is 6-OMe, Y is O, (R^{3b})ₚ is H, R^{3a} is CH₂c-Pr, R^{4a} is Cl and |
| | (R^{4b})_{q} is H. |
| 105 | R^{1b} is H, X¹ is CH, X² is CH, (R²)ₙ is 6-OMe, Y is O, (R^{3b})ₚ is H, R^{3a} is CH₂F, R^{4a} is Cl and |
| | (R^{4b})_{q} is H. |
| 106 | R^{1b} is H, X¹ is CH, X² is CH, (R²)ₙ is 6-OMe, Y is O, (R^{3b})ₚ is H, R^{3a} is CHF₂, R^{4a} is Cl and |
| | (R^{4b})_{q} is H. |
| 107 | R^{1b} is H, X¹ is CH, X² is CH, (R²)ₙ is 6-OMe, Y is O, (R^{3b})ₚ is H, R^{3a} is CF₃, R^{4a} is Cl and |
| | (R^{4b})_{q} is H. |
| 108 | R^{1b} is H, X¹ is CH, X² is CH, (R²)ₙ is 6-OMe, Y is O, (R^{3b})ₚ is H, R^{3a} is CH═CF₂, R^{4a} is Cl and |
| | (R^{4b})_{q} is H. |
| 109 | R^{1b} is H, X¹ is CH, X² is CH, (R²)ₙ is 6-OMe, Y is O, (R^{3b})ₚ is H, R^{3a} is C≡CCF₃, R^{4a} is Cl and |
| | (R^{4b})_{q} is H. |
| 110 | R^{1b} is H, X¹ is CH, X² is CH, (R²)ₙ is 6-OMe, Y is O, (R^{3b})ₚ is H, R^{3a} is J-5, R^{4a} is Cl and (R^{4b})_{q} |
| | is H. |
| 111 | R^{1b} is H, X¹ is CH, X² is CH, (R²)ₙ is 6-OMe, Y is O, (R^{3b})ₚ is H, R^{3a} is CH₂OMe, R^{4a} is Cl |
| | and (R^{4b})_{q} is H. |
| 112 | R^{1b} is H, X¹ is CH, X² is CH, (R²)ₙ is 6-OMe, Y is O, (R^{3b})ₚ is H, R^{3a} is CH₂OCH₂F, R^{4a} is Cl |
| | and (R^{4b})_{q} is H. |
| 113 | R^{1b} is H, X¹ is CH, X² is CH, (R²)ₙ is 6-OMe, Y is O, (R^{3b})ₚ is H, R^{3a} is CH₂OCHF₂, R^{4a} is Cl |
| | and (R^{4b})_{q} is H. |
| 114 | R^{1b} is H, X¹ is CH, X² is CH, (R²)ₙ is 6-OMe, Y is O, (R^{3b})ₚ is H, R^{3a} is CH₂OCF₃, R^{4a} is Cl |
| | and (R^{4b})_{q} is H. |
| 115 | R^{1b} is H, X¹ is CH, X² is CH, (R²)ₙ is 6-OMe, Y is O, (R^{3b})ₚ is H, R^{3a} is CH₂SMe, R^{4a} is Cl and |
| | (R^{4b})_{q} is H. |
| 116 | R^{1b} is H, X¹ is CH, X² is CH, (R²)ₙ is 6-OMe, Y is O, (R^{3b})ₚ is H, R^{3a} is CH₂SCH₂F, R^{4a} is Cl |
| | and (R^{4b})_{q} is H. |
| 117 | R^{1b} is H, X¹ is CH, X² is CH, (R²)ₙ is 6-OMe, Y is O, (R^{3b})ₚ is H, R^{3a} is CH₂SCHF₂, R^{4a} is Cl |
| | and (R^{4b})_{q} is H. |
| 118 | R^{1b} is H, X¹ is CH, X² is CH, (R²)ₙ is 6-OMe, Y is O, (R^{3b})ₚ is H, R^{3a} is CH₂SCF₃, R^{4a} is Cl |
| | and (R^{4b})_{q} is H. |
| 119 | R^{1b} is H, X¹ is CH, X² is CH, (R²)ₙ is 6-OMe, Y is O, (R^{3b})ₚ is H, R^{3a} is CH₂CN, R^{4a} is Cl and |
| | (R^{4b})_{q} is H. |
| 120 | R^{1b} is H, X¹ is CH, X² is CH, (R²)ₙ is 6-OMe, Y is O, (R^{3b})ₚ is H, R^{3a} is CH₂COMe, R^{4a} is Cl |
| | and (R^{4b})_{q} is H. |
| 121 | R^{1b} is H, X¹ is CH, X² is CH, (R²)ₙ is 6-OMe, Y is O, (R^{3b})ₚ is H, R^{3a} is CH₂CO₂Me, R^{4a} is Cl |
| | and (R^{4b})_{q} is H. |
| 122 | R^{1b} is H, X¹ is CH, X² is CH, (R²)ₙ is 6-OMe, Y is O, (R^{3b})ₚ is 2-F, R^{3a} is Me, R^{4a} is Cl and |
| | (R^{4b})_{q} is H. |
| 123 | R^{1b} is H, X¹ is CH, X² is CH, (R²)ₙ is 6-OMe, Y is O, (R^{3b})ₚ is 3,3-di-F, R^{3a} is Me, R^{4a} is Cl |
| | and (R^{4b})_{q} is H. |
| 124 | R^{1b} is H, X¹ is CH, X² is CH, (R²)ₙ is 6-OMe, Y is O, (R^{3b})ₚ is 2,3,3-tri-F, R^{3a} is Me, R^{4a} is Cl |
| | and (R^{4b})_{q} is H. |
| 125 | R^{1b} is H, X¹ is CH, X² is CH, (R²)ₙ is 6-OMe, Y is O, (R^{3b})ₚ is 2,3,3-tri-F, R^{3a} is F, R^{4a} is Cl |
| | and (R^{4b})_{q} is H. |
| 126 | R^{1b} is H, X¹ is CH, X² is CH, (R²)ₙ is 6-OMe, Y is O, (R^{3b})ₚ is H, R^{3a} is Me, R^{4a} is H and (R^{4b})_{q} |
| | is H. |
| 127 | R^{1b} is H, X¹ is CH, X² is CH, (R²)ₙ is 6-OMe, Y is O, (R^{3b})ₚ is H, R^{3a} is Me, R^{4a} is F and (R^{4b})_{q} |
| | is H. |
| 128 | R^{1b} is H, X¹ is CH, X² is CH, (R²)ₙ is 6-OMe, Y is O, (R^{3b})ₚ is H, R^{3a} is Me, R^{4a} is Br and |
| | (R^{4b})_{q} is H. |
| 129 | R^{1b} is H, X¹ is CH, X² is CH, (R²)ₙ is 6-OMe, Y is O, (R^{3b})ₚ is H, R^{3a} is Me, R^{4a} is CN and |
| | (R^{4b})_{q} is H. |
| 130 | R^{1b} is H, X¹ is CH, X² is CH, (R²)ₙ is 6-OMe, Y is O, (R^{3b})ₚ is H, R^{3a} is Me, R^{4a} is Me and |
| | (R^{4b})_{q} is H. |
| 131 | R^{1b} is H, X¹ is CH, X² is CH, (R²)ₙ is 6-OMe, Y is O, (R^{3b})ₚ is H, R^{3a} is Me, R^{4a} is Et and |
| | (R^{4b})_{q} is H. |
| 132 | R^{1b} is H, X¹ is CH, X² is CH, (R²)ₙ is 6-OMe, Y is O, (R^{3b})ₚ is H, R^{3a} is Me, R^{4a} is CH₂CH═CH₂ |
| | and (R^{4b})_{q} is H. |
| 133 | R^{1b} is H, X¹ is CH, X² is CH, (R²)ₙ is 6-OMe, Y is O, (R^{3b})ₚ is H, R^{3a} is Me, R^{4a} is C≡CH and |
| | (R^{4b})_{q} is H. |
| 134 | R^{1b} is H, X¹ is CH, X² is CH, (R²)ₙ is 6-OMe, Y is O, (R^{3b})ₚ is H, R^{3a} is Me, R^{4a} is CH₂C≡CH |
| | and (R^{4b})_{q} is H. |
| 135 | R^{1b} is H, X¹ is CH, X² is CH, (R²)ₙ is 6-OMe, Y is O, (R^{3b})ₚ is H, R^{3a} is Me, R^{4a} is c-Pr and |
| | (R^{4b})_{q} is H. |
| 136 | R^{1b} is H, X¹ is CH, X² is CH, (R²)ₙ is 6-OMe, Y is O, (R^{3b})ₚ is H, R^{3a} is Me, R^{4a} is CH₂F and |
| | (R^{4b})_{q} is H. |
| 137 | R^{1b} is H, X¹ is CH, X² is CH, (R²)ₙ is 6-OMe, Y is O, (R^{3b})ₚ is H, R^{3a} is Me, R^{4a} is CHF₂ and |
| | (R^{4b})_{q} is H. |
| 138 | R^{1b} is H, X¹ is CH, X² is CH, (R²)ₙ is 6-OMe, Y is O, (R^{3b})ₚ is H, R^{3a} is Me, R^{4a} is CF₃ and |
| | (R^{4b})_{q} is H. |
| 139 | R^{1b} is H, X¹ is CH, X² is CH, (R²)ₙ is 6-OMe, Y is O, (R^{3b})ₚ is H, R^{3a} is Me, R^{4a} is CH₂OMe |
| | and (R^{4b})_{q} is H. |
| 140 | R^{1b} is H, X¹ is CH, X² is CH, (R²)ₙ is 6-OMe, Y is O, (R^{3b})ₚ is H, R^{3a} is Me, R^{4a} is CH₂OCF₂H |
| | and (R^{4b})_{q} is H. |
| 141 | R^{1b} is H, X¹ is CH, X² is CH, (R²)ₙ is 6-OMe, Y is O, (R^{3b})ₚ is H, R^{3a} is Me, R^{4a} is CH₂OCF₃ |
| | and (R^{4b})_{q} is H. |
| 142 | R^{1b} is H, X¹ is CH, X² is CH, (R²)ₙ is 6-OMe, Y is O, (R^{3b})ₚ is H, R^{3a} is Me, R^{4a} is OMe and |
| | (R^{4b})_{q} is H. |
| 143 | R^{1b} is H, X¹ is CH, X² is CH, (R²)ₙ is 6-OMe, Y is O, (R^{3b})ₚ is H, R^{3a} is Me, R^{4a} is OCF₂H and |
| | (R^{4b})_{q} is H. |
| 144 | R^{1b} is H, X¹ is CH, X² is CH, (R²)ₙ is 6-OMe, Y is O, (R^{3b})ₚ is H, R^{3a} is Me, R^{4a} is OCF₃ and |
| | (R^{4b})_{q} is H. |
| 145 | R^{1b} is H, X¹ is CH, X² is CH, (R²)ₙ is 6-OMe, Y is O, (R^{3b})ₚ is H, R^{3a} is Me, R^{4a} is SMe and |
| | (R^{4b})_{q} is H. |
| 146 | R^{1b} is H, X¹ is CH, X² is CH, (R²)ₙ is 6-OMe, Y is O, (R^{3b})ₚ is H, R^{3a} is Me, R^{4a} is SCF₂H and |
| | (R^{4b})_{q} is H. |
| 147 | R^{1b} is H, X¹ is CH, X² is CH, (R²)ₙ is 6-OMe, Y is O, (R^{3b})ₚ is H, R^{3a} is Me, R^{4a} is SCF₃ and |
| | (R^{4b})_{q} is H. |
| 148 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 5-F. |
| 149 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-F. |
| 150 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-CN. |
| 151 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-Me. |
| 152 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-CH₂F. |
| 153 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-OMe. |
| 154 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 6-SMe. |
| 155 | R^{1b} is H, X¹ is CH, X² is CH, (R²)ₙ is H, Y is O, (R^{3b})ₚ is H, R^{3a} is Me, R^{4a} is Cl and (R^{4b})_{q} is |
| | 5,6-di-F. |
| 156 | R^{1b} is H, X¹ is CH, X² is CH, (R²)ₙ is H, Y is O, (R^{3b})ₚ is H, R^{3a} is Me, R^{4a} is Me and (R^{4b})_{q} is |
| | H. |
| 157 | R^{1b} is H, X¹ is CH, X² is CH, (R²)ₙ is 6-F, Y is O, (R^{3b})ₚ is H, R^{3a} is Me, R^{4a} is Me and (R^{4b})_{q} |
| | is H. |
| 158 | R^{1b} is H, X¹ is CH, X² is CH, (R²)ₙ is 6-Cl, Y is O, (R^{3b})ₚ is H, R^{3a} is Me, R^{4a} is Me and (R^{4b})_{q} |
| | is H. |
| 159 | R^{1b} is H, X¹ is CH, X² is CH, (R²)ₙ is 6-CN, Y is O, (R^{3b})ₚ is H, R^{3a} is Me, R^{4a} is Me and (R^{4b})_{q} |
| | is H. |
| 160 | R^{1b} is H, X¹ is CH, X² is CH, (R²)ₙ is 6-Me, Y is O, (R^{3b})ₚ is H, R^{3a} is Me, R^{4a} is Me and (R^{4b})_{q} |
| | is H. |
| 161 | R^{1b} is H, X¹ is CH, X² is CH, (R²)ₙ is 6-Et, Y is O, (R^{3b})ₚ is H, R^{3a} is Me, R^{4a} is Me and (R^{4b})_{q} |
| | is H. |
| 162 | R^{1b} is H, X¹ is CH, X² is CH, (R²)ₙ is 6-C≡CH, Y is O, (R^{3b})ₚ is H, R^{3a} is Me, R^{4a} is Me and |
| | (R^{4b})_{q} is H. |
| 163 | R^{1b} is H, X¹ is CH, X² is CH, (R²)ₙ is 6-OCH₂C≡CH, Y is O, (R^{3b})ₙ is H, R^{3a} is Me, R^{4a} is Me |
| | and (R^{4b})_{q} is H. |
| 164 | R^{1b} is H, X¹ is CH, X² is CH, (R²)ₙ is 6-c-Pr, Y is O, (R^{3b})ₚ is H, R^{3a} is Me, R^{4a} is Me and |
| | (R^{4b})_{q} is H. |
| 165 | R^{1b} is H, X¹ is CH, X² is CH, (R²)ₙ is 6-CH₂F, Y is O, (R^{3b})ₚ is H, R^{3a} is Me, R^{4a} is Me and |
| | (R^{4b})_{q} is H. |
| 166 | R^{1b} is H, X¹ is CH, X² is CH, (R²)ₙ is 6-CHF², Y is O, (R^{3b})ₚ is H, R^{3a} is Me, R^{4a} is Me and |
| | (R^{4b})_{q} is H. |
| 167 | R^{1b} is H, X¹ is CH, X² is CH, (R²)ₙ is 6-CH₂CH₂F, Y is O, (R^{3b})ₚ is H, R^{3a} is Me, R^{4a} is Me |
| | and (R^{4b})_{q} is H. |
| 168 | R^{1b} is H, X¹ is CH, X² is CH, (R²)ₙ is 6-CH₂CHF₂, Y is O, (R^{3b})ₚ is H, R^{3a} is Me, R^{4a} is Me |
| | and (R^{4b})_{q} is H. |
| 169 | R^{1b} is H, X¹ is CH, X² is CH, (R²)ₙ is 6-CH₂CF₃, Y is O, (R^{3b})ₚ is H, R^{3a} is Me, R^{4a} is Me and |
| | (R^{4b})_{q} is H. |
| 170 | R^{1b} is H, X¹ is CH, X² is CH, (R²)ₙ is 6-CF₂Me, Y is O, (R^{3b})ₚ is H, R^{3a} is Me, R^{4a} is Me and |
| | (R^{4b})_{q} is H. |
| 171 | R^{1b} is H, X¹ is CH, X² is CH, (R²)ₙ is 6-CH(F)Me, Y is O, (R^{3b})ₚ is H, R^{3a} is Me, R^{4a} is Me and |
| | (R^{4b})_{q} is H. |
| 172 | R^{1b} is H, X¹ is CH, X² is CH, (R²)ₙ is 6-CH₂OMe, Y is O, (R^{3b})ₚ is H, R^{3a} is Me, R^{4a} is Me and |
| | (R^{4b})_{q} is H. |
| 173 | R^{1b} is H, X¹ is CH, X² is CH, (R²)ₙ is 6-OCHF₂, Y is O, (R^{3b})ₚ is H, R^{3a} is Me, R^{4a} is Me and |
| | (R^{4b})_{q} is H. |
| 174 | R^{1b} is H, X¹ is CH, X² is CH, (R²)ₙ is 6-OCF₃, Y is O, (R^{3b})ₚ is H, R^{3a} is Me, R^{4a} is Me and |
| | (R^{4b})_{q} is H. |
| 175 | R^{1b} is H, X¹ is CH, X² is CH, (R²)ₙ is 6-SMe, Y is O, (R^{3b})ₚ is H, R^{3a} is Me, R^{4a} is Me and |
| | (R^{4b})_{q} is H. |
| 176 | R^{1b} is H, X¹ is CH, X² is CH, (R²)ₙ is 6-SCHF₂, Y is O, (R^{3b})ₚ is H, R^{3a} is Me, R^{4a} is Me and |
| | (R^{4b})_{q} is H. |
| 177 | R^{1b} is H, X¹ is CH, X² is CH, (R²)ₙ is 6-SCF₃, Y is O, (R^{3b})ₚ is H, R^{3a} is Me, R^{4a} is Me and |
| | (R^{4b})_{q} is H. |
| 178 | R^{1b} is H, X¹ is CH, X² is CR², (R²)ₙ is 5-F, Y is O, (R^{3b})ₚ is H, R^{3a} is Me, R^{4a} is Me and (R^{4b})_{q} |
| | is H. |
| 179 | R^{1b} is H, X¹ is CH, X² is CR², (R²)ₙ is 5-CN, Y is O, (R^{3b})ₚ is H, R^{3a} is Me, R^{4a} is Me and |
| | (R^{4b})_{q} is H. |
| 180 | R^{1b} is H, X¹ is CH, X² is CR², (R²)ₙ is 5-Me, Y is O, (R^{3b})ₚ is H, R^{3a} is Me, R^{4a} is Me and |
| | (R^{4b})_{q} is H. |
| 181 | R^{1b} is H, X¹ is CH, X² is CR², (R²)ₙ is 5-C≡CH, Y is O, (R^{3b})ₚ is H, R^{3a} is Me, R^{4a} is Me and |
| | (R^{4b})_{q} is H. |
| 182 | R^{1b} is H, X¹ is CH, X² is CR², (R²)ₙ is 5-OCH₂C≡CH, Y is O, (R^{3b})ₚ is H, R^{3a} is Me, R^{4a} is Me |
| | and (R^{4b})_{q} is H. |
| 183 | R^{1b} is H, X¹ is CH, X² is CR², (R²)ₙ is 5-(J-9), Y is O, (R^{3b})ₚ is H, R^{3a} is Me, R^{4a} is Me and |
| | (R^{4b})_{q} is H. |
| 184 | R^{1b} is H, X¹ is CH, X² is CR², (R²)ₙ is 5-(J-10), Y is O, (R^{3b})ₚ is H, R^{3a} is Me, R^{4a} is Me and |
| | (R^{4b})_{q} is H. |
| 185 | R^{1b} is H, X¹ is CH, X² is CR², (R²)ₙ is 5-CH₂F, Y is O, (R^{3b})ₚ is H, R^{3a} is Me, R^{4a} is Me and |
| | (R^{4b})_{q} is H. |
| 186 | R^{1b} is H, X¹ is CH, X² is CR², (R²)ₙ is 5-CHF₂, Y is O, (R^{3b})ₚ is H, R^{3a} is Me, R^{4a} is Me and |
| | (R^{4b})_{q} is H. |
| 187 | R^{1b} is H, X¹ is CH, X² is CR², (R²)ₙ is 5-CH₂OMe, Y is O, (R^{3b})ₚ is H, R^{3a} is Me, R^{4a} is Me |
| | and (R^{4b})_{q} is H. |
| 188 | R^{1b} is H, X¹ is CH, X² is CR², (R²)ₙ is 5-OMe, Y is O, (R^{3b})ₚ is H, R^{3a} is Me, R^{4a} is Me and |
| | (R^{4b})_{q} is H. |
| 189 | R^{1b} is H, X¹ is CH, X² is CR², (R²)ₙ is 5-OEt, Y is O, (R^{3b})ₚ is H, R^{3a} is Me, R^{4a} is Me and |
| | (R^{4b})_{q} is H. |
| 190 | R^{1b} is H, X¹ is CH, X² is CR², (R²)ₙ is 5-Oi-Pro, Y is O, (R^{3b})ₚ is H, R^{3a} is Me, R^{4a} is Me and |
| | (R^{4b})_{q} is H. |
| 191 | R^{1b} is H, X¹ is CH, X² is CR², (R²)ₙ is 5-OCHF₂, Y is O, (R^{3b})ₚ is H, R^{3a} is Me, R^{4a} is Me and |
| | (R^{4b})_{q} is H. |
| 192 | R^{1b} is H, X¹ is CH, X² is CR², (R²)ₙ is 5-OCF₃, Y is O, (R^{3b})ₚ is H, R^{3a} is Me, R^{4a} is Me and |
| | (R^{4b})_{q} is H. |
| 193 | R^{1b} is H, X¹ is CH, X² is CR², (R²)ₙ is 5-SMe, Y is O, (R^{3b})ₚ is H, R^{3a} is Me, R^{4a} is Me and |
| | (R^{4b})_{q} is H. |
| 194 | R^{1b} is H, X¹ is CH, X² is CH, (R²)ₙ is 6-OMe, Y is O, (R^{3b})ₚ is H, R^{3a} is c-Pr, R^{4a} is Me and |
| | (R^{4b})_{q} is H. |
| 195 | R^{1b} is H, X¹ is CH, X² is CH, (R²)ₙ is 6-OMe, Y is O, (R^{3b})ₚ is H, R^{3a} is CH₂F, R^{4a} is Me and |
| | (R^{4b})_{q} is H. |
| 196 | R^{1b} is H, X¹ is CH, X² is CH, (R²)ₙ is 6-OMe, Y is O, (R^{3b})ₚ is H, R^{3a} is CHF₃, R^{4a} is Me and |
| | (R^{4b})_{q} is H. |
| 197 | R^{1b} is H, X¹ is CH, X² is CH, (R²)ₙ is 6-OMe, Y is O, (R^{3b})ₚ is H, R^{3a} is CF³, R^{4a} is Me and |
| | (R^{4b})_{q} is H. |
| 198 | R^{1b} is H, X¹ is CH, X² is CH, (R²)ₙ is 6-OMe, Y is O, (R^{3b})ₚ is H, R^{3a} is CH₂OMe, R^{4a} is Me |
| | and (R^{4b})_{q} is H. |
| 199 | R^{1b} is H, X¹ is CH, X² is CH, (R²)ₙ is 6-Me, Y is O, (R^{3b})ₚ is H, R^{3a} is c-Pr, R^{4a} is Cl and (R^{4b})_{q} |
| | is H. |
| 200 | R^{1b} is H, X¹ is CH, X² is CH, (R²)ₙ is 6-Me, Y is O, (R^{3b})ₚ is H, R^{3a} is CH₂F, R^{4a} is Cl and |
| | (R^{4b})_{q} is H. |
| 201 | R^{1b} is H, X¹ is CH, X² is CH, (R²)ₙ is 6-Me, Y is O, (R^{3b})ₚ is H, R^{3a} is CHF₂, R^{4a} is Cl and |
| | (R^{4b})_{q} is H. |
| 202 | R^{1b} is H, X¹ is CH, X² is CH, (R²)ₙ is 6-Me, Y is O, (R^{3b})ₚ is H, R^{3a} is CF₃, R^{4a} is Cl and (R^{4b})_{q} |
| | is H. |
| 203 | R^{1b} is H, X¹ is CH, X² is CH, (R²)ₙ is 6-Me, Y is O, (R^{3b})ₚ is H, R^{3a} is CH₂OMe, R^{4a} is Cl and |
| | (R^{4b})_{q} is H. |
| 204 | R^{1b} is H, X¹ is CH, X² is CH, (R²)ₙ is 6-Me, Y is O, (R^{3b})ₚ is H, R^{3a} is c-Pr, R^{4a} is Me and |
| | (R^{4b})_{q} is H. |
| 205 | R^{1b} is H, X¹ is CH, X² is CH, (R²)ₙ is 6-Me, Y is O, (R^{3b})ₚ is H, R^{3a} is CH₂F, R^{4a} is Me and |
| | (R^{4b})_{q} is H. |
| 206 | R^{1b} is H, X¹ is CH, X² is CH, (R²)ₙ is 6-Me, Y is O, (R^{3b})ₚ is H, R^{3a} is CHF₂, R^{4a} is Me and |
| | (R^{4b})_{q} is H. |
| 207 | R^{1b} is H, X¹ is CH, X² is CH, (R²)ₙ is 6-Me, Y is O, (R^{3b})ₚ is H, R^{3a} is CF₃, R^{4a} is Me and |
| | (R^{4b})_{q} is H. |
| 208 | R^{1b} is H, X¹ is CH, X² is CH, (R²)ₙ is 6-Me, Y is O, (R^{3b})ₚ is H, R^{3a} is CH₂OMe, R^{4a} is Me and |
| | (R^{4b})_{q} is H. |
| 209 | R^{1b} is H, X¹ is CH, X² is CH, (R²)ₙ is 6-OMe, Y is S, (R^{3b})ₚ is H, R^{3a} is Me, R^{4a} is Cl and (R^{4b})_{q} |
| | is H. |
| 210 | R^{1b} is H, X¹ is CH, X² is CH, (R²)ₙ is H, Y is S, (R^{3b})ₚ is H, R^{3a} is Me, R^{4a} is Cl and (R^{4b})_{q} is |
| | H. |
| 211 | R^{1b} is H, X¹ is CH, X² is CH, (R²)ₙ is 6-Me, Y is S, (R^{3b})ₚ is H, R^{3a} is Me, R^{4a} is Cl and (R^{4b})_{q} |
| | is H. |
| 212 | R^{1b} is H, X¹ is CH, X² is CH, (R²)ₙ is 6-Et, Y is S, (R^{3b})ₚ is H, R^{3a} is Me, R^{4a} is Cl and (R^{4b})_{q} |
| | is H. |
| 213 | R^{1b} is H, X¹ is CH, X² is CR², (R²)ₙ is 5-F, Y is S, (R^{3b})ₚ is H, R^{3a} is Me, R^{4a} is Cl and (R^{4b})_{q} |
| | is H. |
| 214 | R^{1b} is H, X¹ is CH, X² is CR², (R²)ₙ is 5-OMe, Y is S, (R^{3b})ₚ is H, R^{3a} is Me, R^{4a} is Cl and |
| | (R^{4b})_{q} is H. |
| 215 | R^{1b} is H, X¹ is CH, X² is CH, (R²)ₙ is 6-OMe, Y is S, (R^{3b})ₚ is H, R^{3a} is c-Pr, R^{4a} is Cl and |
| | (R^{4b})_{q} is H. |
| 216 | R^{1b} is H, X¹ is CH, X² is CH, (R²)ₙ is 6-OMe, Y is S, (R^{3b})ₚ is H, R^{3a} is CH₂F, R^{4a} is Cl and |
| | (R^{4b})_{q} is H. |
| 217 | R^{1b} is H, X¹ is CH, X² is CH, (R²)ₙ is 6-OMe, Y is S, (R^{3b})ₚ is H, R^{3a} is CHF₂, R^{4a} is Cl and |
| | (R^{4b})_{q} is H. |
| 218 | R^{1b} is H, X¹ is CH, X² is CH, (R²)ₙ is 6-OMe, Y is S, (R^{3b})ₚ is H, R^{3a} is CF₃, R^{4a} is Cl and |
| | (R^{4b})_{q} is H. |
| 219 | R^{1b} is H, X¹ is CH, X² is CH, (R²)ₙ is 6-OMe, Y is S, (R^{3b})ₚ is H, R^{3a} is CH₂OMe, R^{4a} is Cl and |
| | (R^{4b})_{q} is H. |
| 220 | R^{1b} is H, X¹ is CH, X² is CH, (R²)ₙ is 6-OMe, Y is S, (R^{3b})ₚ is H, R^{3a} is Me, R^{4a} is Me and |
| | (R^{4b})_{q} is H. |
| 221 | R^{1b} is H, X¹ is CH, X² is CH, (R²)ₙ is H, Y is S, (R^{3b})ₚ is H, R^{3a} is Me, R^{4a} is Me and (R^{4b})_{q} is |
| | H. |
| 222 | R^{1b} is H, X¹ is CH, X² is CH, (R²)ₙ is 6-Me, Y is S, (R^{3b})ₚ is H, R^{3a} is Me, R^{4a} is Me and (R^{4b})_{q} |
| | is H. |
| 223 | R^{1b} is H, X¹ is CH, X² is CH, (R²)ₙ is 6-Et, Y is S, (R^{3b})ₚ is H, R^{3a} is Me, R^{4a} is Me and (R^{4b})_{q} |
| | is H. |
| 224 | R^{1b} is H, X¹ is CH, X² is CR², (R²)ₙ is 5-F, Y is S, (R^{3b})ₚ is H, R^{3a} is Me, R^{a} is Me and (R^{4b})_{q} |
| | is H. |
| 225 | R^{1b} is H, X¹ is CH, X² is CR², (R²)ₙ is 5-OMe, Y is S, (R^{3b})ₚ is H, R^{3a} is Me, R^{4a} is Me and |
| | (R^{4b})_{q} is H. |
| 226 | R^{1b} is H, X¹ is CH, X² is CH, (R²)ₙ is 6-OMe, Y is S, (R^{3b})ₚ is H, R^{3a} is c-Pr, R^{4a} is Me and |
| | (R^{4b})_{q} is H. |
| 227 | R^{1b} is H, X¹ is CH, X² is CH, (R²)ₙ is 6-OMe, Y is S, (R^{3b})ₚ is H, R^{3a} is CH₂F, R^{4a} is Me and |
| | (R^{4b})_{q} is H. |
| 228 | R^{1b} is H, X¹ is CH, X² is CH, (R²)ₙ is 6-OMe, Y is S, (R^{3b})ₚ is H, R^{3a} is CHF₂, R^{4a} is Me and |
| | (R^{4b})_{q} is H. |
| 229 | R^{1b} is H, X¹ is CH, X² is CH, (R²)ₙ is 6-OMe, Y is S, (R^{3b})ₚ is H, R^{3a} is CF₃, R^{4a} is Me and |
| | (R^{4b})_{q} is H. |
| 230 | R^{1b} is H, X¹ is CH, X² is CH, (R²)ₙ is 6-OMe, Y is S, (R^{3b})ₚ is H, R^{3a} is CH₂OMe, R^{4a} is Me |
| | and (R^{4b})_{q} is H. |

TABLE 231

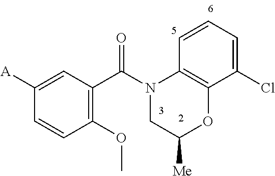

A
1,2,4-triazol-4-yl
3-fluoro-1,2,4-triazol-4-yl
tetrazol-1-yl
2-methyl-1,2,3-triazol-4-yl
2-ethyl-1,2,3-triazol-4-yl
2-isopropyl-1,2,3-triazol-4-yl
2-tert-butyl-1,2,3-triazol-4-yl
2-cyclopropyl-1,2,3-triazol-4-yl
2-cyclobutyl-1,2,3-triazol-4-yl
2-(difluoromethyl)-1,2,3-triazol-4-yl
1-methylpyrazol-4-yl
1-ethylpyrazol-4-yl
1-isopropylpyrazol-4-yl
1-tert-butylpyrazol-4-yl
1-cyclopropylpyrazol-4-yl
1-cyclobutylpyrazol-4-yl
1-(difluoromethyl)pyrazol-4-yl
5-fluoro-1-isopropyl-pyrazol-4-yl
1-methylpyrazol-3-yl
1-ethylpyrazol-3-yl
1-isopropylpyrazol-3-yl
1-tert-butylpyrazol-3-yl
5-fluoro-1-isopropyl-pyrazol-3-yl
3-methyl-1,2,4-oxadiazol-5-yl
3-ethyl-1,2,4-oxadiazol-5-yl
3-isopropyl-1,2,4-oxadiazol-5-yl
2-tert-butylthiazol-5-yl
2-cyclopropylthiazol-5-yl
2-cyclobutylthiazol-5-yl
2-methylpyrimidin-4-yl
2-ethylpyrimidin-4-yl
2-isopropylpyrimidin-4-yl
2-tert-butylpyrimidin-4-yl
2-cyclopropylpyrimidin-4-yl
2-cyclobutylpyrimidin-4-yl
3-methyl-1,2,4-thiadiazol-5-yl
3-ethyl-1,2,4-thiadiazol-5-yl
3-tert-butyl-1,2,4-oxadiazol-5-yl
5-methyl-1,2,4-oxadiazol-3-yl
5-ethyl-1,2,4-oxadiazol-3-yl
5-isopropyl-1,2,4-oxadiazol-3-yl
5-tert-butyl-1,2,4-oxadiazol-3-yl
2-methylthiazol-4-yl
2-ethylthiazol-4-yl
2-isopropylthiazol-4-yl
2-tert-butylthiazol-4-yl
4-methylthiazol-2-yl
4-ethylthiazol-2-yl
4-isopropylthiazol-2-yl
4-tert-butylthiazol-2-yl
4-cyanopyrazol-1-yl
4-(methoxycarbonyl)pyrazol-1-yl
4-cyano-3-methylpyrazol-1-yl
4-cyano-3-ethylpyrazol-1-yl
4-cyano-3-isopropylpyrazol-1-yl
4-cyano-3-tert-butylpyrazol-1-yl
1-methyl-5-oxo-1,2,4-triazol-4-yl
1-ethyl-5-oxo-1,2,4-triazol-4-yl
1-isopropyl-5-oxo-1,2,4-triazol-4-yl
1-tert-butyl-5-oxo-1,2,4-triazol-4-yl
2-methylthiazol-5-yl
2-ethylthiazol-5-yl
2-isopropylthiazol-5-yl
3-isopropyl-1,2,4-thiadiazol-5-yl
3-tert-butyl-1,2,4-thiadiazol-5-yl
3-cyclopropyl-1,2,4-thiadiazol-5-yl
3-cyclobutyl-1,2,4-thiadiazol-5-yl
3-methylisothiazol-5-yl
3-ethylisothiazol-5-yl
3-isopropylisothiazol-5-yl
3-tert-butylisothiazol-5-yl
3-cyclopropylisothiazol-5-yl
3-cyclobutylisothiazol-5-yl

Table 232 is constructed in the same manner as Tables 1 except that the Row Heading “R^{1b} is H, X¹ is CH, X² is CH, (R²)ₙ is 6-OMe, Y is O, (R^{3b})ₚ is H, R^{3a} is Me, R^{4a} is Cl and (R^{4b})_{q} is H.” is replaced with the Row Heading listed for Table 232 below (i.e. “R^{1b} is H, X¹ is CH, X² is taken together with C6 to form J-34, Y is O, (R^{3b})ₚ is H, R^{3a} is Me, R^{4a} is Cl and (R^{4b})_{q} is H.”). Therefore the first entry in Table 232 is a compound of Formula 1 wherein R^{1b}is H, X¹ is CH, X² is taken together with C6 to form J-34, Y is O, (R^{3b})ₚ is H, R^{3a} is Me, R^{4a} is Cl, (R^{4b})_{q} is H and R^{1a} is H. Tables 233 through 236 are constructed similarly.

| Table | Row Heading |
|---|---|
| 232 | R^{1b} is H, X¹ is CH, X² is taken together with C6 to form J-34, |
| | Y is O, (R^{3b})ₚ is H, R^{3a} is Me, R^{4a} is Cl and (R^{4b})_{q} is H. |
| 233 | R^{1b} is H, X¹ is CH, X² is taken together with C6 to form J-35, |
| | Y is O, (R^{3b})ₚ is H, R^{3a} is Me, R^{4a} is Cl and (R^{4b})_{q} is H. |
| 234 | R^{1b} is H, X¹ is CH, X² is taken together with C6 to form J-36, |
| | Y is O, (R^{3b})ₚ is H, R^{3a} is Me, R^{4a} is Cl and (R^{4b})_{q} is H. |
| 235 | R^{1b} is H, X¹ is CH, X² is taken together with C6 to form J-37, |
| | Y is O, (R^{3b})ₚ is H, R^{3a} is Me, R^{4a} is Cl and (R^{4b})_{q} is H. |
| 236 | R^{1b} is H, X¹ is CH, X² is taken together with C6 to form J-38, |
| | Y is O, (R^{3b})ₚ is H, R^{3a} is Me, R^{4a} is Cl and (R^{4b})_{q} is H. |

### Formulation/Utility

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

### Weight Percent

| | Active | | |
|---|---|---|---|
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox-Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Tables A-F. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

| High Strength Concentrate | |
|---|---|
| Compound 1 | 98.5% |
| silica aerogel | 0.5% |
| synthetic amorphous fine silica | 1.0% |

### Example B

| Wettable Powder | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

| Granule | |
|---|---|
| Compound 1 | 10.0% |
| attapulgite granules (low volatile matter, | 90.0% |
| 0.71/0.30 mm; U.S.S. No. 25-50 sieves) | |

### Example D

| Extruded Pellet | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

| Emulsifiable Concentrate | |
|---|---|
| Compound 1 | 10.0% |
| polyoxyethylene sorbitol hexoleate | 20.0% |
| C₆-C₁₀ fatty acid methyl ester | 70.0% |

### Example F

| Microemulsion | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

| Suspension Concentrate | |
|---|---|
| Compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example H

| Emulsion in Water | |
|---|---|
| Compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example I

| Oil Dispersion | |
|---|---|
| Compound 1 | 25% |
| polyoxyethylene sorbitol hexaoleate | 15% |
| organically modified bentonite clay | 2.5% |
| fatty acid methyl ester | 57.5% |

The present disclosure also includes Examples A through I above except that “Compound 1” is replaced with any one of “Compound 2” through “Compound 144” and “Compound 145” through “Compound 414”

Test results indicate that the compounds of the present invention are highly active preemergent and/or postemergent herbicides and/or plant growth regulants. The compounds of the mention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation. Undesired vegetation includes at least one selected from the group consisting of grass weeds and broadleaf weeds. Undesired vegetation is selected from the group consisting of annual bluegrass, Benghal dayflower, blackgrass, black nightshade, broadleaf signalgrass, Canada thistle, cheat, common cocklebur (Xanthium pensylvanicum), common ragweed, corn poppies, field violet, giant foxtail, goosegrass, green foxtail, guinea grass, hairy beggarticks, herbicide-resistant black grass, horseweed, Italian rye grass, jimsonweed, Johnson grass (Sorghum halepense), large crabgrass, little seed canary grass, morning glory, Pennsylvania smartweed, pitted morning glory, prickly sida, quackgrass, redroot pigweed, shattercane, shepherd's purse, silky windgrass, sunflower (as weed in potato), wild buckwheat (Polygonum convolvulus), wild mustard (Brassica kaber), wild oat (Avena fatua), wild pointsettia, yellow foxtail, and yellow nutsedge (Cyperus esculentus).

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Although most typically, compounds of the invention are used to control undesired vegetation, contact of desired vegetation in the treated locus with compounds of the invention may result in super-additive or enhanced effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-1H-indol-6-yl)-2-Pyridinecarboxylic cyanomethyl ester (CAS No. 2251111-18-7), aminopyralid, amitrole, ammonium sulfamate, 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6), anilofos, anisiflupurin, asulam, atrazine, azimsulfuron, bixlozone, beflubutamid, beflubutamid-M, benazolin, benazolin-ethyl, bencarbazone, benfluralin, benfuresate, benquitrione, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, bipyrazone, cafenstrole, carbetamide, 1-(2-carboxyethyl)-4-(2-pyrimidinyl)pyridazinium (CAS No. 2285384-11-2) and salts thereof, carfentrazone-ethyl, catechin, chlomethoxyfen, chloramben, chlorbromuron, chlorflurenol-methyl, chloridazon, chlorimuron-ethyl, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-Isoxazolecarboxylic ethyl ester (CAS No. 1949837-17-5), chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, cyprafluone, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimesulfazet, dimethachlor, dimethametryn, dimethenamid, dimethenamid-P, dimethipin, dimethylarsinic acid and its sodium salt, dinitramine, dinoterb, dioxopyritrione, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenpyrazone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, fluchloraminopyr, flufenacet, flufenoximacil, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, flusulfinam, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriflubenzoxim, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimisoxafen, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tripyrasulfone, tritosulfuron, vernolate, 3-(2-chloro-3,6-difluorophenyl)-4-hydroxy-1-methyl-1,5-naphthyridin-2(1H)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Preferred for better control of undesired vegetation (e.g., lower use rate such as from enhanced effects, broader spectrum of weeds controlled, or enhanced crop safety) or for preventing the development of resistant weeds are mixtures of a compound of this invention with a herbicide selected from the group consisting of atrazine, azimsulfuron, S-beflubutamid, benzisothiazolinone, carfentrazone-ethyl, chlorimuron-ethyl, chlorsulfuron-methyl, clomazone, clopyralid potassium, cloransulam-methyl, 2-[(2,4-dichlorophenyl)methyl]-4,4-dimethyl-3-isoxazolidinone, 2-[(2,5-dichlorophenyl)methyl]-4,4-dimethyl-3-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. enhanced) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When the enhanced effects of herbicidal mixtures of active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumyluron, cyometrinil, cyprosulfonamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention can also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Table A1 lists specific combinations of a Component (a) with Component (b) illustrative of the mixtures, compositions and methods of the present invention. Compound No. (Compound Number) (i.e. Compound 1) in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 1 in Index Table A) with 2,4-D is typically applied in a weight ratio between 1:384-6:1. The remaining lines of Table A1 are to be construed similarly.

TABLE A1

| Component (a) | | Typical | More Typical | Most Typical |
|---|---|---|---|---|
| (Compound No.) | Component (b) | Weight Ratio | Weight Ratio | Weight Ratio |
| 1 | 2,4-D | 1:384-6:1 | 1:128-2:1 | 1:12-1:2 |
| 1 | Acetochlor | 1:1536-2:1 | 1:512-1:2 | 1:48-1:6 |
| 1 | Acifluorfen | 1:192-12:1 | 1:64-4:1 | 1:6-2:1 |
| 1 | Aclonifen | 1:1714-2:1 | 1:571-1:3 | 1:53-1:6 |
| 1 | Alachlor | 1:1536-2:1 | 1:512-1:2 | 1:48-1:6 |
| 1 | Ametryn | 1:768-3:1 | 1:256-1:1 | 1:24-1:3 |
| 1 | Amicarbazone | 1:384-6:1 | 1:128-2:1 | 1:12-1:2 |
| 1 | Amidosulfuron | 1:13-168:1 | 1:4-56:1 | 2:1-21:1 |
| 1 | Aminocyclopyrachlor | 1:96-24:1 | 1:32-8:1 | 1:3-3:1 |
| 1 | Aminopyralid | 1:41-56:1 | 1:13-19:1 | 1:1-7:1 |
| 1 | Amitrole | 1:1536-2:1 | 1:512-1:2 | 1:48-1:6 |
| 1 | Anilofos | 1:192-12:1 | 1:64-4:1 | 1:6-2:1 |
| 1 | Asulam | 1:1920-2:1 | 1:640-1:3 | 1:60-1:7 |
| 1 | Atrazine | 1:384-6:1 | 1:128-2:1 | 1:12-1:2 |
| 1 | Azimsulfuron | 1:13-168:1 | 1:4-56:1 | 2:1-21:1 |
| 1 | Beflubutamid | 1:685-4:1 | 1:228-2:1 | 1:21-1:3 |
| 1 | Benfuresate | 1:1234-2:1 | 1:411-1:2 | 1:38-1:5 |
| 1 | Bensulfuron-methyl | 1:51-45:1 | 1:17-15:1 | 1:1-6:1 |
| 1 | Bentazone | 1:384-6:1 | 1:128-2:1 | 1:12-1:2 |
| 1 | Benzobicyclon | 1:171-14:1 | 1:57-5:1 | 1:5-2:1 |
| 1 | Benzofenap | 1:514-5:1 | 1:171-2:1 | 1:16-1:2 |
| 1 | Bicyclopyrone | 1:85-27:1 | 1:28-9:1 | 1:2-4:1 |
| 1 | Bifenox | 1:514-5:1 | 1:171-2:1 | 1:16-1:2 |
| 1 | Bispyribac-sodium | 1:20-112:1 | 1:6-38:1 | 1:1-14:1 |
| 1 | Bromacil | 1:768-3:1 | 1:256-1:1 | 1:24-1:3 |
| 1 | Bromobutide | 1:768-3:1 | 1:256-1:1 | 1:24-1:3 |
| 1 | Bromoxynil | 1:192-12:1 | 1:64-4:1 | 1:6-2:1 |
| 1 | Butachlor | 1:1536-2:1 | 1:512-1:2 | 1:48-1:6 |
| 1 | Butafenacil | 1:85-27:1 | 1:28-9:1 | 1:2-4:1 |
| 1 | Butylate | 1:3085-1:2 | 1:1028-1:5 | 1:96-1:11 |
| 1 | Carfenstrole | 1:384-6:1 | 1:128-2:1 | 1:12-1:2 |
| 1 | Carfentrazone-ethyl | 1:257-9:1 | 1:85-3:1 | 1:8-2:1 |
| 1 | Chlorimuron-ethyl | 1:17-135:1 | 1:5-45:1 | 1:1-17:1 |
| 1 | Chlorotoluron | 1:1536-2:1 | 1:512-1:2 | 1:48-1:6 |
| 1 | Chlorsulfuron | 1:13-168:1 | 1:4-56:1 | 2:1-21:1 |
| 1 | Cincosulfuron | 1:34-68:1 | 1:11-23:1 | 1:1-9:1 |
| 1 | Cinidon-ethyl | 1:768-3:1 | 1:256-1:1 | 1:24-1:3 |
| 1 | Cinmethylin | 1:68-34:1 | 1:22-12:1 | 1:2-5:1 |
| 1 | Clacyfos | 1:384-6:1 | 1:128-2:1 | 1:12-1:2 |
| 1 | Clethodim | 1:96-24:1 | 1:32-8:1 | 1:3-3:1 |
| 1 | Clodinafop-propargyl | 1:41-56:1 | 1:13-19:1 | 1:1-7:1 |
| 1 | Clomazone | 1:768-3:1 | 1:256-1:1 | 1:24-1:3 |
| 1 | Clomeprop | 1:342-7:1 | 1:114-3:1 | 1:10-1:2 |
| 1 | Clopyralid | 1:384-6:1 | 1:128-2:1 | 1:12-1:2 |
| 1 | Cloransulam-methyl | 1:24-96:1 | 1:8-32:1 | 1:1-12:1 |
| 1 | Cumyluron | 1:768-3:1 | 1:256-1:1 | 1:24-1:3 |
| 1 | Cyanazine | 1:768-3:1 | 1:256-1:1 | 1:24-1:3 |
| 1 | Cyclopyrimorate | 1:34-68:1 | 1:11-23:1 | 1:1-9:1 |
| 1 | Cyclosulfamuron | 1:34-68:1 | 1:11-23:1 | 1:1-9:1 |
| 1 | Cycloxydim | 1:192-12:1 | 1:64-4:1 | 1:6-2:1 |
| 1 | Cyhalofop | 1:51-45:1 | 1:17-15:1 | 1:1-6:1 |
| 1 | Daimuron | 1:384-6:1 | 1:128-2:1 | 1:12-1:2 |
| 1 | Desmedipham | 1:644-4:1 | 1:214-2:1 | 1:20-1:3 |
| 1 | Dicamba | 1:384-6:1 | 1:128-2:1 | 1:12-1:2 |
| 1 | Dichlobenil | 1:2742-1:2 | 1:914-1:4 | 1:85-1:10 |
| 1 | Dichlorprop | 1:1851-2:1 | 1:617-1:3 | 1:57-1:7 |
| 1 | Diclofop-methyl | 1:768-3:1 | 1:256-1:1 | 1:24-1:3 |
| 1 | Diclosulam | 1:20-112:1 | 1:6-38:1 | 1:1-14:1 |
| 1 | Difenzoquat | 1:576-4:1 | 1:192-2:1 | 1:18-1:2 |
| 1 | Diflufenican | 1:1714-2:1 | 1:571-1:3 | 1:53-1:6 |
| 1 | Diflufenzopyr | 1:24-96:1 | 1:8-32:1 | 1:1-12:1 |
| 1 | Dimethachlor | 1:1536-2:1 | 1:512-1:2 | 1:48-1:6 |
| 1 | Dimethametryn | 1:384-6:1 | 1:128-2:1 | 1:12-1:2 |
| 1 | Dimethenamid-P | 1:768-3:1 | 1:256-1:1 | 1:24-1:3 |
| 1 | Dithiopyr | 1:384-6:1 | 1:128-2:1 | 1:12-1:2 |
| 1 | Diuron | 1:768-3:1 | 1:256-1:1 | 1:24-1:3 |
| 1 | EPTC | 1:1536-2:1 | 1:512-1:2 | 1:48-1:6 |
| 1 | Esprocarb | 1:2742-1:2 | 1:914-1:4 | 1:85-1:10 |
| 1 | Ethalfluralin | 1:768-3:1 | 1:256-1:1 | 1:24-1:3 |
| 1 | Ethametsulfuron-methyl | 1:34-68:1 | 1:11-23:1 | 1:1-9:1 |
| 1 | Ethoxyfen | 1:17-135:1 | 1:5-45:1 | 1:1-17:1 |
| 1 | Ethoxysulfuron | 1:41-56:1 | 1:13-19:1 | 1:1-7:1 |
| 1 | Etobenzanid | 1:514-5:1 | 1:171-2:1 | 1:16-1:2 |
| 1 | Fenoxaprop-ethyl | 1:240-10:1 | 1:80-4:1 | 1:7-2:1 |
| 1 | Fenoxasulfone | 1:171-14:1 | 1:57-5:1 | 1:5-2:1 |
| 1 | Fenquinotrione | 1:85-27:1 | 1:28-9:1 | 1:2-4:1 |
| 1 | Fentrazamide | 1:34-68:1 | 1:11-23:1 | 1:1-9:1 |
| 1 | Flazasulfuron | 1:34-68:1 | 1:11-23:1 | 1:1-9:1 |
| 1 | Florasulam | 1:5-420:1 | 1:1-140:1 | 5:1-53:1 |
| 1 | Fluazifop-butyl | 1:384-6:1 | 1:128-2:1 | 1:12-1:2 |
| 1 | Flucarbazone | 1:17-135:1 | 1:5-45:1 | 1:1-17:1 |
| 1 | Flucetosulfuron | 1:17-135:1 | 1:5-45:1 | 1:1-17:1 |
| 1 | Flufenacet | 1:514-5:1 | 1:171-2:1 | 1:16-1:2 |
| 1 | Flumetsulam | 1:48-48:1 | 1:16-16:1 | 1:1-6:1 |
| 1 | Flumiclorac-pentyl | 1:20-112:1 | 1:6-38:1 | 1:1-14:1 |
| 1 | Flumioxazin | 1:51-45:1 | 1:17-15:1 | 1:1-6:1 |
| 1 | Fluometuron | 1:768-3:1 | 1:256-1:1 | 1:24-1:3 |
| 1 | Flupyrsulfuron-methyl | 1:6-336:1 | 1:2-112:1 | 4:1-42:1 |
| 1 | Fluridone | 1:768-3:1 | 1:256-1:1 | 1:24-1:3 |
| 1 | Fluroxypyr | 1:192-12:1 | 1:64-4:1 | 1:6-2:1 |
| 1 | Flurtamone | 1:1714-2:1 | 1:571-1:3 | 1:53-1:6 |
| 1 | Fluthiacet-methyl | 1:96-42:1 | 1:32-14:1 | 1:1-6:1 |
| 1 | Fomesafen | 1:192-12:1 | 1:64-4:1 | 1:6-2:1 |
| 1 | Foramsulfuron | 1:27-84:1 | 1:9-28:1 | 1:1-11:1 |
| 1 | Glufosinate | 1:576-4:1 | 1:192-2:1 | 1:18-1:2 |
| 1 | Glyphosate | 1:576-4:1 | 1:192-2:1 | 1:18-1:2 |
| 1 | Halosulfuron-methyl | 1:34-68:1 | 1:11-23:1 | 1:1-9:1 |
| 1 | Halauxifen | 1:41-56:1 | 1:13-19:1 | 1:1-7:1 |
| 1 | Halauxifen methyl | 1:41-56:1 | 1:13-19:1 | 1:1-7:1 |
| 1 | Haloxyfop-methyl | 1:68-34:1 | 1:22-12:1 | 1:2-5:1 |
| 1 | Hexazinone | 1:384-6:1 | 1:128-2:1 | 1:12-1:2 |
| 1 | Imazamox | 1:27-84:1 | 1:9-28:1 | 1:1-11:1 |
| 1 | Imazapic | 1:41-56:1 | 1:13-19:1 | 1:1-7:1 |
| 1 | Imazapyr | 1:171-14:1 | 1:57-5:1 | 1:5-2:1 |
| 1 | Imazaquin | 1:68-34:1 | 1:22-12:1 | 1:2-5:1 |
| 1 | Imazethabenz-methyl | 1:342-7:1 | 1:114-3:1 | 1:10-1:2 |
| 1 | Imazethapyr | 1:48-48:1 | 1:16-16:1 | 1:1-6:1 |
| 1 | Imazosulfuron | 1:54-42:1 | 1:18-14:1 | 1:1-6:1 |
| 1 | Indanofan | 1:685-4:1 | 1:228-2:1 | 1:21-1:3 |
| 1 | Indaziflam | 1:51-45:1 | 1:17-15:1 | 1:1-6:1 |
| 1 | Iodosulfuron-methyl | 1:6-336:1 | 1:2-112:1 | 4:1-42:1 |
| 1 | Ioxynil | 1:384-6:1 | 1:128-2:1 | 1:12-1:2 |
| 1 | Ipfencarbazone | 1:171-14:1 | 1:57-5:1 | 1:5-2:1 |
| 1 | Isoproturon | 1:768-3:1 | 1:256-1:1 | 1:24-1:3 |
| 1 | Isoxaben | 1:576-4:1 | 1:192-2:1 | 1:18-1:2 |
| 1 | Isoxaflutole | 1:120-20:1 | 1:40-7:1 | 1:3-3:1 |
| 1 | Lactofen | 1:85-27:1 | 1:28-9:1 | 1:2-4:1 |
| 1 | Lenacil | 1:768-3:1 | 1:256-1:1 | 1:24-1:3 |
| 1 | Linuron | 1:768-3:1 | 1:256-1:1 | 1:24-1:3 |
| 1 | MCPA | 1:384-6:1 | 1:128-2:1 | 1:12-1:2 |
| 1 | MCPB | 1:576-4:1 | 1:192-2:1 | 1:18-1:2 |
| 1 | Mecoprop | 1:1536-2:1 | 1:512-1:2 | 1:48-1:6 |
| 1 | Mefenacet | 1:768-3:1 | 1:256-1:1 | 1:24-1:3 |
| 1 | Mefluidide | 1:384-6:1 | 1:128-2:1 | 1:12-1:2 |
| 1 | Mesosulfuron-methyl | 1:10-224:1 | 1:3-75:1 | 3:1-28:1 |
| 1 | Mesotrione | 1:85-27:1 | 1:28-9:1 | 1:2-4:1 |
| 1 | Metamifop | 1:85-27:1 | 1:28-9:1 | 1:2-4:1 |
| 1 | Metazachlor | 1:768-3:1 | 1:256-1:1 | 1:24-1:3 |
| 1 | Metazosulfuron | 1:51-45:1 | 1:17-15:1 | 1:1-6:1 |
| 1 | Methabenzthiazuron | 1:1536-2:1 | 1:512-1:2 | 1:48-1:6 |
| 1 | Metolachlor | 1:1536-2:1 | 1:512-1:2 | 1:48-1:6 |
| 1 | Metosulam | 1:17-135:1 | 1:5-45:1 | 1:1-17:1 |
| 1 | Metribuzin | 1:384-6:1 | 1:128-2:1 | 1:12-1:2 |
| 1 | Metsulfuron-methyl | 1:4-560:1 | 1:1-187:1 | 7:1-70:1 |
| 1 | Molinate | 1:2057-2:1 | 1:685-1:3 | 1:64-1:8 |
| 1 | Napropamide | 1:768-3:1 | 1:256-1:1 | 1:24-1:3 |
| 1 | Napropamide-M | 1:384-6:1 | 1:128-2:1 | 1:12-1:2 |
| 1 | Naptalam | 1:384-6:1 | 1:128-2:1 | 1:12-1:2 |
| 1 | Nicosulfuron | 1:24-96:1 | 1:8-32:1 | 1:1-12:1 |
| 1 | Norflurazon | 1:2304-1:1 | 1:768-1:3 | 1:72-1:8 |
| 1 | Orbencarb | 1:2742-1:2 | 1:914-1:4 | 1:85-1:10 |
| 1 | Orthosulfamuron | 1:41-56:1 | 1:13-19:1 | 1:1-7:1 |
| 1 | Oryzalin | 1:1028-3:1 | 1:342-1:2 | 1:32-1:4 |
| 1 | Oxadiargyl | 1:768-3:1 | 1:256-1:1 | 1:24-1:3 |
| 1 | Oxadiazon | 1:1097-3:1 | 1:365-1:2 | 1:34-1:4 |
| 1 | Oxasulfuron | 1:54-42:1 | 1:18-14:1 | 1:1-6:1 |
| 1 | Oxaziclomefone | 1:85-27:1 | 1:28-9:1 | 1:2-4:1 |
| 1 | Oxyfluorfen | 1:768-3:1 | 1:256-1:1 | 1:24-1:3 |
| 1 | Paraquat | 1:384-6:1 | 1:128-2:1 | 1:12-1:2 |
| 1 | Pendimethalin | 1:768-3:1 | 1:256-1:1 | 1:24-1:3 |
| 1 | Penoxsulam | 1:20-112:1 | 1:6-38:1 | 1:1-14:1 |
| 1 | Penthoxamid | 1:768-3:1 | 1:256-1:1 | 1:24-1:3 |
| 1 | Pentoxazone | 1:205-12:1 | 1:68-4:1 | 1:6-2:1 |
| 1 | Phenmedipham | 1:205-12:1 | 1:68-4:1 | 1:6-2:1 |
| 1 | Picloram | 1:192-12:1 | 1:64-4:1 | 1:6-2:1 |
| 1 | Picolinafen | 1:68-34:1 | 1:22-12:1 | 1:2-5:1 |
| 1 | Pinoxaden | 1:51-45:1 | 1:17-15:1 | 1:1-6:1 |
| 1 | Pretilachlor | 1:384-6:1 | 1:128-2:1 | 1:12-1:2 |
| 1 | Primisulfuron-methyl | 1:17-135:1 | 1:5-45:1 | 1:1-17:1 |
| 1 | Prodiamine | 1:768-3:1 | 1:256-1:1 | 1:24-1:3 |
| 1 | Profoxydim | 1:85-27:1 | 1:28-9:1 | 1:2-4:1 |
| 1 | Prometryn | 1:768-3:1 | 1:256-1:1 | 1:24-1:3 |
| 1 | Propachlor | 1:2304-1:1 | 1:768-1:3 | 1:72-1:8 |
| 1 | Propanil | 1:768-3:1 | 1:256-1:1 | 1:24-1:3 |
| 1 | Propaquizafop | 1:96-24:1 | 1:32-8:1 | 1:3-3:1 |
| 1 | Propoxycarbazone | 1:34-68:1 | 1:11-23:1 | 1:1-9:1 |
| 1 | Propyrisulfuron | 1:34-68:1 | 1:11-23:1 | 1:1-9:1 |
| 1 | Propyzamide | 1:768-3:1 | 1:256-1:1 | 1:24-1:3 |
| 1 | Prosulfocarb | 1:2400-1:2 | 1:800-1:4 | 1:75-1:9 |
| 1 | Prosulfuron | 1:13-168:1 | 1:4-56:1 | 2:1-21:1 |
| 1 | Pyraclonil | 1:85-27:1 | 1:28-9:1 | 1:2-4:1 |
| 1 | Pyraflufen-ethyl | 1:10-224:1 | 1:3-75:1 | 3:1-28:1 |
| 1 | Pyrasulfotole | 1:27-84:1 | 1:9-28:1 | 1:1-11:1 |
| 1 | Pyrazolynate | 1:1714-2:1 | 1:571-1:3 | 1:53-1:6 |
| 1 | Pyrazosulfuron-ethyl | 1:20-112:1 | 1:6-38:1 | 1:1-14:1 |
| 1 | Pyrazoxyfen | 1:10-224:1 | 1:3-75:1 | 3:1-28:1 |
| 1 | Pyribenzoxim | 1:20-112:1 | 1:6-38:1 | 1:1-14:1 |
| 1 | Pyributicarb | 1:768-3:1 | 1:256-1:1 | 1:24-1:3 |
| 1 | Pyridate | 1:576-4:1 | 1:192-2:1 | 1:18-1:2 |
| 1 | Pyriftalid | 1:20-112:1 | 1:6-38:1 | 1:1-14:1 |
| 1 | Pyriminobac-methyl | 1:41-56:1 | 1:13-19:1 | 1:1-7:1 |
| 1 | Pyrimisulfan | 1:34-68:1 | 1:11-23:1 | 1:1-9:1 |
| 1 | Pyrithiobac | 1:48-48:1 | 1:16-16:1 | 1:1-6:1 |
| 1 | Pyroxasulfone | 1:171-14:1 | 1:57-5:1 | 1:5-2:1 |
| 1 | Pyroxsulam | 1:10-224:1 | 1:3-75:1 | 3:1-28:1 |
| 1 | Quinclorac | 1:384-6:1 | 1:128-2:1 | 1:12-1:2 |
| 1 | Quizalofop-ethyl | 1:85-27:1 | 1:28-9:1 | 1:2-4:1 |
| 1 | Rimsulfuron | 1:27-84:1 | 1:9-28:1 | 1:1-11:1 |
| 1 | Saflufenacil | 1:51-45:1 | 1:17-15:1 | 1:1-6:1 |
| 1 | Sethoxydim | 1:192-12:1 | 1:64-4:1 | 1:6-2:1 |
| 1 | Simazine | 1:768-3:1 | 1:256-1:1 | 1:24-1:3 |
| 1 | Sulcotrione | 1:240-10:1 | 1:80-4:1 | 1:7-2:1 |
| 1 | Sulfentrazone | 1:294-8:1 | 1:98-3:1 | 1:9-1:2 |
| 1 | Sulfometuron-methyl | 1:68-34:1 | 1:22-12:1 | 1:2-5:1 |
| 1 | Sulfosulfuron | 1:17-135:1 | 1:5-45:1 | 1:1-17:1 |
| 1 | Tebuthiuron | 1:768-3:1 | 1:256-1:1 | 1:24-1:3 |
| 1 | Tefuryltrione | 1:85-27:1 | 1:28-9:1 | 1:2-4:1 |
| 1 | Tembotrione | 1:63-37:1 | 1:21-13:1 | 1:1-5:1 |
| 1 | Tepraloxydim | 1:51-45:1 | 1:17-15:1 | 1:1-6:1 |
| 1 | Terbacil | 1:576-4:1 | 1:192-2:1 | 1:18-1:2 |
| 1 | Terbuthylazine | 1:1714-2:1 | 1:571-1:3 | 1:53-1:6 |
| 1 | Terbutryn | 1:384-6:1 | 1:128-2:1 | 1:12-1:2 |
| 1 | Thenylchlor | 1:171-14:1 | 1:57-5:1 | 1:5-2:1 |
| 1 | Thiazopyr | 1:768-3:1 | 1:256-1:1 | 1:24-1:3 |
| 1 | Thiencarbazone | 1:6-336:1 | 1:2-112:1 | 4:1-42:1 |
| 1 | Thifensulfuron-methyl | 1:10-224:1 | 1:3-75:1 | 3:1-28:1 |
| 1 | Tiafenacil | 1:85-27:1 | 1:28-9:1 | 1:2-4:1 |
| 1 | Thiobencarb | 1:1536-2:1 | 1:512-1:2 | 1:48-1:6 |
| 1 | Topramezone | 1:13-168:1 | 1:4-56:1 | 2:1-21:1 |
| 1 | Tralkoxydim | 1:137-17:1 | 1:45-6:1 | 1:4-3:1 |
| 1 | Triallate | 1:1536-2:1 | 1:512-1:2 | 1:48-1:6 |
| 1 | Triasulfuron | 1:10-224:1 | 1:3-75:1 | 3:1-28:1 |
| 1 | Triaziflam | 1:342-7:1 | 1:114-3:1 | 1:10-1:2 |
| 1 | Tribenuron-methyl | 1:6-336:1 | 1:2-112:1 | 4:1-42:1 |
| 1 | Triclopyr | 1:384-6:1 | 1:128-2:1 | 1:12-1:2 |
| 1 | Trifloxysulfuron | 1:5-420:1 | 1:1-140:1 | 5:1-53:1 |
| 1 | Trifluralin | 1:576-4:1 | 1:192-2:1 | 1:18-1:2 |
| 1 | Triflusulfuron-methyl | 1:34-68:1 | 1:11-23:1 | 1:1-9:1 |
| 1 | Tritosulfuron | 1:27-84:1 | 1:9-28:1 | 1:1-11:1 |

Table A2 is constructed the same as Table A1 above except that those entries below the “Component (a)” column heading are replaced with the respective Component (a) Column Entry shown below. Compound No. in the Component (a) column is identified in Index Table A. Thus, for example, in Table A2 the entries below the “Component (a)” column heading all recite “Compound 2” (i.e. Compound 2 identified in Index Table A), and the first line below the column headings in Table A2 specifically discloses a mixture of Compound 2 with 2,4-D. Tables A3 through A414 are constructed similarly.

| Table Number | Component (a) Column Entries |
|---|---|
| A2 | Compound 2 |
| A3 | Compound 3 |
| A4 | Compound 4 |
| A5 | Compound 5 |
| A6 | Compound 6 |
| A7 | Compound 7 |
| A8 | Compound 8 |
| A9 | Compound 9 |
| A10 | Compound 10 |
| A11 | Compound 11 |
| A12 | Compound 12 |
| A13 | Compound 13 |
| A14 | Compound 14 |
| A15 | Compound 15 |
| A16 | Compound 16 |
| A17 | Compound 17 |
| A18 | Compound 18 |
| A19 | Compound 19 |
| A20 | Compound 20 |
| A21 | Compound 21 |
| A22 | Compound 22 |
| A23 | Compound 23 |
| A24 | Compound 24 |
| A25 | Compound 25 |
| A26 | Compound 26 |
| A27 | Compound 27 |
| A28 | Compound 28 |
| A29 | Compound 29 |
| A30 | Compound 30 |
| A31 | Compound 31 |
| A32 | Compound 32 |
| A33 | Compound 33 |
| A34 | Compound 34 |
| A35 | Compound 35 |
| A36 | Compound 36 |
| A37 | Compound 37 |
| A38 | Compound 38 |
| A39 | Compound 39 |
| A40 | Compound 40 |
| A41 | Compound 31 |
| A42 | Compound 32 |
| A43 | Compound 33 |
| A44 | Compound 34 |
| A45 | Compound 35 |
| A46 | Compound 36 |
| A47 | Compound 37 |
| A48 | Compound 38 |
| A49 | Compound 39 |
| A50 | Compound 50 |
| A51 | Compound 51 |
| A52 | Compound 52 |
| A53 | Compound 53 |
| A54 | Compound 54 |
| A55 | Compound 55 |
| A56 | Compound 56 |
| A57 | Compound 57 |
| A58 | Compound 58 |
| A59 | Compound 59 |
| A60 | Compound 60 |
| A61 | Compound 61 |
| A62 | Compound 62 |
| A63 | Compound 63 |
| A64 | Compound 64 |
| A65 | Compound 65 |
| A66 | Compound 66 |
| A67 | Compound 67 |
| A68 | Compound 68 |
| A69 | Compound 69 |
| A70 | Compound 70 |
| A71 | Compound 71 |
| A72 | Compound 72 |
| A73 | Compound 73 |
| A74 | Compound 74 |
| A75 | Compound 75 |
| A76 | Compound 76 |
| A77 | Compound 77 |
| A78 | Compound 78 |
| A79 | Compound 79 |
| A80 | Compound 80 |
| A81 | Compound 81 |
| A82 | Compound 82 |
| A83 | Compound 83 |
| A84 | Compound 84 |
| A85 | Compound 85 |
| A86 | Compound 86 |
| A87 | Compound 87 |
| A88 | Compound 88 |
| A89 | Compound 89 |
| A90 | Compound 90 |
| A91 | Compound 91 |
| A92 | Compound 92 |
| A93 | Compound 93 |
| A94 | Compound 94 |
| A95 | Compound 95 |
| A96 | Compound 96 |
| A97 | Compound 97 |
| A98 | Compound 98 |
| A99 | Compound 99 |
| A100 | Compound 100 |
| A101 | Compound 101 |
| A102 | Compound 102 |
| A103 | Compound 103 |
| A104 | Compound 104 |
| A105 | Compound 105 |
| A106 | Compound 106 |
| A107 | Compound 107 |
| A108 | Compound 108 |
| A109 | Compound 109 |
| A110 | Compound 100 |
| A111 | Compound 111 |
| A112 | Compound 112 |
| A113 | Compound 113 |
| A114 | Compound 114 |
| A115 | Compound 115 |
| A116 | Compound 116 |
| A117 | Compound 117 |
| A118 | Compound 118 |
| A119 | Compound 119 |
| A120 | Compound 120 |
| A121 | Compound 121 |
| A122 | Compound 122 |
| A123 | Compound 123 |
| A124 | Compound 124 |
| A125 | Compound 125 |
| A126 | Compound 126 |
| A127 | Compound 127 |
| A128 | Compound 128 |
| A129 | Compound 129 |
| A130 | Compound 130 |
| A131 | Compound 131 |
| A132 | Compound 132 |
| A133 | Compound 133 |
| A134 | Compound 134 |
| A135 | Compound 135 |
| A136 | Compound 136 |
| A137 | Compound 137 |
| A138 | Compound 138 |
| A139 | Compound 139 |
| A140 | Compound 140 |
| A141 | Compound 141 |
| A142 | Compound 142 |
| A143 | Compound 143 |
| A144 | Compound 143 |
| A145 | Compound 145 |
| A146 | Compound 146 |
| A147 | Compound 147 |
| A148 | Compound 148 |
| A149 | Compound 149 |
| A150 | Compound 150 |
| A151 | Compound 151 |
| A152 | Compound 152 |
| A153 | Compound 153 |
| A154 | Compound 154 |
| A155 | Compound 155 |
| A156 | Compound 156 |
| A157 | Compound 157 |
| A158 | Compound 158 |
| A159 | Compound 159 |
| A160 | Compound 160 |
| A161 | Compound 161 |
| A162 | Compound 162 |
| A163 | Compound 163 |
| A164 | Compound 164 |
| A165 | Compound 165 |
| A166 | Compound 166 |
| A167 | Compound 167 |
| A168 | Compound 168 |
| A169 | Compound 169 |
| A170 | Compound 170 |
| A171 | Compound 171 |
| A172 | Compound 172 |
| A173 | Compound 173 |
| A174 | Compound 174 |
| A175 | Compound 175 |
| A176 | Compound 176 |
| A177 | Compound 177 |
| A178 | Compound 178 |
| A179 | Compound 179 |
| A180 | Compound 180 |
| A181 | Compound 181 |
| A182 | Compound 182 |
| A183 | Compound 183 |
| A184 | Compound 184 |
| A185 | Compound 185 |
| A186 | Compound 186 |
| A187 | Compound 187 |
| A188 | Compound 188 |
| A189 | Compound 189 |
| A190 | Compound 190 |
| A191 | Compound 191 |
| A192 | Compound 192 |
| A193 | Compound 193 |
| A194 | Compound 194 |
| A195 | Compound 195 |
| A196 | Compound 196 |
| A197 | Compound 197 |
| A198 | Compound 198 |
| A199 | Compound 199 |
| A200 | Compound 200 |
| A201 | Compound 201 |
| A202 | Compound 202 |
| A203 | Compound 203 |
| A204 | Compound 204 |
| A205 | Compound 205 |
| A206 | Compound 206 |
| A207 | Compound 207 |
| A208 | Compound 208 |
| A209 | Compound 209 |
| A210 | Compound 210 |
| A211 | Compound 211 |
| A212 | Compound 212 |
| A213 | Compound 213 |
| A214 | Compound 214 |
| A215 | Compound 215 |
| A216 | Compound 216 |
| A217 | Compound 217 |
| A218 | Compound 218 |
| A219 | Compound 219 |
| A220 | Compound 220 |
| A221 | Compound 221 |
| A222 | Compound 222 |
| A223 | Compound 223 |
| A224 | Compound 224 |
| A225 | Compound 225 |
| A226 | Compound 226 |
| A227 | Compound 227 |
| A228 | Compound 228 |
| A229 | Compound 229 |
| A230 | Compound 230 |
| A231 | Compound 231 |
| A232 | Compound 232 |
| A233 | Compound 233 |
| A234 | Compound 234 |
| A235 | Compound 235 |
| A236 | Compound 236 |
| A237 | Compound 237 |
| A238 | Compound 238 |
| A239 | Compound 239 |
| A240 | Compound 240 |
| A241 | Compound 241 |
| A242 | Compound 242 |
| A243 | Compound 243 |
| A244 | Compound 244 |
| A245 | Compound 245 |
| A246 | Compound 246 |
| A247 | Compound 247 |
| A248 | Compound 248 |
| A249 | Compound 249 |
| A250 | Compound 250 |
| A251 | Compound 251 |
| A252 | Compound 252 |
| A253 | Compound 253 |
| A254 | Compound 254 |
| A255 | Compound 255 |
| A256 | Compound 256 |
| A257 | Compound 257 |
| A258 | Compound 258 |
| A259 | Compound 259 |
| A260 | Compound 260 |
| A261 | Compound 261 |
| A262 | Compound 262 |
| A263 | Compound 263 |
| A264 | Compound 264 |
| A265 | Compound 265 |
| A266 | Compound 266 |
| A267 | Compound 267 |
| A268 | Compound 268 |
| A269 | Compound 269 |
| A270 | Compound 270 |
| A271 | Compound 271 |
| A272 | Compound 272 |
| A273 | Compound 273 |
| A274 | Compound 274 |
| A275 | Compound 275 |
| A276 | Compound 276 |
| A277 | Compound 277 |
| A278 | Compound 278 |
| A279 | Compound 279 |
| A280 | Compound 280 |
| A281 | Compound 281 |
| A282 | Compound 282 |
| A283 | Compound 283 |
| A284 | Compound 284 |
| A285 | Compound 285 |
| A286 | Compound 286 |
| A287 | Compound 287 |
| A288 | Compound 288 |
| A289 | Compound 289 |
| A290 | Compound 290 |
| A291 | Compound 291 |
| A292 | Compound 292 |
| A293 | Compound 293 |
| A294 | Compound 294 |
| A295 | Compound 295 |
| A296 | Compound 296 |
| A297 | Compound 297 |
| A298 | Compound 298 |
| A299 | Compound 299 |
| A300 | Compound 300 |
| A301 | Compound 301 |
| A302 | Compound 302 |
| A303 | Compound 303 |
| A304 | Compound 304 |
| A305 | Compound 305 |
| A306 | Compound 306 |
| A307 | Compound 307 |
| A308 | Compound 308 |
| A309 | Compound 309 |
| A310 | Compound 310 |
| A311 | Compound 311 |
| A312 | Compound 312 |
| A313 | Compound 313 |
| A314 | Compound 314 |
| A315 | Compound 315 |
| A316 | Compound 316 |
| A317 | Compound 317 |
| A318 | Compound 318 |
| A319 | Compound 319 |
| A320 | Compound 320 |
| A321 | Compound 321 |
| A322 | Compound 322 |
| A323 | Compound 323 |
| A324 | Compound 324 |
| A325 | Compound 325 |
| A326 | Compound 326 |
| A327 | Compound 327 |
| A328 | Compound 328 |
| A329 | Compound 329 |
| A330 | Compound 330 |
| A331 | Compound 331 |
| A332 | Compound 332 |
| A333 | Compound 333 |
| A334 | Compound 334 |
| A335 | Compound 335 |
| A336 | Compound 336 |
| A337 | Compound 337 |
| A338 | Compound 338 |
| A339 | Compound 339 |
| A340 | Compound 340 |
| A341 | Compound 341 |
| A342 | Compound 342 |
| A343 | Compound 343 |
| A344 | Compound 344 |
| A345 | Compound 345 |
| A346 | Compound 346 |
| A347 | Compound 347 |
| A348 | Compound 348 |
| A349 | Compound 349 |
| A350 | Compound 350 |
| A351 | Compound 351 |
| A352 | Compound 352 |
| A353 | Compound 353 |
| A354 | Compound 354 |
| A355 | Compound 355 |
| A356 | Compound 356 |
| A357 | Compound 357 |
| A358 | Compound 358 |
| A359 | Compound 359 |
| A360 | Compound 360 |
| A361 | Compound 361 |
| A362 | Compound 362 |
| A363 | Compound 363 |
| A364 | Compound 364 |
| A365 | Compound 365 |
| A366 | Compound 366 |
| A367 | Compound 367 |
| A368 | Compound 368 |
| A369 | Compound 369 |
| A370 | Compound 370 |
| A371 | Compound 371 |
| A372 | Compound 372 |
| A373 | Compound 373 |
| A374 | Compound 374 |
| A375 | Compound 375 |
| A376 | Compound 376 |
| A377 | Compound 377 |
| A378 | Compound 378 |
| A379 | Compound 379 |
| A380 | Compound 380 |
| A381 | Compound 381 |
| A382 | Compound 382 |
| A383 | Compound 383 |
| A384 | Compound 384 |
| A385 | Compound 385 |
| A386 | Compound 386 |
| A387 | Compound 387 |
| A388 | Compound 388 |
| A389 | Compound 389 |
| A390 | Compound 390 |
| A391 | Compound 391 |
| A392 | Compound 392 |
| A393 | Compound 393 |
| A394 | Compound 394 |
| A395 | Compound 395 |
| A396 | Compound 396 |
| A397 | Compound 397 |
| A398 | Compound 398 |
| A399 | Compound 399 |
| A400 | Compound 400 |
| A401 | Compound 401 |
| A402 | Compound 402 |
| A403 | Compound 403 |
| A404 | Compound 404 |
| A405 | Compound 405 |
| A406 | Compound 406 |
| A407 | Compound 407 |
| A408 | Compound 408 |
| A409 | Compound 409 |
| A410 | Compound 410 |
| A411 | Compound 411 |
| A412 | Compound 412 |
| A413 | Compound 413 |
| A414 | Compound 414 |

Preferred for better control of undesired vegetation (e.g., lower use rate such as from enhanced effects, broader spectrum of weeds controlled, or enhanced crop safety) or for preventing the development of resistant weeds are mixtures of a compound of this invention with a herbicide selected from the group consisting of chlorimuron-ethyl, nicosulfuron, mesotrione, thifensulfuron-methyl, flupyrsulfuron-methyl, tribenuron, pyroxasulfone, pinoxaden, tembotrione, pyroxsulam, metolachlor and S-metolachlor.

The following Tests demonstrate the control efficacy of the compounds of this invention against specific weeds. The weed control afforded by the compounds is not limited, however, to these species. See Index Tables A through F for compound descriptions. The following abbreviations are used in the Index Tables which follow: t is tertiary, s is secondary, n is normal, i is iso, c is cyclo, Me is methyl, Et is ethyl, Pro is propyl, i-Pro is isopropyl, Bu is butyl, c-Pro is cyclopropyl, c-Bu is cyclobutyl, c-Pen is cyclopentyl, t-Bu is tert-butyl, i-Bu is iso-butyl, s-Bu is sec-butyl,Ph is phenyl, OMe is methoxy, OEt is ethoxy, SMe is methylthio, SEt is ethylthio, —CN is cyano, —NO₂ is nitro, TMS is trimethylsilyl, allyl is CH₂CH═CH₂, propargyl is CH₂C≡CH and naphthyl means naphthalenyl. Some other structures are defined in the table below.

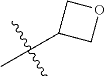 [SMILES: *C1COC1]

3-oxetanyl

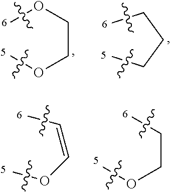

5 and 6 represent the carbon ring
member at the 5 and 6 position
of the Ph ring (bearing R2
substiturents) of Formula 1.

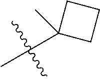 [SMILES: *C1(C)CCC1]

1-Me-c-Bu

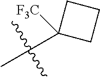 [SMILES: *C1(C(F)(F)F)CCC1]

1-CF₃-c-Bu

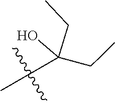 [SMILES: *C(O)(CC)CC]

3-OH-Pen-3-yl

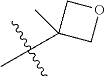 [SMILES: *C1(C)COC1]

3-Me-3-oxetanyl

(1′) or (1″) denotes the absolute chirality of the asymmetric carbon center (*). (1′) and (1″) are defined as below.

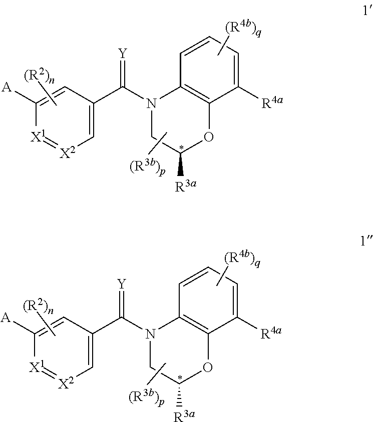

The abbreviation “(d)” indicates that the compound appeared to decompose on melting. The abbreviation “Cmpd. #” stands for “Compound Number”. The abbreviation “Ex.” stands for “Example” and is followed by a number indicating in which example the compound is prepared. Mass spectra are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+).

INDEX TABLE A

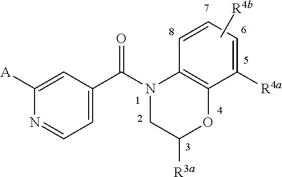

| Cmpd | | | | | | | | M.P. |
|---|---|---|---|---|---|---|---|---|
| # | A | R^{1a} | R^{1b} | R^{3a} | R^{4a} | R^{4b} | M.S. | (° C.) |
| 1 | A-1 | H | H | Me | Cl | H | 356.1 | |
| 2 | A-1 | H | H | Pro | Cl | H | 384.1 | |
| 3 | A-1 | H | H | i-Pro | Cl | H | 384.1 | |
| 4 | A-1 | H | H | (1′) or | Cl | H | 370.1 | |
| | | | | (1″)-Et | | | | |
| 5 | A-1 | H | H | (1′) or | Cl | H | 370.1 | |
| | | | | (1″)-Et | | | | |
| 8 | A-1 | H | H | Me | H | H | 322 | |
| 9 | A-1 | H | H | (1″)-Me | Cl | H | 356 | |
| 10 | A-1 | H | H | (1′)-Me | Cl | H | 356 | |
| 11 | A-1 | H | H | Et | Me | H | 349.9 | |
| 12 | A-1 | H | H | Et | H | 7-Cl | 370 | |
| 13 | A-1 | H | H | Et | Br | H | 414.1 | |
| 14 | A-1 | H | H | Et | H | 8-Cl | 370.1 | |
| 15 | A-1 | H | H | Et | F | H | 354.1 | |
| 16 | A-1 | H | H | Et | H | 6-Cl | 370 | |
| 17 | A-1 | H | H | Et | I | H | 461.9 | |
| 19 | A-1 | H | H | (1′)-Me | H | H | 322 | |
| 34 | A-1 | H | H | CF₃ | Cl | H | | 131-134 |
| 35 | A-1 | H | H | CH₂OMe | Cl | H | 386.15 | |
| 36 | A-1 | H | H | CH₂F | Cl | H | | 86-89 |
| 116 | A-13 | — | H | (1′)-Me | Cl | H | 366.2 | |
| 144 | A-1 | H | H | CF₃ | Me | H | | 169-172 |
| 197 | A-25 | — | H | (1′)-Me | Cl | H | | |
| 198 | A-26 | — | H | (1′)-Me | Cl | H | | |
| 286 | A-18 | — | H | Et | Cl | H | 380 | |
| 318 | A-1 | Et | H | CF₃ | Me | H | | 70-73 |
| 319 | A-1 | t-Bu | H | CF₃ | Me | H | 446 | |
| 320 | A-1 | i-Pro | H | (1′) or | Me | H | | 62-65 |
| | | | | (1″)-CF₃ | | | | |
| 321 | A-1 | Me | H | CF₃ | Cl | H | | 147-150 |
| 322 | A-1 | t-Bu | H | CF₃ | Cl | H | | 177-180 |
| 327 | A-1 | i-Pro | H | CF₃ | Me | H | 432 | |
| 328 | A-1 | i-Pro | H | (1′) or | Me | H | 432 | |
| | | | | (1″)-CF₃ | | | | |
| 329 | A-1 | i-Pro | H | CF₃ | Cl | H | | 83-86 |
| 330 | A-1 | i-Pro | H | (1′) or | Cl | H | | 80-83 |
| | | | | (1″)-CF₃ | | | | |
| 331 | A-1 | i-Pro | H | (1′) or | Cl | H | | 82-85 |
| | | | | (1″)-CF₃ | | | | |
| 333 | A-1 | H | Me | CF₃ | Cl | H | | 132-135 |
| 334 | A-1 | Et | H | CF₃ | Cl | H | | 87-90 |
| 375 | A-1 | i-Pro | H | (1′)-Me | Cl | H | 398 | |

*(1′) and (1″) are defined as below.

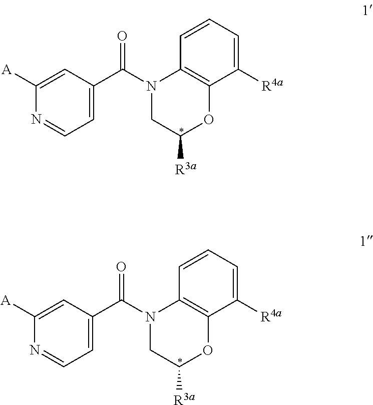

INDEX TABLE B

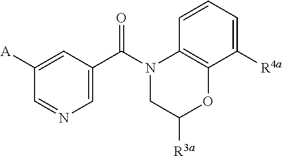

| Cmpd # | A | R^{1a} | R^{1b} | R^{3a} | R^{4a} | M.S. | M.P. (° C.) |
|---|---|---|---|---|---|---|---|
| 6 | A-1 | H | H | Et | Cl | | 151.2-155.1 |
| 404 | A-11 | — | 4-Me | (1′)-Me | Cl | 368 | |

INDEX TABLE C

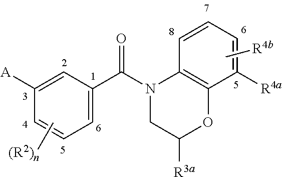

| Cmpd | | | | | | | | | | M.P |
|---|---|---|---|---|---|---|---|---|---|---|
| # | A | R^{1a} | R^{1b} | R^{1c} | R² | R^{3a} | R^{4a} | R^{4b} | M.S | (° C.) |
| 7 | A-1 | H | H | — | H | Et | Cl | H | 369 | |
| 18 | A-1 | H | H | — | H | (1′)-Me | Cl | H | 355.2 | |
| 20 | A-1 | H | H | — | H | (1′)-Me | F | H | 339 | |
| 21 | A-1 | H | H | — | H | (1′)-Me | Me | H | 335 | |
| 22 | A-1 | H | H | — | H | c-Pro | Cl | H | 381 | |
| 23 | A-1 | H | H | — | H | (1′)-Me | H | 6-F | 339 | |
| 24 | A-1 | H | H | — | H | (1′)-Me | H | 7-F | 339 | |
| 25 | A-1 | H | H | — | H | (1′)-Me | Br | H | 399 | |
| 26 | A-1 | H | H | — | H | (1′)-Me | CH═CH₂ | H | 347 | |
| 27 | A-1 | H | H | — | H | (1′)-Me | C≡CSiMe₃ | H | 417 | |
| 28 | A-1 | H | H | — | H | (1′)-Me | H | 8-F | 339 | |
| 29 | A-1 | H | H | — | 4-CN | (1′)-Me | Me | H | 360 | |
| 30 | A-1 | H | H | — | H | (1′)-Me | C≡CH | H | 345 | |
| 31 | A-1 | H | H | — | H | (1′)-Me | H | H | 321 | |
| 32 | A-1 | H | H | — | 5-Me | (1′)-Me | Cl | H | | 97-99 |
| 33 | A-1 | H | H | — | 6-Me | (1′)-Me | Cl | H | | 95-97 |
| 37 | A-1 | H | H | — | 5-F | (1′)-Me | Cl | H | | 105-107 |
| 38 | A-1 | H | H | — | 4-Me | (1′)-Me | Cl | H | | 91-93 |
| 39 | A-1 | H | H | — | 6-F | (1′)-Me | Cl | H | | 86-88 |
| 40 | A-1 | H | H | — | 2-Me | (1′)-Me | Cl | H | | 89-91 |
| 41 | A-1 | H | H | — | H | (1′)-Me | OMe | H | 351.1 | |
| 42 | A-1 | H | H | — | H | (1′)-Me | c-Pro | H | 361 | |
| 43 | A-1 | H | H | — | H | (1′)-Me | Et | H | 349 | |
| 44 | A-1 | H | H | — | H | (1′)-Me | Me | 7-F | 353 | |
| 45 | A-1 | H | H | — | H | (1′)-Me | F | 7-F | 357 | |
| 46 | A-1 | H | H | — | H | (1′)-Me | CN | H | 346.1 | |
| 47 | A-1 | H | H | — | H | (1′)-Me | CF₃ | H | 389.1 | |
| 48 | A-1 | H | H | — | H | (1′)-Me | OCF₃ | H | 405 | |
| 49 | A-1 | H | H | — | H | (1′)-Me | H | 7-NO₂ | 366 | |
| 50 | A-1 | H | H | — | 6-F | (1′)-Me | Me | H | 353.3 | |
| 51 | A-1 | Me | H | — | H | (1′)-Me | Me | H | | 115-117 |
| 52 | A-1 | H | H | — | 4-F | (1′)-Me | Me | H | | 154-156 |
| 53 | A-1 | H | H | — | 2-F | (1′)-Me | Me | H | 353 | |
| 54 | A-1 | c-Pro | H | — | H | (1′)-Me | Me | H | 375 | |
| 55 | A-1 | i-Pro | H | — | H | (1′)-Me | Me | H | 377 | |
| 56 | A-1 | H | i-Pro | — | H | (1′)-Me | Me | H | | 120-122 |
| 57 | A-1 | H | H | — | 6-Me | (1′)-Me | Me | H | 349 | |
| 58 | A-1 | H | H | — | H | (1′) or (1″) -CF₃ | Me | H | 389.3 | |
| 59 | A-1 | H | H | — | H | CF₃ | Cl | H | | 64-67 |
| 60 | A-1 | H | H | — | 6-Cl | (1′)-Me | Me | H | 369 | |
| 61 | A-1 | Br | H | — | H | (1′)-Me | Me | H | 413.3 | |
| 62 | A-1 | Br | H | — | 6-Me | (1′)-Me | Cl | H | 447.1 | |
| 63 | A-1 | H | H | — | 6-OMe | (1′)-Me | Me | H | 365 | |
| 64 | A-1 | CN | H | — | H | (1′)-Me | Me | H | 359.3 | |
| 65 | A-1 | t-Bu | H | — | H | (1′)-Me | Me | H | 391.9 | |
| 66 | A-1 | CF₃ | H | — | H | (1′)-Me | Me | H | 403.3 | |
| 67 | A-1 | c-Pro | H | — | 6-Me | (1′)-Me | Cl | H | 409.5 | |
| 68 | A-1 | H | H | — | H | (1′) or (1″)-CF₃ | Me | H | 389.3 | |
| 69 | A-1 | Cl | H | — | H | (1′)-Me | Me | H | 369.2 | |
| 70 | A-1 | H | H | — | 6-CF₃ | (1′)-Me | Cl | H | 423 | |
| 71 | A-1 | Et | H | — | H | (1′)-Me | Me | H | 363.8 | |
| 72 | A-1 | Pro | H | — | H | (1′)-Me | Me | H | 377.8 | |
| 73 | A-1 | i-Pro | H | — | 6-Me | (1′)-Me | Cl | H | 411.4 | |
| 74 | A-1 | Me | H | — | 6-Me | (1′)-Me | Cl | H | 383.4 | |
| 75 | A-1 | Et | H | — | 6-Me | (1′)-Me | Cl | H | 398.1 | |
| 76 | A-1 | t-Bu | H | — | 6-Me | (1′)-Me | Cl | H | 426.1 | |
| 77 | A-1 | c-Bu | H | — | H | (1′)-Me | Me | H | 389.8 | |
| 78 | A-1 | i-Pro | H | — | H | (1′)-Me | Cl | H | 397.9 | |
| 79 | A-1 | H | H | — | 6-Et | (1′)-Me | Cl | H | 383 | |
| 80 | A-1 | c-Pen | H | — | H | (1′)-Me | Me | H | 403.6 | |
| 81 | A-1 | H | H | — | 5-Cl | (1′)-Me | Cl | H | 389 | |
| 82 | A-1 | c-Hex | H | — | H | (1′)-Me | Me | H | 418 | |
| 83 | A-1 | t-Bu | H | — | 6-OMe | (1′)-Me | Cl | H | 441.5 | |
| 84 | A-1 | i-Pro | H | — | 6-OMe | (1′)-Me | Cl | H | 427.4 | |
| 85 | A-1 | Br | H | — | H | (1′)-Me | Cl | H | 435.3 | |
| 86 | A-1 | Bu | H | — | H | (1′)-Me | Me | H | 391.4 | |
| 87 | A-1 | s-Bu | H | — | H | (1′)-Me | Me | H | 391.4 | |
| 88 | A-1 | Et | H | — | 6-OMe | (1′)-Me | Cl | H | 413.4 | |
| 89 | A-1 | Me | H | — | 6-OMe | (1′)-Me | Cl | H | 399.4 | |
| 90 | A-1 | CH₂c-hex | H | — | H | (1′)-Me | Cl | H | 451.5 | |
| 91 | A-1 | Et | H | — | H | (1′)-Me | Cl | H | 383 | |
| 92 | A-1 | t-Bu | H | — | H | (1′)-Me | Cl | H | 411 | |
| 93 | A-1 | c-Bu | H | — | H | (1′)-Me | Cl | H | 409 | |
| 94 | A-1 | i-Pro | H | — | 6-Et | (1′)-Me | Cl | H | 425 | |
| 95 | A-1 | Et | H | — | 6-Et | (1′)-Me | Cl | H | 411 | |
| 96 | A-1 | c-Bu | H | — | 6-Me | (1′)-Me | Cl | H | 423 | |
| 97 | A-1 | c-Bu | H | — | 6-Me | (1′)-Me | Me | H | 403 | |
| 98 | A-1 | Et | H | — | 6-Me | (1′)-Me | Me | H | 377 | |
| 99 | A-1 | i-Pro | H | — | 6-Me | (1′)-Me | Me | H | 391 | |
| 100 | A-1 | t-Bu | H | — | 6-Me | (1′)-Me | Me | H | 405 | |
| 101 | A-1 | CH₂OMe | H | — | H | (1′)-Me | Me | H | 379.3 | |
| 102 | A-1 | t-Bu | H | — | 6-Et | (1′)-Me | Cl | H | 439 | |
| 103 | A-1 | c-Pro | H | — | 6-Et | (1′)-Me | Cl | H | 423 | |
| 104 | A-1 | c-Bu | H | — | 6-Et | (1′)-Me | Cl | H | 437 | |
| 105 | A-1 | i-Pro | H | — | 6-OH | (1′)-Me | Cl | H | 413 | |
| 106 | A-1 | i-Pro | H | — | 6-OEt | (1′)-Me | Cl | H | 441 | |
| 107 | A-1 | i-Pro | H | — | 6-O-Propargyl | (1′)-Me | Cl | H | 451 | |
| 108 | A-1 | i-Pro | H | — | 6-O-i-Pro | (1′)-Me | Cl | H | 455 | |
| 109 | A-1 | 3-oxetanyl | H | — | H | (1′)-Me | Me | H | 391.5 | |
| 110 | A-1 | i-Pro | H | — | 6-O-Allyl | (1′)-Me | Cl | H | 453 | |
| 111 | A-1 | i-Pro | H | — | 6-OCF₃ | (1′)-Me | Cl | H | 481 | |
| 112 | A-1 | i-Bu | H | — | H | (1′)-Me | Cl | H | 412.1 | |
| 113 | A-1 | CH₂CH₂OMe | H | — | H | (1′)-Me | Me | H | 393.9 | |
| 114 | A-1 | H | H | — | 5-Br | (1′)-Me | Cl | H | 433 | |
| 115 | A-3 | — | H | — | H | (1′)-Me | Cl | H | 356.3 | |
| 117 | A-12 | — | H | Me | H | (1′)-Me | Me | H | 365 | |
| 118 | A-12 | — | H | i-Pro | H | (1′)-Me | Me | H | 393 | |
| 119 | A-5 | — | H | Me | H | (1′)-Me | Cl | H | 368.3 | |
| 120 | A-7 | — | Me | — | H | (1′)-Me | Cl | H | 370.3 | |
| 121 | A-8 | — | Me | — | H | (1′)-Me | Cl | H | 370.2 | |
| 122 | A-12 | — | H | H | H | (1′)-Me | Me | H | 351 | |
| 123 | A-13 | — | H | — | H | (1′)-Me | Cl | H | 365.2 | |
| 124 | A-10 | — | 4-Me | — | H | (1′)-Me | Cl | H | 385.3 | |
| 125 | A-9 | — | 2-Me | — | H | (1′)-Me | Cl | H | 385.3 | |
| 126 | A-1 | H | Me | — | 6-Me | (1′)-Me | Cl | H | 383.4 | |
| 127 | A-4 | — | H | Me | H | (1′)-Me | Cl | H | 369.3 | |
| 128 | A-2 | — | H | — | H | (1′)-Me | Cl | H | 355.3 | |
| 129 | A-11 | — | H | — | H | (1′)-Me | Cl | H | 354.2 | |
| 130 | A-11 | — | 4-CF₃ | — | H | (1′)-Me | Me | H | 402 | |
| 131 | A-11 | — | 4-F | — | H | (1′)-Me | Me | H | 352 | |
| 132 | A-11 | — | 4-Cl | — | H | (1′)-Me | Me | H | 368 | |
| 133 | A-11 | — | 4-CO₂Et | — | H | (1′)-Me | Me | H | 406 | |
| 134 | A-11 | — | 4-CN | — | H | (1′)-Me | Me | H | 359 | |
| 135 | A-1 | CH₂O-i-Pro | H | — | H | (1′)-Me | Me | H | 407.6 | |
| 136 | A-1 | H | H | — | 5-c-Pro | (1′)-Me | Cl | H | | 92-95 |
| 137 | A-1 | H | H | — | 5-I | (1′)-Me | Cl | H | | 212-215 |
| 138 | A-1 | H | H | — | 5-OMe | (1′)-Me | Cl | H | | 89-92 |
| 140 | A-1 | i-Pro | H | — | 6-CN | (1′)-Me | Cl | H | 422 | |
| 141 | A-1 | i-Pro | H | — | 6-OCH₂CF₃ | (1′)-Me | Cl | H | 495 | |
| 142 | A-1 | Me | Me | — | H | (1′)-Me | Cl | H | 383 | |
| 143 | A-1 | i-Pro | H | — | 6-OCHF₂ | (1′)-Me | Cl | H | 463 | |
| 148 | A-1 | HOCH₂ | H | — | 6-Me | (1′)-Me | Cl | H | | 84-87 |
| 149 | A-1 | HOC(Me)₂ | H | — | 6-Me | (1′)-Me | Cl | H | | 85-87 |
| 150 | A-1 | i-Pro | H | — | 5,6-diOCHF₂ | (1′)-Me | Cl | H | | 116-119 |
| 151 | A-1 | i-Pro | H | — | 5-F-6-Me | (1′)-Me | Cl | H | 429 | |
| 152 | A-1 | i-Pro | H | — | 5-F-6-Et | (1′)-Me | Cl | H | 443 | |
| 153 | A-1 | H | H | — | 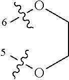 | (1′)-Me | Cl | H | 413 | |
| 154 | A-1 | t-Bu | H | — | 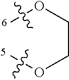 | (1′)-Me | Cl | H | 469 | |
| 155 | A-1 | Et | H | — | 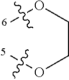 | (1′)-Me | Cl | H | 441 | |
| 156 | A-1 | c-Pro | H | — | 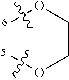 | (1′)-Me | Cl | H | 453 | |
| 157 | A-1 | c-Bu | H | — | 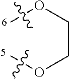 | (1′)-Me | Cl | H | 467 | |
| 158 | A-1 | i-Pro | H | — | 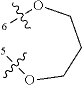 | (1′)-Me | Cl | H | 469 | |
| 159 | A-18 | — | 6-Me | — | H | (1′)-Me | Me | H | 359 | |
| 160 | A-19 | — | 6-Me | — | H | (1′)-Me | Me | H | 360 | |
| 161 | A-20 | Me | H | — | H | (1′)-Me | Me | H | 360 | |
| 162 | A-21 | — | 5-Me | — | H | (1′)-Me | Me | H | 360 | |
| 163 | A-13 | — | 2-Me | — | H | (1′)-Me | Me | H | 359 | |
| 164 | A-1 | Et | H | — | 5-OMe | (1′)-Me | Cl | H | 413 | |
| 165 | A-22 | — | H | Et | H | (1′)-Me | Me | H | 363 | |
| 166 | A-23 | — | — | — | H | (1′)-Me | Cl | H | 373 | |
| 167 | A-1 | 1-Me-c-Bu | H | — | 6-Me | (1′)-Me | Cl | H | | 127-130 |
| 168 | A-1 | 1-CF₃-c-Bu | H | — | 6-Me | (1′)-Me | Cl | H | | 133-136 |
| 169 | A-1 | CMe₂OH | H | — | H | (1′)-Me | Cl | H | | 87-90 |
| 170 | A-1 | i-Pro | H | — | 6-Me | (1′)-Me | CF₃ | H | 445 | |
| 171 | A-1 | i-Pro | H | — | 6-OMe | (1′)-Me | CF₃ | H | 461 | |
| 172 | A-22 | — | H | i-Pro | H | (1′)-Me | Cl | H | 397.2 | |
| 173 | A-22 | — | H | Et | H | (1′)-Me | Cl | H | 383.2 | |
| 174 | A-1 | i-Pro | H | — | 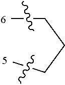 | (1′)-Me | Cl | H | 437 | |
| 175 | A-1 | 1-CF₃-c-Pro | H | — | 6-Me | (1′)-Me | Cl | H | 477.23 | |
| 176 | A-1 | Br | H | — | H | (1′)-CH₂OMe | Cl | H | | 87-90 |
| 177 | A-1 | i-Pro | H | — | 5-F-6-OMe | (1′)-Me | Cl | H | | 86-89 |
| 178 | A-1 | CH₂SMe | H | — | 6-Me | (1′)-Me | Cl | H | 429 | |
| 179 | A-1 | i-Pro | H | — | 5-Me-6-Et | (1′)-Me | Cl | H | | 73-76 |
| 180 | A-1 | CEt₂OH | H | — | 6-Me | (1′)-Me | Cl | H | 456 | |
| 181 | A-24 | — | Me | — | H | (1′)-Me | Cl | H | 366 | |
| 182 | A-1 | COMe | H | — | 6-Et | (1′)-Me | Cl | H | 425 | |
| 183 | A-1 | COOEt | H | — | 6-Et | (1′)-Me | Me | H | 435 | |
| 184 | A-1 | COMe | H | — | 6-Et | (1′)-Me | Me | H | | 89-92 |
| 185 | A-1 | COMe | H | — | 6-OMe | (1′)-Me | Me | H | 407 | |
| 186 | A-1 | COOMe | H | — | 6-Me | (1′)-CH₂OMe | Cl | H | 457 | |
| 187 | A-1 | COOEt | H | — | 6-Et | (1′)-Me | Cl | H | 455 | |
| 188 | A-1 | COMe | H | — | 6-OMe | (1′)-Me | Cl | H | 427 | |
| 189 | A-1 | COOEt | H | — | 6-OMe | (1′)-Me | Me | H | 437 | |
| 190 | A-1 | COMe | H | — | 6-Me | (1′)-Me | Me | H | 391 | |
| 191 | A-1 | i-Pro | H | — | 6-(S═O)CF₃ | (1′)-Me | Cl | H | 51 | |
| 192 | A-1 | t-Bu | H | — | 6-Me | (1′) or | Cl | H | 479 | |
| | | | | | | (1″)-CF₃ | | | | |
| 193 | A-1 | 1-OH-c-Pen | H | — | 6-Me | (1′)-Me | Cl | H | 453 | |
| 194 | A-1 | i-Pro | H | — | 5,6-diOMe | (1′)-Me | Cl | H | 457 | |
| 195 | A-1 | i-Pro | H | — | 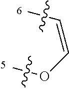 | (1′)-Me | Me | H | 417 | |
| 196 | A-1 | H | c-Pro | — | H | Me | Me | H | | 104-106 |
| 199 | A-27 | — | H | — | H | (1′)-Me | Cl | H | 355 | |
| 200 | A-18 | — | H | — | H | (1′)-Me | Cl | H | 365 | |
| 203 | A-28 | — | H | — | H | (1′)-Me | Cl | H | 366.4 | |
| 204 | A-29 | — | 5-Me | — | H | (1′)-Me | Cl | H | 368.4 | |
| 205 | A-6 | — | H | Me | H | (1′)-Me | Cl | H | 368.4 | |
| 206 | A-11 | — | 3-Me | — | H | (1′)-Me | Cl | H | 368.4 | |
| 207 | A-11 | — | 4-Me | — | H | (1′)-Me | Cl | H | 368.4 | |
| 208 | A-21 | — | H | — | H | (1′)-Me | Cl | H | 366.4 | |
| 209 | A-30 | — | 6-Me | — | H | (1′)-Me | Me | H | 360.4 | |
| 210 | A-1 | i-Pro | H | — | 6-OMe | (1′)-Me | Me | H | 407 | |
| 211 | A-1 | Et | H | — | 6-OMe | (1′)-Me | Me | H | 393 | |
| 212 | A-1 | i-Pro | H | — | 5-OMe | (1′)-Me | Cl | H | 427 | |
| 213 | A-1 | t-Bu | H | — | 5-OMe | (1′)-Me | Cl | H | 441 | |
| 214 | A-1 | c-Bu | H | — | 5-OMe | (1′)-Me | Cl | H | 439 | |
| 215 | A-1 | Me | H | — | 5-OMe | (1′)-Me | Cl | H | 399 | |
| 216 | A-1 | c-Pro | H | — | 5-OMe | (1′)-Me | Cl | H | 425 | |
| 217 | A-1 | i-Pro | H | — | 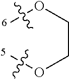 | (1′)-Me | Cl | H | 455.27 | |
| 218 | A-1 | Et | H | — | 6-Me | (1′) or (1″)-CF₃ | Cl | H | | 79-82 |
| 219 | A-1 | c-Bu | H | — | 6-Me | (1′) or (1″)-CF₃ | Cl | H | 477 | |
| 220 | A-1 | i-Pro | H | — | 5-Me-6-Cl | (1′)-Me | Cl | H | | 150-153 |
| 221 | A-22 | — | H | i-Pro | H | (1″)-Me | Me | H | 377 | |
| 222 | A-31 | — | H | — | H | (1′)-Me | Cl | H | 353 | |
| 223 | A-1 | i-Pro | H | — | 5-Me-6-F | (1′)-Me | Cl | H | | 77-80 |
| 224 | A-1 | CH₂SOMe | H | — | 6-Me | (1′)-Me | Cl | H | | 173-176 |
| 225 | A-1 | CH₂SO₂Me | H | — | 6-Me | (1′)-Me | Cl | H | | 171-174 |
| 226 | A-1 | NMe₂ | H | — | H | (1′)-Me | Me | H | 378 | |
| 227 | A-1 | i-Pro | H | — | 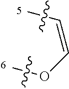 | (1′)-Me | Me | H | 417 | |
| 228 | A-1 | H | H | — | 6-OMe | (1′)-Me | H | H | 351 | |
| 229 | A-1 | i-Pro | H | — | 6-SO₂CF₃ | (1′)-Me | Cl | H | 529 | |
| 230 | A-1 | t-Bu | H | — | 6-OMe | (1′)-CH₂OMe | Cl | H | 471 | |
| 231 | A-1 | Et | H | — | 6-OMe | (1′)-CH₂OMe | Cl | H | 443 | |
| 232 | A-25 | — | 4-Me | — | H | (1′)-Me | Me | H | 348 | |
| 233 | A-25 | — | 4-CN | — | H | (1′)-Me | Me | H | 359 | |
| 234 | A-1 | H | H | — | 6-OMe | (1′)-CH₂OMe | Cl | H | 415 | |
| 235 | A-1 | c-Pro | H | — | 6-OMe | (1′)-CH₂OMe | Cl | H | 455 | |
| 236 | A-1 | c-Bu | H | — | 6-OMe | (1′)-CH₂OMe | Cl | H | 469 | |
| 237 | A-1 | Si(Et)₃ | H | — | H | (1′)-Me | Me | H | 449 | |
| 238 | A-30 | — | H | — | H | (1′)-Me | Me | H | 366 | |
| 239 | A-33 | — | H | Me | H | (1′)-Me | Cl | H | 369 | |
| 240 | A-1 | i-Pro | H | — | 6-Et | (1′)-Me | Me | H | 405 | |
| 241 | A-1 | H | H | — | 6-OMe | (1′)-Me | Cl | H | 385 | |
| 242 | A-1 | i-Pro | H | — | 6-Me | (1′)-Me | F | H | 395 | |
| 243 | A-10 | i-Pro | 5-Me | — | H | (1′)-Me | Me | H | 365 | |
| 244 | A-34 | — | 5-Me | — | H | (1′)-Me | Me | H | 364 | |
| 245 | A-34 | — | 4-Me | — | H | (1′)-Me | Me | H | 364 | |
| 246 | A-35 | — | 5-Me | — | H | (1′)-Me | Me | H | 364 | |
| 247 | A-36 | — | H | Me | H | (1′)-Me | Me | H | 349 | |
| 248 | A-1 | CMeF₂ | H | — | 6-Me | (1′)-Me | Cl | H | 433 | |
| 249 | A-1 | i-Pro | H | — | 6-SCF₃ | (1′)-Me | Cl | H | 497 | |
| 250 | A-14 | — | 2-Me | — | H | (1′)-Me | Me | H | 360 | |
| 251 | A-21 | — | 6-Me | — | H | (1′)-Me | Me | H | 360 | |
| 252 | A-1 | 1-Me-c-Pro | H | — | 6-Me | (1′)-Me | Cl | H | 423 | |
| 253 | A-1 | HOC(Me)₂ | H | — | H | (1′)-CH₂OMe | Cl | H | 443 | |
| 254 | A-1 | HOC(Me)₂ | H | — | 6-OMe | (1′)-Me | Cl | H | 443 | |
| 255 | A-1 | CO₂Et | H | — | 6-OMe | (1′)-Me | Cl | H | 457 | |
| 256 | A-1 | HOC(Me)₂ | H | — | 6-Me | (1′)-Me | Me | H | 407 | |
| 257 | A-1 | CO₂Et | H | — | H | (1′)-Me | Me | H | 407 | |
| 258 | A-1 | i-Pro | H | — | 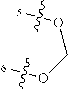 | (1′)-Me | Cl | H | 441 | |
| 259 | A-1 | i-Pro | H | — | 5,6-diMe | (1′)-Me | Cl | H | 425 | |
| 260 | A-1 | i-Pro | H | — | 5-Me-6-OMe | (1′)-Me | Cl | H | 441 | |
| 261 | A-1 | CO₂Et | H | — | H | (1′)-Me | Cl | H | 427 | |
| 262 | A-1 | CMe═CMe₂ | H | — | H | (1′)-Me | Me | H | 403 | |
| 263 | A-1 | H | H | — | 6-Me | (1′)-CH₂OMe | Cl | H | 399 | |
| 264 | A-1 | t-Bu | H | — | 6-Me | (1′)-CH₂OMe | Cl | H | 455 | |
| 265 | A-1 | Et | H | — | 6-Me | (1′)-CH₂OMe | Cl | H | 427 | |
| 266 | A-1 | c-Pro | H | — | 6-Me | (1′)-CH₂OMe | Cl | H | 439 | |
| 267 | A-1 | c-Bu | H | — | 6-Me | (1′)-CH₂OMe | Cl | H | 453 | |
| 268 | A-1 | i-Pro | H | — | 6-OMe | (1′)-Me | Me | H | 423 | |
| 269 | A-1 | i-Pro | H | — | 6-OMe | (1′)-Me | F | H | 411 | |
| 270 | A-1 | H | H | — | 6-OMe | (1′)-Me | F | H | 369 | |
| 272 | A-1 | i-Pro | H | — | 5,6-diOH | (1′)-Me | Cl | H | 429 | |
| 273 | A-1 | CH₂-c-Pro | H | — | 6-Me | (1′)-Me | Cl | H | 423 | |
| 274 | A-1 | H | c-Bu | — | 6-Me | (1′)-Me | Cl | H | 423 | |
| 275 | A-1 | 3-Me-3-oxetanyl | H | — | 6-Me | (1′)-Me | Cl | H | 439 | |
| 276 | A-1 | HOC(Me)₂ | H | — | 6-Me | (1′)-CH₂OMe | Cl | H | 457 | |
| 277 | A-1 | HOC(Me)₂ | H | — | 6-Et | (1′)-Me | Cl | H | 441 | |
| 278 | A-1 | HOC(Me)₂ | H | — | H | (1′)-Me | Me | H | 393 | |
| 279 | A-1 | HOC(Me)₂ | H | — | 6-Et | (1′)-Me | Me | H | 421 | |
| 280 | A-1 | HOC(Me)₂ | H | — | 6-OMe | (1′)-Me | Me | H | 423 | |
| 281 | A-1 | CO₂Et | H | — | 6-Me | (1′)-Me | Me | H | 421 | |
| 282 | A-37 | — | H | — | H | (1′)-Me | Me | H | 335 | |
| 283 | A-1 | Ph | H | — | H | (1′)-Me | Me | H | 411 | |
| 284 | A-1 | CCSiMe₃ | H | — | H | (1′)-Me | Me | H | 431 | |
| 287 | A-20 | H | H | — | H | (1′)-Me | Cl | H | 366 | |
| 288 | A-1 | i-Pro | H | — | H | (1′) or (1″)-CF₃ | Cl | H | | 66-69 |
| 289 | A-1 | i-Pro | H | — | H | (1′) or (1″)-CF₃ | Cl | H | 451 | |
| 290 | A-1 | Et | H | — | 6-Me | CF₃ | Cl | H | | 73-76 |
| 291 | A-1 | i-Pro | H | — | 6-Me | (1′) or (1″)-CF₃ | Cl | H | 465 | |
| 292 | A-1 | i-Pro | H | — | 6-Me | (1′) or (1″)-CF₃ | Cl | H | | 82-85 |
| 293 | A-32 | — | — | Me— | H | (1′)-Me | Cl | H | | 93-96 |
| 294 | A-1 | CH₂OEt | H | — | 6-Me | (1′)-Me | Cl | H | 427 | |
| 295 | A-1 | CH₂Cl | H | — | 6-Me | (1′)-Me | Cl | H | 417 | |
| 296 | A-15 | Me | H | — | H | (1′)-Me | Cl | H | 385 | |
| 297 | A-25 | — | 5-Br | — | H | (1′)-Me | Me | H | 412 | |
| 298 | A-25 | — | 5-CO₂Et | — | H | (1′)-Me | Me | H | 406 | |
| 299 | A-1 | NMeSO₂Me | H | — | H | (1′)-Me | Me | H | 442 | |
| 300 | A-1 | NMeCO₂Me | H | — | H | (1′)-Me | Me | H | 422 | |
| 301 | A-1 | i-Pro | H | — | H | CF₃ | Me | H | 431 | |
| 302 | A-1 | i-Pro | H | — | H | (1′) or (1″)-CF₃ | Me | H | 431 | |
| 303 | A-1 | i-Pro | H | — | H | (1′) or (1″)-CF₃ | Me | H | 431 | |
| 304 | A-25 | — | 4-Cl | — | H | (1′)-Me | Me | H | 368 | |
| 306 | A-1 | NO₂ | H | — | H | (1′)-Me | Me | H | 380 | |
| 307 | A-1 | OMe | H | — | H | (1′)-Me | Me | H | 365 | |
| 308 | A-1 | NH₂ | H | — | H | (1′)-Me | Me | H | 350 | |
| 309 | A-25 | — | 5-CN | — | H | (1′)-Me | Me | H | 359 | |
| 310 | A-1 | OMe | H | — | H | (1′)-Me | Cl | H | | 124-129 |
| 311 | A-1 | OEt | H | — | H | (1′)-Me | Cl | H | | 75-78 |
| 312 | A-1 | O-i-Pro | H | — | H | (1′)-Me | Cl | H | | 136-138 |
| 313 | A-1 | i-Pro | H | — | 6-Me | (1′)-CH₂OMe | Cl | H | | 149-152 |
| 314 | A-1 | i-Pro | H | — | 6-OMe | (1′)-CH₂OMe | Cl | H | | 143-146 |
| 315 | A-1 | CO₂Me | H | — | 6-Me | (1′)-Me | Cl | H | | 182-185 |
| 316 | A-1 | H | H | — | 5-i-Pro | (1′)-Me | Cl | H | | 85-88 |
| 317 | A-1 | H | H | — | 5-CMe═CH | (1′)-Me | Cl | H | | 76-78 |
| 323 | A-1 | c-Bu | H | — | 6-OMe | (1′)-Me | Cl | H | 439 | |
| 324 | A-1 | c-Pro | H | — | 6-OMe | (1′)-Me | Cl | H | 425 | |
| 325 | A-1 | c-Pro | H | — | H | (1′)-Me | Cl | H | 395 | |
| 326 | A-1 | Me | H | — | H | (1′)-Me | Cl | H | 369 | |
| 332 | A-1 | H | H | — | 5-Et | (1′)-Me | Cl | H | | 77-80 |
| 335 | A-1 | H | H | — | 5-CF₃ | (1′)-Me | Cl | H | 423 | |
| 336 | A-1 | NMeCOMe | H | — | H | (1′)-Me | Me | H | 406 | |
| 337 | A-1 | NHMe | H | — | H | (1′)-Me | Me | H | 364 | |
| 338 | A-1 | i-Pro | H | — | 6-OSO₂Me | (1′)-Me | Cl | H | 491 | |
| 339 | A-1 | i-Pro | H | — | 6-OSO₂CF₃ | (1′)-Me | Cl | H | 545 | |
| 340 | A-1 | i-Pro | H | — | 6-Me | (1′)-CH₂OEt | Cl | H | 455 | |
| 341 | A-1 | i-Pro | H | — | 6-OMe | (1′)-CH₂OEt | Cl | H | 471 | |
| 342 | A-1 | CO₂Et | H | — | 6-Me | (1′)-Me | Cl | H | | 186-189 |
| 343 | A-1 | CMe₂OMe | H | — | 6-Me | (1′)-Me | Cl | H | 441 | |
| 344 | A-1 | Et | H | — | H | CF₃ | Me | H | 417 | |
| 345 | A-1 | Et | H | — | H | CF₃ | Cl | H | | 82-85 |
| 346 | A-1 | H | H | — | 6-Me | CF₃ | Cl | H | | 81-84 |
| 347 | A-1 | t-Bu | H | — | H | CF₃ | Me | H | | 120-123 |
| 348 | A-1 | i-Pro | H | — | H | CF₃ | Cl | H | | 73-76 |
| 349 | A-1 | Me | H | — | 6-Me | CF₃ | Cl | H | 437 | |
| 350 | A-1 | t-Bu | H | — | 6-Me | CF₃ | Cl | H | 47 | |
| 351 | A-1 | H | Me | — | 6-Me | CF₃ | Cl | H | 437 | |
| 352 | A-38 | Me | H | — | H | (1′)-Me | Cl | H | 369 | |
| 353 | A-1 | t-Bu | H | — | H | CF₃ | Cl | H | | 87-90 |
| 354 | A-1 | i-Pro | H | — | 6-Me | CF₃ | Cl | H | | 88-91 |
| 355 | A-39 | Me | — | — | H | (1′)-Me | Cl | H | | 140-143 |
| 356 | A-1 | OSO₂Me | H | — | H | (1′)-Me | Cl | H | | 175-177 |
| 357 | A-1 | CH₂OMe | H | — | 6-Me | (1′)-Me | Cl | H | | 95-97 |
| 358 | A-1 | CH(OH)Me | H | — | 6-Me | (1′)-Me | Cl | H | | 83-86 |
| 359 | A-1 | CH(OMe)Me | H | — | 6-Me | (1′)-Me | Cl | H | | 76-79 |
| 360 | A-1 | COMe | H | — | 6-Me | (1′)-Me | Cl | H | | 122-125 |
| 361 | A-1 | Me | H | — | 6-F | (1′)-Me | Me | H | 367.21 | |
| 363 | A-1 | CN | H | — | 6-Me | (1′)-Me | Cl | H | 394.18 | |
| 364 | A-1 | CH₂CN | H | — | 6-Me | (1′)-Me | Cl | H | 408.21 | |
| 365 | A-1 | CMe₂F | H | — | 6-Me | (1′)-Me | Me | H | 409.2 | |
| 366 | A-1 | t-Bu | H | — | 6-Me | (1′)-Me | CF₃ | H | 459 | |
| 367 | A-1 | c-Bu | H | — | 6-Me | (1′)-Me | CF₃ | H | 457 | |
| 368 | A-1 | c-Bu | H | — | 6-OMe | (1′)-Me | F | H | 423 | |
| 371 | A-1 | t-Bu | H | — | 6-OMe | (1′)-Me | F | H | 425 | |
| 372 | A-1 | t-Bu | H | — | 6-Me | (1′)-Me | F | H | 409 | |
| 373 | A-1 | CO₂Et | H | — | 6-Me | (1′) or | Cl | H | 495 | |
| | | | | | | (1″)-CF₃ | | | | |
| 374 | A-1 | c-Bu | H | — | 6-Me | (1′)-Me | F | H | 407 | |
| 377 | A-1 | c-Bu | H | — | 6-OMe | (1′)-CH₂OEt | Cl | H | 483 | |
| 378 | A-1 | Et | H | — | 6-OMe | (1′)-CH₂OEt | Cl | H | 457 | |
| 379 | A-1 | t-Bu | H | — | 6-OMe | (1′)-CH₂OEt | Cl | H | 485 | |
| 380 | A-1 | c-Bu | H | — | 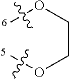 | (1′)-CH₂OMe | Cl | H | 497 | |
| 381 | A-1 | Et | H | — | 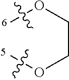 | (1′)-CH₂OMe | Cl | H | 471 | |
| 382 | A-1 | t-Bu | H | — | 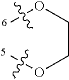 | (1′)-CH₂OMe | Cl | H | 499 | |
| 383 | A-18 | — | 6-Cl | — | H | (1′)-Me | Cl | H | 399 | |
| 384 | A-18 | — | 5-Cl | — | H | (1′)-Me | Cl | H | 399 | |
| 385 | A-18 | — | 6-OMe | — | H | (1′)-Me | Cl | H | 395 | |
| 386 | A-1 | c-Bu | H | — | 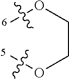 | (1′)-CH₂OEt | Cl | H | 511 | |
| 387 | A-1 | Et | H | — | 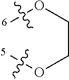 | (1′)-CH₂OEt | Cl | H | 485 | |
| 388 | A-1 | t-Bu | H | — | 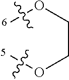 | (1′)-CH₂OEt | Cl | H | 513 | |
| 389 | A-40 | — | 3-Me | — | H | (1′)-Me | Cl | H | 385 | |
| 390 | A-17 | — | Me | — | H | (1′)-Me | Cl | H | 386 | |
| 391 | A-1 | i-Pro | H | — | 6-SMe | (1′)-Me | Cl | H | 443 | |
| 392 | A-1 | i-Pro | H | — | 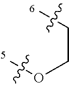 | (1′)-Me | Me | H | 419 | |
| 393 | A-1 | i-Pro | H | — | 6-SO₂Me | (1′)-Me | Cl | H | 475 | |
| 394 | A-1 | i-Pro | H | — | 6-SOMe | (1′)-Me | Cl | H | 459 | |
| 395 | A-1 | i-Pro | H | — | 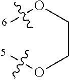 | (1′)-CH₂OMe | Cl | H | 485 | |
| 396 | A-1 | i-Pro | H | — | 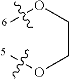 | (1′)-CH₂OEt | Cl | H | 499 | |
| 397 | A-1 | CMe₂CN | H | — | 6-Me | (1′)-Me | Cl | H | 436 | |
| 398 | A-1 | 1-CN-c-Pro | H | — | 6-Me | (1′)-Me | Cl | H | 434 | |
| 399 | A-1 | c-Bu | H | — | 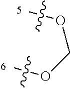 | (1′)-CH₂OEt | Cl | H | 497 | |
| 400 | A-1 | i-Pro | H | — | 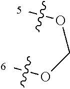 | (1′)-CH₂OEt | Cl | H | 485 | |
| 401 | A-1 | t-Bu | H | — | 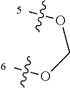 | (1′)-CH₂OEt | Cl | H | 499 | |
| 402 | A-1 | 1-CN-c-Bu | H | — | 6-Me | (1′)-Me | Cl | H | 448 | |
| 403 | A-1 | CH₂S(O)MeNH | H | — | 6-Me | (1′)-Me | Cl | H | 460 | |
| 405 | A-1 | c-Bu | H | — | 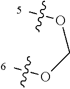 | (1′)-CH₂OMe | Cl | H | 483 | |
| 406 | A-1 | i-Pro | H | — | 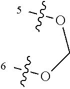 | (1′)-CH₂OMe | Cl | H | 471 | |
| 407 | A-1 | t-Bu | H | — | 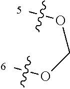 | (1′)-CH₂OMe | Cl | H | 485 | |
| 408 | A-1 | CMe₂OH | H | — | 6-OMe | (1′)-CH₂OMe | Cl | H | 473 | |
| 409 | A-1 | CO₂Me | H | — | 6-OMe | (1′)-CH₂OMe | Cl | H | | 110-113 |
| 410 | A-1 | CMe₂F | H | — | H | (1′)-Me | Cl | H | 395 | |
| 411 | A-1 | c-Bu | H | — | 6-Me | (1′)-CH₂OMe | Cl | H | 467 | |
| 412 | A-1 | Et | H | — | 6-Me | (1′)-CH₂OEt | Cl | H | 441 | |
| 413 | A-1 | t-Bu | H | — | 6-Me | (1′)-CH₂OEt | Cl | H | 469 | |
| 414 | A-1 | CMe₂OH | H | — | 6-OMe | (1′) or (1″)-CF₃ | Cl | H | 497 | |

*(1′) and (1″) are defined as below.

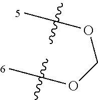 [SMILES: *OCO*]

INDEX TABLE D

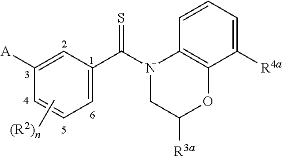

| Cmpd # | A | R^{1a} | R^{1b} | R^{1c} | R² | R^{3a} | R^{4a} | M.S |
|---|---|---|---|---|---|---|---|---|
| 139 | A-1 | H | H | — | H | (1′)-Me | Cl | 371 |
| 271 | A-1 | Et | H | — | 6-OMe | (1′)-Me | Me | 409 |
| 362 | A-1 | Me | H | — | 6-F | (1′)-Me | Me | 383 |

INDEX TABLE E

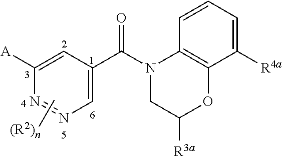

| Cmpd # | A | R^{1a} | R^{1b} | R^{1c} | R² | R^{3a} | R^{4a} | M.S | M.P (° C.) |
|---|---|---|---|---|---|---|---|---|---|
| 305 | A-1 | i-Pro | H | — | H | (1′)-Me | Cl | 399 | |

INDEX TABLE F

| Cmpd | | | M.P |
|---|---|---|---|
| # | A | M.S | (° C.) |
| 145 | 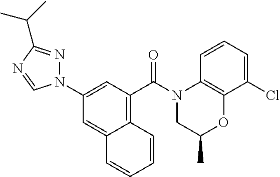 | | 110-113 |
| 146 | 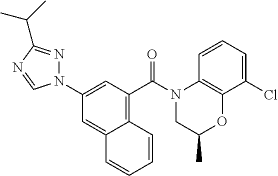 | | 81-84 |
| 147 | 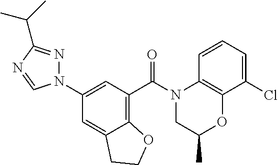 | 439 | |
| 201 | 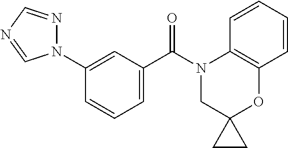 | 333 | |
| 202 | 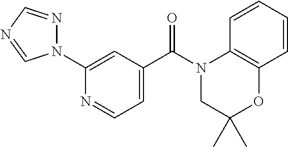 | 336 | |
| 285 | 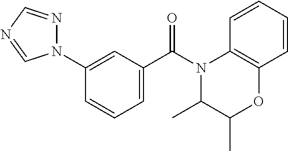 | 335 | |
| 369 | 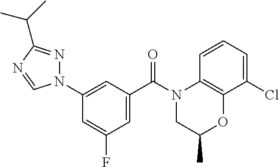 | 416 | |
| 370 | 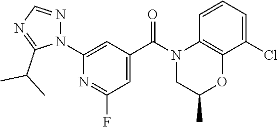 | 416 | |
| 376 | 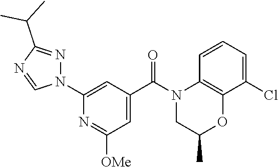 | 428 | |

### BIOLOGICAL EXAMPLES OF THE INVENTION

### Test A

Seeds of plant species selected from barnyardgrass (Echinochloa crusgalli), blackgrass (Alopecurus myosuroides), corn (Zea mays), foxtail, giant (giant foxtail, Setaria faberi), goosegrass (Eleusine indica), kochia (Bassia scoparia), oat, wild (wild oat, Avena fatua), pigweed, palmer (palmer amaranth, palmer pigweed, Amaranthus palmeri), Pigweed, Redroot (redroot pigweed, Amaranthus retroflexus), ragweed (common ragweed, Ambrosia artemisiifolia), ryegrass, Italian (italian ryegrass, Lolium multiflorum), soybean (Glycine max), and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crops and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| 1000 g ai/ha | Compounds | | | | | | | | | | | | | |
|---|---|---|---|---|---|---|---|---|---|---|---|---|---|---|
| Preemergence | 1 | 2 | 3 | 4 | 5 | 8 | 9 | 10 | 11 | 12 | 13 | 14 | 15 | 16 |
| Barnyardgrass | 80 | 60 | 0 | 0 | 70 | 0 | 0 | 90 | 30 | 0 | 0 | 0 | 60 | 0 |
| Blackgrass | — | — | — | — | — | — | — | — | — | — | — | — | — | — |
| Foxtail, Giant | 100 | 100 | 0 | 0 | 100 | 90 | 0 | 100 | 100 | 20 | 90 | 30 | 100 | 0 |
| Goosegrass | — | — | — | — | — | — | — | — | — | — | — | — | — | — |
| Kochia | 40 | 0 | 0 | 0 | 30 | 50 | 0 | 30 | 0 | 0 | 60 | 30 | 60 | 20 |
| Pigweed, Palmer | — | — | — | — | — | — | — | — | — | — | — | — | — | — |
| Pigweed, Redroot | 40 | 20 | 0 | 0 | 80 | 10 | 0 | 40 | 90 | 0 | 50 | 10 | 90 | 20 |
| Ragweed | 80 | 0 | 0 | 0 | 0 | 0 | 0 | 80 | 0 | 0 | 0 | 0 | 0 | 0 |
| Ryegrass, Italian | 90 | 60 | 0 | 0 | 80 | 40 | 0 | 80 | 50 | 0 | 30 | 20 | 60 | 0 |
| 1000 g ai/ha | Compounds | | | | | | | | | | | | | |
| Preemergence | 17 | 18 | 19 | 20 | 21 | 22 | 23 | 24 | 25 | 26 | 29 | 30 | 31 | 32 |
| Barnyardgrass | 0 | 90 | 90 | 90 | 100 | 90 | 30 | 50 | 90 | 0 | 90 | 30 | 80 | — |
| Blackgrass | — | — | — | — | — | — | — | — | — | — | — | — | — | 80 |
| Foxtail, Giant | 0 | 100 | 100 | 100 | 100 | 90 | 50 | 30 | 100 | 0 | 100 | 80 | 100 | 100 |
| Goosegrass | — | — | — | — | — | — | — | — | — | — | — | — | — | 100 |
| Kochia | 30 | 70 | 60 | 80 | 70 | 50 | 0 | 0 | 30 | 0 | 20 | 20 | 30 | 30 |
| Pigweed, Palmer | — | — | — | — | — | — | — | — | — | — | — | — | — | 90 |
| Pigweed, Redroot | 100 | 100 | 0 | 80 | 100 | 60 | 0 | 0 | 100 | 0 | 60 | 100 | 0 | — |
| Ragweed | 0 | 80 | 80 | 80 | 70 | 20 | 0 | 0 | 0 | 0 | 0 | 0 | 20 | 0 |
| Ryegrass, Italian | 0 | 90 | 80 | 100 | 100 | 90 | 30 | 100 | 100 | 0 | 90 | 100 | 90 | 100 |
| 1000 g ai/ha | Compounds | | | | | | | | | | | | | |
| Preemergence | 33 | 34 | 35 | 36 | 41 | 49 | 58 | 68 | 115 | 116 | 119 | 120 | 121 | 123 |
| Barnyardgrass | — | — | — | — | — | — | — | — | 70 | 40 | — | — | — | 40 |
| Blackgrass | 100 | 80 | 70 | 70 | 30 | 0 | 40 | 80 | — | — | 70 | 10 | 0 | — |
| Foxtail, Giant | 100 | 100 | 100 | 100 | 90 | 20 | 80 | 100 | 90 | 90 | 100 | 20 | 60 | 0 |
| Goosegrass | 100 | 100 | 90 | 90 | 90 | 30 | 90 | 100 | — | — | 90 | 10 | 0 | — |
| Kochia | 70 | 90 | 50 | 20 | 0 | 0 | 20 | 90 | 40 | 80 | 10 | 0 | 0 | 30 |
| Pigweed, Palmer | 100 | 100 | 90 | 40 | 30 | 0 | 80 | 100 | — | — | 90 | 0 | 60 | — |
| Pigweed, Redroot | — | — | — | — | — | — | — | — | 80 | 90 | — | — | — | 30 |
| Ragweed | 100 | 20 | 20 | 30 | 0 | 0 | 0 | 70 | 30 | 20 | 90 | 0 | 0 | 30 |
| Ryegrass, Italian | 100 | 90 | 90 | 50 | 70 | 30 | 90 | 100 | 90 | 0 | 50 | 10 | 0 | 30 |
| 1000 g ai/ha | Compounds | | | | | | | | | | | | | |
| Preemergence | 124 | 125 | 128 | 129 | 130 | | 131 | | 132 | | 133 | | 134 | |
| Barnyardgrass | — | — | 0 | 20 | 10 | | 10 | | 10 | | 60 | | 30 | |
| Blackgrass | 0 | 0 | — | — | 10 | | 10 | | 0 | | 0 | | 40 | |
| Foxtail, Giant | 80 | 20 | 50 | 0 | 0 | | 60 | | 0 | | 80 | | 70 | |
| Goosegrass | 60 | 20 | — | — | — | | — | | — | | — | | — | |
| Kochia | 0 | 0 | 40 | 20 | 0 | | 0 | | 0 | | 0 | | 20 | |
| Pigweed, Palmer | 30 | 30 | — | — | 0 | | 0 | | 0 | | 40 | | 90 | |
| Pigweed, Redroot | — | — | 0 | 20 | — | | — | | — | | — | | — | |
| Ragweed | 0 | — | 0 | 0 | 0 | | 0 | | 0 | | 30 | | 50 | |
| Ryegrass, Italian | 60 | 60 | 60 | 50 | 0 | | 60 | | 0 | | 70 | | 80 | |
| 500 g ai/ha | Compounds | | | | | | | | | | | | | |
| Preemergence | 6 | 7 | 28 | 37 | 38 | 39 | 40 | 42 | 43 | 44 | 45 | 46 | 47 | 48 |
| Barnyardgrass | 0 | 0 | — | — | — | — | — | — | — | — | — | — | — | — |
| Blackgrass | — | — | 60 | 80 | 70 | 60 | 0 | 60 | 40 | 80 | 80 | 90 | 90 | 80 |
| Corn | — | — | 30 | 20 | 30 | 30 | 0 | 0 | 20 | 30 | 20 | 0 | 0 | 30 |
| Foxtail, Giant | 60 | 80 | 90 | 100 | 100 | 100 | 0 | 90 | 90 | 100 | 100 | 100 | 90 | 90 |
| Goosegrass | — | — | 80 | 100 | 100 | 100 | 0 | 90 | 90 | 100 | 100 | 100 | 100 | 80 |
| Kochia | 0 | 80 | 20 | 20 | 30 | 50 | 0 | 20 | 0 | 20 | 20 | 40 | 50 | 30 |
| Oat, Wild | — | — | 0 | 60 | 60 | 70 | 0 | 50 | 90 | 50 | 80 | 70 | 30 | 30 |
| Pigweed, Palmer | — | — | 50 | 90 | 70 | 90 | 0 | 80 | 80 | 20 | 70 | 90 | 90 | 90 |
| Pigweed, Redroot | 20 | 40 | — | — | — | — | — | — | — | — | — | — | — | — |
| Ragweed | 30 | 0 | 20 | 0 | 0 | 20 | 0 | 0 | 20 | 0 | 0 | 0 | 0 | 0 |
| Ryegrass, Italian | 50 | 50 | 90 | 90 | 80 | 100 | 0 | 100 | 90 | 100 | 90 | 80 | 90 | 100 |
| Soybean | — | — | 0 | 70 | 30 | 70 | 40 | 0 | 40 | 70 | 90 | 60 | 40 | 0 |
| Wheat | — | — | 10 | 20 | 10 | 30 | 0 | 20 | 10 | 40 | 60 | 30 | 20 | 0 |
| 500 g ai/ha | Compounds | | | | | | | | | | | | | |
| Preemergence | 50 | 51 | 52 | 53 | 54 | 55 | 56 | 57 | 59 | 60 | 61 | 63 | 64 | 65 |
| Barnyardgrass | — | — | — | — | — | — | — | — | — | — | — | — | — | — |
| Blackgrass | 80 | 60 | 60 | 0 | 80 | 90 | 0 | 90 | 60 | 70 | 70 | 90 | 90 | 90 |
| Corn | 0 | 50 | 40 | 0 | 60 | 30 | 0 | 40 | 20 | 30 | 0 | 80 | 50 | 50 |
| Foxtail, Giant | 100 | 100 | 100 | 0 | 100 | 100 | 0 | 100 | 100 | 100 | 90 | 100 | 100 | 100 |
| Goosegrass | 100 | 100 | 100 | 40 | 100 | 100 | 0 | 100 | 100 | 100 | 90 | 100 | 100 | 100 |
| Kochia | 10 | 90 | 70 | 20 | 90 | 100 | 0 | 30 | 90 | 30 | 60 | 40 | 90 | 100 |
| Oat, Wild | 90 | 90 | 80 | 0 | 50 | 90 | 0 | 90 | 90 | 0 | 30 | 90 | 70 | 80 |
| Pigweed, Palmer | 100 | 60 | 100 | 0 | 100 | 90 | 0 | 100 | 100 | 90 | 90 | 90 | 100 | 100 |
| Pigweed, Redroot | — | — | — | — | — | — | — | — | — | — | — | — | — | — |
| Ragweed | 30 | 0 | 70 | 0 | 0 | 40 | 0 | 50 | 0 | 20 | 40 | 40 | 0 | 40 |
| Ryegrass, Italian | 90 | 100 | 100 | 70 | 100 | 100 | 0 | 100 | 100 | 100 | 100 | 100 | 100 | 100 |
| Soybean | 90 | 100 | 90 | 0 | 40 | 100 | 0 | 100 | 90 | 100 | 30 | 100 | 30 | 90 |
| Wheat | 50 | 60 | 60 | 10 | 30 | 60 | 0 | 80 | 50 | 30 | 20 | 90 | 40 | 70 |
| 500 g ai/ha | Compounds | | | | | | | | | | | | | |
| Preemergence | 66 | 67 | 69 | 70 | 71 | 72 | 73 | 74 | 75 | 76 | 77 | 78 | 79 | 80 |
| Barnyardgrass | — | — | — | — | — | — | — | — | — | — | — | — | — | — |
| Blackgrass | 90 | 90 | 90 | 80 | 90 | 70 | 90 | 90 | — | — | — | — | 70 | 40 |
| Corn | 50 | 50 | 30 | 20 | 60 | 50 | 80 | 90 | 50 | 50 | 30 | 30 | 40 | 20 |
| Foxtail, Giant | 100 | 100 | 90 | 100 | 100 | 100 | 100 | 100 | 100 | 100 | 100 | 100 | 100 | 80 |
| Goosegrass | 100 | 100 | 100 | 100 | 100 | 100 | 100 | 100 | 100 | 100 | 100 | 100 | 100 | 100 |
| Kochia | 90 | 100 | 70 | 0 | 100 | 80 | 100 | 100 | 90 | 100 | 90 | 100 | 40 | 80 |
| Oat, Wild | 90 | 90 | 70 | 70 | 90 | 90 | 90 | 90 | — | — | — | 90 | 80 | 10 |
| Pigweed, Palmer | 100 | 100 | 90 | 90 | 100 | 100 | 100 | 100 | 100 | 100 | 100 | 100 | 100 | 80 |
| Pigweed, Redroot | — | — | — | — | — | — | — | — | — | — | — | — | — | — |
| Ragweed | 0 | 30 | 0 | 0 | 90 | 0 | 90 | 100 | 100 | 30 | 0 | 90 | 30 | 0 |
| Ryegrass, Italian | 100 | 100 | 80 | 90 | 100 | 100 | 100 | 100 | 100 | 100 | 100 | 100 | 100 | 100 |
| Soybean | 40 | 70 | 0 | 0 | 90 | 60 | 100 | 100 | 100 | 100 | 50 | 100 | 90 | 0 |
| Wheat | 80 | 30 | 20 | 10 | 90 | 30 | 80 | 90 | 80 | 90 | 50 | 80 | 50 | 10 |
| 500 g ai/ha | Compounds | | | | | | | | | | | | | |
| Preemergence | 81 | 82 | 83 | 84 | 85 | 86 | 88 | 89 | 90 | 91 | 92 | 93 | 94 | 95 |
| Barnyardgrass | — | — | — | — | — | — | — | — | — | — | — | — | — | — |
| Blackgrass | 60 | 0 | 50 | 80 | 30 | 30 | 80 | 90 | 0 | 90 | 90 | 90 | 90 | 80 |
| Corn | 0 | 10 | 20 | 20 | 0 | 20 | 40 | 60 | 0 | 60 | 30 | 10 | 90 | 100 |
| Foxtail, Giant | 90 | 20 | 100 | 100 | 90 | 70 | 100 | 100 | 0 | 100 | 100 | 100 | 100 | 100 |
| Goosegrass | 90 | 90 | 100 | 100 | 100 | 90 | 100 | 100 | 0 | 100 | 100 | 100 | 100 | 100 |
| Kochia | 30 | 0 | 100 | 100 | 80 | 70 | 100 | 100 | — | 100 | 100 | 100 | 100 | 100 |
| Oat, Wild | 80 | 20 | 90 | 90 | 10 | 80 | 90 | 90 | 0 | 90 | 90 | 90 | 90 | 90 |
| Pigweed, Palmer | 80 | 40 | 100 | 100 | 60 | 90 | 100 | 100 | 0 | 100 | 100 | 100 | 100 | 100 |
| Pigweed, Redroot | — | — | — | — | — | — | — | — | — | — | — | — | — | — |
| Ragweed | 0 | 0 | 90 | 100 | 0 | 0 | 100 | 90 | 0 | 30 | 30 | 0 | 0 | 70 |
| Ryegrass, Italian | 80 | 100 | 100 | 100 | 90 | 100 | 100 | 100 | 0 | 100 | 100 | 100 | 100 | 100 |
| Soybean | 0 | 0 | 100 | 100 | 0 | 0 | 100 | 100 | 0 | 70 | 90 | 30 | 100 | 100 |
| Wheat | 10 | 0 | 90 | 90 | 0 | 10 | 40 | 60 | 0 | 20 | 80 | 10 | 90 | 90 |
| 500 g ai/ha | Compounds | | | | | | | | | | | | | |
| Barnyardgrass | 96 | 97 | 98 | 99 | 100 | 101 | 102 | 103 | 104 | 105 | 106 | 107 | 108 | 109 |
| Blackgrass | 80 | 40 | 80 | 90 | 70 | 90 | 50 | 80 | 40 | 20 | 0 | 40 | 40 | 90 |
| Corn | 50 | 20 | 30 | 50 | 40 | 60 | 20 | 20 | 0 | 0 | 50 | 50 | 30 | 70 |
| Foxtail, Giant | 100 | 100 | 100 | 100 | 100 | 100 | 100 | 100 | 100 | 100 | 100 | 100 | 100 | 100 |
| Goosegrass | 100 | 100 | 100 | 100 | 100 | 100 | 100 | 100 | 100 | 100 | 100 | 100 | 100 | — |
| Kochia | 100 | 80 | 100 | 100 | 100 | 90 | 100 | 90 | 100 | 90 | 100 | 80 | 80 | 90 |
| Oat, Wild | 80 | 90 | 90 | 90 | 90 | 90 | 80 | 40 | 70 | 70 | 90 | 70 | 30 | 90 |
| Pigweed, Palmer | 100 | 100 | 100 | 100 | 100 | 100 | 100 | 100 | 100 | 90 | 100 | 100 | 100 | 100 |
| Pigweed, Redroot | — | — | — | — | — | — | — | — | — | — | — | — | — | — |
| Ragweed | 30 | 80 | 90 | 80 | 100 | 20 | 0 | 0 | 40 | 0 | 100 | 9 90 | 90 | 80 |
| Ryegrass, Italian | 100 | 100 | 100 | 100 | 100 | 90 | 100 | 90 | 90 | 80 | 100 | 100 | 100 | 100 |
| Soybean | 50 | 100 | 100 | 100 | 100 | 40 | 100 | 60 | 90 | 0 | 100 | 70 | 100 | 80 |
| Wheat | 50 | 40 | 60 | 80 | 60 | 50 | 0 | 0 | 0 | 10 | 40 | 40 | 40 | 90 |
| 500 g ai/ha | Compounds | | | | | | | | | | | | | |
| Preemergence | 110 | 111 | 112 | 113 | 114 | 117 | 118 | 122 | 126 | 127 | 135 | 136 | 137 | 138 |
| Barnyardgrass | 90 | 100 | 100 | 100 | 90 | — | — | — | — | — | 70 | 90 | 40 | 100 |
| Blackgrass | 70 | 70 | 80 | 90 | 70 | 90 | 70 | 50 | — | 70 | 80 | 90 | 30 | 90 |
| Corn | 50 | 40 | 0 | 90 | 0 | 10 | 30 | 10 | 40 | 0 | 10 | 30 | 50 | 10 |
| Foxtail, Giant | 100 | 100 | 90 | 100 | 100 | 90 | 100 | 90 | 100 | 100 | 100 | — | — | 100 |
| Goosegrass | — | — | — | — | — | 100 | 100 | 90 | 90 | 100 | — | — | — | — |
| Kochia | 20 | 90 | 60 | 100 | 20 | 60 | 70 | 10 | 0 | 60 | 0 | 70 | 0 | 50 |
| Oat, Wild | 50 | 90 | 20 | 90 | 20 | 70 | 90 | 20 | — | 20 | 90 | 30 | 0 | 90 |
| Pigweed, Palmer | 100 | 100 | 100 | 100 | 90 | 80 | 100 | 30 | 60 | 90 | 30 | 100 | 80 | 100 |
| Pigweed, Redroot | — | — | — | — | — | — | — | — | — | — | — | — | — | — |
| Ragweed | 40 | 90 | 40 | 70 | 0 | 40 | 30 | 0 | 0 | 0 | 0 | 20 | 0 | 0 |
| Ryegrass, Italian | 100 | 100 | 90 | 100 | 90 | 100 | 100 | 70 | 70 | 90 | 90 | 90 | 80 | 100 |
| Soybean | 60 | 100 | 50 | 80 | 0 | 70 | 60 | 0 | 50 | 0 | 30 | 60 | 0 | 100 |
| Wheat | 30 | 90 | 40 | 90 | 50 | 20 | 30 | 0 | 30 | 0 | 30 | 30 | 0 | 90 |
| 500 g ai/ha | Compounds | | | | | | | | | | | | | |
| Preemergence | 139 | 140 | 141 | 142 | 143 | 144 | 145 | 146 | 147 | | 148 | | 149 | |
| Barnyardgrass | 90 | 90 | 90 | 50 | 100 | 90 | 40 | 70 | 100 | | 100 | | 100 | |
| Blackgrass | 90 | 70 | 70 | 30 | 70 | 80 | 80 | 90 | 90 | | 90 | | 90 | |
| Corn | 40 | 10 | 10 | 10 | 40 | 50 | 0 | 0 | 10 | | 60 | | 90 | |
| Foxtail, Giant | 100 | 100 | 100 | 90 | 100 | 100 | 80 | 100 | — | | 100 | | 100 | |
| Goosegrass | — | — | — | — | — | — | — | — | — | | — | | — | |
| Kochia | 0 | 90 | 50 | 40 | 100 | 60 | 50 | 30 | 90 | | 70 | | 100 | |
| Oat, Wild | 90 | 70 | 80 | 0 | 90 | 60 | 50 | 20 | 90 | | 80 | | 90 | |
| Pigweed, Palmer | 100 | 100 | 100 | 20 | 100 | 100 | 100 | 100 | 100 | | 100 | | 100 | |
| Pigweed, Redroot | — | — | — | — | — | — | — | — | — | | — | | — | |
| Ragweed | 0 | 30 | 50 | 0 | 90 | 30 | 0 | 0 | 0 | | 90 | | 90 | |
| Ryegrass, Italian | 80 | 70 | 100 | 50 | 90 | 100 | 90 | 70 | 100 | | 100 | | 100 | |
| Soybean | 40 | 100 | 80 | 0 | 100 | 100 | 0 | 20 | 100 | | 100 | | 100 | |
| Wheat | 20 | 50 | 30 | 0 | 50 | 50 | 0 | 0 | 50 | | 90 | | 90 | |
| 125 g ai/ha | Compounds | | | | | | | | | | | | | |
| Preemergence | 6 | 7 | 28 | 37 | 38 | 39 | 40 | 42 | 43 | 44 | 45 | 46 | 47 | 48 |
| Barnyardgrass | 0 | 0 | — | — | — | — | — | — | — | — | — | — | — | — |
| Blackgrass | — | — | 0 | 60 | 30 | 0 | 0 | 30 | 10 | 10 | 50 | 60 | 70 | 20 |
| Corn | — | — | 0 | 0 | 20 | 0 | 0 | 0 | 10 | 10 | 0 | 0 | 0 | 0 |
| Foxtail, Giant | 0 | 10 | 30 | 90 | 70 | 90 | 0 | 10 | 60 | 70 | 70 | 60 | 60 | 20 |
| Goosegrass | — | — | 20 | 90 | 70 | 90 | 0 | 30 | 60 | 90 | 70 | 60 | 80 | 40 |
| Kochia | 0 | 40 | 0 | 0 | 0 | 0 | 0 | 0 | 0 | 0 | 0 | — | 0 | 0 |
| Oat, Wild | — | — | 0 | 0 | 10 | 0 | 0 | 0 | 20 | 10 | 0 | 0 | 10 | 20 |
| Pigweed, Palmer | — | — | 0 | 50 | 10 | 50 | 0 | 10 | 0 | 10 | 30 | 40 | 60 | 30 |
| Pigweed, Redroot | 0 | 0 | — | — | — | — | — | — | — | — | — | — | — | — |
| Ragweed | 0 | 0 | 0 | 0 | 0 | 10 | 0 | 0 | 0 | 0 | 0 | 0 | 0 | 0 |
| Ryegrass, Italian | 20 | 50 | 40 | 80 | 70 | 40 | 0 | 50 | 60 | 80 | 70 | 60 | 80 | 70 |
| Soybean | — | — | 0 | 0 | 0 | 30 | 0 | 0 | — | 40 | 20 | 20 | 0 | 0 |
| Wheat | — | — | 0 | 0 | 0 | 0 | 0 | 0 | 0 | 0 | 0 | 0 | 0 | 0 |
| 125 g ai/ha | Compounds | | | | | | | | | | | | | |
| Preemergence | 50 | 51 | 52 | 53 | 54 | 55 | 56 | 57 | 59 | 60 | 61 | 62 | 63 | 64 |
| Barnyardgrass | — | — | — | — | — | — | — | — | — | — | — | — | — | — |
| Blackgrass | 40 | 30 | 50 | 0 | 40 | 80 | 0 | 60 | 0 | 20 | 50 | 40 | 80 | 80 |
| Corn | 0 | 20 | 20 | 0 | 30 | 0 | 0 | 0 | — | 20 | 0 | 0 | 60 | 20 |
| Foxtail, Giant | 90 | 100 | 90 | 0 | 90 | 100 | 0 | 100 | 90 | 100 | 90 | 90 | 100 | 90 |
| Goosegrass | 90 | 90 | 90 | 0 | 90 | 100 | 0 | 90 | 90 | 100 | 90 | 100 | 100 | 60 |
| Kochia | 0 | 70 | 0 | 0 | 80 | 90 | 0 | 40 | 0 | 0 | 20 | 80 | 30 | 80 |
| Oat, Wild | 20 | 30 | 40 | 0 | 20 | 70 | 0 | 50 | 20 | 0 | 30 | 0 | 60 | 30 |
| Pigweed, Palmer | 0 | 0 | 0 | 0 | 60 | 80 | 0 | 30 | 10 | 30 | 60 | 90 | 40 | 40 |
| Pigweed, Redroot | — | — | — | — | — | — | — | — | — | — | — | — | — | — |
| Ragweed | 0 | 0 | 0 | 0 | 0 | 20 | 0 | 0 | 0 | 0 | 0 | 20 | 0 | 0 |
| Ryegrass, Italian | 80 | 90 | 80 | 30 | 70 | 80 | 0 | 80 | 90 | 100 | 90 | 70 | 90 | 90 |
| Soybean | 30 | 90 | 30 | 0 | 0 | 30 | 0 | 100 | — | 40 | 0 | 0 | 100 | 20 |
| Wheat | 0 | 20 | 30 | 0 | 20 | 40 | 0 | 20 | 0 | 0 | 0 | 0 | 40 | 20 |
| 125 g ai/ha | Compounds | | | | | | | | | | | | | |
| Preemergence | 65 | 66 | 67 | 69 | 70 | 71 | 72 | 73 | 74 | 75 | 76 | 77 | 78 | 79 |
| Barnyardgrass | — | — | — | — | — | — | — | — | — | — | — | — | — | — |
| Blackgrass | 80 | 70 | 80 | 40 | 30 | 80 | 40 | 70 | 70 | — | — | — | — | 50 |
| Corn | 40 | 30 | 40 | 30 | 10 | 40 | 30 | 60 | 40 | 40 | 40 | 0 | 30 | 20 |
| Foxtail, Giant | 100 | 90 | 100 | 90 | 20 | 100 | 100 | 100 | 100 | 100 | 90 | 90 | 100 | 100 |
| Goosegrass | 100 | 100 | 100 | 90 | 70 | 100 | 90 | 100 | 100 | 100 | 100 | 100 | 90 | 100 |
| Kochia | 80 | 10 | 80 | 60 | 0 | 80 | 30 | 100 | 40 | 80 | 70 | 90 | 90 | 20 |
| Oat, Wild | 70 | 30 | 30 | 20 | 0 | 90 | 10 | 80 | 60 | — | — | — | 80 | 90 |
| Pigweed, Palmer | 90 | 50 | 100 | 20 | 20 | 100 | 70 | 100 | 90 | 100 | 100 | 90 | 100 | 40 |
| Pigweed, Redroot | — | — | — | — | — | — | — | — | — | — | — | — | — | — |
| Ragweed | 0 | 0 | 0 | 0 | 0 | 0 | 0 | 30 | 0 | 0 | 30 | 0 | 0 | 0 |
| Ryegrass, Italian | 100 | 100 | 100 | 80 | 50 | 100 | 100 | 90 | 100 | 100 | 100 | 80 | 90 | 60 |
| Soybean | 30 | 30 | 50 | 0 | 0 | 50 | 30 | 60 | 70 | 80 | 50 | 0 | 0 | 60 |
| Wheat | 40 | 20 | 10 | 10 | 0 | 30 | 0 | 20 | 20 | 30 | 60 | 20 | 20 | 10 |
| 125 g ai/ha | Compounds | | | | | | | | | | | | | |
| Preemergence | 80 | 81 | 82 | 83 | 84 | 85 | 86 | 87 | 88 | 89 | 90 | 91 | 92 | 93 |
| Barnyardgrass | — | — | — | — | — | — | — | — | — | — | — | — | — | — |
| Blackgrass | 20 | 30 | 0 | 40 | 80 | 0 | 0 | 40 | 80 | 70 | 0 | 80 | 80 | 80 |
| Corn | 0 | 0 | 0 | 0 | 20 | 0 | 0 | 10 | 40 | 20 | 0 | 30 | 30 | 10 |
| Foxtail, Giant | 20 | 20 | 0 | 100 | 100 | 30 | 0 | 50 | 100 | 100 | 0 | 90 | 70 | 80 |
| Goosegrass | 90 | 70 | 20 | 100 | 100 | 80 | 60 | 80 | 100 | 100 | 0 | 100 | 100 | 90 |
| Kochia | 10 | 0 | 0 | 90 | 80 | 20 | 60 | 30 | 90 | 70 | — | 90 | 70 | 50 |
| Oat, Wild | 0 | 50 | 10 | 90 | 90 | 0 | 0 | 20 | 90 | 70 | 0 | 10 | 80 | 20 |
| Pigweed, Palmer | 10 | 30 | 0 | 100 | 100 | 70 | 20 | 30 | 100 | 90 | 0 | 90 | 100 | 90 |
| Pigweed, Redroot | — | — | — | — | — | — | — | — | — | — | — | — | — | — |
| Ragweed | 0 | 0 | 0 | 50 | 70 | 0 | 0 | 0 | 60 | 40 | 0 | 0 | 0 | 0 |
| Ryegrass, Italian | 90 | 50 | 60 | 100 | 100 | 90 | 70 | 100 | 100 | 90 | 0 | 90 | 100 | 90 |
| Soybean | 0 | 0 | 0 | 50 | 90 | 0 | 0 | 0 | 50 | 100 | 0 | 20 | 50 | 30 |
| Wheat | 0 | 10 | 0 | 10 | 60 | 0 | 0 | 0 | 20 | 20 | 0 | 0 | 30 | 0 |
| 125 g ai/ha | Compounds | | | | | | | | | | | | | |
| Preemergence | 94 | 95 | 96 | 97 | 98 | 99 | 100 | 101 | 102 | 103 | 104 | 105 | 106 | 107 |
| Barnyardgrass | — | — | — | — | — | — | — | — | — | — | — | — | — | — |
| Blackgrass | 90 | 70 | 40 | 10 | 60 | 80 | 60 | 50 | 30 | 50 | 30 | 10 | 0 | 20 |
| Corn | 60 | 60 | 30 | 10 | 10 | 10 | 0 | 0 | 10 | 20 | 0 | 0 | 10 | 10 |
| Foxtail, Giant | 100 | 100 | 100 | 100 | 100 | 100 | 100 | 90 | 90 | 100 | 100 | 70 | 100 | 100 |
| Goosegrass | 100 | 100 | 100 | 100 | 100 | 100 | 100 | 90 | 100 | 100 | 100 | 60 | 100 | 100 |
| Kochia | 90 | 60 | 30 | 60 | 80 | 100 | 90 | 20 | 80 | 50 | 50 | 50 | 50 | 20 |
| Oat, Wild | 40 | 80 | 50 | 90 | 70 | 70 | 40 | 40 | 40 | 40 | 0 | 70 | 20 | 60 |
| Pigweed, Palmer | 100 | 90 | 100 | 100 | 100 | 100 | 100 | 90 | 100 | 90 | 90 | 30 | 100 | 100 |
| Pigweed, Redroot | — | — | — | — | — | — | — | — | — | — | — | — | — | — |
| Ragweed | 0 | 0 | 30 | 30 | 30 | 40 | 80 | 0 | 0 | 0 | 0 | 0 | 30 | 30 |
| Ryegrass, Italian | 90 | 100 | 100 | 70 | 100 | 90 | 90 | 80 | 80 | 80 | 50 | 40 | 40 | 80 |
| Soybean | 50 | 50 | 50 | 40 | 90 | 80 | 80 | 20 | 50 | 30 | 60 | 0 | 50 | 50 |
| Wheat | 50 | 90 | 20 | 10 | 50 | 30 | 50 | 0 | 0 | 0 | 0 | 0 | 0 | 0 |
| 125 g ai/ha | Compounds | | | | | | | | | | | | | |
| Preemergence | 108 | 109 | 110 | 111 | 112 | 113 | 114 | 117 | 118 | 122 | 126 | 127 | 135 | 136 |
| Barnyardgrass | — | 90 | 50 | 90 | 40 | 90 | 30 | — | — | — | — | — | 30 | 30 |
| Blackgrass | 0 | 80 | 40 | 30 | 20 | 70 | 30 | 30 | 20 | 10 | — | 20 | 60 | 70 |
| Corn | 0 | 40 | 0 | 30 | 0 | 20 | 0 | 0 | 0 | 0 | 0 | 0 | 0 | 0 |
| Foxtail, Giant | 90 | 100 | 90 | 100 | 50 | 100 | 80 | 60 | 20 | 20 | 60 | 90 | 10 | — |
| Goosegrass | 100 | — | — | — | — | — | — | 50 | 90 | 0 | 20 | 60 | — | — |
| Kochia | 0 | 60 | 0 | 80 | 0 | 60 | 0 | 0 | 0 | 0 | 0 | 0 | 0 | 0 |
| Oat, Wild | 30 | 50 | 0 | 70 | 0 | 80 | 0 | 20 | 30 | 0 | — | 0 | 60 | 20 |
| Pigweed, Palmer | 100 | 90 | 90 | 90 | 50 | 90 | 50 | 20 | 40 | 0 | 20 | 40 | 10 | 70 |
| Pigweed, Redroot | — | — | — | — | — | — | — | — | — | — | — | — | — | — |
| Ragweed | 30 | 40 | 0 | 20 | 0 | 0 | 0 | 0 | 0 | 0 | 0 | 0 | 0 | 0 |
| Ryegrass, Italian | 80 | 80 | 70 | 70 | 40 | 90 | 50 | 40 | 70 | 20 | 40 | 20 | 60 | 80 |
| Soybean | 50 | 30 | 40 | 70 | 0 | 30 | 0 | 30 | 40 | 0 | 0 | 0 | 20 | 10 |
| Wheat | 30 | 40 | 20 | 60 | 20 | 80 | 20 | 0 | 0 | 0 | 0 | 0 | 0 | 10 |
| 125 g ai/ha | Compounds | | | | | | | | | | | | | |
| Preemergence | 137 | 138 | 139 | 140 | 141 | 142 | 143 | 144 | 145 | 146 | 147 | 148 | 149 | |
| Barnyardgrass | 0 | 90 | 70 | 90 | 70 | 30 | 90 | 50 | 0 | 20 | 40 | 70 | 100 | |
| Blackgrass | 0 | 80 | 70 | 40 | 20 | 0 | 40 | 50 | 40 | 70 | 20 | 80 | 80 | |
| Corn | 0 | 10 | 0 | 0 | 10 | 0 | 10 | 30 | 0 | 0 | 0 | 60 | 60 | |
| Foxtail, Giant | — | 100 | 90 | 90 | 90 | 30 | 100 | 100 | 70 | 80 | — | 100 | 100 | |
| Goosegrass | — | — | — | — | — | — | — | — | — | — | — | — | — | |
| Kochia | 0 | 0 | 0 | 80 | 0 | 0 | 80 | 0 | 70 | 10 | 70 | 20 | 100 | |
| Oat, Wild | 0 | 40 | 20 | 30 | 60 | 0 | 60 | 30 | 0 | 0 | 20 | 20 | 90 | |
| Pigweed, Palmer | 10 | 90 | 40 | 100 | 100 | 0 | 100 | 10 | 80 | 90 | 90 | 100 | 90 | |
| Pigweed, Redroot | — | — | — | — | — | — | — | — | — | — | — | — | — | |
| Ragweed | 0 | 0 | 0 | 0 | 0 | 0 | 30 | 0 | 0 | 0 | 0 | 30 | 100 | |
| Ryegrass, Italian | 70 | 80 | 50 | 50 | 90 | 30 | 80 | 90 | 40 | 30 | 90 | 80 | 100 | |
| Soybean | 0 | 70 | 0 | 50 | 80 | 0 | 100 | 0 | 0 | 0 | 0 | 70 | 100 | |
| Wheat | 0 | 40 | 20 | 20 | 20 | 0 | 90 | 30 | 0 | 0 | 20 | 30 | 30 | |
| 31 g ai/ha | Compound | | | | | | | | | | | | | |
| Preemergence | 62 | | | | | | | | | | | | | |
| Blackgrass | 0 | | | | | | | | | | | | | |
| Corn | 0 | | | | | | | | | | | | | |
| Foxtail, Giant | 20 | | | | | | | | | | | | | |
| Goosegrass | 70 | | | | | | | | | | | | | |
| Kochia | 10 | | | | | | | | | | | | | |
| Oat, Wild | 0 | | | | | | | | | | | | | |
| Pigweed, Palmer | 70 | | | | | | | | | | | | | |
| Ragweed | 0 | | | | | | | | | | | | | |
| Ryegrass, Italian | 0 | | | | | | | | | | | | | |
| Soybean | 0 | | | | | | | | | | | | | |
| Wheat | 0 | | | | | | | | | | | | | |
| 1000 g ai/ha | Compounds | | | | | | | | | | | | | |
| Preemergence | 1 | 2 | 3 | 4 | 5 | 8 | 9 | 10 | 11 | 12 | 13 | 14 | 15 | 16 |
| Barnyardgrass | 60 | 40 | 0 | 0 | 50 | 40 | 20 | 60 | 30 | 0 | 0 | 0 | 30 | 10 |
| Blackgrass | 60 | 0 | 0 | 0 | 40 | 0 | 0 | 50 | 30 | 0 | 10 | 0 | 0 | 0 |
| Corn | 20 | 40 | 0 | 10 | 10 | 20 | 0 | 30 | 20 | 0 | 0 | 0 | 30 | 0 |
| Foxtail, Giant | 70 | 70 | 0 | 0 | 70 | 40 | 0 | 80 | 50 | 0 | 0 | 10 | 30 | 0 |
| Galium | 80 | 50 | 0 | 20 | 70 | 40 | 20 | 100 | 90 | 0 | 30 | 20 | 80 | 20 |
| Goosegrass | — | — | — | — | — | — | — | — | — | — | — | — | — | — |
| Horseweed | — | — | — | — | — | — | — | — | — | — | — | — | — | — |
| Kochia | 30 | 0 | 0 | 0 | 20 | 0 | 0 | 40 | 20 | 0 | 0 | 0 | 30 | 0 |
| Pigweed, Palmer | — | — | — | — | — | — | — | — | — | — | — | — | — | — |
| Pigweed, Redroot | 50 | 30 | 0 | 0 | 30 | 30 | 0 | 40 | 40 | 0 | 0 | 0 | 0 | 0 |
| Ragweed | 0 | 0 | 30 | 0 | 0 | 0 | 0 | 60 | 20 | 0 | 0 | 0 | 30 | 0 |
| Ryegrass, Italian | 40 | 0 | 0 | 0 | 20 | 0 | 0 | 50 | 0 | 0 | 0 | 0 | 10 | 0 |
| Wheat | 40 | 0 | 40 | 0 | 40 | 0 | 0 | 40 | 60 | 0 | 40 | 0 | 10 | 0 |
| 1000 g ai/ha | Compounds | | | | | | | | | | | | | |
| Postemergence | 17 | 18 | 19 | 20 | 21 | 22 | 23 | 24 | 25 | 26 | 29 | 30 | 31 | 32 |
| Barnyardgrass | 0 | 70 | 80 | 90 | 90 | 50 | 10 | 30 | 50 | 0 | 70 | 20 | 30 | — |
| Blackgrass | 0 | 70 | 70 | 70 | 80 | 80 | 30 | 60 | 90 | 0 | 70 | 70 | 50 | 80 |
| Corn | 0 | 40 | 50 | 60 | 90 | 70 | 30 | 10 | 70 | 0 | 80 | 10 | 30 | — |
| Foxtail, Giant | 0 | 90 | 60 | 90 | 90 | 80 | 10 | 50 | 90 | 0 | 90 | 40 | 80 | 100 |
| Galium | 0 | 90 | 90 | 90 | 80 | 90 | — | — | — | — | — | — | — | 100 |
| Goosegrass | — | — | — | — | — | — | — | — | — | — | — | — | — | 80 |
| Horseweed | — | — | — | — | — | — | — | — | — | — | — | — | — | 0 |
| Kochia | 10 | 50 | 30 | 30 | 40 | 70 | 10 | 10 | 50 | 0 | 10 | 10 | 20 | 60 |
| Pigweed, Palmer | — | — | — | — | — | — | — | — | — | — | — | — | — | 70 |
| Pigweed, Redroot | 0 | 40 | 40 | 30 | 60 | 60 | 0 | 0 | 40 | 0 | 50 | 30 | 0 | — |
| Ragweed | 0 | 20 | 0 | 30 | 40 | 50 | 20 | 0 | 0 | 0 | 0 | 0 | 0 | 20 |
| Ryegrass, Italian | 0 | 80 | 60 | 70 | 80 | 60 | 0 | 60 | 90 | 0 | 50 | 70 | 70 | 90 |
| Wheat | 0 | 0 | 70 | 70 | 70 | 60 | 20 | 30 | 70 | 0 | 60 | 40 | 40 | — |
| 1000 g ai/ha | Compounds | | | | | | | | | | | | | |
| Postemergence | 33 | 34 | 35 | 36 | 41 | 49 | 58 | 68 | 115 | 116 | 119 | 120 | 121 | 123 |
| Barnyardgrass | — | — | — | — | — | — | — | — | 50 | 0 | — | — | — | 20 |
| Blackgrass | 90 | 70 | 60 | 60 | 50 | 0 | 20 | 80 | 50 | 0 | 70 | 0 | 0 | 20 |
| Corn | — | — | — | — | — | — | — | — | 40 | 0 | — | — | — | 0 |
| Foxtail, Giant | 100 | 100 | 90 | 90 | 60 | 10 | 30 | 90 | 40 | 0 | 90 | 20 | 20 | 10 |
| Galium | 100 | 100 | 80 | 90 | 70 | 30 | 60 | 100 | 70 | 90 | 90 | 40 | 80 | 50 |
| Goosegrass | 90 | 50 | 50 | 30 | 0 | 10 | 20 | 90 | — | — | 70 | 10 | 0 | — |
| Horseweed | 30 | 0 | 20 | 0 | 0 | 0 | 0 | 0 | — | — | 0 | 0 | 0 | — |
| Kochia | 70 | 100 | 50 | 60 | 20 | 10 | 60 | 80 | 0 | 70 | 90 | 50 | 70 | 20 |
| Pigweed, Palmer | 100 | 80 | 50 | 60 | 20 | 0 | 20 | 50 | — | — | 80 | 40 | 50 | — |
| Pigweed, Redroot | — | — | — | — | — | — | — | — | 30 | 90 | — | — | — | 60 |
| Ragweed | 80 | 50 | 50 | 60 | 0 | 30 | 10 | 60 | 0 | 0 | 70 | 0 | 0 | 0 |
| Ryegrass, Italian | 100 | 90 | 80 | 40 | 60 | 30 | 30 | 90 | 60 | 0 | 20 | 0 | 0 | 30 |
| Wheat | — | — | — | — | — | — | — | — | 50 | 0 | — | — | — | 0 |
| 1000 g ai/ha | Compounds | | | | | | | | | | | | | |
| Postemergence | 124 | 125 | 128 | 129 | 130 | | 131 | | 132 | | 133 | | 134 | |
| Barnyardgrass | — | — | 30 | 20 | 0 | | 10 | | 0 | | 20 | | 10 | |
| Blackgrass | 0 | 20 | 30 | 30 | 0 | | 0 | | 0 | | 0 | | 0 | |
| Corn | — | — | 0 | 0 | — | | — | | — | | — | | — | |
| Foxtail, Giant | 40 | 20 | 20 | 10 | 0 | | 10 | | 10 | | 30 | | 50 | |
| Galium | 30 | 40 | 30 | 30 | 0 | | 20 | | 0 | | 60 | | 20 | |
| Goosegrass | 0 | 0 | — | — | — | | — | | — | | — | | — | |
| Horseweed | 0 | 0 | — | — | 0 | | 0 | | 0 | | 0 | | 0 | |
| Kochia | 30 | 50 | 0 | 10 | 0 | | 40 | | 20 | | 40 | | 50 | |
| Pigweed, Palmer | 60 | 40 | — | — | 0 | | 0 | | 0 | | 30 | | 30 | |
| Pigweed, Redroot | — | — | 0 | 70 | — | | — | | — | | — | | — | |
| Ragweed | 0 | 0 | 30 | 0 | 0 | | 0 | | 0 | | 40 | | 0 | |
| Ryegrass, Italian | 0 | 0 | 0 | 0 | 0 | | 0 | | 0 | | 60 | | 20 | |
| Wheat | — | — | 0 | 0 | — | | — | | — | | — | | — | |
| 500 g ai/ha | Compounds | | | | | | | | | | | | | |
| Postemergence | 6 | 7 | 28 | 37 | 38 | 39 | 40 | 42 | 43 | 44 | 45 | 46 | 47 | 48 |
| Barnyardgrass | 10 | 20 | — | — | — | — | — | — | — | — | — | — | — | — |
| Blackgrass | 0 | 30 | 80 | 80 | 70 | 60 | 0 | 40 | 0 | 60 | 60 | 70 | 60 | 30 |
| Corn | 0 | 0 | 20 | 80 | 80 | 70 | 0 | 40 | 30 | 70 | 20 | 30 | 60 | 60 |
| Foxtail, Giant | 10 | 20 | 70 | 90 | 90 | 90 | 0 | 70 | 70 | 90 | 90 | 90 | 90 | 70 |
| Galium | 30 | 60 | 50 | 100 | 50 | 100 | 0 | 70 | 60 | 70 | 90 | 100 | 90 | 60 |
| Goosegrass | — | — | 50 | 90 | 70 | 60 | 0 | 30 | 40 | 70 | 60 | 60 | 20 | 40 |
| Horseweed | — | — | 0 | — | — | 20 | 0 | — | — | — | — | — | — | — |
| Kochia | 0 | 20 | 20 | 90 | 60 | 60 | 10 | 80 | 30 | 20 | 80 | 80 | 90 | 50 |
| Oat, Wild | — | — | 30 | 30 | 40 | 90 | 0 | 10 | 20 | 80 | 20 | 40 | 30 | 30 |
| Pigweed, Palmer | — | — | 20 | 60 | 50 | 40 | 0 | 10 | 20 | 10 | 10 | 50 | 90 | 70 |
| Pigweed, Redroot | 30 | 30 | — | — | — | — | — | — | — | — | — | — | — | — |
| Ragweed | 0 | 0 | 0 | 0 | 0 | 60 | 0 | 0 | 0 | 0 | 30 | 0 | 0 | 20 |
| Ryegrass, Italian | 40 | 0 | 70 | 90 | 60 | 100 | 0 | 50 | 60 | 80 | 80 | 80 | 90 | 40 |
| Soybean | — | — | 90 | 100 | 50 | 100 | 20 | 90 | 100 | 100 | 100 | 100 | 100 | 100 |
| Wheat | 0 | 40 | 30 | 80 | 70 | 50 | 0 | 20 | 30 | 80 | 40 | 50 | 40 | 30 |
| 500 g ai/ha | Compounds | | | | | | | | | | | | | |
| Postemergence | 50 | 51 | 52 | 53 | 54 | 55 | 56 | 57 | 59 | 60 | 61 | 63 | 64 | 65 |
| Barnyardgrass | — | — | — | — | — | — | — | — | — | — | — | — | — | — |
| Blackgrass | 70 | 50 | 60 | 20 | 50 | 80 | 0 | 80 | 80 | 60 | 40 | 90 | 70 | 80 |
| Corn | 60 | 50 | 70 | 0 | 40 | 80 | 0 | 80 | 20 | 80 | 10 | 80 | 80 | 70 |
| Foxtail, Giant | 90 | 90 | 90 | 10 | 90 | 100 | 0 | 90 | 90 | 90 | 80 | 90 | 90 | 50 |
| Galium | 100 | 100 | 90 | 0 | 100 | 100 | 0 | 100 | 100 | 100 | 100 | 100 | 100 | 100 |
| Goosegrass | 90 | 80 | 60 | 40 | 50 | 90 | 0 | 90 | 50 | 80 | 50 | 80 | 30 | 40 |
| Horseweed | 10 | 20 | 0 | 0 | 20 | 30 | 0 | 20 | 0 | 0 | — | 40 | 40 | 20 |
| Kochia | 70 | 70 | 60 | 10 | 90 | 90 | 0 | 30 | 70 | 70 | 70 | 20 | 80 | 90 |
| Oat, Wild | 50 | 70 | 80 | 0 | 40 | 80 | 0 | 90 | 50 | 90 | 20 | 80 | 70 | 20 |
| Pigweed, Palmer | 30 | 50 | 60 | 30 | 60 | 60 | 0 | 50 | 50 | 50 | 50 | 40 | 50 | 70 |
| Pigweed, Redroot | — | — | — | — | — | — | — | — | — | — | — | — | — | — |
| Ragweed | 40 | 0 | 30 | 0 | 50 | 70 | 0 | 50 | 40 | 50 | 40 | 40 | 40 | 80 |
| Ryegrass, Italian | 80 | 90 | 80 | 0 | 60 | 100 | 0 | 100 | 60 | 60 | 40 | 100 | 100 | 100 |
| Soybean | 100 | 100 | 100 | 50 | 100 | 100 | 10 | 100 | 100 | 100 | 100 | 100 | 100 | 100 |
| Wheat | 70 | 80 | 60 | 0 | 40 | 60 | 0 | 90 | 40 | 60 | 20 | 90 | 40 | 20 |
| 500 g ai/ha | Compounds | | | | | | | | | | | | | |
| Postemergence | 66 | 67 | 69 | 70 | 71 | 72 | 73 | 74 | 75 | 76 | 77 | 78 | 79 | 80 |
| Barnyardgrass | — | — | — | — | — | — | — | — | — | — | — | — | — | — |
| Blackgrass | 90 | 90 | 60 | 70 | 90 | 80 | 90 | 90 | 90 | 70 | 70 | 90 | 80 | 40 |
| Corn | 70 | 60 | 30 | 10 | 90 | 90 | 70 | 90 | 90 | 60 | 80 | 60 | 80 | 20 |
| Foxtail, Giant | 90 | 90 | 80 | 30 | 90 | 50 | 90 | 90 | 100 | 90 | 90 | 90 | 90 | 50 |
| Galium | 100 | 100 | 100 | 80 | 100 | 100 | 100 | 100 | 100 | 100 | 100 | 100 | — | — |
| Goosegrass | 50 | 70 | 20 | 10 | 50 | 90 | 60 | 0 | 90 | 80 | 60 | 80 | 80 | 30 |
| Horseweed | 0 | 30 | 0 | 0 | 80 | 50 | 50 | 0 | 70 | 80 | 50 | 0 | 20 | 0 |
| Kochia | 80 | 100 | 60 | 60 | 90 | 90 | 100 | 90 | 90 | 90 | 90 | 100 | 60 | 70 |
| Oat, Wild | 70 | 60 | 30 | 10 | 80 | 50 | 80 | 60 | 90 | 90 | 10 | 90 | 80 | 20 |
| Pigweed, Palmer | 30 | 90 | 40 | 70 | 70 | 70 | 90 | 70 | 100 | 100 | 70 | 90 | 90 | 70 |
| Pigweed, Redroot | — | — | — | — | — | — | — | — | — | — | — | — | — | — |
| Ragweed | 40 | 70 | 30 | 0 | 80 | 50 | 80 | 50 | 80 | 60 | 0 | 80 | 50 | 60 |
| Ryegrass, Italian | 70 | 100 | 70 | 30 | 100 | 70 | 100 | 100 | 100 | 80 | 60 | 100 | 100 | 70 |
| Soybean | 100 | 100 | 100 | 100 | 100 | 100 | 100 | 100 | 100 | 100 | 100 | 100 | 100 | 100 |
| Wheat | 20 | 30 | 10 | 20 | 80 | 30 | 80 | 80 | 60 | 40 | 30 | 50 | 50 | 10 |
| 500 g ai/ha | Compounds | | | | | | | | | | | | | |
| Postemergence | 81 | 82 | 83 | 84 | 85 | 86 | 88 | 89 | 90 | 91 | 92 | 93 | 94 | 95 |
| Barnyardgrass | — | — | — | — | — | — | — | — | — | — | — | — | — | — |
| Blackgrass | 60 | 10 | 60 | 70 | 50 | 30 | 90 | 80 | 0 | 100 | 90 | 90 | 90 | 90 |
| Corn | 50 | 10 | 50 | 90 | 10 | 10 | 90 | 70 | 0 | 80 | 70 | 90 | 90 | 90 |
| Foxtail, Giant | 90 | 10 | 90 | 100 | 40 | 20 | 90 | 100 | 0 | 90 | 80 | 90 | 90 | 90 |
| Galium | — | — | — | — | — | — | — | — | 0 | 100 | 100 | 100 | 100 | 100 |
| Goosegrass | 30 | 20 | 100 | 100 | 30 | 50 | 100 | 100 | 0 | 90 | 50 | 70 | 90 | 90 |
| Horseweed | 0 | 0 | 40 | 50 | 50 | 0 | 90 | 40 | 0 | 20 | 50 | 40 | 50 | 30 |
| Kochia | 90 | 80 | 100 | 90 | 90 | 90 | 100 | 90 | 20 | 100 | 90 | 90 | 100 | 90 |
| Oat, Wild | 20 | 0 | 70 | 90 | 30 | 10 | 90 | 90 | 0 | 90 | 70 | 70 | 80 | 80 |
| Pigweed, Palmer | 90 | 50 | 90 | 100 | 80 | 40 | 90 | 100 | 50 | 90 | 90 | 100 | 90 | 90 |
| Pigweed, Redroot | — | — | — | — | — | — | — | — | — | — | — | — | — | — |
| Ragweed | 20 | 50 | 100 | 100 | 20 | 50 | 100 | 80 | 0 | 70 | 70 | 50 | 50 | 50 |
| Ryegrass, Italian | 100 | 30 | 100 | 100 | 70 | 50 | 100 | 100 | 0 | 100 | 100 | 90 | 90 | 100 |
| Soybean | 100 | 90 | 100 | 100 | 100 | 100 | 100 | 100 | 20 | 100 | 100 | 100 | 100 | 100 |
| Wheat | 20 | 10 | 40 | 50 | 10 | 20 | 90 | 50 | 10 | 80 | 50 | 20 | 70 | 80 |
| 500 g ai/ha | Compounds | | | | | | | | | | | | | |
| Postemergence | 96 | 97 | 98 | 99 | 100 | 101 | 102 | 103 | 104 | 105 | 106 | 107 | 108 | 109 |
| Barnyardgrass | — | — | — | — | — | — | — | — | — | — | — | — | — | 90 |
| Blackgrass | 90 | 80 | 90 | 90 | 90 | 100 | 60 | 90 | 50 | 30 | 80 | 80 | 30 | 90 |
| Corn | 60 | 70 | 90 | 90 | 70 | 80 | 80 | 80 | 80 | 50 | 90 | 80 | 60 | 90 |
| Foxtail, Giant | 90 | 90 | 90 | 90 | 90 | 90 | 90 | 90 | 90 | 80 | 100 | 100 | 90 | 90 |
| Galium | 100 | 100 | 100 | 100 | 100 | 90 | 100 | 100 | 100 | 70 | 100 | 100 | 90 | 100 |
| Goosegrass | 60 | 90 | 100 | 100 | 100 | 80 | 80 | 100 | 80 | 50 | 90 | 100 | 90 | — |
| Horseweed | 80 | 90 | 60 | 80 | 70 | 30 | 0 | 10 | 0 | 0 | 80 | 30 | 20 | 30 |
| Kochia | 100 | 100 | 90 | 90 | 90 | 80 | 90 | 100 | 100 | 90 | 90 | 90 | 90 | 90 |
| Oat, Wild | 80 | 70 | 90 | 90 | 70 | 90 | 60 | 80 | 40 | 70 | 60 | 70 | 70 | 80 |
| Pigweed, Palmer | 90 | 100 | 100 | 100 | 100 | 60 | 100 | 100 | 100 | 60 | 90 | 100 | 70 | 60 |
| Pigweed, Redroot | — | — | — | — | — | — | — | — | — | — | — | — | — | — |
| Ragweed | 70 | 80 | 60 | 90 | 70 | 50 | 40 | 40 | 30 | 0 | 70 | 70 | 60 | 50 |
| Ryegrass, Italian | 100 | 80 | 100 | 100 | 80 | 60 | 50 | 60 | 60 | 20 | 80 | 60 | 40 | 100 |
| Soybean | 100 | 100 | 100 | 100 | 100 | 100 | 100 | 100 | 100 | 100 | 100 | 100 | 100 | 100 |
| Wheat | 40 | 70 | 90 | 90 | 40 | 70 | 30 | 30 | 10 | 10 | 40 | 30 | 30 | 90 |
| 500 g ai/ha | Compounds | | | | | | | | | | | | | |
| Postemergence | 110 | 111 | 112 | 113 | 114 | 117 | 118 | 122 | 126 | 127 | 135 | 136 | 137 | 138 |
| Barnyardgrass | 50 | 80 | 40 | 90 | 70 | — | — | — | — | — | 10 | 70 | 10 | 80 |
| Blackgrass | 50 | 50 | 60 | 90 | 90 | 90 | 70 | 50 | 40 | 80 | 70 | 100 | 70 | 90 |
| Corn | 50 | 80 | 40 | 90 | 80 | 50 | 70 | 60 | 10 | 50 | 60 | 90 | 10 | 90 |
| Foxtail, Giant | 90 | 90 | 70 | 90 | 90 | 70 | 50 | 50 | 70 | 90 | 90 | — | — | 90 |
| Galium | 100 | 100 | 100 | 100 | 70 | 0 | 30 | 50 | 70 | 100 | 80 | 100 | 90 | 100 |
| Goosegrass | — | — | — | — | — | 70 | 40 | 60 | 0 | 70 | — | — | — | — |
| Horseweed | 0 | 50 | 0 | 30 | 0 | 10 | 0 | 0 | 40 | 0 | 0 | 0 | 30 | 0 |
| Kochia | 90 | 100 | 90 | 90 | 80 | 70 | 80 | 50 | 20 | 90 | 50 | 70 | 70 | 80 |
| Oat, Wild | 30 | 50 | 30 | 80 | 40 | 40 | 90 | 20 | 50 | 10 | 20 | 30 | 0 | 80 |
| Pigweed, Palmer | 50 | 90 | 90 | 80 | 100 | 30 | 20 | 20 | 30 | 90 | 20 | 90 | 60 | 90 |
| Pigweed, Redroot | — | — | — | — | — | — | — | — | — | — | — | — | — | — |
| Ragweed | 60 | 70 | 0 | 50 | 0 | 0 | 30 | 0 | 50 | 10 | 30 | 30 | 30 | 40 |
| Ryegrass, Italian | 80 | 60 | 60 | 100 | 70 | 60 | 60 | 30 | 50 | 50 | 50 | 90 | 30 | 100 |
| Soybean | 100 | 100 | 100 | 100 | 100 | 100 | 80 | 90 | 60 | 100 | 100 | 100 | 90 | 100 |
| Wheat | 20 | 40 | 20 | 80 | 40 | 40 | 30 | 10 | 30 | 20 | 50 | 30 | 10 | 80 |
| 500 g ai/ha | Compounds | | | | | | | | | | | | | |
| Postemergence | 139 | 140 | 141 | 142 | 143 | 144 | 145 | 146 | | 147 | | 148 | | 149 |
| Barnyardgrass | 70 | 70 | 70 | 10 | 90 | 90 | 20 | 20 | | 70 | | 80 | | 100 |
| Blackgrass | 90 | 80 | 80 | 60 | 90 | 90 | 70 | 20 | | 100 | | 90 | | 90 |
| Corn | 60 | 90 | 90 | 10 | 90 | 70 | 20 | 30 | | 90 | | 90 | | 100 |
| Foxtail, Giant | 100 | 90 | 100 | 80 | 90 | 90 | 50 | 50 | | — | | — | | — |
| Galium | 90 | 70 | 100 | 30 | 100 | 100 | 70 | 80 | | 100 | | 100 | | 100 |
| Goosegrass | — | — | — | — | — | — | — | — | | — | | — | | — |
| Horseweed | 0 | 0 | 0 | 0 | 50 | 0 | 0 | 0 | | 0 | | 50 | | 70 |
| Kochia | 80 | 90 | 90 | 50 | 90 | 60 | 80 | 90 | | 90 | | 80 | | 90 |
| Oat, Wild | 60 | 50 | 20 | 0 | 70 | 40 | 30 | 10 | | 80 | | 70 | | 90 |
| Pigweed, Palmer | 80 | 90 | 100 | 20 | 80 | 40 | 90 | 70 | | 90 | | 100 | | 90 |
| Pigweed, Redroot | — | — | — | — | — | — | — | — | | — | | — | | — |
| Ragweed | 30 | 40 | 60 | 20 | 70 | 0 | 10 | 0 | | 30 | | 70 | | 90 |
| Ryegrass, Italian | 100 | 50 | 90 | 20 | 80 | 90 | 40 | 40 | | 80 | | 80 | | 100 |
| Soybean | 100 | 100 | 100 | 40 | 100 | 100 | 100 | 100 | | 100 | | 100 | | 100 |
| Wheat | 50 | 60 | 20 | 10 | 40 | 60 | 30 | 30 | | 60 | | 70 | | 90 |
| 125 g ai/ha | Compounds | | | | | | | | | | | | | |
| Postemergence | 6 | 7 | 28 | 37 | 38 | 39 | 40 | 42 | 43 | 44 | 45 | 46 | 47 | 48 |
| Barnyardgrass | 0 | 10 | — | — | — | — | — | — | — | — | — | — | — | — |
| Blackgrass | 0 | 0 | 30 | 30 | 0 | 60 | 0 | 0 | 0 | 30 | 30 | 30 | 10 | 0 |
| Corn | 0 | 0 | 0 | 10 | 20 | 0 | 0 | 10 | 10 | 30 | 10 | 10 | 30 | 10 |
| Foxtail, Giant | 0 | 10 | 0 | 50 | 60 | 10 | 0 | 20 | 10 | 40 | 20 | 20 | 30 | 20 |
| Galium | 0 | 0 | 50 | 40 | 30 | 90 | 30 | 50 | 30 | 50 | 80 | 60 | 60 | 40 |
| Goosegrass | — | — | 0 | 40 | 30 | 30 | 0 | 20 | 0 | 30 | 0 | 10 | 0 | 0 |
| Horseweed | — | — | 0 | — | — | 0 | 0 | — | — | — | — | — | — | — |
| Kochia | 0 | 0 | 0 | 50 | 20 | 30 | 0 | 0 | 10 | 10 | 10 | 20 | 50 | 20 |
| Oat, Wild | — | — | 20 | 0 | 0 | 40 | 0 | 0 | 0 | 0 | 0 | 0 | 10 | 10 |
| Pigweed, Palmer | — | — | 0 | 30 | 30 | 20 | 0 | 30 | 50 | 30 | 30 | 20 | 70 | 30 |
| Pigweed, Redroot | 0 | 0 | — | — | — | — | — | — | — | — | — | — | — | — |
| Ragweed | 0 | 0 | 0 | 0 | 0 | 0 | 0 | 0 | 0 | 0 | 0 | 0 | 0 | 0 |
| Ryegrass, Italian | 50 | 0 | 0 | 30 | 10 | 50 | 0 | 0 | 30 | 50 | 50 | 40 | 30 | 20 |
| Soybean | — | — | 90 | 100 | — | 100 | 0 | 70 | 80 | 100 | 100 | 90 | 80 | 70 |
| Wheat | 0 | 30 | 10 | 40 | 10 | 10 | 0 | 0 | 10 | 30 | 10 | 10 | 20 | 10 |
| 125 g ai/ha | Compounds | | | | | | | | | | | | | |
| Postemergence | 50 | 51 | 52 | 53 | 54 | 55 | 56 | 57 | 59 | 60 | 61 | 62 | 63 | 64 |
| Barnyardgrass | — | — | — | — | — | — | — | — | — | — | — | — | — | — |
| Blackgrass | 50 | 40 | 50 | 0 | 0 | 60 | 0 | 50 | 20 | 10 | 20 | 50 | 70 | 40 |
| Corn | 30 | 30 | 40 | 0 | 30 | 40 | 0 | 70 | —- | 30 | 10 | 10 | 80 | 10 |
| Foxtail, Giant | 70 | 90 | 60 | 0 | 70 | 100 | 0 | 90 | 40 | 90 | 60 | 90 | 90 | 20 |
| Galium | 60 | 80 | 30 | 0 | 100 | 100 | 0 | 80 | 90 | 90 | 70 | — | 100 | 90 |
| Goosegrass | 50 | 40 | 50 | 20 | 40 | 50 | 0 | 40 | 20 | 10 | 10 | 20 | 70 | 10 |
| Horseweed | 0 | 0 | 0 | 0 | 0 | 0 | 0 | 0 | 0 | 0 | — | 0 | 20 | 0 |
| Kochia | 10 | 60 | 10 | 0 | 80 | 80 | 0 | 10 | 10 | 20 | 20 | 90 | 10 | 60 |
| Oat, Wild | 20 | 30 | 20 | 0 | 20 | 40 | 0 | 80 | 10 | 50 | 10 | 20 | 70 | 50 |
| Pigweed, Palmer | 30 | 20 | 30 | 0 | 50 | 40 | 0 | 20 | 20 | 20 | 40 | 50 | 30 | 30 |
| Pigweed, Redroot | — | — | — | — | — | — | — | — | — | — | — | — | — | — |
| Ragweed | 20 | 0 | 0 | 0 | 0 | 50 | 0 | 30 | 40 | 20 | 30 | 20 | 40 | 0 |
| Ryegrass, Italian | 50 | 70 | 40 | 0 | 30 | 50 | 0 | 70 | 0 | 0 | 40 | 40 | 80 | 40 |
| Soybean | 100 | 100 | 100 | 20 | 100 | 100 | 0 | 100 | — | 100 | 100 | 100 | 100 | 80 |
| Wheat | 40 | 30 | 30 | 0 | 20 | 40 | 0 | 70 | 10 | 30 | 10 | 10 | 70 | 10 |
| 125 g ai/ha | Compounds | | | | | | | | | | | | | |
| Postemergence | 65 | 66 | 67 | 69 | 70 | 71 | 72 | 73 | 74 | 75 | 76 | 77 | 78 | 79 |
| Barnyardgrass | — | — | — | — | — | — | — | — | — | — | — | — | — | — |
| Blackgrass | 30 | 60 | 70 | 0 | 30 | 80 | 0 | 90 | 70 | 90 | 70 | 50 | 50 | 80 |
| Corn | 10 | 0 | 60 | 10 | 10 | 60 | 30 | 70 | 60 | 60 | 60 | 40 | 10 | 40 |
| Foxtail, Giant | 10 | 20 | 50 | 20 | 10 | 90 | 10 | 80 | 90 | 90 | 90 | 70 | 50 | 90 |
| Galium | 100 | 80 | 100 | 60 | 50 | 100 | 70 | 100 | 100 | 100 | 100 | 100 | 100 | — |
| Goosegrass | 10 | 10 | 30 | 0 | 10 | 10 | 50 | 20 | 0 | 70 | 50 | 20 | 40 | 20 |
| Horseweed | 0 | 0 | 30 | 0 | 0 | 30 | 20 | 30 | 0 | 50 | 70 | 50 | 0 | 0 |
| Kochia | 60 | 30 | 70 | 20 | 10 | 60 | 70 | 90 | 70 | 90 | 90 | 90 | 70 | 30 |
| Oat, Wild | 10 | 20 | 10 | 10 | 10 | 30 | 10 | 80 | 60 | 90 | 30 | 0 | 80 | 70 |
| Pigweed, Palmer | 60 | 20 | 70 | 20 | 40 | 60 | 40 | 90 | 30 | 70 | 90 | 60 | 50 | 30 |
| Pigweed, Redroot | — | — | — | — | — | — | — | — | — | — | — | — | — | — |
| Ragweed | 50 | 0 | 40 | 0 | 0 | 50 | 30 | 50 | 40 | 70 | 60 | — | 40 | 0 |
| Ryegrass, Italian | 30 | 50 | 60 | 0 | 0 | 60 | 0 | 70 | 50 | 50 | 50 | 30 | 50 | 90 |
| Soybean | 60 | 50 | 100 | 60 | 50 | 100 | 90 | 100 | 100 | 100 | 100 | 100 | 100 | 100 |
| Wheat | 10 | 10 | 10 | 10 | 10 | 40 | 10 | 50 | 40 | 30 | 20 | 10 | 20 | 40 |
| 125 g ai/ha | Compounds | | | | | | | | | | | | | |
| Postemergence | 80 | 81 | 82 | 83 | 84 | 85 | 86 | 87 | 88 | 89 | 90 | 91 | 92 | 93 |
| Barnyardgrass | — | — | — | — | — | — | — | — | — | — | — | — | — | — |
| Blackgrass | 0 | 0 | 0 | 10 | 70 | 30 | 0 | 10 | 60 | 70 | 0 | 90 | 70 | 70 |
| Corn | 10 | 0 | 0 | 30 | 70 | 10 | 0 | 10 | 90 | 70 | 0 | 60 | 30 | 60 |
| Foxtail, Giant | 10 | 20 | 10 | 20 | 80 | 20 | 10 | 10 | 90 | 90 | 0 | 80 | 30 | 60 |
| Galium | — | — | — | — | — | — | — | — | — | — | 0 | 100 | 100 | 100 |
| Goosegrass | 10 | 10 | 0 | 70 | 100 | 0 | 10 | 30 | 100 | 90 | 0 | 80 | 30 | 20 |
| Horseweed | 0 | 0 | 0 | 40 | 40 | 20 | 0 | 0 | 40 | 20 | 0 | 0 | 0 | 0 |
| Kochia | 40 | 50 | 70 | 100 | 90 | 60 | 50 | 70 | 90 | 70 | 0 | 80 | 80 | 80 |
| Oat, Wild | 0 | 10 | 0 | 20 | 90 | 20 | 0 | 20 | 90 | 80 | 0 | 50 | 50 | 20 |
| Pigweed, Palmer | 30 | 60 | 0 | 100 | 90 | 70 | 20 | 30 | 90 | 70 | 0 | 70 | 90 | 90 |
| Pigweed, Redroot | — | — | — | — | — | — | — | — | — | — | — | — | — | — |
| Ragweed | 40 | 20 | 0 | 70 | 100 | 0 | 50 | 50 | 70 | 60 | 0 | 30 | 60 | 50 |
| Ryegrass, Italian | 20 | 30 | 0 | 40 | 100 | 10 | 10 | 10 | 80 | 100 | 0 | 80 | 50 | 30 |
| Soybean | 70 | 100 | 90 | 100 | 100 | 100 | 50 | 40 | 100 | 100 | 20 | 100 | 90 | 100 |
| Wheat | 10 | 10 | 10 | 10 | 40 | 0 | 10 | 10 | 40 | 50 | 10 | 30 | 40 | 20 |
| 125 g ai/ha | Compounds | | | | | | | | | | | | | |
| Postemergence | 94 | 95 | 96 | 97 | 98 | 99 | 100 | 101 | 102 | 103 | 104 | 105 | 106 | 107 |
| Barnyardgrass | — | — | — | — | — | — | — | — | — | — | — | — | — | — |
| Blackgrass | 60 | 80 | 50 | 30 | 70 | 90 | 30 | 70 | 0 | 60 | 0 | 30 | 60 | 60 |
| Corn | 80 | 60 | 10 | 30 | 50 | 70 | 70 | 70 | 20 | 10 | 40 | 20 | 60 | 70 |
| Foxtail, Giant | 90 | 90 | 90 | 90 | 90 | 90 | 50 | 90 | 80 | 90 | 90 | 20 | 90 | 90 |
| Galium | 100 | 100 | 100 | 80 | 100 | 100 | 80 | 50 | 50 | 100 | 60 | 60 | 70 | 100 |
| Goosegrass | 60 | 80 | 30 | 70 | 80 | 90 | 70 | 50 | 80 | 70 | 50 | 0 | 70 | 70 |
| Horseweed | 0 | 0 | 40 | 30 | 30 | 70 | 40 | 0 | 0 | 0 | 0 | 0 | 10 | 20 |
| Kochia | 100 | 80 | 90 | 70 | 80 | 80 | 80 | 70 | 90 | 80 | 80 | 90 | 90 | 80 |
| Oat, Wild | 80 | 70 | 30 | 30 | 80 | 70 | 30 | 40 | 10 | 30 | 20 | 70 | 30 | 30 |
| Pigweed, Palmer | 90 | 50 | 90 | 90 | 90 | 90 | 70 | 50 | 90 | 90 | 80 | 40 | 70 | 90 |
| Pigweed, Redroot | — | — | — | — | — | — | — | — | — | — | — | — | — | — |
| Ragweed | 50 | 0 | 60 | 50 | 60 | 40 | 60 | 50 | 20 | 0 | 0 | 0 | 50 | 50 |
| Ryegrass, Italian | 50 | 60 | 50 | 40 | 70 | 50 | 50 | 60 | 50 | 50 | 30 | 0 | 20 | 20 |
| Soybean | 100 | 100 | 100 | 100 | 100 | 100 | 100 | 70 | 100 | 100 | 100 | 70 | 100 | 100 |
| Wheat | 30 | 40 | 30 | 20 | 80 | 80 | 30 | 20 | 10 | 10 | 10 | 0 | 20 | 30 |
| 125 g ai/ha | Compounds | | | | | | | | | | | | | |
| Postemergence | 108 | 109 | 110 | 111 | 112 | 113 | 114 | 117 | 118 | 122 | 126 | 127 | 135 | 136 |
| Barnyardgrass | — | 50 | 20 | 30 | 10 | 60 | 10 | — | — | — | — | — | 0 | 20 |
| Blackgrass | 30 | 70 | 0 | 40 | 0 | 80 | 50 | 50 | 0 | 0 | 0 | 0 | 0 | 90 |
| Corn | 40 | 60 | 10 | 40 | 20 | 90 | 50 | 0 | 0 | 10 | 0 | 20 | 10 | 30 |
| Foxtail, Giant | 70 | 70 | 40 | 90 | 20 | 60 | 70 | 20 | 10 | 20 | 0 | 50 | 10 | — |
| Galium | 80 | 70 | 100 | 100 | 90 | 90 | 60 | 0 | 10 | 50 | 50 | 60 | 50 | 80 |
| Goosegrass | 80 | — | — | — | — | — | — | 30 | 20 | 0 | 0 | 10 | — | — |
| Horseweed | 10 | 10 | 0 | 10 | 0 | 10 | 0 | 0 | 0 | 0 | 0 | 0 | 0 | 0 |
| Kochia | 70 | 70 | 70 | 80 | 70 | 40 | 40 | 30 | 30 | 30 | 10 | 60 | 0 | 30 |
| Oat, Wild | 20 | 80 | 0 | 40 | 10 | 70 | 0 | 10 | 10 | 0 | 0 | 0 | 0 | 10 |
| Pigweed, Palmer | 70 | 60 | 40 | 70 | 50 | 30 | 40 | 10 | 0 | 20 | 0 | 60 | 10 | 30 |
| Pigweed, Redroot | — | — | — | — | — | — | — | — | — | — | — | — | — | — |
| Ragweed | 60 | 0 | 10 | 60 | 0 | 50 | 0 | 0 | 0 | 0 | 30 | 0 | 0 | 20 |
| Ryegrass, Italian | 20 | 50 | 20 | 30 | 10 | 50 | 50 | 30 | 10 | 0 | 0 | 10 | 0 | 60 |
| Soybean | 100 | 100 | 100 100 | | 90 | 100 | 60 | 60 | 40 | 50 | 40 | 100 | 90 | 100 |
| Wheat | 10 | 40 | 10 | 10 | 10 | 50 | 10 | 10 | 10 | 10 | 0 | 10 | 10 | 20 |

| 125 g ai/ha | Compounds | | | | | | | | | | | | |
|---|---|---|---|---|---|---|---|---|---|---|---|---|---|
| Postemergence | 137 | 138 | 139 | 140 | 141 | 142 | 143 | 144 | 145 | 146 | 147 | 148 | 149 |
| Barnyardgrass | 10 | 60 | 20 | 50 | 30 | 10 | 50 | 20 | 20 | 20 | 10 | 30 | 50 |
| Blackgrass | 20 | 80 | 80 | 70 | 30 | 0 | 80 | 70 | 40 | 10 | 70 | 40 | 90 |
| Corn | 10 | 80 | 40 | 70 | 90 | 10 | 90 | 40 | 10 | 10 | 10 | 80 | 90 |
| Foxtail, Giant | — | 90 | 80 | 90 | 60 | 30 | 90 | 80 | 20 | 30 | — | — | — |
| Galium | 80 | 80 | 80 | 70 | 80 | — | 100 | 70 | 60 | 50 | 100 | 90 | 100 |
| Goosegrass | — | — | — | — | — | — | — | — | — | — | — | — | — |
| Horseweed | 0 | 0 | 0 | 0 | 0 | 0 | 20 | 0 | 0 | 0 | 0 | 50 | 50 |
| Kochia | 30 | 80 | 50 | 90 | 80 | 10 | 90 | 10 | 70 | 80 | 60 | 50 | 80 |
| Oat, Wild | 0 | 50 | 0 | 0 | 10 | 0 | 10 | 0 | 10 | 0 | 30 | 20 | 90 |
| Pigweed, Palmer | 30 | 30 | 40 | 70 | 70 | — | 60 | 20 | 70 | 70 | 80 | 90 | 80 |
| Pigweed, Redroot | — | — | — | — | — | — | — | — | — | — | — | — | — |
| Ragweed | 30 | 0 | 0 | 20 | 30 | 0 | 40 | 0 | 0 | 0 | 0 | 50 | 80 |
| Ryegrass, Italian | 20 | 70 | 60 | 40 | 30 | 0 | 50 | 50 | 30 | 20 | 50 | 50 | 80 |
| Soybean | 80 | 100 | 90 | 100 | 100 | 40 | 100 | 100 | 40 | 50 | 100 | 100 | 100 |
| Wheat | 10 | 40 | 20 | 30 | 10 | 10 | 30 | 20 | 30 | 20 | 10 | 30 | 90 |

| 31 g ai/ha | Compound |
|---|---|
| Postemergence | 62 |
| Blackgrass | 40 |
| Corn | 10 |
| Foxtail, Giant | 20 |
| Goosegrass | 10 |
| Horseweed | 0 |
| Kochia | 80 |
| Oat, Wild | 0 |
| Pigweed, Palmer | 40 |
| Ragweed | 10 |
| Ryegrass, Italian | 0 |
| Soybean | 100 |
| Wheat | 10 |

### Test B

Plant species in the flooded paddy test selected from barnyardgrass (Echinochloa crus-galli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 days, after which time all species were compared to controls and visually evaluated. Plant response ratings, summarized in Table B, are based on a scale of 0 to 100 where 0 is no effect and 100 is complete control. A dash (-) response means no test result.

TABLE B

| | Compounds | | | | | | | | | | | | | |
|---|---|---|---|---|---|---|---|---|---|---|---|---|---|---|
| 250 g ai/ha | 1 | 5 | 6 | 7 | 10 | 18 | 19 | 20 | 21 | 22 | 25 | 28 | 29 | 30 |
| Flood | | | | | | | | | | | | | | |
| Barnyardgrass | 0 | 25 | 0 | 0 | 0 | 0 | 0 | 0 | 0 | 0 | 0 | 10 | 0 | 0 |
| Ducksalad | 0 | 35 | 0 | 0 | 0 | 0 | 0 | 85 | 65 | 0 | 35 | 80 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 0 | 0 | 0 | 0 | 0 | 0 | 35 | 35 | 0 | 0 | 0 | 0 | 0 |
| | Compounds | | | | | | | | | | | | | |
| 250 g ai/ha | 32 | 33 | 34 | 35 | 37 | 38 | 39 | 40 | 41 | 42 | 43 | 44 | 45 | 46 |
| Flood | | | | | | | | | | | | | | |
| Barnyardgrass | 0 | 30 | 0 | 0 | 0 | 0 | 0 | 0 | 0 | 0 | 0 | 0 | 0 | 0 |
| Ducksalad | 0 | 30 | 0 | 30 | 0 | 0 | 0 | 0 | 50 | 0 | 0 | 0 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 65 | 0 | 40 | 0 | 0 | 0 | 0 | 55 | 0 | 0 | 0 | 0 | 0 |
| | Compounds | | | | | | | | | | | | | |
| 250 g ai/ha | 47 | 48 | 50 | 51 | 52 | 53 | 54 | 55 | 56 | 57 | 58 | 59 | 60 | 61 |
| Flood | | | | | | | | | | | | | | |
| Barnyardgrass | 0 | 0 | 0 | 25 | 25 | 0 | 25 | 30 | 0 | 15 | 0 | 0 | 15 | 0 |
| Ducksalad | 0 | 0 | 0 | 65 | 50 | 0 | 30 | 75 | 0 | 65 | 0 | 0 | 45 | 15 |
| Rice | 0 | 0 | 0 | 15 | 10 | 0 | 30 | 0 | 0 | 0 | 0 | 0 | 0 | 0 |
| Sedge, Umbrella | 0 | 0 | 0 | 80 | 75 | 0 | 90 | 95 | 0 | 90 | 0 | 0 | 35 | 45 |
| | Compounds | | | | | | | | | | | | | |
| 250 g ai/ha | 62 | 63 | 64 | 65 | 66 | 67 | 68 | 69 | 70 | 71 | 72 | 73 | 74 | 75 |
| Flood | | | | | | | | | | | | | | |
| Barnyardgrass | 0 | 0 | 0 | 0 | 0 | 0 | 0 | 0 | 0 | 0 | 15 | 45 | 10 | 50 |
| Ducksalad | 0 | 90 | 25 | 45 | 0 | 30 | 55 | 0 | 0 | 65 | 55 | 70 | 60 | 70 |
| Rice | 0 | 00 | 0 | 0 | 0 | 0 | 0 | 0 | 0 | 0 | 0 | 20 | 20 | 10 |
| Sedge, Umbrella | 0 | 98 | 75 | 95 | 0 | 75 | 65 | 0 | 0 | 95 | 15 | 70 | 95 | 98 |
| | Compounds | | | | | | | | | | | | | |
| 250 g ai/ha | 76 | 77 | 78 | 79 | 80 | 81 | 82 | 83 | 84 | 85 | 86 | 88 | 89 | 90 |
| Flood | | | | | | | | | | | | | | |
| Barnyardgrass | 25 | 0 | 0 | 0 | 0 | 0 | 0 | 40 | 65 | 0 | 0 | 45 | 60 | 0 |
| Ducksalad | 20 | 20 | 75 | 45 | 0 | 0 | 0 | 30 | 60 | 0 | 65 | 55 | 100 | 0 |
| Rice | 0 | 0 | 0 | 0 | 0 | 0 | 0 | 0 | 0 | 0 | 0 | 10 | 15 | 0 |
| Sedge, Umbrella | 98 | 85 | 0 | 98 | 0 | 0 | 0 | 100 | 100 | 0 | 90 | 100 | 100 | 0 |
| | Compounds | | | | | | | | | | | | | |
| 250 g ai/ha | 91 | 92 | 93 | 94 | 95 | 96 | 97 | 98 | 99 | 100 | 101 | 102 | 103 | 104 |
| Flood | | | | | | | | | | | | | | |
| Barnyardgrass | 25 | 25 | 0 | 30 | 65 | 60 | 0 | 45 | 35 | 0 | 15 | 10 | 20 | 60 |
| Ducksalad | 90 | 90 | 0 | 70 | 95 | 65 | 0 | 65 | 40 | 40 | 55 | 65 | 35 | 25 |
| Rice | 0 | 0 | 0 | 0 | 0 | 0 | 0 | 30 | 10 | 0 | 0 | 10 | 10 | 20 |
| Sedge, Umbrella | 35 | 0 | 0 | 98 | 98 | 98 | 0 | 90 | 95 | 55 | 85 | 95 | 100 | 98 |
| | Compounds | | | | | | | | | | | | | |
| 250 g ai/ha | 105 | 106 | 107 | 108 | 109 | 110 | 111 | 112 | 113 | 114 | 115 | 116 | 117 | 118 |
| Flood | | | | | | | | | | | | | | |
| Barnyardgrass | 0 | 55 | 70 | 55 | 40 | 55 | 55 | 0 | 45 | 0 | 0 | 0 | 0 | 0 |
| Ducksalad | 0 | 10 | 25 | 20 | 75 | 0 | 65 | 0 | 65 | 0 | 0 | 0 | 0 | 0 |
| Rice | 0 | 15 | 20 | 10 | 0 | 0 | 0 | 0 | 0 | 0 | 0 | 0 | 0 | 0 |
| Sedge, Umbrella | 0 | 90 | 90 | 80 | 98 | 65 | 98 | 0 | 80 | 0 | 0 | 0 | 0 | 0 |
| | Compounds | | | | | | | | | | | | | |
| 250 g ai/ha | 119 | 122 | 126 | 127 | 136 | 137 | 138 | 139 | 140 | 141 | 142 | 143 | 144 | 145 |
| Flood | | | | | | | | | | | | | | |
| Barnyardgrass | 0 | 0 | 0 | 0 | 0 | 0 | 35 | 15 | 20 | 25 | 0 | 40 | 0 | 45 |
| Ducksalad | 0 | 0 | 0 | 0 | 0 | 0 | 70 | 75 | 40 | 30 | 0 | 65 | 65 | 40 |
| Rice | 0 | 0 | 0 | 0 | 0 | 0 | 35 | 0 | 0 | 10 | 0 | 0 | 0 | 0 |
| Sedge, Umbrella | 0 | 0 | 0 | 0 | 0 | 0 | 75 | 75 | 75 | 95 | 0 | 98 | 60 | 0 |

| | Compound | | | |
|---|---|---|---|---|
| 250 g ai/ha | 146 | 147 | 148 | 149 |
| Flood | | | | |
| Barnyardgrass | 0 | 0 | 25 | 35 |
| Ducksalad | 0 | 0 | 55 | 75 |
| Rice | 0 | 0 | 15 | 20 |
| Sedge, Umbrella | 0 | 95 | 65 | 100 |

### Test C

Seeds of plant species selected from barnyardgrass (Echinochloa crusgalli), blackgrass (Alopecurus myosuroides), corn (Zea mays), foxtail, giant (giant foxtail, Setaria faberi), green foxtail (Setaria viridis), goosegrass (Eleusine indica), kochia (Bassia scoparia), oat, wild (wild oat, Avena fatua), pigweed, palmer (palmer amaranth, palmer pigweed, Amaranthus palmeri), Pigweed, Redroot (redroot pigweed, Amaranthus retroflexus), ragweed (common ragweed, Ambrosia artemisiifolia), ryegrass, Italian (179allium ryegrass, Lolium multiflorum), soybean (Glycine max), and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crops and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 days, after which time all treated plants were compared to untreated controls and visually evaluated for injury. Plant response ratings, summarized in Table C, are based on a 0 to 100 scale where 0 is no effect and 100 is complete control. A dash (-) response means no test result.

TABLE C

| | Compounds | | | | | | | | | | | | | |
|---|---|---|---|---|---|---|---|---|---|---|---|---|---|---|
| 1000 g ai/ha | 159 | 160 | 161 | 162 | 163 | 166 | 181 | 197 | 198 | 199 | 200 | 201 | 202 | 204 |
| Preemergence | | | | | | | | | | | | | | |
| Barnyardgrass | 0 | 30 | 50 | 60 | 0 | 0 | 0 | 0 | 10 | 0 | 0 | 0 | 0 | 0 |
| Blackgrass | 0 | 0 | 0 | 30 | 10 | 0 | 0 | — | — | — | — | — | — | 0 |
| Corn | — | — | — | — | — | — | — | — | — | — | — | — | — | — |
| Foxtail, Giant | — | — | — | — | — | — | — | 30 | 0 | 50 | 0 | 10 | 0 | — |
| Foxtail, Green | 20 | 30 | 70 | 90 | 10 | 0 | 0 | — | — | — | — | — | — | 0 |
| Kochia | 0 | 70 | 30 | 0 | 0 | 0 | 0 | 0 | 0 | 0 | 0 | 0 | 0 | 20 |
| Pigweed, Palmer | 20 | 20 | 0 | 30 | 0 | 0 | 0 | — | — | — | — | — | — | 10 |
| Pigweed, Redroot | — | — | — | — | — | — | — | 30 | 0 | 20 | 0 | — | 0 | — |
| Ragweed | 0 | 0 | 80 | 0 | 0 | 0 | 0 | 0 | 0 | 0 | 0 | 0 | 0 | 0 |
| Ryegrass, Italian | 20 | 40 | 80 | 70 | 20 | 0 | 50 | 0 | 0 | 30 | 0 | 0 | 0 | 40 |
| | Compounds | | | | | | | | | | | | | |
| 1000 g ai/ha | 205 | 206 | 207 | 208 | 209 | 222 | 238 | 239 | 243 | 244 | 245 | 246 | 247 | 250 |
| Preemergence | | | | | | | | | | | | | | |
| Barnyardgrass | 0 | 30 | 90 | 50 | 0 | 50 | 10 | 50 | 60 | 50 | 20 | 0 | 0 | 90 |
| Blackgrass | 0 | 0 | 20 | 0 | 0 | 70 | 0 | 30 | 0 | 0 | 0 | 0 | 0 | 30 |
| Corn | — | — | — | — | — | — | 0 | 0 | — | — | — | — | — | — |
| Foxtail, Giant | — | — | — | — | — | — | — | — | — | — | — | — | — | — |
| Foxtail, Green | 40 | 20 | 90 | 100 | 0 | 100 | 90 | 100 | 70 | 50 | 30 | 0 | 0 | 100 |
| Kochia | 50 | 10 | 30 | 10 | 0 | 20 | 0 | 30 | 0 | 0 | 0 | 0 | 0 | 80 |
| Pigweed, Palmer | 60 | 30 | 90 | 70 | 0 | 50 | 30 | 80 | 0 | 0 | 20 | 0 | — | 80 |
| Pigweed, Redroot | — | — | — | — | — | — | — | — | — | — | — | — | — | — |
| Ragweed | 0 | 0 | 0 | 0 | 0 | 0 | 10 | 0 | 30 | 50 | — | — | 0 | 0 |
| Ryegrass, Italian | 40 | 80 | 90 | 70 | 30 | 70 | 70 | 70 | 70 | 90 | 60 | 30 | 30 | 90 |
| | Compounds | | | | | | | | | | | | | |
| 1000 g ai/ha | 251 | 286 | 287 | 293 | 296 | 297 | 298 | 304 | 309 | 383 | 384 | 385 | 389 | 390 |
| Preemergence | | | | | | | | | | | | | | |
| Barnyardgrass | 70 | 0 | 60 | 0 | 100 | 0 | 0 | 60 | 0 | 0 | 0 | 30 | 70 | 80 |
| Blackgrass | 10 | — | — | 0 | 50 | 0 | 0 | 70 | 20 | 0 | 0 | 0 | 30 | 20 |
| Corn | — | — | — | — | — | — | — | — | — | — | — | — | — | — |
| Foxtail, Giant | — | 0 | 0 | — | — | — | — | — | — | — | — | — | — | — |
| Foxtail, Green | 90 | — | — | 0 | 100 | 0 | 30 | 100 | 90 | 20 | 30 | 90 | 100 | 100 |
| Kochia | 0 | 0 | 30 | 0 | 90 | 0 | 50 | 10 | 10 | 0 | 0 | 0 | 90 | 50 |
| Pigweed, Palmer | 40 | — | — | 10 | 100 | 0 | 40 | 70 | 70 | 10 | 30 | 50 | 70 | 90 |
| Pigweed, Redroot | — | 0 | 0 | — | — | — | — | — | — | — | — | — | — | — |
| Ragweed | 0 | 0 | 0 | 0 | 70 | 0 | 0 | 0 | 0 | 0 | 0 | 0 | 20 | 30 |
| Ryegrass, Italian | 70 | 0 | 20 | 0 | 80 | 0 | 0 | 90 | 70 | 0 | 0 | 50 | 60 | 80 |
| | Compounds | | | | | | | | | | | | | |
| 500 g ai/ha | 150 | 151 | 152 | 153 | 154 | 155 | 156 | 157 | 158 | 164 | 165 | 167 | 168 | 169 |
| Preemergence | | | | | | | | | | | | | | |
| Barnyardgrass | 0 | 100 | 90 | 100 | 100 | 100 | 90 | 90 | 90 | 100 | 0 | 100 | 70 | 100 |
| Blackgrass | 0 | 90 | 60 | 60 | 60 | 90 | 90 | 30 | 60 | 80 | 0 | 70 | 60 | 90 |
| Corn | 0 | 50 | 50 | 30 | 30 | 50 | 10 | 0 | 20 | 30 | 0 | 0 | — | 40 |
| Foxtail, Giant | — | — | — | — | — | — | — | — | — | — | — | — | — | — |
| Foxtail, Green | 0 | 100 | 100 | 100 | 100 | 100 | 100 | 100 | 100 | 100 | 0 | 100 | 100 | 100 |
| Goosegrass | — | — | — | — | — | — | — | — | — | — | — | — | — | — |
| Kochia | 0 | 100 | 90 | 60 | 100 | 90 | 100 | 90 | 90 | 100 | 0 | 100 | 50 | 100 |
| Oat, Wild | 0 | 90 | 70 | 80 | 30 | 50 | 40 | 50 | 80 | — | 0 | 50 | 20 | 90 |
| Pigweed, Palmer | 0 | 100 | 90 | 100 | 100 | 100 | 100 | 100 | 100 | 100 | 0 | 100 | 100 | 100 |
| Ragweed | 20 | 80 | 0 | 100 | 100 | 100 | 90 | 80 | 90 | 60 | 20 | 80 | 60 | 100 |
| Ryegrass, Italian | 0 | 90 | 90 | 90 | 90 | 90 | 90 | 90 | 80 | 100 | 0 | 90 | 90 | 100 |
| Soybean | 0 | 90 | 70 | 100 | 100 | 100 | 100 | 60 | 80 | 100 | 0 | 80 | 60 | 90 |
| Wheat | 0 | 80 | 40 | 90 | 60 | 70 | 30 | 30 | 40 | 60 | 0 | 0 | 20 | 90 |
| | Compounds | | | | | | | | | | | | | |
| 500 g ai/ha | 170 | 171 | 172 | 173 | 174 | 175 | 177 | 178 | 179 | 191 | 192 | 194 | 195 | 196 |
| Preemergence | | | | | | | | | | | | | | |
| Barnyardgrass | 100 | 100 | 0 | 0 | 100 | 100 | 100 | 90 | 0 | 0 | 90 | 70 | 100 | — |
| Blackgrass | 80 | 70 | 0 | 0 | 80 | 10 | 60 | 90 | 0 | 20 | 80 | 80 | 40 | 0 |
| Corn | 20 | 40 | 0 | 0 | 50 | 20 | 30 | 50 | 0 | 0 | 20 | 30 | 0 | 20 |
| Foxtail, Giant | — | — | — | — | — | — | — | — | — | — | — | — | — | 0 |
| Foxtail, Green | 100 | 100 | 0 | 0 | 100 | 100 | 100 | 100 | 50 | 0 | 100 | 100 | 100 | — |
| Goosegrass | — | — | — | — | — | — | — | — | — | — | — | — | — | 0 |
| Kochia | 100 | 90 | 0 | 0 | 90 | 100 | 100 | 100 | 0 | 0 | 100 | 0 | 100 | 0 |
| Oat, Wild | 80 | 80 | 0 | 0 | 80 | 50 | 90 | 90 | 0 | 0 | 90 | 90 | 80 | 0 |
| Pigweed, Palmer | 100 | 100 | 0 | 0 | 100 | 100 | 100 | 100 | 10 | 0 | 100 | 30 | 100 | 0 |
| Ragweed | 70 | 90 | 0 | 60 | 10 | 70 | 90 | 50 | 0 | 0 | 80 | 20 | 100 | 0 |
| Ryegrass, Italian | 100 | 90 | 20 | 50 | 90 | 90 | 100 | — | — | 20 | 90 | 90 | 100 | 0 |
| Soybean | 100 | 100 | 0 | 0 | 100 | 90 | 90 | 80 | 0 | 0 | 90 | 10 | 100 | 0 |
| Wheat | 90 | 90 | 0 | 0 | 10 | 20 | 60 | 70 | 0 | 0 | 30 | 50 | 80 | 0 |
| | Compounds | | | | | | | | | | | | | |
| 500 g ai/ha | 203 | 210 | 211 | 212 | 213 | 214 | 215 | 216 | 217 | 218 | 219 | 220 | 221 | 223 |
| Preemergence | | | | | | | | | | | | | | |
| Barnyardgrass | 0 | 100 | 100 | 100 | 100 | 100 | 100 | 90 | 100 | 100 | 100 | 80 | 0 | 100 |
| Blackgrass | 0 | 100 | 90 | 90 | 80 | 70 | 90 | 90 | 90 | 80 | 40 | 70 | 0 | 90 |
| Corn | 0 | 60 | 70 | 70 | 10 | 20 | 60 | 30 | 40 | 60 | 20 | 10 | 0 | 40 |
| Foxtail, Giant | — | — | — | — | — | — | — | — | — | — | — | — | — | — |
| Foxtail, Green | 60 | 100 | 100 | 100 | 100 | 100 | 100 | 100 | 100 | 100 | 100 | 100 | 0 | 100 |
| Goosegrass | — | — | — | — | — | — | — | — | — | — | — | — | — | — |
| Kochia | 30 | 100 | 100 | 90 | 100 | 100 | 100 | 100 | 100 | 100 | 100 | 90 | 0 | 80 |
| Oat, Wild | 0 | 90 | 90 | 90 | 70 | 80 | 90 | 40 | 90 | 90 | 70 | 70 | 0 | 70 |
| Pigweed, Palmer | 70 | 100 | 100 | 100 | 100 | 100 | 100 | 100 | 100 | 100 | 100 | 100 | 0 | 100 |
| Ragweed | 0 | 80 | 100 | 50 | 30 | 0 | 30 | 40 | 100 | 80 | 70 | 0 | 0 | 40 |
| Ryegrass, Italian | 20 | 100 | 100 | 100 | 90 | 90 | 100 | 90 | 100 | 90 | 80 | 90 | 0 | 90 |
| Soybean | 0 | 100 | 100 | 100 | 100 | 80 | 100 | 100 | 100 | 100 | 90 | 80 | 0 | 70 |
| Wheat | 0 | 90 | 80 | 30 | 90 | 30 | 90 | 20 | 90 | 80 | 50 | 70 | 0 | 90 |
| | Compounds | | | | | | | | | | | | | |
| 500 g ai/ha | 224 | 225 | 226 | 227 | 228 | 229 | 231 | 232 | 233 | 234 | 235 | 236 | 240 | 241 |
| Preemergence | | | | | | | | | | | | | | |
| Barnyardgrass | 90 | 100 | 90 | 90 | 100 | 0 | 100 | 0 | 60 | 100 | 90 | 80 | 100 | 100 |
| Blackgrass | 70 | 80 | 60 | 90 | 90 | 0 | 90 | 0 | 30 | 80 | 80 | 10 | 90 | 70 |
| Corn | 20 | 90 | 0 | 60 | 60 | 0 | 30 | 0 | 20 | 10 | 0 | 0 | 60 | 70 |
| Foxtail, Giant | — | — | — | — | — | — | — | — | — | — | — | — | — | — |
| Foxtail, Green | 100 | 100 | 100 | 100 | 100 | 0 | 100 | 0 | 80 | 100 | 100 | 100 | 100 | 100 |
| Goosegrass | — | — | — | — | — | — | — | — | — | — | — | — | — | — |
| Kochia | 70 | 100 | 90 | 100 | 60 | 0 | 100 | 0 | 0 | 60 | 90 | 90 | 100 | 80 |
| Oat, Wild | 90 | 90 | 60 | 90 | 90 | 0 | 80 | 0 | 0 | 70 | 80 | 30 | 90 | 90 |
| Pigweed, Palmer | 100 | 100 | 90 | 100 | 100 | 0 | 100 | 0 | 0 | 90 | 100 | 100 | 100 | 100 |
| Ragweed | 40 | 90 | 20 | 50 | 50 | 0 | 90 | 0 | 70 | 50 | 60 | 80 | 20 | 10 |
| Ryegrass, Italian | 80 | 80 | 70 | 90 | 90 | 10 | 90 | 20 | 60 | 90 | 90 | 50 | 100 | 90 |
| Soybean | 100 | 90 | 30 | 90 | 90 | 0 | 70 | 0 | 10 | 100 | 70 | 70 | 100 | 100 |
| Wheat | 50 | 80 | 10 | 30 | 30 | 0 | 80 | 0 | 0 | 80 | 30 | 10 | 90 | 90 |
| | Compounds | | | | | | | | | | | | | |
| 500 g ai/ha | 242 | 248 | 249 | 252 | 253 | 254 | 256 | 258 | 259 | 260 | 262 | 263 | 264 | 265 |
| Preemergence | | | | | | | | | | | | | | |
| Barnyardgrass | 100 | 100 | 20 | 90 | 60 | 100 | 100 | 100 | 90 | 90 | 90 | 90 | 100 | 90 |
| Blackgrass | 90 | 70 | 60 | 20 | 50 | 80 | 90 | 60 | 70 | 80 | 90 | 90 | 80 | 90 |
| Corn | 70 | 50 | 0 | 0 | 10 | 50 | 90 | 20 | 30 | 10 | 40 | 60 | 50 | 90 |
| Foxtail, Giant | — | — | — | — | — | — | — | — | — | — | — | — | — | — |
| Foxtail, Green | 100 | 100 | 90 | 100 | 100 | 100 | 100 | 100 | 100 | 100 | 100 | 100 | 100 | 100 |
| Goosegrass | — | — | — | — | — | — | — | — | — | — | — | — | — | — |
| Kochia | 100 | 100 | 0 | 100 | 80 | 90 | 90 | 100 | 100 | 80 | 80 | 70 | 100 | 90 |
| Oat, Wild | 90 | 90 | 0 | 70 | 90 | 90 | 90 | 90 | 90 | 90 | 80 | 70 | 90 | 90 |
| Pigweed, Palmer | 100 | 100 | 30 | 100 | 100 | 100 | 100 | 100 | 100 | 100 | 90 | 100 | 100 | 100 |
| Ragweed | 100 | 90 | 0 | 30 | 80 | 90 | 90 | 50 | 0 | 40 | 60 | 30 | 80 | 70 |
| Ryegrass, Italian | 100 | 100 | 60 | 80 | 60 | 90 | 100 | 100 | 100 | 90 | 90 | 90 | 90 | 90 |
| Soybean | 100 | 100 | 20 | 80 | 80 | 100 | 90 | 100 | 100 | 40 | 70 | 90 | 80 | 90 |
| Wheat | 90 | 90 | 0 | 10 | 50 | 90 | 90 | 60 | 90 | 20 | 70 | 40 | 60 | 80 |
| | Compounds | | | | | | | | | | | | | |
| 500 g ai/ha | 266 | 267 | 269 | 270 | 271 | 272 | 273 | 275 | 276 | 277 | 278 | 279 | 280 | 282 |
| Preemergence | | | | | | | | | | | | | | |
| Barnyardgrass | 80 | 80 | 100 | 100 | 100 | 0 | 100 | 100 | 100 | 100 | 100 | 100 | 100 | — |
| Blackgrass | 60 | 60 | 90 | 90 | 60 | 0 | 50 | 90 | 90 | 90 | 90 | 90 | 80 | 0 |
| Corn | 40 | 10 | 90 | 40 | 0 | 0 | 10 | 60 | 90 | 80 | 80 | 80 | 60 | 0 |
| Foxtail, Giant | — | — | — | — | — | — | — | — | — | — | — | — | — | 0 |
| Foxtail, Green | 100 | 100 | 100 | 100 | 100 | 0 | 100 | 100 | 100 | 100 | 100 | 100 | 100 | — |
| Goosegrass | — | — | — | — | — | — | — | — | — | — | — | — | — | 0 |
| Kochia | 90 | 90 | 100 | 90 | 100 | 0 | 90 | 100 | 90 | 90 | 100 | 90 | 100 | 0 |
| Oat, Wild | 90 | 60 | 90 | 90 | 80 | 0 | 80 | 90 | 90 | 90 | 90 | 90 | 90 | 0 |
| Pigweed, Palmer | 100 | 100 | 100 | 100 | 100 | 90 | 100 | 100 | 100 | 100 | 100 | 100 | 100 | 0 |
| Ragweed | 30 | 60 | 100 | 60 | 100 | 30 | 0 | 80 | 80 | 70 | 80 | 90 | 100 | 0 |
| Ryegrass, Italian | 90 | 90 | 100 | 100 | 90 | 0 | 90 | 100 | 90 | 100 | 100 | 90 | 90 | 0 |
| Soybean | 100 | 90 | 90 | 100 | 100 | 0 | 70 | 100 | 100 | 100 | 100 | 100 | 100 | 0 |
| Wheat | 70 | 30 | 100 | 90 | 80 | 0 | 40 | 70 | 90 | 80 | 100 | 90 | 90 | 0 |
| | Compounds | | | | | | | | | | | | | |
| 500 g ai/ha | 283 | 284 | 285 | 288 | 289 | 290 | 291 | 292 | 294 | 299 | 30 0 | 301 | 302 | 303 |
| Preemergence | | | | | | | | | | | | | | |
| Barnyardgrass | — | — | — | 30 | 100 | 100 | 100 | 90 | 100 | 80 | 50 | 90 | 50 | 100 |
| Blackgrass | 40 | 60 | 0 | 60 | 90 | 70 | 70 | 90 | 90 | 70 | 20 | 100 | 60 | 100 |
| Corn | 0 | 0 | 0 | 10 | 20 | 40 | 50 | 10 | 50 | 30 | 10 | 0 | 0 | 50 |
| Foxtail, Giant | 90 | 90 | 0 | — | — | — | — | — | — | — | — | — | — | — |
| Foxtail, Green | — | — | — | 70 | 100 | 100 | 100 | 100 | 100 | 100 | 100 | 100 | 80 | 100 |
| Goosegrass | 100 | 90 | 0 | — | — | — | — | — | — | — | — | — | — | — |
| Kochia | 50 | 10 | — | 50 | 100 | 90 | 100 | 60 | 80 | 80 | 70 | 90 | 80 | 100 |
| Dat, Wild | 10 | 70 | 0 | 20 | 90 | 90 | 80 | 80 | — | 90 | 30 | 90 | 80 | 90 |
| Pigweed, Palmer | 100 | 0 | 0 | 80 | 100 | 100 | 100 | 100 | 90 | 90 | 80 | 100 | 80 | 100 |
| Ragweed | 0 | 0 | 0 | 0 | 70 | 70 | 60 | 20 | 0 | 100 | 20 | 50 | — | 70 |
| Ryegrass, Italian | 100 | 90 | 30 | 80 | 100 | 100 | 100 | 100 | 100 | 90 | 80 | 100 | 100 | 100 |
| Soybean | 30 | 0 | 0 | 40 | 90 | 80 | 100 | 90 | 90 | 0 | 20 | 100 | 20 | 100 |
| Wheat | 10 | 10 | 0 | 20 | 50 | 50 | 50 | 50 | 20 | 30 | 20 | 90 | 40 | 100 |
| | Compounds | | | | | | | | | | | | | |
| 500 g ai/ha | 305 | 306 | 307 | 308 | 310 | 311 | 312 | 313 | 314 | 315 | 316 | 317 | 318 | 319 |
| Preemergence | | | | | | | | | | | | | | |
| Barnyardgrass | 100 | 0 | 90 | 50 | 80 | 60 | 40 | 100 | 100 | 90 | 90 | 50 | 100 | 90 |
| Blackgrass | 90 | 0 | 80 | 30 | 50 | 80 | 80 | 60 | 70 | 50 | 90 | 50 | 90 | 70 |
| Corn | 30 | 0 | 10 | 0 | 0 | 0 | 10 | 0 | 0 | 0 | 40 | 10 | 80 | 20 |
| Foxtail, Giant | — | — | — | — | — | — | — | — | — | — | 100 | 90 | 100 | 100 |
| Foxtail, Green | 100 | 50 | 100 | 90 | 100 | 100 | 90 | 100 | 100 | 90 | — | — | — | — |
| Goosegrass | — | — | — | — | — | — | — | — | — | — | — | — | — | — |
| Kochia | 100 | 0 | 80 | 20 | 80 | 80 | 20 | 100 | 100 | 0 | 0 | 0 | 90 | 0 |
| Oat, Wild | 80 | 0 | 90 | 70 | 60 | 30 | 70 | 90 | 80 | 60 | 40 | 40 | 90 | 40 |
| Pigweed, Palmer | 100 | 10 | 100 | 80 | 100 | 100 | 70 | 100 | 100 | 100 | 90 | 90 | 90 | 90 |
| Ragweed | 90 | 20 | 50 | 20 | 0 | — | 0 | 70 | 70 | 30 | 0 | 0 | 40 | 0 |
| Ryegrass, Italian | 90 | 40 | 80 | 70 | 80 | 80 | 80 | 100 | 100 | 90 | 90 | 60 | 100 | 100 |
| Soybean | 90 | 0 | 100 | 0 | 0 | 10 | 10 | 100 | 90 | 0 | 50 | 0 | 100 | 100 |
| Wheat | 70 | 0 | 90 | 30 | 0 | 20 | 10 | 90 | 40 | 30 | 20 | 0 | 90 | 30 |
| | Compounds | | | | | | | | | | | | | |
| 500 g ai/ha | 320 | 321 | 322 | 323 | 324 | 325 | 326 | 327 | 328 | 329 | 330 | 331 | 332 | 333 |
| Preemergence | | | | | | | | | | | | | | |
| Barnyardgrass | 80 | 100 | 90 | 100 | 100 | 100 | 90 | 90 | 100 | 100 | 100 | 0 | 90 | 0 |
| Blackgrass | 60 | 90 | 40 | 80 | 90 | 80 | 90 | 90 | 80 | 80 | 90 | 70 | 80 | 0 |
| Corn | 30 | 40 | 30 | — | — | 10 | 40 | 70 | 90 | 40 | 60 | 0 | 20 | 0 |
| Foxtail, Giant | 90 | 100 | 100 | 100 | 100 | — | 100 | 100 | 100 | 100 | 100 | 60 | 100 | 80 |
| Foxtail, Green | — | — | — | — | — | 100 | — | — | — | — | — | — | — | — |
| Goosegrass | — | — | — | — | — | — | — | — | — | — | — | — | — | — |
| Kochia | 10 | 90 | 40 | 100 | 90 | 90 | 90 | 90 | 70 | 100 | 90 | 20 | 50 | 10 |
| Oat, Wild | 40 | 80 | 30 | 70 | 90 | 90 | 80 | 70 | 90 | 80 | 60 | 20 | 30 | 0 |
| Pigweed, Palmer | 100 | 100 | 90 | 100 | 100 | 100 | 100 | 90 | 100 | 100 | 100 | 100 | 100 | 30 |
| Ragweed | 0 | 80 | ) | 100 | 90 | 70 | 0 | 30 | 50 | 60 | 50 | 0 | 0 | 0 |
| Ryegrass, Italian | 90 | 100 | 80 | 80 | 100 | 90 | 100 | 100 | 100 | 100 | 100 | 60 | 90 | 40 |
| Soybean | 100 | 40 | 70 | 80 | 90 | 40 | 80 | 100 | 100 | 80 | 80 | 0 | 0 | 0 |
| Wheat | 90 | 30 | 20 | 80 | 90 | 30 | 80 | 90 | 90 | 30 | 30 | 0 | 0 | 0 |
| | Compounds | | | | | | | | | | | | | |
| 500 g ai/ha | 334 | 335 | 336 | 337 | 338 | 339 | 340 | 341 | 342 | 343 | 344 | 345 | 346 | 347 |
| Preemergence | | | | | | | | | | | | | | |
| Barnyardgrass | 100 | 60 | 100 | 90 | 100 | 0 | 100 | 100 | 60 | 100 | 100 | 100 | 100 | 100 |
| Blackgrass | 90 | 80 | 40 | 50 | 80 | 0 | 90 | 80 | 50 | 90 | 100 | 90 | 90 | 30 |
| Corn | 50 | 10 | 20 | 40 | 50 | 0 | 40 | 30 | 20 | 60 | 30 | 30 | 90 | 10 |
| Foxtail, Giant | 100 | 90 | — | — | — | — | — | — | — | — | 100 | 100 | 100 | — |
| Foxtail, Green | — | — | 100 | 100 | 100 | 0 | 100 | 100 | 90 | 100 | — | — | — | 100 |
| Goosegrass | — | — | — | — | — | — | — | — | — | — | — | — | — | — |
| Kochia | 80 | 20 | 90 | 80 | 100 | 50 | 100 | 100 | 0 | 100 | 100 | 100 | 80 | 60 |
| Oat, Wild | 30 | 10 | 30 | 20 | 50 | 0 | 80 | 80 | 80 | 90 | 90 | 80 | 40 | 70 |
| Pigweed, Palmer | 100 | 60 | 90 | 100 | 100 | 0 | 100 | 100 | 70 | 100 | 100 | 100 | 100 | 70 |
| Ragweed | 30 | 0 | 0 | 50 | 0 | 0 | 100 | 100 | 20 | 70 | 50 | 0 | 10 | 30 |
| Ryegrass, Italian | 90 | 80 | 90 | 90 | 100 | 80 | 100 | 100 | 80 | 100 | 100 | 90 | 90 | 100 |
| Soybean | 60 | 30 | 30 | 40 | 80 | 0 | 100 | 60 | 10 | 100 | 50 | 100 | 100 | 30 |
| Wheat | 20 | 0 | 40 | 10 | 20 | 0 | 80 | 30 | 30 | 90 | 80 | 10 | 80 | 40 |
| | Compounds | | | | | | | | | | | | | |
| 500 g ai/ha | 348 | 349 | 350 | 352 | 353 | 354 | 355 | 356 | 357 | 358 | 359 | 360 | 361 | 362 |
| Preemergence | | | | | | | | | | | | | | |
| Barnyardgrass | 100 | 100 | 100 | 0 | 70 | 100 | 0 | 50 | 100 | 100 | 100 | 100 | 90 | 100 |
| Blackgrass | 90 | 80 | 50 | 0 | 50 | 90 | 0 | 20 | 100 | 90 | 100 | 100 | 60 | 80 |
| Corn | 0 | 60 | 0 | 0 | 10 | 40 | 0 | 10 | 20 | 70 | 30 | 70 | 10 | 20 |
| Foxtail, Giant | — | — | — | — | — | — | — | — | — | — | — | — | — | — |
| Foxtail, Green | 100 | 100 | 100 | 40 | 100 | 100 | 20 | 90 | 100 | 100 | 100 | 100 | 100 | 100 |
| Goosegrass | — | — | — | — | — | — | — | — | — | — | — | — | — | — |
| Kochia | 100 | 90 | 80 | 0 | 70 | 80 | 0 | 90 | 90 | 100 | 100 | 80 | 90 | 80 |
| Oat, Wild | 90 | 90 | 50 | 0 | 80 | 90 | 0 | — | 90 | — | 90 | — | 50 | 70 |
| Pigweed, Palmer | 100 | 100 | 100 | 10 | 100 | 100 | 0 | 100 | 100 | 100 | 100 | 90 | 100 | 100 |
| Ragweed | 20 | 80 | 60 | 0 | 0 | 100 | 0 | 0 | 60 | 90 | 100 | 100 | 70 | 60 |
| Ryegrass, Italian | 100 | 100 | 100 | 40 | 90 | 100 | 20 | 80 | 100 | 100 | 100 | 90 | 80 | 90 |
| Soybean | 80 | 100 | 90 | 0 | 40 | 100 | 0 | 0 | 100 | 100 | 100 | 100 | 100 | 70 |
| Wheat | 80 | 70 | 30 | 0 | 30 | 90 | 0 | 0 | 70 | 90 | 80 | 80 | 70 | 40 |
| | Compounds | | | | | | | | | | | | | |
| 500 g ai/ha | 363 | 364 | 377 | 378 | 379 | 380 | 382 | 386 | 387 | 388 | 391 | 392 | 393 | 394 |
| Preemergence | | | | | | | | | | | | | | |
| Barnyardgrass | 60 | 100 | 100 | 100 | 100 | 80 | 100 | 70 | 100 | 90 | ) | 100 | 70 | 100 |
| Blackgrass | 30 | 90 | 20 | 50 | 0 | 30 | 50 | 0 | 20 | 40 | 80 | 80 | 20 | 90 |
| Corn | 0 | 30 | 20 | 50 | 30 | 50 | 70 | 20 | 30 | 10 | 20 | 70 | 20 | 30 |
| Foxtail, Giant | — | — | — | — | — | — | — | — | — | — | — | — | — | — |
| Foxtail, Green | 100 | 100 | 100 | 100 | 100 | 100 | 100 | 90 | 100 | 100 | 100 | 100 | 100 | 100 |
| Goosegrass | — | — | — | — | — | — | — | — | — | — | — | — | — | — |
| Kochia | 0 | 90 | 100 | 100 | 100 | 90 | 100 | 70 | 100 | 100 | 100 | 90 | 90 | 100 |
| Oat, Wild | 0 | 30 | 20 | 60 | 80 | 40 | 80 | 40 | 70 | 40 | 80 | 90 | 40 | 90 |
| Pigweed, Palmer | 100 | 100 | 100 | 100 | 100 | 100 | 100 | 100 | 100 | 100 | 100 | 100 | 100 | 100 |
| Ragweed | 80 | 80 | 70 | 80 | 80 | 90 | 100 | 80 | 100 | 60 | 60 | 80 | 0 | 50 |
| Ryegrass, Italian | 70 | 90 | 70 | 90 | 90 | 80 | 100 | 80 | 80 | 60 | 90 | 100 | 90 | 90 |
| Soybean | 0 | 100 | 90 | 90 | 90 | 90 | 80 | 100 | 100 | 90 | 90 | 100 | 40 | 100 |
| Wheat | 0 | 40 | 20 | 30 | 40 | 10 | 40 | 0 | 30 | 20 | 30 | 60 | 30 | 80 |
| | Compounds | | | | | | | | | | | | | |
| 500 g ai/ha | 395 | 396 | 397 | 398 | 399 | 40 0 | 401 | 402 | 403 | 408 | 410 | 411 | 412 | 413 |
| Preemergence | | | | | | | | | | | | | | |
| Barnyardgrass | 100 | 90 | 100 | 90 | 70 | 100 | 70 | 100 | 70 | 100 | 100 | 70 | 80 | 90 |
| Blackgrass | 90 | 40 | 90 | 40 | 0 | 50 | 30 | 30 | 80 | 80 | 100 | 60 | 90 | 60 |
| Corn | 50 | 40 | 20 | 0 | 0 | 0 | 20 | 20 | 50 | 60 | 80 | 0 | 30 | 10 |
| Foxtail, Giant | — | — | — | — | — | — | — | — | — | — | — | — | — | — |
| Foxtail, Green | 100 | 100 | 100 | 100 | 100 | 100 | 100 | 100 | 100 | 100 | 100 | 100 | 100 | 100 |
| Goosegrass | — | — | — | — | — | — | — | — | — | — | — | — | — | — |
| Kochia | 100 | 90 | 100 | 100 | 70 | 90 | 90 | 90 | 100 | 100 | 100 | 60 | 80 | 80 |
| Oat, Wild | 90 | 90 | 90 | 40 | 30 | 30 | 40 | 40 | 40 | 60 | 90 | 0 | 0 | 0 |
| Pigweed, Palmer | 100 | 100 | 100 | 100 | 100 | 100 | 100 | 100 | 100 | 100 | 100 | 100 | 100 | 100 |
| Ragweed | 90 | 90 | 80 | 30 | 80 | 70 | 60 | 50 | 80 | 90 | 100 | 90 | 100 | 70 |
| Ryegrass, Italian | 90 | 90 | 90 | 80 | 70 | 90 | 90 | 80 | 90 | 80 | 100 | 90 | 90 | 100 |
| Soybean | 100 | 100 | 100 | 60 | 20 | 100 | 40 | 40 | 90 | 100 | 100 | 100 | 90 | 100 |
| Wheat | 80 | 30 | 30 | 20 | 10 | 40 | 20 | 30 | 50 | 60 | 90 | 0 | 30 | 0 |
| | Compounds | | | | | | | | | | | | | |
| 125 g ai/ha | 150 | 151 | 152 | 153 | 154 | 155 | 156 | 157 | 158 | 164 | 165 | 167 | 168 | 169 |
| Preemergence | | | | | | | | | | | | | | |
| Barnyardgrass | 0 | 90 | 70 | 80 | 70 | 90 | 70 | 50 | 60 | 50 | 0 | 90 | 50 | 80 |
| Blackgrass | 0 | 50 | 50 | 20 | 20 | 50 | 50 | 30 | 40 | 50 | 0 | 30 | 20 | 90 |
| Corn | 0 | 20 | 30 | 10 | 0 | 10 | 0 | 0 | 0 | 30 | 0 | 0 | 0 | 0 |
| Foxtail, Giant | — | — | — | — | — | — | — | — | — | — | — | — | — | — |
| Foxtail, Green | 0 | 100 | 100 | 100 | 100 | 100 | 100 | 100 | 100 | 100 | 0 | 100 | 50 | 100 |
| Goosegrass | — | — | — | — | — | — | — | — | — | — | — | — | — | — |
| Kochia | 0 | 100 | 40 | 0 | 100 | 80 | 80 | 20 | 70 | 40 | 0 | 90 | 0 | 100 |
| Oat, Wild | 0 | 30 | 60 | 40 | 20 | 30 | 20 | 0 | 20 | — | 0 | 20 | 0 | 80 |
| Pigweed, Palmer | 0 | 100 | 90 | 100 | 100 | 100 | 100 | 100 | 100 | 100 | 0 | 100 | 100 | 100 |
| Ragweed | 0 | 30 | 0 | 80 | 50 | 60 | 70 | 90 | 80 | 70 | 0 | 10 | 30 | 50 |
| Ryegrass, Italian | 0 | 80 | 70 | 90 | 70 | 70 | 60 | 70 | 80 | 90 | 0 | 80 | 70 | 90 |
| Soybean | 0 | 50 | 50 | 100 | 40 | 70 | 80 | 50 | 50 | 90 | 0 | 40 | 60 | 90 |
| Wheat | 0 | 80 | 20 | 20 | 10 | 20 | 10 | 0 | 10 | 20 | 0 | 0 | 0 | 30 |
| | Compounds | | | | | | | | | | | | | |
| 125 g ai/ha | 170 | 171 | 172 | 173 | 174 | 175 | 177 | 178 | 179 | 180 | 191 | 192 | 193 | 194 |
| Preemergence | | | | | | | | | | | | | | |
| Barnyardgrass | 90 | 90 | 0 | 0 | 80 | 70 | 100 | 20 | 0 | 60 | 0 | 60 | 80 | 10 |
| Blackgrass | 50 | 70 | 0 | 0 | 70 | 0 | 40 | 60 | 0 | 20 | 0 | 70 | 0 | 20 |
| Corn | 20 | 30 | 0 | 0 | 10 | 0 | 30 | 20 | 0 | 0 | 0 | 20 | 0 | 0 |
| Foxtail, Giant | — | — | — | — | — | — | — | — | — | — | — | — | — | — |
| Foxtail, Green | 100 | 100 | 0 | 0 | 100 | 90 | 100 | 100 | 0 | 70 | 0 | 100 | 0 | 70 |
| Goosegrass | — | — | — | — | — | — | — | — | — | — | — | — | — | — |
| Kochia | 90 | 90 | 0 | 0 | 70 | 40 | 80 | 90 | 0 | 0 | — | 60 | 100 | 0 |
| Oat, Wild | 60 | 70 | 0 | 0 | 20 | 20 | 80 | 30 | 0 | 50 | 0 | 40 | 20 | 20 |
| Pigweed, Palmer | 100 | 100 | 0 | 0 | 100 | 100 | 100 | 90 | 0 | 60 | 0 | 90 | 90 | 20 |
| Ragweed | 20 | 60 | 0 | 30 | 0 | 20 | 90 | 0 | 0 | 20 | 0 | 10 | 60 | 20 |
| Ryegrass, Italian | 80 | 50 | 0 | 20 | 80 | 80 | 100 | — | — | — | 0 | 90 | 80 | 70 |
| Soybean | 70 | 60 | 0 | 0 | 70 | 60 | 60 | 60 | 0 | 50 | 0 | 30 | 90 | 10 |
| Wheat | 50 | 40 | 0 | 0 | 0 | 20 | 30 | 30 | 0 | 0 | 0 | 0 | 0 | 0 |
| | Compounds | | | | | | | | | | | | | |
| 125 g ai/ha | 195 | 196 | 203 | 210 | 211 | 212 | 213 | 214 | 215 | 216 | 217 | 218 | 219 | 220 |
| Preemergence | | | | | | | | | | | | | | |
| Barnyardgrass | 90 | — | 0 | 100 | 100 | 80 | 60 | 50 | 80 | 40 | 90 | 100 | 80 | 50 |
| Blackgrass | 30 | 0 | 0 | 80 | 90 | 60 | 60 | 20 | 70 | 20 | 60 | 40 | 0 | 40 |
| Corn | 0 | 0 | 0 | 30 | 70 | 20 | 0 | 10 | 30 | 0 | 30 | 20 | 10 | 0 |
| Foxtail, Giant | — | 0 | — | — | — | — | — | — | — | — | — | — | — | — |
| Foxtail, Green | 100 | — | 20 | 100 | 0 | 100 | 100 | 100 | 100 | 100 | 100 | 100 | 100 | 100 |
| Goosegrass | — | 0 | — | — | — | — | — | — | — | — | — | — | — | — |
| Kochia | 70 | 0 | 0 | 90 | 100 | 90 | 30 | 90 | 90 | 70 | 80 | 90 | 90 | 80 |
| Oat, Wild | 20 | 0 | — | 60 | 70 | 70 | 10 | 0 | 50 | 0 | 90 | 60 | 30 | 60 |
| Pigweed, Palmer | 100 | 0 | 20 | 100 | 100 | 100 | 100 | 100 | 100 | 100 | 100 | 100 | 100 | 90 |
| Ragweed | 60 | 0 | 0 | 30 | 100 | 0 | 0 | 10 | 0 | 0 | 70 | 20 | 30 | 0 |
| Ryegrass, Italian | 80 | 0 | 0 | 100 | 100 | 90 | 90 | 80 | 90 | 80 | 90 | 90 | 90 | 80 |
| Soybean | 70 | 0 | 0 | 90 | 100 | 90 | 50 | 30 | 80 | 50 | 70 | 90 | 70 | 0 |
| Wheat | 0 | 0 | 0 | 90 | 80 | 40 | 10 | 10 | 20 | 0 | 30 | 30 | 20 | 20 |
| | Compounds | | | | | | | | | | | | | |
| 125 g ai/ha | 221 | 223 | 224 | 225 | 226 | 227 | 228 | 229 | 230 | 231 | 232 | 233 | 234 | 235 |
| Preemergence | | | | | | | | | | | | | | |
| Barnyardgrass | 0 | 90 | 40 | 50 | 50 | 80 | 90 | 0 | 60 | 60 | 0 | 0 | 90 | 50 |
| Blackgrass | 0 | 40 | 20 | 60 | 30 | 80 | 80 | 0 | 70 | 60 | 0 | 10 | 70 | 50 |
| Corn | 0 | 0 | 0 | 20 | 0 | 50 | 10 | 0 | 20 | 10 | 0 | 0 | 10 | 20 |
| Foxtail, Giant | — | — | — | — | — | — | — | — | — | — | — | — | — | — |
| Foxtail, Green | 0 | 100 | 100 | 100 | 100 | 100 | 100 | 0 | 100 | 100 | 0 | 0 | 100 | 100 |
| Goosegrass | — | — | — | — | — | — | — | — | — | — | — | — | — | — |
| Kochia | 0 | 60 | 40 | 20 | 50 | 90 | 0 | 0 | 0 | 90 | 0 | 0 | 30 | 80 |
| Oat, Wild | 0 | 50 | 30 | 60 | 40 | 90 | 40 | 0 | 50 | 40 | 0 | 0 | 50 | 20 |
| Pigweed, Palmer | — | 100 | 90 | 100 | 50 | 90 | 100 | 0 | 100 | 100 | — | 0 | 60 | 90 |
| Ragweed | 0 | 50 | 0 | 70 | 20 | 0 | 40 | 0 | 60 | 50 | 0 | 0 | 50 | 30 |
| Ryegrass, Italian | 0 | 80 | 70 | 60 | 70 | 90 | 90 | 0 | 80 | 70 | 20 | 10 | 80 | 80 |
| Soybean | 0 | 10 | 60 | 80 | 20 | 70 | 70 | 0 | 90 | 60 | 0 | 0 | 90 | 60 |
| Wheat | 0 | 20 | 0 | 20 | 0 | 0 | 20 | 0 | 30 | 40 | 0 | 0 | 40 | 30 |
| | Compounds | | | | | | | | | | | | | |
| 125 g ai/ha | 236 | 240 | 241 | 242 | 248 | 249 | 252 | 253 | 254 | 256 | 258 | 259 | 260 | 262 |
| Preemergence | | | | | | | | | | | | | | |
| Barnyardgrass | 20 | 90 | 90 | 100 | 100 | 20 | 80 | 50 | 70 | 100 | 80 | 30 | 70 | 60 |
| Blackgrass | 0 | 60 | 50 | 90 | 30 | 20 | 0 | 20 | 40 | 70 | 40 | 50 | 60 | 60 |
| Corn | 0 | 70 | 20 | 30 | 20 | 0 | 0 | 0 | 50 | 50 | 10 | 30 | 0 | 40 |
| Foxtail, Giant | — | — | — | — | — | — | — | — | — | — | — | — | — | — |
| Foxtail, Green | 90 | 100 | 100 | 100 | 100 | 90 | 100 | 80 | 100 | 100 | 100 | 100 | 100 | 100 |
| Goosegrass | — | — | — | — | — | — | — | — | — | — | — | — | — | — |
| Kochia | 40 | 70 | 20 | 90 | 100 | 0 | 100 | 60 | 90 | 80 | 90 | 90 | 60 | 40 |
| Oat, Wild | 10 | 90 | 50 | 90 | 40 | 0 | 20 | 30 | 90 | 90 | 40 | 60 | 30 | 50 |
| Pigweed, Palmer | 80 | 100 | 100 | 100 | 100 | 30 | 100 | 90 | 100 | 100 | 100 | 100 | 40 | 70 |
| Ragweed | 20 | 10 | 10 | 0 | 80 | 0 | 0 | 20 | 80 | 70 | 40 | 30 | 0 | 0 |
| Ryegrass, Italian | 50 | 100 | 90 | 100 | 90 | 40 | 60 | 40 | 90 | 90 | 90 | 90 | 80 | 70 |
| Soybean | 40 | 100 | 100 | 90 | 60 | 0 | 20 | 40 | 100 | 100 | 50 | 20 | 10 | 30 |
| Wheat | 0 | 60 | 60 | 60 | 40 | 0 | 0 | 0 | 70 | 80 | 50 | 50 | 20 | 20 |
| | Compounds | | | | | | | | | | | | | |
| 125 g ai/ha | 263 | 264 | 265 | 266 | 267 | 268 | 269 | 270 | 271 | 273 | 275 | 276 | 277 | 278 |
| Preemergence | | | | | | | | | | | | | | |
| Barnyardgrass | 70 | 70 | 90 | 50 | 60 | 100 | 100 | 70 | 100 | 40 | 100 | 90 | 100 | 100 |
| Blackgrass | 50 | 40 | 70 | 30 | 20 | 70 | 90 | 80 | 20 | 0 | 50 | 90 | 80 | 90 |
| Corn | 20 | 30 | 30 | 20 | 0 | 30 | 20 | 0 | 0 | 0 | 30 | 50 | 50 | 3 30 |
| Foxtail, Giant | — | — | — | — | — | — | — | — | — | — | — | — | — | — |
| Foxtail, Green | 90 | 100 | 100 | 100 | 100 | 100 | 100 | 100 | 100 | 100 | 100 | 100 | 100 | 100 |
| Goosegrass | — | — | — | — | — | — | — | — | — | — | — | — | — | — |
| Kochia | — | 90 | 80 | 90 | 90 | 100 | 90 | 60 | 30 | 60 | 80 | 90 | 70 | 90 |
| Oat, Wild | 30 | 90 | 60 | 40 | 40 | 40 | 60 | 40 | 30 | 20 | 50 | 80 | 80 | 80 |
| Pigweed, Palmer | 70 | 100 | 100 | 100 | 90 | 100 | 100 | 90 | 100 | 90 | 100 | 100 | 100 | 100 |
| Ragweed | 0 | 60 | 50 | 0 | 40 | 90 | 100 | 10 | 10 | 0 | 60 | 60 | 30 | 70 |
| Ryegrass, Italian | 80 | 90 | 80 | 80 | 70 | 90 | 100 | 90 | 80 | 70 | 80 | 90 | 80 | 80 |
| Soybean | 80 | 90 | 70 | 80 | 90 | 80 | 90 | 90 | 70 | 10 | 80 | 100 | 90 | 90 |
| Wheat | 20 | 0 | 70 | 30 | 20 | 40 | 100 | 40 | 20 | 0 | 30 | 80 | 50 | 50 |
| | Compounds | | | | | | | | | | | | | |
| 125 g ai/ha | 279 | 280 | 282 | 283 | 284 | 285 | 288 | 289 | 290 | 291 | 292 | 294 | 299 | 30 0 |
| Preemergence | | | | | | | | | | | | | | |
| Barnyardgrass | 90 | 100 | — | — | — | — | 0 | 60 | 90 | 100 | 20 | 40 | 10 | 0 |
| Blackgrass | 70 | 70 | 0 | 40 | 0 | 0 | 10 | 80 | 70 | 50 | 30 | 70 | 30 | 0 |
| Corn | 50 | 30 | 0 | 0 | 0 | 0 | 0 | 10 | 30 | 30 | 10 | 50 | 0 | 0 |
| Foxtail, Giant | — | — | 0 | 60 | 0 | 0 | — | — | — | — | — | — | — | — |
| Foxtail, Green | 100 | 100 | — | — | — | — | 0 | 100 | 100 | 100 | 90 | 90 | 60 | 80 |
| Goosegrass | — | — | 0 | 80 | 10 | 0 | — | — | — | — | — | — | — | — |
| Kochia | 70 | 90 | 0 | 20 | 0 | — | 10 | 70 | 90 | 80 | 30 | 20 | 0 | 50 |
| Oat, Wild | 80 | 60 | 0 | 20 | 20 | 0 | 20 | 80 | 70 | 60 | 40 | — | 0 | 20 |
| Pigweed, Palmer | 100 | 100 | 0 | 30 | 0 | 0 | 50 | 100 | 100 | 100 | 100 | 60 | 50 | 20 |
| Ragweed | 70 | 90 | 0 | 0 | 0 | 0 | 0 | 50 | 60 | 40 | 20 | 0 | 70 | 0 |
| Ryegrass, Italian | 90 | 90 | 0 | 70 | 50 | 20 | 80 | 100 | 90 | 90 | 90 | 90 | 70 | 60 |
| Soybean | 90 | 90 | 0 | 0 | 0 | 0 | 40 | 90 | 90 | 90 | 40 | 20 | 0 | 0 |
| Wheat | 60 | 70 | 0 | 0 | 0 | 0 | 0 | 30 | 30 | 30 | 20 | 0 | 0 | 0 |
| | Compounds | | | | | | | | | | | | | |
| 125 g ai/ha | 301 | 302 | 303 | 305 | 306 | 307 | 308 | 310 | 311 | 312 | 313 | 314 | 315 | 316 |
| Preemergence | | | | | | | | | | | | | | |
| Barnyardgrass | 50 | 0 | 40 | 70 | 0 | 40 | 0 | 20 | 0 | 0 | 80 | 70 | 0 | 30 |
| Blackgrass | 90 | 30 | 90 | 50 | — | 40 | 0 | 10 | 10 | 50 | 50 | 20 | 0 | 50 |
| Corn | 0 | — | 0 | 0 | 0 | 0 | 0 | 0 | 0 | 0 | 0 | 0 | 0 | 10 |
| Foxtail, Giant | — | — | — | — | — | — | — | — | — | — | — | — | — | 80 |
| Foxtail, Green | 100 | 20 | 100 | 100 | 0 | 100 | 30 | 100 | 90 | 80 | 100 | 100 | 30 | — |
| Goosegrass | — | — | — | — | — | — | — | — | — | — | — | — | — | — |
| Kochia | 80 | 0 | 70 | 80 | 0 | 40 | 0 | 50 | 20 | 0 | 80 | 90 | 0 | 0 |
| Oat, Wild | 70 | 0 | 50 | 20 | 0 | 50 | 0 | 0 | 0 | 0 | 90 | 60 | 0 | 20 |
| Pigweed, Palmer | 90 | 70 | 100 | 80 | 0 | 60 | 20 | 80 | 50 | 20 | 100 | 100 | 10 | 60 |
| Ragweed | 0 | — | 0 | 70 | 20 | 0 | 30 | 0 | — | 0 | 0 | 50 | 0 | 0 |
| Ryegrass, Italian | 100 | 80 | 90 | 60 | 30 | 80 | 30 | 30 | 30 | 70 | 80 | 100 | 40 | 70 |
| Soybean | 10 | 10 | 30 | 0 | 0 | 20 | 0 | 40 | 0 | 0 | 40 | 40 | 0 | 0 |
| Wheat | 40 | 20 | 70 | 0 | 0 | 10 | 0 | 0 | 0 | 0 | 20 | 20 | 0 | 0 |
| | Compounds | | | | | | | | | | | | | |
| 125 g ai/ha | 317 | 318 | 319 | 320 | 321 | 322 | 323 | 324 | 325 | 326 | 327 | 328 | 329 | 330 |
| Preemergence | | | | | | | | | | | | | | |
| Barnyardgrass | 0 | 90 | 40 | 30 | 40 | 40 | 70 | 90 | 40 | 60 | 50 | 90 | 70 | 70 |
| Blackgrass | 0 | 80 | 50 | 0 | 80 | 40 | 70 | 70 | 60 | 80 | 90 | 80 | 60 | 70 |
| Corn | 0 | 30 | 0 | 30 | 10 | 30 | — | 0 | 10 | — | 20 | 30 | 20 | 10 |
| Foxtail, Giant | 10 | 100 | 100 | 20 | 100 | 90 | 90 | 90 | — | 100 | 100 | 100 | 100 | 100 |
| Foxtail, Green | — | — | — | — | — | — | — | — | 100 | — | — | — | — | — |
| Goosegrass | — | — | — | — | — | — | — | — | — | — | — | — | — | — |
| Kochia | 0 | 70 | 0 | 10 | 20 | 20 | 90 | 90 | 40 | 80 | 20 | 0 | 40 | 30 |
| Oat, Wild | 0 | 50 | 20 | 10 | 0 | 0 | 20 | 0 | 60 | 30 | 20 | 30 | 30 | 30 |
| Pigweed, Palmer | 20 | 50 | 80 | 20 | 50 | 40 | 100 | 100 | 100 | 60 | 50 | 40 | 90 | 100 |
| Ragweed | 0 | 0 | 0 | 0 | 0 | 0 | 30 | 50 | 20 | 0 | 0 | 0 | 0 | 0 |
| Ryegrass, Italian | 40 | 90 | 100 | 80 | 90 | 60 | 70 | 80 | 60 | 90 | 90 | 90 | 70 | 100 |
| Soybean | 0 | 30 | 20 | 10 | 0 | 0 | 50 | 100 | 40 | 70 | 100 | 100 | 0 | 40 |
| Wheat | 0 | 30 | 0 | 20 | 10 | 0 | 60 | 10 | 10 | 20 | 40 | 20 | 10 | 20 |
| | Compounds | | | | | | | | | | | | | |
| 125 g ai/ha | 331 | 332 | 333 | 334 | 335 | 336 | 337 | 338 | 339 | 340 | 341 | 342 | 343 | 344 |
| Preemergence | | | | | | | | | | | | | | |
| Barnyardgrass | 0 | 50 | 0 | 60 | 20 | 20 | 30 | 60 | 0 | 90 | 90 | 20 | 100 | 80 |
| Blackgrass | 20 | 80 | 0 | 80 | 20 | 30 | 20 | 20 | 0 | 50 | 20 | 20 | 50 | 90 |
| Corn | 0 | 20 | 0 | 0 | 0 | 0 | 0 | 10 | 0 | 20 | 20 | 0 | 40 | 20 |
| Foxtail, Giant | 20 | 70 | 0 | 90 | 30 | — | — | — | — | — | — | — | — | 100 |
| Foxtail, Green | — | — | — | — | — | 80 | 90 | 90 | 0 | 100 | 100 | 40 | 100 | — |
| Goosegrass | — | — | — | — | — | — | — | — | — | — | — | — | — | — |
| Kochia | 0 | 0 | 0 | 40 | 0 | 0 | 0 | 60 | 0 | 90 | 100 | 0 | 90 | 60 |
| Oat, Wild | 0 | 30 | 0 | 0 | 0 | 0 | 0 | 30 | 0 | 70 | 50 | 20 | 90 | 0 |
| Pigweed, Palmer | 30 | 20 | 0 | 90 | 0 | 10 | 0 | 100 | 0 | 100 | 100 | 30 | 100 | 90 |
| Ragweed | 0 | 0 | 0 | 0 | 0 | 0 | 30 | 0 | 0 | 80 | 80 | 0 | 30 | 0 |
| Ryegrass, Italian | 60 | 60 | 30 | 70 | 30 | 70 | 50 | 90 | 20 | 90 | 80 | 70 | 100 | 90 |
| Soybean | 0 | 0 | 0 | 0 | 0 | 0 | 0 | 30 | 0 | 30 | 100 | 10 | 30 | 0 |
| Wheat | 0 | 0 | 0 | 0 | 0 | 0 | 0 | 0 | 0 | 50 | 30 | 0 | 30 | 40 |
| | Compounds | | | | | | | | | | | | | |
| 125 g ai/ha | 345 | 346 | 347 | 348 | 349 | 350 | 352 | 353 | 354 | 355 | 356 | 357 | 358 | 359 |
| Preemergence | | | | | | | | | | | | | | |
| Barnyardgrass | 60 | 70 | 20 | 60 | 70 | 50 | 0 | 20 | 90 | 0 | 10 | 80 | 100 | 100 |
| Blackgrass | 80 | 80 | 10 | 50 | 50 | 20 | 0 | 0 | 80 | 0 | 0 | 80 | 80 | 80 |
| Corn | 10 | 0 | 0 | 0 | 40 | 0 | 0 | 10 | 20 | 0 | 0 | 10 | 50 | 20 |
| Foxtail, Giant | 90 | 100 | — | — | — | — | — | — | — | — | — | — | — | — |
| Foxtail, Green | — | — | 90 | 100 | 100 | 100 | 0 | 90 | 100 | 20 | 30 | 100 | 100 | 100 |
| Goosegrass | — | — | — | — | — | — | — | — | — | — | — | — | — | — |
| Kochia | 50 | 10 | 50 | 90 | 80 | 50 | 0 | 0 | 0 | 0 | 0 | 50 | 20 | 100 |
| Oat, Wild | 10 | 0 | 60 | 60 | 20 | 30 | 0 | 30 | 80 | 0 | — | 60 | — | 90 |
| Pigweed, Palmer | 100 | 100 | 80 | 100 | 100 | 100 | — | 90 | 100 | — | 40 | 90 | 100 | 90 |
| Ragweed | 0 | 0 | 0 | 20 | 20 | 20 | 40 | 0 | 10 | 0 | 0 | 10 | 70 | 0 |
| Ryegrass, Italian | 80 | 80 | 100 | 90 | 100 | 100 | — | 80 | 90 | 20 | 70 | 100 | 90 | 90 |
| Soybean | 30 | 30 | 30 | 40 | 50 | 90 | 0 | 20 | 50 | 20 | 0 | 100 | 100 | 80 |
| Wheat | 0 | 0 | 20 | 50 | 20 | 30 | 0 | 10 | 30 | 0 | 0 | 0 | 50 | 30 |
| | Compounds | | | | | | | | | | | | | |
| 125 g ai/ha | 360 | 361 | 362 | 363 | 364 | 377 | 378 | 379 | 380 | 382 | 386 | 387 | 388 | 391 |
| Preemergence | | | | | | | | | | | | | | |
| Barnyardgrass | 100 | 80 | 80 | 40 | 50 | 40 | 80 | 60 | 20 | 50 | 40 | 50 | 50 | 60 |
| Blackgrass | 80 | 40 | 50 | 0 | 60 | 0 | 10 | 0 | 0 | 10 | 0 | 0 | 10 | 50 |
| Corn | 60 | 0 | — | 0 | 10 | 0 | 0 | 30 | 0 | 10 | 0 | 0 | . 10 | 20 |
| Foxtail, Giant | — | — | — | — | — | — | — | — | — | — | — | — | — | — |
| Foxtail, Green | 100 | 100 | 100 | 100 | 100 | 50 | 90 | 80 | 50 | 90 | 40 | 50 | 60 | 100 |
| Goosegrass | — | — | — | — | — | — | — | — | — | — | — | — | — | — |
| Kochia | 20 | 80 | 70 | 0 | 70 | 70 | 100 | 80 | 0 | 80 | 70 | 20 | 40 | 90 |
| Oat, Wild | — | 0 | 0 | 0 | 10 | 20 | 20 | 30 | 20 | 20 | 30 | 40 | 20 | 30 |
| Pigweed, Palmer | 30 | 90 | 90 | 80 | 100 | 100 | 100 | 100 | 100 | 100 | 100 | 100 | 100 | 100 |
| Ragweed | 80 | 50 | 20 | 20 | 20 | 30 | 60 | 0 | 70 | 60 | 20 | 80 | 60 | 10 |
| Ryegrass, Italian | 80 | 80 | 80 | 30 | 60 | 60 | 60 | 70 | 70 | 80 | 50 | 60 | 50 | 20 |
| Soybean | 80 | 100 | 50 | 20 | 100 | 60 | 60 | 90 | 50 | 60 | 50 | 90 | 90 | 50 |
| Wheat | 40 | 50 | 10 | 0 | 0 | 0 | 10 | 20 | 0 | 0 | 0 | 10 | 20 | 0 |
| | Compounds | | | | | | | | | | | | | |
| 125 g ai/ha | 392 | 393 | 394 | 395 | 396 | 397 | 398 | 399 | 400 | 401 | 402 | 403 | 408 | 410 |
| Preemergence | | | | | | | | | | | | | | |
| Barnyardgrass | 90 | 40 | 70 | 80 | 50 | 90 | 60 | 10 | 50 | 30 | 60 | 40 | 60 | 90 |
| Blackgrass | 70 | 20 | 30 | 50 | 20 | 70 | 30 | 0 | 0 | 30 | 30 | 60 | 10 | 90 |
| Corn | 30 | 0 | 20 | 30 | 10 | 10 | 0 | 0 | 0 | 0 | 0 | 10 | 0 | 10 |
| Foxtail, Giant | — | — | — | — | — | — | — | — | — | — | — | — | — | — |
| Foxtail, Green | 100 | 80 | 100 | 100 | 90 | 100 | 100 | 20 | 70 | 20 | 100 | 90 | 100 | 100 |
| Goosegrass | — | — | — | — | — | — | — | — | — | — | — | — | — | — |
| Kochia | 90 | 80 | 90 | 100 | 70 | 90 | 90 | 0 | 50 | 0 | 90 | 60 | 70 | 90 |
| Oat, Wild | 50 | 0 | 30 | 50 | 30 | 60 | 0 | 0 | 0 | 0 | 20 | 0 | 30 | 40 |
| Pigweed, Palmer | 100 | 80 | 100 | 100 | 100 | 100 | 100 | 100 | 100 | 100 | 100 | 30 | 100 | 100 |
| Ragweed | 80 | 0 | 0 | 90 | 80 | 50 | 0 | 30 | 30 | 0 | 0 | 30 | 60 | 10 |
| Ryegrass, Italian | 90 | 0 | 60 | 80 | 60 | 80 | 40 | 50 | 60 | 70 | 70 | 40 | 60 | 80 |
| Soybean | 90 | 20 | 20 | 70 | 60 | 70 | 40 | 0 | 20 | 0 | 20 | 80 | 80 | 100 |
| Wheat | 20 | 0 | 20 | 30 | 0 | 10 | 0 | 0 | 0 | 0 | 0 | 0 | 20 | 70 |

| | Compounds | | |
|---|---|---|---|
| 125 g ai/ha | 411 | 412 | 413 |
| Preemergence | | | |
| Barnyardgrass | 60 | 60 | 50 |
| Blackgrass | 10 | 50 | 30 |
| Corn | 0 | 0 | 0 |
| Foxtail, Giant | — | — | — |
| Foxtail, Green | 70 | 100 | 80 |
| Goosegrass | — | — | — |
| Kochia | 0 | 30 | 10 |
| Oat, Wild | 0 | 0 | 0 |
| Pigweed, Palmer | 100 | 100 | 90 |
| Ragweed | 60 | 50 | 60 |
| Ryegrass, Italian | 60 | 80 | 80 |
| Soybean | 60 | 60 | 70 |
| Wheat | 0 | 30 | 0 |

| | Compounds | |
|---|---|---|
| 31 g ai/ha | 180 | 193 |
| Preemergence | | |
| Barnyardgrass | 0 | 10 |
| Blackgrass | 0 | 0 |
| Corn | 0 | 0 |
| Foxtail, Green | 30 | 0 |
| Kochia | 0 | 60 |
| Oat, Wild | 0 | 0 |
| Pigweed, Palmer | 0 | 60 |
| Ragweed | 0 | 0 |
| Ryegrass, Italian | — | 60 |
| Soybean | 0 | 0 |
| Wheat | 0 | 0 |

| | Compounds | | | | | | | | | | | | | |
|---|---|---|---|---|---|---|---|---|---|---|---|---|---|---|
| 1000 g ai/ha | 159 | 160 | 161 | 162 | 163 | 166 | 181 | 197 | 198 | 199 | 200 | 201 | 202 | 204 |
| Postemergence | | | | | | | | | | | | | | |
| Barnyardgrass | 10 | 10 | 10 | 10 | 0 | 0 | 10 | 0 | 20 | 30 | 10 | 10 | 0 | 0 |
| Blackgrass | 0 | 20 | 50 | 0 | 20 | 0 | 20 | 0 | 0 | 20 | 0 | 0 | 0 | 0 |
| Corn | — | — | — | — | — | — | — | 0 | 0 | 10 | 20 | 0 | 0 | — |
| Foxtail, Giant | — | — | — | — | — | — | — | 0 | 0 | 20 | 0 | 10 | 0 | — |
| Foxtail, Green | 30 | 40 | 10 | 10 | 10 | 0 | 0 | — | — | — | — | — | — | 0 |
| Galium | 50 | 50 | 50 | 50 | 0 | 20 | 30 | 30 | 0 | 20 | 30 | 40 | 0 | 50 |
| Horseweed | 0 | 0 | 10 | 0 | 0 | 0 | 0 | — | — | — | — | — | — | 0 |
| Kochia | 40 | 70 | 30 | 70 | 50 | 0 | 20 | 10 | 0 | 10 | 0 | 10 | 0 | 0 |
| Pigweed, Palmer | 20 | 0 | 0 | 20 | 30 | 10 | 10 | — | — | — | — | — | — | 10 |
| Pigweed, Redroot | — | — | — | — | — | — | — | 50 | 0 | 40 | 20 | 0 | 0 | — |
| Ragweed | 60 | 0 | 50 | 20 | 20 | 0 | 0 | 0 | 0 | 0 | 0 | 0 | 0 | 20 |
| Ryegrass, Italian | 40 | 0 | 20 | 40 | 0 | 0 | 10 | 0 | 0 | 40 | 0 | 0 | 0 | 0 |
| Wheat | — | — | — | — | — | — | — | 0 | 20 | 0 | 0 | 0 | 30 | — |
| | Compounds | | | | | | | | | | | | | |
| 1000 g ai/ha | 205 | 206 | 207 | 208 | 209 | 222 | 238 | 239 | 243 | 244 | 245 | 246 | 247 | 250 |
| Postemergence | | | | | | | | | | | | | | |
| Barnyardgrass | 10 | 0 | 30 | 20 | 0 | 20 | 10 | 30 | 10 | 20 | 0 | 0 | 10 | 50 |
| Blackgrass | 30 | 0 | 10 | 50 | 0 | 50 | 0 | 10 | 0 | 20 | 0 | 0 | 0 | 20 |
| Corn | — | — | — | — | — | — | 0 | 0 | — | — | — | — | — | — |
| Foxtail, Giant | — | — | — | — | — | — | — | — | — | — | — | — | — | — |
| Foxtail, Green | 30 | 0 | 10 | 30 | 0 | 40 | 10 | 30 | 0 | 20 | 0 | 0 | 30 | 80 |
| Galium | 0 | 40 | 10 | 60 | 40 | — | 50 | 70 | 60 | 60 | 0 | 0 | 20 | 50 |
| Horseweed | 0 | 0 | 10 | 20 | 20 | 0 | 40 | 30 | 40 | 90 | 30 | 30 | 20 | 0 |
| Kochia | 50 | 20 | 20 | 20 | 0 | 60 | 30 | 40 | 10 | 50 | 0 | 20 | 0 | 70 |
| Pigweed, Palmer | 30 | 50 | 90 | 40 | 0 | 60 | 60 | 70 | 60 | 60 | 0 | 0 | 10 | 50 |
| Pigweed, Redroot | — | — | — | — | — | — | — | — | — | — | — | — | — | — |
| Ragweed | 20 | 0 | 10 | 30 | 0 | 0 | 50 | 50 | 0 | 20 | 10 | 30 | 0 | 50 |
| Ryegrass, Italian | 40 | 40 | 70 | 50 | 0 | 50 | 0 | 0 | 50 | 80 | 0 | 0 | 0 | 40 |
| Wheat | — | — | — | — | — | — | — | — | — | — | — | — | — | — |
| | Compounds | | | | | | | | | | | | | |
| 1000 g ai/ha | 251 | 286 | 287 | 293 | 296 | 297 | 298 | 304 | 309 | 383 | 384 | 385 | 389 | 390 |
| Postemergence | | | | | | | | | | | | | | |
| Barnyardgrass | 30 | 0 | 20 | 0 | 50 | 0 | 0 | 10 | 10 | 10 | 10 | 20 | 70 | 50 |
| Blackgrass | 20 | 0 | 0 | 0 | 80 | 0 | 0 | 30 | 0 | 10 | 10 | 30 | 10 | 50 |
| Corn | — | 0 | 0 | — | — | — | — | — | — | — | — | — | — | — |
| Foxtail, Giant | — | 0 | 10 | — | — | — | — | — | — | — | — | — | — | — |
| Foxtail, Green | 50 | — | — | 0 | 80 | 0 | 0 | 20 | 10 | 0 | 10 | 20 | 70 | 20 |
| Galium | 50 | 0 | 20 | 0 | 100 | 0 | 20 | 70 | 70 | 0 | 40 | 60 | 90 | 80 |
| Horseweed | 0 | — | — | 30 | 60 | 20 | 0 | 0 | 30 | 0 | 0 | 0 | 0 | 0 |
| Kochia | 30 | 0 | 10 | 0 | 90 | 30 | 0 | 40 | 40 | 50 | 50 | 70 | 80 | 70 |
| Pigweed, Palmer | 20 | — | — | 10 | 90 | 0 | 0 | 20 | 70 | 50 | 60 | 70 | 90 | 80 |
| Pigweed, Redroot | — | 0 | 20 | — | — | — | — | — | — | — | — | — | — | — |
| Ragweed | 30 | 0 | 0 | 0 | 80 | 0 | 40 | 0 | 30 | 0 | 0 | 50 | 30 | 30 |
| Ryegrass, Italian | 20 | 0 | 0 | 0 | 30 | 0 | 0 | 0 | 10 | 0 | 0 | 10 | 0 | 20 |
| Wheat | — | 0 | 0 | — | — | — | — | — | — | — | — | — | — | — |
| | Compounds | | | | | | | | | | | | | |
| 500 g ai/ha | 150 | 151 | 152 | 153 | 154 | 155 | 156 | 157 | 158 | 164 | 165 | 167 | 168 | 169 |
| Postemergence | | | | | | | | | | | | | | |
| Barnyardgrass | 10 | 100 | 90 | 90 | 50 | 90 | 90 | 70 | 70 | 50 | 10 | 60 | 30 | 70 |
| Blackgrass | 0 | 80 | 70 | 90 | 90 | 100 | 90 | 70 | 90 | 90 | 10 | 70 | 0 | 100 |
| Corn | 0 | 90 | 70 | 90 | 50 | 90 | 50 | 20 | 60 | 90 | 0 | 20 | 10 | 90 |
| Foxtail, Giant | — | — | — | — | — | — | — | — | — | — | — | — | — | — |
| Foxtail, Green | 0 | 90 | 90 | 90 | 90 | 90 | 90 | 90 | 90 | 80 | 0 | 90 | 30 | 90 |
| Galium | 0 | 100 | 100 | 100 | 100 | 100 | 100 | 100 | 90 | 100 | 0 | 100 | 90 | 100 |
| Goosegrass | — | — | — | — | — | — | — | — | — | — | — | — | — | — |
| Horseweed | 0 | 80 | 0 | 50 | 70 | 70 | 60 | 90 | 20 | 50 | 60 | 30 | 10 | 40 |
| Kochia | 20 | 100 | 80 | 90 | 100 | 90 | 90 | 100 | 90 | 90 | 0 | 100 | 80 | 90 |
| Oat, Wild | 0 | 50 | 30 | 70 | 20 | 50 | 30 | 20 | 60 | 50 | 0 | 20 | 0 | 90 |
| Pigweed, Palmer | 30 | 100 | 90 | 100 | 100 | 100 | 100 | 100 | 100 | 100 | 10 | 100 | 90 | 90 |
| Ragweed | 0 | 90 | 60 | 100 | 100 | 100 | 100 | 100 | 80 | 90 | 0 | 90 | 70 | 90 |
| Ryegrass, Italian | 0 | 80 | 60 | 100 | 70 | 100 | 90 | 80 | 70 | 90 | 0 | 80 | 40 | 100 |
| Soybean | 50 | 100 | 100 | 100 | 100 | 100 | 100 | 100 | 100 | 100 | 30 | 100 | 100 | 100 |
| Wheat | 0 | 30 | 20 | 90 | 30 | 80 | 80 | 30 | 70 | 30 | 0 | 20 | 20 | 90 |
| | Compounds | | | | | | | | | | | | | |
| 500 g ai/ha | 170 | 171 | 172 | 173 | 174 | 175 | 177 | 178 | 179 | 191 | 192 | 194 | 195 | 196 |
| Postemergence | | | | | | | | | | | | | | |
| Barnyardgrass | 50 | 40 | 0 | 10 | 60 | 50 | 70 | 60 | 10 | 10 | 50 | 30 | 60 | — |
| Blackgrass | 80 | 80 | 0 | 0 | 90 | 50 | 90 | 90 | 0 | 0 | 30 | 30 | 90 | 0 |
| Corn | 50 | 20 | 0 | 0 | 80 | 20 | 60 | 90 | 10 | 0 | 20 | 20 | 80 | 0 |
| Foxtail, Giant | — | — | — | — | — | — | — | — | — | — | — | — | — | 0 |
| Foxtail, Green | 90 | 50 | 0 | 40 | 90 | 70 | 90 | 90 | 20 | 0 | 80 | 50 | 90 | — |
| Galium | 100 | 100 | 0 | 50 | 100 | 90 | 100 | 100 | 90 | 50 | 100 | — | 100 | 0 |
| Goosegrass | — | — | — | — | — | — | — | — | — | — | — | — | — | 0 |
| Horseweed | 90 | 80 | 0 | 10 | 30 | 60 | 80 | 90 | 40 | 0 | 30 | 0 | 70 | 0 |
| Kochia | 100 | 90 | 30 | 30 | 100 | 100 | 100 | 90 | 30 | 20 | 100 | 80 | 100 | 0 |
| Oat, Wild | 60 | 50 | 0 | 0 | 70 | 20 | 80 | 90 | 0 | 0 | 50 | 20 | 90 | 0 |
| Pigweed, Palmer | 100 | 100 | 0 | 20 | 100 | 90 | 100 | 90 | 60 | 30 | 100 | 20 | 100 | 0 |
| Ragweed | 90 | 70 | 0 | 30 | 70 | 70 | 70 | 90 | 10 | 0 | 100 | 50 | 70 | 0 |
| Ryegrass, Italian | 90 | 80 | 0 | 0 | 70 | 60 | 90 | 100 | 20 | 0 | 50 | 70 | 80 | 0 |
| Soybean | 100 | 100 | 0 | 30 | 100 | 100 | 100 | 100 | 40 | 90 | 100 | 90 | 100 | 0 |
| Wheat | 50 | 40 | 0 | 0 | 40 | 10 | 50 | 50 | 10 | 10 | 20 | 20 | 50 | 0 |
| | Compounds | | | | | | | | | | | | | |
| 500 g ai/ha | 203 | 210 | 211 | 212 | 213 | 214 | 215 | 216 | 217 | 218 | 219 | 220 | 221 | 223 |
| Postemergence | | | | | | | | | | | | | | |
| Barnyardgrass | 0 | 100 | 100 | 50 | 30 | 20 | 70 | 60 | 80 | 100 | 100 | 90 | 10 | 40 |
| Blackgrass | 0 | 90 | 100 | 100 | 70 | 80 | 90 | 100 | 100 | 100 | 50 | 50 | 0 | 70 |
| Corn | 10 | 90 | 90 | 90 | 20 | 70 | 90 | 20 | 70 | 70 | 30 | 20 | 0 | 80 |
| Foxtail, Giant | — | — | — | — | — | — | — | — | — | — | — | — | — | — |
| Foxtail, Green | 10 | 90 | 90 | 90 | 70 | 80 | 90 | 90 | 90 | 90 | 90 | 50 | 0 | 60 |
| Galium | ·— | 100 | — | — | — | — | — | — | — | 100 | 100 | 100 | 0 | — |
| Goosegrass | — | — | — | — | — | — | — | — | — | — | — | — | — | — |
| Horseweed | 0 | 80 | 90 | 50 | 30 | 40 | 70 | 50 | 100 | 70 | 70 | 30 | 20 | 30 |
| Kochia | 40 | 90 | 90 | 90 | 90 | 100 | 90 | 90 | 90 | 100 | 90 | 90 | 0 | 90 |
| Oat, Wild | 10 | 80 | 90 | 80 | 20 | 20 | 50 | 20 | 40 | 80 | 40 | 0 | 0 | 30 |
| Pigweed, Palmer | 40 | 100 | 100 | 90 | 100 | 100 | 100 | 90 | 100 | 90 | 100 | 100 | 0 | 90 |
| Ragweed | 20 | 80 | 70 | 70 | 80 | 40 | 70 | 80 | 100 | 80 | 90 | 0 | 0 | 10 |
| Ryegrass, Italian | 0 | 90 | 90 | 70 | 60 | 40 | 90 | 60 | 90 | 90 | 70 | 70 | 0 | 90 |
| Soybean | 100 | 10 | 100 | 100 | 100 | 100 | 100 | 100 | 100 | 100 | 100 | 100 | 0 | 100 |
| Wheat | 10 | 90 | 90 | 80 | 20 | 30 | 70 | 30 | 50 | 90 | 40 | 20 | 0 | 30 |
| | Compounds | | | | | | | | | | | | | |
| 500 g ai/ha | 224 | 225 | 226 | 227 | 228 | 229 | 231 | 232 | 233 | 234 | 235 | 236 | 240 | 241 |
| Postemergence | | | | | | | | | | | | | | |
| Barnyardgrass | 50 | 70 | 80 | 70 | 90 | 0 | 90 | 20 | 20 | 100 | 90 | 90 | 80 | 100 |
| Blackgrass | 70 | 90 | 90 | 80 | 90 | 0 | 90 | 50 | 40 | 90 | 90 | 90 | 90 | 100 |
| Corn | 90 | 90 | 60 | 80 | 80 | 0 | 90 | 0 | 20 | 90 | 90 | 80 | 80 | 100 |
| Foxtail, Giant | — | — | — | — | — | — | — | — | — | — | — | — | — | — |
| Foxtail, Green | 70 | 90 | — | 90 | 90 | 0 | — | 30 | 20 | — | — | — | 90 | 90 |
| Galium | — | — | 100 | 100 | 100 | 0 | 100 | — | — | 100 | 100 | 100 | 100 | 100 |
| Goosegrass | — | — | — | — | — | — | — | — | — | — | — | — | — | — |
| Horseweed | 50 | 80 | 30 | 40 | 30 | 0 | 90 | 0 | 0 | 20 | 70 | 70 | 70 | 90 |
| Kochia | 90 | 90 | 80 | 100 | 70 | 0 | 90 | 60 | 10 | 80 | 90 | 90 | 90 | 70 |
| Oat, Wild | 70 | 70 | 60 | 60 | 80 | 0 | 90 | 0 | 20 | 80 | 60 | 40 | 70 | 90 |
| Pigweed, Palmer | 90 | 100 | 80 | 100 | 90 | 0 | 90 | 10 | 10 | 70 | 90 | 90 | 100 | 90 |
| Ragweed | 70 | 90 | 60 | 50 | 30 | 0 | 70 | 30 | 20 | 70 | 80 | 90 | 80 | 90 |
| Ryegrass, Italian | 100 | 90 | 90 | 100 | 100 | — | 100 | 60 | 60 | 90 | 100 | 90 | 90 | 100 |
| Soybean | 100 | 100 | 100 | 100 | 100 | 40 | 100 | 100 | 80 | 100 | 100 | 100 | 100 | 100 |
| Wheat | 60 | 90 | 30 | 60 | 90 | 0 | 90 | 10 | 10 | 80 | 30 | 30 | 50 | 90 |
| | Compounds | | | | | | | | | | | | | |
| 500 g ai/ha | 242 | 248 | 249 | 252 | 253 | 254 | 256 | 258 | 259 | 260 | 262 | 263 | 264 | 265 |
| Postemergence | | | | | | | | | | | | | | |
| Barnyardgrass | 100 | 70 | 10 | 80 | 30 | 80 | 90 | 90 | 70 | 50 | 90 | 90 | 90 | 100 |
| Blackgrass | 100 | 100 | 0 | 80 | 80 | 90 | 90 | 100 | 80 | 30 | 90 | 90 | 60 | 90 |
| Corn | 90 | 70 | 10 | 80 | 80 | 90 | 90 | 80 | 30 | 60 | 80 | 90 | 80 | 70 |
| Foxtail, Giant | — | — | — | — | — | — | — | — | — | — | — | — | — | — |
| Foxtail, Green | 90 | 90 | 30 | — | — | — | — | 90 | 90 | 60 | 90 | 90 | 90 | 90 |
| Galium | 100 | — | — | 100 | 100 | 100 | 100 | 100 | 100 | 100 | 100 | 100 | 100 | 100 |
| Goosegrass | — | — | — | — | — | — | — | — | — | — | — | — | — | — |
| Horseweed | 80 | 40 | 0 | 50 | 20 | 20 | 40 | 90 | 0 | 10 | 10 | 20 | 60 | 60 |
| Kochia | 80 | 100 | 80 | 100 | 80 | 90 | 80 | 100 | 80 | 90 | 90 | 70 | 100 | 100 |
| Oat, Wild | 90 | 70 | 0 | 10 | 90 | 90 | 90 | 90 | 30 | 50 | 80 | 50 | 50 | 80 |
| Pigweed, Palmer | 90 | 100 | 70 | 100 | 70 | 100 | 100 | 100 | 90 | 50 | 90 | 80 | 90 | 100 |
| Ragweed | 90 | 90 | 0 | 80 | 70 | 100 | 80 | 90 | 50 | 0 | 70 | 90 | 90 | 90 |
| Ryegrass, Italian | 100 | 100 | 0 | 80 | 50 | 80 | 100 | 80 | 50 | 60 | 90 | 90 | 90 | 90 |
| Soybean | 100 | 100 | 100 | 100 | 100 | 100 | 100 | 100 | 100 | 100 | 100 | 100 | 100 | 100 |
| Wheat | 90 | 90 | 10 | 30 | 70 | 80 | 90 | 50 | 20 | 20 | 50 | 80 | 30 | 70 |
| | Compounds | | | | | | | | | | | | | |
| 500 g ai/ha | 266 | 267 | 269 | 270 | 271 | 272 | 273 | 275 | 276 | 277 | 278 | 279 | 280 | 282 |
| Postemergence | | | | | | | | | | | | | | |
| Barnyardgrass | 90 | 90 | 100 | 100 | 90 | 0 | 70 | 90 | 90 | 100 | 90 | 100 | 90 | — |
| Blackgrass | 80 | 50 | 90 | 90 | 90 | 0 | 80 | 90 | 90 | 90 | 90 | 90 | 100 | 0 |
| Corn | 70 | 90 | 100 | 90 | 90 | 0 | 30 | 90 | 90 | 100 | 90 | 90 | 90 | 0 |
| Foxtail, Giant | — | — | — | — | — | — | — | — | — | — | — | — | — | 0 |
| Foxtail, Green | 90 | 90 | 90 | 90 | 90 | 0 | 90 | 90 | 90 | 90 | 90 | 90 | 90 | — |
| Galium | 100 | 100 | 100 | 100 | 100 | 0 | 100 | 100 | 100 | 100 | 100 | 100 | 100 | 0 |
| Goosegrass | — | — | — | — | — | — | — | — | — | — | — | — | — | 0 |
| Horseweed | 80 | 50 | 90 | 60 | 90 | 10 | 40 | 90 | 40 | 30 | 50 | 20 | 60 | 0 |
| Kochia | 100 | 100 | 90 | 60 | 90 | 0 | 90 | 90 | 90 | 80 | 80 | 70 | 90 | 0 |
| Oat, Wild | 60 | 40 | 90 | 90 | 90 | 0 | 30 | 80 | 90 | 90 | 90 | 90 | 90 | 0 |
| Pigweed, Palmer | 100 | 100 | 100 | 90 | 100 | 20 | 100 | 100 | 90 | 90 | 90 | 90 | 100 | 10 |
| Ragweed | 80 | 80 | 80 | 50 | 70 | 0 | 60 | 100 | 90 | 70 | 80 | 60 | 80 | 0 |
| Ryegrass, Italian | 90 | 90 | 100 | 90 | 80 | 0 | 50 | 100 | 100 | 100 | 90 | 100 | 90 | 0 |
| Soybean | 100 | 100 | 100 | 100 | 100 | 50 | 100 | 100 | 100 | 100 | 100 | 100 | 100 | 10 |
| Wheat | 40 | 30 | 90 | 90 | 80 | 0 | 40 | 80 | 90 | 90 | 90 | 90 | 90 | 0 |
| | Compounds | | | | | | | | | | | | | |
| 500 g ai/ha | 283 | 284 | 285 | 288 | 289 | 290 | 291 | 292 | 294 | 299 | 30 0 | 301 | 302 | 303 |
| Postemergence | | | | | | | | | | | | | | |
| Barnyardgrass | — | — | — | 10 | 60 | 90 | 90 | 20 | 50 | 50 | 50 | 40 | 10 | 70 |
| Blackgrass | 40 | 40 | 0 | 10 | 100 | 90 | 50 | 20 | 80 | 90 | 80 | 100 | 30 | 100 |
| Corn | 20 | 10 | 0 | 10 | 30 | 30 | 80 | 20 | 90 | 20 | 70 | 80 | 10 | 80 |
| Foxtail, Giant | 90 | 20 | 0 | — | — | — | — | — | — | — | — | — | — | — |
| Foxtail, Green | — | — | — | 20 | 90 | 90 | 90 | 40 | 70 | 40 | 50 | 70 | 10 | 80 |
| Galium | 80 | 90 | 30 | 50 | 100 | 100 | 100 | 80 | 90 | 90 | 90 | 100 | 90 | 100 |
| Goosegrass | 40 | 30 | 0 | — | — | — | — | — | — | — | — | — | — | — |
| Horseweed | — | 50 | 0 | 0 | 40 | 40 | 60 | 60 | 70 | 30 | 30 | 0 | 50 | 20 |
| Kochia | 80 | 50 | 0 | 80 | 90 | 90 | 100 | 90 | 60 | 70 | 60 | 80 | 70 | 80 |
| Oat, Wild | 10 | 10 | 0 | 0 | 60 | 90 | 80 | 50 | 10 | 60 | 40 | 50 | 10 | 70 |
| Pigweed, Palmer | 80 | 60 | 0 | 50 | 100 | 100 | 100 | 80 | 60 | 60 | 40 | 60 | 20 | 80 |
| Ragweed | 50 | 0 | 0 | 30 | 70 | 80 | 80 | 20 | 70 | 0 | 50 | 60 | 20 | 60 |
| Ryegrass, Italian | 50 | 0 | 0 | 20 | 90 | 90 | 80 | 60 | 90 | 90 | 80 | 80 | 50 | 90 |
| Soybean | 90 | 30 | 10 | 80 | 90 | 100 | 100 | 100 | 100 | 100 | 100 | 100 | 90 | 100 |
| Wheat | 10 | 10 | 0 | 20 | 40 | 60 | 70 | 30 | 40 | 40 | 30 | 50 | 10 | 50 |
| | Compounds | | | | | | | | | | | | | |
| 500 g ai/ha | 305 | 306 | 307 | 308 | 310 | 311 | 312 | 313 | 314 | 315 | 316 | 317 | 318 | 319 |
| Postemergence | | | | | | | | | | | | | | |
| Barnyardgrass | 90 | 10 | 80 | 30 | 80 | 40 | 10 | 70 | 60 | 80 | 50 | 70 | 70 | 60 |
| Blackgrass | 90 | 10 | 90 | 60 | 90 | 90 | 40 | 100 | 100 | 90 | 70 | 60 | 90 | 80 |
| Corn | 90 | 10 | 50 | 40 | 60 | 40 | 10 | 90 | 90 | 40 | 70 | 40 | 80 | 50 |
| Foxtail, Giant | — | — | — | — | — | — | — | — | — | — | 90 | 70 | 90 | 80 |
| Foxtail, Green | 90 | 50 | 90 | 50 | 50 | 40 | 20 | 90 | 90 | 90 | — | — | — | — |
| Galium | 100 | 50 | 90 | 80 | 90 | 90 | 80 | 100 | 100 | 70 | 90 | 70 | 100 | 70 |
| Goosegrass | — | — | — | — | — | — | — | — | — | — | — | — | — | — |
| Horseweed | 100 | 0 | 30 | 0 | 30 | 50 | 0 | 90 | 90 | 30 | 0 | 0 | 20 | 30 |
| Kochia | 80 | 10 | 70 | 20 | 90 | 70 | 80 | 90 | 90 | 50 | 80 | 70 | 80 | 90 |
| Oat, Wild | 60 | 0 | 80 | 0 | 40 | 10 | 0 | 60 | 40 | 90 | 20 | 30 | 80 | 30 |
| Pigweed, Palmer | 80 | 20 | 50 | 50 | 90 | 60 | 50 | 100 | 90 | 100 | 60 | 50 | 60 | 80 |
| Ragweed | 80 | 0 | 50 | 30 | 50 | 40 | 40 | 90 | 90 | 70 | 0 | 0 | 30 | 70 |
| Ryegrass, Italian | 60 | 0 | 70 | 20 | 80 | 30 | 30 | 90 | 90 | 80 | 80 | 40 | 100 | 50 |
| Soybean | 100 | 90 | 100 | 100 | 100 | 100 | 100 | 100 | 100 | 100 | 100 | 100 | 100 | 100 |
| Wheat | 90 | 10 | 80 | 20 | 50 | 10 | 10 | 50 | 30 | 90 | 20 | 20 | 50 | 10 |
| | Compounds | | | | | | | | | | | | | |
| 500 g ai/ha | 320 | 321 | 322 | 323 | 324 | 325 | 326 | 327 | 328 | 329 | 330 | 331 | 332 | 333 |
| Postemergence | | | | | | | | | | | | | | |
| Barnyardgrass | 20 | 90 | 50 | 60 | 80 | 80 | 80 | 90 | 90 | 60 | 70 | 30 | 70 | 10 |
| Blackgrass | 10 | 90 | 40 | 90 | 90 | 70 | 80 | 90 | 80 | 90 | 90 | 20 | 70 | 20 |
| Corn | 40 | 90 | 10 | 50 | 90 | 50 | 80 | 90 | 70 | 80 | 90 | 40 | 30 | 20 |
| Foxtail, Giant | 40 | 90 | 90 | 90 | 90 | — | 90 | 90 | 90 | 90 | 90 | 40 | 90 | 40 |
| Foxtail, Green | — | — | — | — | — | 90 | — | — | — | — | — | — | — | — |
| Galium | 70 | 100 | 70 | 100 | 100 | 100 | 100 | 100 | 100 | 100 | 100 | 50 | 100 | 60 |
| Goosegrass | — | — | — | — | — | — | — | — | — | — | — | — | — | — |
| Horseweed | 40 | 0 | 0 | 40 | 80 | 60 | 50 | 60 | 60 | 0 | 20 | 0 | 0 | 0 |
| Kochia | 80 | 80 | 80 | 90 | 100 | 90 | 80 | 90 | 80 | 90 | 100 | 70 | 60 | 50 |
| Oat, Wild | 10 | 90 | 30 | 20 | 70 | 70 | 50 | 90 | 40 | 80 | 70 | 0 | 30 | 0 |
| Pigweed, Palmer | 50 | 60 | 90 | 100 | 100 | 100 | 60 | 50 | 90 | 80 | 100 | 50 | 70 | 20 |
| Ragweed | 50 | 40 | 50 | 70 | 70 | 70 | 60 | 40 | 60 | 40 | 40 | 0 | 20 | 10 |
| Ryegrass, Italian | 30 | 80 | 40 | 60 | 50 | 80 | 80 | 80 | 90 | 70 | 90 | 10 | 70 | 10 |
| Soybean | 100 | 100 | 100 | 100 | 100 | 100 | 100 | 100 | 100 | 100 | 100 | 100 | 100 | 50 |
| Wheat | 30 | 80 | 20 | 20 | 30 | 60 | 40 | 30 | 30 | 50 | 30 | 20 | 20 | 10 |
| | Compounds | | | | | | | | | | | | | |
| 500 g ai/ha | 334 | 335 | 336 | 337 | 338 | 339 | 340 | 341 | 342 | 343 | 344 | 345 | 346 | 347 |
| Postemergence | | | | | | | | | | | | | | |
| Barnyardgrass | 90 | 20 | 60 | 60 | 70 | 10 | 60 | 70 | 70 | 70 | 70 | 60 | 70 | 20 |
| Blackgrass | 80 | 20 | 90 | 90 | 70 | 0 | 60 | 50 | 80 | 90 | 90 | 90 | 90 | 60 |
| Corn | 90 | 10 | 60 | 60 | 80 | 0 | 60 | 80 | 70 | 100 | 90 | 80 | 90 | 10 |
| Foxtail, Giant | 90 | 50 | — | — | — | — | — | — | — | — | 90 | 90 | 90 | — |
| Foxtail, Green | — | — | 60 | 40 | 70 | 10 | 70 | 70 | 90 | 90 | — | — | — | 60 |
| Galium | 100 | 50 | 90 | 90 | 100 | 50 | 100 | 100 | 80 | 100 | 100 | 100 | 100 | 80 |
| Goosegrass | — | — | — | — | — | — | — | — | — | — | — | — | — | — |
| Horseweed | 40 | 0 | 0 | 20 | 20 | 10 | 60 | 60 | 50 | 80 | 30 | 30 | 0 | 20 |
| Kochia | 80 | 30 | 80 | 70 | 100 | 60 | 90 | 90 | 60 | 90 | 80 | 90 | 80 | 70 |
| Oat, Wild | 40 | 0 | 70 | 30 | 20 | 0 | 20 | 20 | 80 | 90 | 90 | 30 | 60 | 10 |
| Pigweed, Palmer | 50 | 60 | 70 | 70 | 100 | 70 | 100 | 100 | 100 | 100 | 40 | 80 | 70 | 80 |
| Ragweed | 60 | 20 | 10 | 60 | 40 | 10 | 80 | 90 | 80 | 80 | 70 | 60 | 40 | 50 |
| Ryegrass, Italian | 70 | 30 | 80 | 70 | 80 | 20 | 80 | 60 | 70 | 90 | 100 | 80 | 80 | 30 |
| Soybean | 100 | 100 | 100 | 100 | 100 | 20 | 100 | 100 | 90 | 100 | 100 | 100 | 100 | 100 |
| Wheat | 50 | 20 | 40 | 20 | 40 | 0 | 30 | 20 | 80 | 60 | 80 | 30 | 70 | 10 |
| | Compounds | | | | | | | | | | | | | |
| 500 g ai/ha | 348 | 349 | 350 | 352 | 353 | 354 | 355 | 356 | 357 | 358 | 359 | 360 | 361 | 362 |
| Postemergence | | | | | | | | | | | | | | |
| Barnyardgrass | 40 | 90 | 30 | 10 | 30 | 70 | 10 | 20 | 100 | 100 | 90 | 100 | 90 | 90 |
| Blackgrass | 100 | 100 | 20 | 0 | 30 | 20 | 0 | 50 | 100 | 100 | 100 | 90 | 90 | 90 |
| Corn | 30 | 90 | 20 | 0 | 10 | 60 | 10 | 20 | 80 | 90 | 80 | 90 | 50 | 40 |
| Foxtail, Giant | — | — | — | — | — | — | — | — | — | — | — | — | — | — |
| Foxtail, Green | 70 | 90 | 80 | 10 | 40 | 90 | 10 | 30 | 90 | 90 | 80 | 90 | 90 | 90 |
| Galium | 100 | 100 | 100 | 50 | 90 | 100 | 10 | 100 | 100 | 100 | 100 | 100 | 100 | 100 |
| Goosegrass | — | — | — | — | — | — | — | — | — | — | — | — | — | — |
| Horseweed | 40 | 50 | 40 | 10 | 0 | 80 | 0 | 40 | 80 | 90 | 90 | 30 | 10 | 10 |
| Kochia | 100 | 90 | 90 | 50 | 70 | 90 | 0 | 80 | 70 | 70 | 80 | 70 | 80 | 90 |
| Oat, Wild | 60 | 70 | 10 | 0 | 0 | 90 | 0 | 20 | 90 | 80 | 90 | 90 | 80 | 60 |
| Pigweed, Palmer | 100 | 90 | 100 | 20 | 100 | 100 | 10 | 70 | 100 | 80 | 100 | 90 | 60 | 80 |
| Ragweed | 60 | 40 | 50 | 20 | 60 | 70 | 0 | 30 | 60 | 80 | 80 | 80 | 60 | 60 |
| Ryegrass, Italian | 70 | 80 | 30 | 10 | 40 | 70 | 0 | 80 | 100 | 100 | 100 | 100 | 90 | 90 |
| Soybean | 100 | 100 | 100 | 90 | 80 | 100 | 50 | 100 | 100 | 100 | 100 | 100 | 100 | 100 |
| Wheat | 30 | 80 | 10 | 10 | 20 | 60 | 10 | 20 | 80 | 80 | 90 | 50 | 80 | 60 |
| | Compounds | | | | | | | | | | | | | |
| 500 g ai/ha | 363 | 364 | 377 | 378 | 379 | 380 | 382 | 386 | 387 | 388 | 391 | 392 | 393 | 394 |
| Postemergence | | | | | | | | | | | | | | |
| Barnyardgrass | 50 | 80 | 50 | 80 | 60 | 50 | 50 | 30 | 60 | 20 | 60 | 80 | 50 | 70 |
| Blackgrass | 60 | 100 | 20 | 50 | 30 | 70 | 40 | 50 | 90 | 0 | 80 | 90 | 0 | 70 |
| Corn | 10 | 50 | 30 | 80 | 50 | 50 | 50 | 20 | 70 | 30 | 60 | 80 | 50 | 50 |
| Foxtail, Giant | — | — | — | — | — | — | — | — | — | — | — | — | — | — |
| Foxtail, Green | 50 | 90 | 40 | 90 | 20 | 80 | 60 | 50 | 80 | 90 | 80 | 90 | 70 | 90 |
| Galium | 100 | 100 | 100 | 100 | 100 | 100 | 100 | 100 | 100 | 100 | 100 | 100 | 100 | 100 |
| Goosegrass | — | — | — | — | — | — | — | — | — | — | — | — | — | — |
| Horseweed | 0 | 0 | 30 | 70 | 30 | 60 | 50 | 60 | 60 | 30 | 60 | 90 | 20 | 20 |
| Kochia | 80 | 90 | 90 | 100 | 90 | 100 | 100 | 90 | 100 | 90 | 100 | 90 | 90 | 90 |
| Oat, Wild | 20 | 70 | 0 | 30 | 10 | 0 | 0 | 0 | 20 | 40 | 80 | 90 | 40 | 70 |
| Pigweed, Palmer | 90 | 90 | 100 | 100 | 100 | 100 | 100 | 100 | 100 | 100 | 100 | 100 | 90 | 80 |
| Ragweed | 50 | 80 | 90 | 90 | 90 | 100 | 90 | 80 | 80 | 50 | 90 | 90 | 70 | 70 |
| Ryegrass, Italian | 50 | 100 | 50 | 60 | 50 | 60 | 60 | 60 | 80 | 30 | 100 | 100 | 80 | 90 |
| Soybean | 100 | 100 | 100 | 100 | 100 | 100 | 100 | 100 | 100 | 100 | 100 | 100 | 100 | 100 |
| Wheat | 20 | 80 | 30 | 40 | 20 | 20 | 40 | 10 | 30 | 10 | 40 | 60 | 20 | 50 |
| | Compounds | | | | | | | | | | | | | |
| 500 g ai/ha | 395 | 396 | 397 | 398 | 399 | 400 | 401 | 402 | 403 | 408 | 410 | 411 | 412 | 413 |
| Postemergence | | | | | | | | | | | | | | |
| Barnyardgrass | 70 | 50 | 60 | 50 | 30 | 50 | 20 | 50 | 70 | 60 | 90 | 50 | 80 | 40 |
| Blackgrass | 90 | 70 | 90 | 90 | 20 | 70 | 30 | 60 | 80 | 70 | 90 | 30 | 90 | 20 |
| Corn | 90 | 60 | 70 | 80 | 10 | 50 | 40 | 60 | 80 | 80 | 80 | 10 | 20 | 20 |
| Foxtail, Giant | — | — | — | — | — | — | — | — | — | — | — | — | — | — |
| Foxtail, Green | 80 | 80 | 90 | 90 | 40 | 40 | 20 | 90 | 90 | 60 | 90 | 70 | 90 | 60 |
| Galium | 100 | 100 | — | — | — | — | — | — | 100 | — | 100 | 100 | 100 | 100 |
| Goosegrass | — | — | — | — | — | — | — | — | — | — | — | — | — | — |
| Horseweed | 60 | 60 | 80 | 60 | 70 | 20 | 70 | 70 | 60 | 60 | 60 | 20 | 30 | 60 |
| Kochia | 100 | 100 | 100 | 90 | 90 | 90 | 100 | 90 | 80 | 90 | 90 | 90 | 90 | 90 |
| Oat, Wild | 60 | 40 | 70 | 60 | 0 | 20 | 0 | 20 | 90 | 90 | 90 | 0 | 50 | 10 |
| Pigweed, Palmer | 100 | 90 | 100 | 100 | 100 | 100 | 90 | 100 | 100 | 80 | 100 | 90 | 90 | 100 |
| Ragweed | 90 | 90 | 70 | 90 | 80 | 100 | 70 | 100 | 90 | 80 | 100 | 70 | 100 | 70 |
| Ryegrass, Italian | 80 | 50 | 100 | 80 | 50 | 60 | 0 | 70 | 90 | 100 | 100 | 50 | 90 | 60 |
| Soybean | 100 | 100 | 100 | 70 | 60 | 50 | 20 | 60 | 100 | 100 | 100 | 100 | 100 | 100 |
| Wheat | 60 | 30 | 80 | 80 | 20 | 30 | 20 | 20 | 70 | 90 | 90 | 10 | 20 | 10 |
| | Compounds | | | | | | | | | | | | | |
| 125 g ai/ha | 150 | 151 | 152 | 153 | 154 | 155 | 156 | 157 | 158 | 164 | 165 | 167 | 168 | 169 |
| Postemergence | | | | | | | | | | | | | | |
| Barnyardgrass | 10 | 70 | 60 | 90 | 20 | 90 | 70 | 30 | 40 | 40 | 0 | 20 | 10 | 20 |
| Blackgrass | 0 | 80 | 50 | 90 | 20 | 90 | 80 | 60 | 40 | 70 | 0 | 0 | 0 | 90 |
| Corn | 0 | 60 | 40 | 80 | 20 | 40 | 30 | 10 | 50 | 30 | 0 | 10 | 10 | 80 |
| Foxtail, Giant | — | — | — | — | — | — | — | — | — | — | — | — | — | — |
| Foxtail, Green | 0 | 80 | 40 | 90 | 40 | 90 | 90 | 70 | 60 | 50 | 0 | 50 | 20 | 30 |
| Galium | 0 | 100 | 100 | 90 | 100 | 100 | 100 | 100 | 100 | 100 | 0 | 90 | 80 | 100 |
| Goosegrass | — | — | — | — | — | — | — | — | — | — | — | — | — | — |
| Horseweed | 0 | 40 | 0 | 60 | 80 | 70 | 50 | 30 | 20 | 20 | 0 | 10 | 0 | 0 |
| Kochia | 0 | 90 | 80 | 60 | 100 | 80 | 80 | 90 | 70 | 80 | 0 | 90 | 80 | 90 |
| Oat, Wild | 0 | 20 | 0 | 50 | 10 | 20 | 20 | 20 | 30 | 30 | 0 | 0 | 0 | 80 |
| Pigweed, Palmer | 0 | 90 | 80 | 70 | 90 | 90 | 80 | 90 | 80 | 90 | 0 | 90 | 70 | 70 |
| Ragweed | 0 | 70 | 40 | 80 | 100 | 100 | 80 | 100 | 90 | 50 | 0 | 90 | 60 | 50 |
| Ryegrass, Italian | 0 | 60 | 30 | 90 | 30 | 70 | 50 | 30 | 40 | 50 | 0 | 20 | 20 | 60 |
| Soybean | 20 | 100 | 100 | 100 | 90 | 100 | 100 | 100 | 100 | 100 | 30 | 100 | 100 | 100 |
| Wheat | 0 | 20 | 20 | 90 | 10 | 60 | 60 | 20 | 30 | 30 | 0 | 10 | 10 | 6 |
| | Compounds | | | | | | | | | | | | | |
| 125 g ai/ha | 170 | 171 | 172 | 173 | 174 | 175 | 177 | 178 | 179 | 180 | 191 | 192 | 193 | 194 |
| Postemergence | | | | | | | | | | | | | | |
| Barnyardgrass | 20 | 30 | 0 | 10 | 50 | 20 | 30 | 10 | 10 | 20 | 10 | 20 | 40 | 10 |
| Blackgrass | 30 | 30 | 0 | 0 | 60 | 10 | 90 | 60 | 0 | 10 | 0 | 30 | 60 | 0 |
| Corn | 30 | 20 | 0 | 0 | 70 | 0 | 40 | 60 | 0 | 10 | 0 | 10 | 0 | 10 |
| Foxtail, Giant | — | — | — | — | — | — | — | — | — | — | — | — | — | — |
| Foxtail, Green | 20 | 40 | 0 | 0 | 70 | 30 | 90 | 20 | 10 | 20 | 0 | 10 | 80 | 10 |
| Galium | 100 | 100 | 0 | 20 | 100 | 90 | 90 | 100 | 50 | 90 | 0 | 100 | 100 | — |
| Goosegrass | — | — | — | — | — | — | — | — | — | — | — | — | — | — |
| Horseweed | 80 | 20 | 0 | 10 | 0 | 80 | 30 | 70 | 0 | 0 | 0 | 0 | 0 | 0 |
| Kochia | 80 | 90 | 0 | 0 | 90 | 90 | 90 | 80 | 30 | 50 | 20 | 80 | 80 | 10 |
| Oat, Wild | 20 | 10 | 0 | 0 | 50 | 10 | 60 | 30 | 0 | 70 | 0 | 20 | 0 | 0 |
| Pigweed, Palmer | 100 | 100 | 0 | 0 | 90 | 90 | 90 | 90 | 10 | 70 | 0 | 90 | 70 | 0 |
| Ragweed | 70 | 60 | 0 | 0 | 60 | 20 | 80 | 60 | — | 60 | 0 | 90 | 60 | 0 |
| Ryegrass, Italian | 60 | 60 | 0 | 0 | 50 | 50 | 80 | 80 | 0 | 50 | 0 | 40 | 50 | 30 |
| Soybean | 100 | 100 | 0 | 0 | 100 | 100 | 100 | 100 | — | 100 | 40 | 100 | 100 | 90 |
| Wheat | 10 | 10 | 0 | 0 | 20 | 10 | 30 | 20 | 10 | 20 | 10 | 20 | 10 | 20 |
| | Compounds | | | | | | | | | | | | | |
| 125 g ai/ha | 195 | 196 | 203 | 210 | 211 | 212 | 213 | 214 | 215 | 216 | 217 | 218 | 219 | 220 |
| Postemergence | | | | | | | | | | | | | | |
| Barnyardgrass | 30 | — | 0 | 50 | 80 | 30 | 20 | 20 | 60 | 10 | 40 | 100 | 70 | 30 |
| Blackgrass | 50 | 0 | 0 | 90 | 90 | 50 | 0 | 0 | 90 | 30 | 90 | 90 | 40 | 40 |
| Corn | 50 | 0 | 0 | 80 | 70 | 20 | 10 | 60 | 70 | 10 | 70 | 70 | 20 | 10 |
| Foxtail, Giant | — | 0 | — | — | — | — | — | — | — | — | — | — | — | — |
| Foxtail, Green | 50 | — | 0 | 90 | 90 | 80 | 40 | 30 | 90 | 70 | 90 | 90 | 80 | 20 |
| Galium | 90 | 0 | — | 100 | — | — | — | — | — | — | — | 100 | 100 | 100 |
| Goosegrass | — | 0 | — | — | — | — | — | — | — | — | — | — | — | — |
| Horseweed | 90 | 0 | 0 | 80 | 80 | 0 | 20 | 0 | 0 | 0 | 80 | 0 | — | 60 |
| Kochia | 90 | 0 | 0 | 70 | 70 | 80 | 90 | 70 | 80 | 80 | 80 | 90 | 80 | 70 |
| Oat, Wild | 10 | 0 | 0 | 80 | 70 | 40 | 10 | 20 | 30 | 20 | 20 | 30 | 10 | 0 |
| Pigweed, Palmer | 90 | 30 | 0 | 90 | 90 | 90 | 100 | 100 | 90 | 80 | 100 | 90 | 100 | 90 |
| Ragweed | 70 | 0 | 0 | 60 | 50 | 20 | 30 | 40 | 60 | 60 | 90 | 80 | 50 | 0 |
| Ryegrass, Italian | 40 | 0 | 0 | 60 | 70 | 50 | 30 | 20 | 70 | 20 | 70 | 80 | 50 | 30 |
| Soybean | 100 | 0 | 100 | 0 | 100 | 100 | 100 | 100 | 100 | 100 | 100 | 100 | 100 | 100 |
| Wheat | 20 | 0 | 0 | 50 | 70 | 20 | 20 | 20 | 30 | 20 | 50 | 60 | 20 | 20 |
| | Compounds | | | | | | | | | | | | | |
| 125 g ai/ha | 221 | 223 | 224 | 225 | 226 | 227 | 228 | 229 | 230 | 231 | 232 | 233 | 234 | 235 |
| Postemergence | | | | | | | | | | | | | | |
| Barnyardgrass | 0 | 20 | 10 | 20 | 50 | 40 | 60 | 0 | 60 | 80 | 10 | 10 | 60 | 90 |
| Blackgrass | 0 | 30 | 30 | 50 | 60 | 50 | 80 | 0 | 40 | 80 | 0 | 0 | 80 | 70 |
| Corn | 0 | 20 | 50 | 80 | 20 | 20 | 40 | 0 | 60 | 70 | 0 | 0 | 70 | 50 |
| Foxtail, Giant | — | — | — | — | — | — | — | — | — | — | — | — | — | — |
| Foxtail, Green | 0 | 30 | 20 | 20 | — | 80 | 30 | 0 | — | — | 0 | 20 | — | — |
| Galium | 0 | 100 | 100 | — | 80 | 100 | 100 | 0 | 100 | 100 | — | 50 | 100 | 100 |
| Goosegrass | — | — | — | — | — | — | — | — | — | — | — | — | — | — |
| Horseweed | 0 | 20 | 30 | 20 | 10 | 20 | 10 | 0 | 40 | 60 | 0 | 0 | 20 | 30 |
| Kochia | 0 | 90 | 90 | 80 | 70 | 90 | 50 | 0 | 90 | 80 | 0 | 0 | 60 | 90 |
| Oat, Wild | 0 | 10 | 50 | 60 | 10 | 30 | 30 | 0 | 20 | 50 | 0 | 0 | 50 | 40 |
| Pigweed, Palmer | 0 | 80 | 90 | 90 | 40 | 100 | 70 | 0 | 100 | 90 | 0 | 0 | 30 | 70 |
| Ragweed | 0 | 0 | 50 | 90 | 30 | 10 | 10 | 0 | 90 | 70 | 0 | 0 | 50 | 70 |
| Ryegrass, Italian | 0 | 50 | 50 | 70 | 50 | 90 | 100 | 0 | 60 | 90 | 30 | 0 | 90 | 70 |
| Soybean | 0 | 100 | 100 | 100 | 100 | 100 | 100 | 40 | 100 | 100 | 100 | 70 | 100 | 100 |
| Wheat | 0 | 10 | 20 | 30 | 10 | 20 | 50 | 0 | 40 | 50 | 10 | 10 | 50 | 30 |
| | Compounds | | | | | | | | | | | | | |
| 125 g ai/ha | 236 | 240 | 241 | 242 | 248 | 249 | 252 | 253 | 254 | 256 | 258 | 259 | 260 | 262 |
| Postemerence | | | | | | | | | | | | | | |
| Barnyardgrass | 40 | 40 | 100 | 90 | 50 | 10 | 60 | 20 | 50 | 60 | 40 | 20 | 10 | 60 |
| Blackgrass | 20 | 80 | 90 | 80 | 90 | 0 | 80 | 40 | 80 | 90 | 50 | 70 | 20 | — |
| Corn | 30 | 60 | 60 | 60 | 60 | 0 | 60 | 20 | 80 | 90 | 70 | 10 | 10 | 60 |
| Foxtail, Giant | — | — | — | — | — | — | — | — | — | — | — | — | — | — |
| Foxtail, Green | — | 80 | 90 | 80 | 90 | 30 | — | — | — | — | 50 | 40 | 10 | 70 |
| Galium | 100 | 100 | 100 | 100 | 100 | 90 | 100 | 100 | 100 | 100 | 100 | 100 | 90 | 100 |
| Goosegrass | — | — | — | — | — | — | — | — | — | — | — | — | — | — |
| Horseweed | 70 | 0 | 50 | 50 | 10 | 0 | 20 | 0 | 30 | 30 | 70 | 0 | 0 | 10 |
| Kochia | 90 | 30 | 20 | 30 | 90 | 50 | 90 | 80 | 80 | 60 | 70 | 70 | 60 | 50 |
| Oat, Wild | 10 | 60 | 60 | 80 | 60 | 0 | 10 | 50 | 80 | 90 | 30 | 30 | 30 | 60 |
| Pigweed, Palmer | 90 | 90 | 70 | 80 | 90 | 40 | 90 | — | 90 | 80 | 100 | 90 | 10 | 70 |
| Ragweed | 80 | 50 | 50 | 60 | 90 | 0 | 70 | 70 | 90 | 70 | 70 | 20 | 0 | 40 |
| Ryegrass, Italian | 60 | 70 | 90 | 90 | 90 | 0 | 80 | 50 | 70 | 90 | 50 | 30 | 60 | 80 |
| Soybean | 100 | 100 | 100 | 100 | 100 | 100 | 100 | 100 | 100 | 100 | 100 | 100 | 100 | 100 |
| Wheat | 20 | 20 | 50 | 80 | 30 | 10 | 20 | 20 | 50 | 80 | 20 | 20 | 20 | 30 |
| | Compounds | | | | | | | | | | | | | |
| 125 g ai/ha | 263 | 264 | 265 | 266 | 267 | 268 | 269 | 270 | 271 | 273 | 275 | 276 | 277 | 278 |
| Postemergence | | | | | | | | | | | | | | |
| Barnyardgrass | 70 | 50 | 70 | 70 | 60 | 60 | 80 | 90 | 70 | 30 | 50 | 30 | 100 | 50 |
| Blackgrass | 80 | 40 | 90 | 50 | 30 | 70 | 80 | 80 | 70 | 10 | 80 | 60 | 80 | 90 |
| Corn | 30 | 30 | 60 | 70 | 50 | 50 | 80 | 40 | 50 | 10 | 10 | 50 | 90 | 50 |
| Foxtail, Giant | — | — | — | — | — | — | — | — | — | — | — | — | — | — |
| Foxtail, Green | 80 | 20 | 90 | 90 | 70 | 90 | 90 | 80 | 90 | 40 | 70 | 40 | 90 | 50 |
| Galium | 100 | 100 | 100 | 100 | 100 | 100 | 90 | 100 | 90 | 70 | 100 | 100 | 100 | 100 |
| Goosegrass | — | — | — | — | — | — | — | — | — | — | — | — | — | — |
| Horseweed | 0 | 20 | 40 | 20 | 60 | 70 | 20 | 20 | 50 | 0 | 60 | 50 | 0 | 0 |
| Kochia | 70 | 80 | 90 | 80 | 90 | 90 | 80 | 30 | 60 | 70 | 80 | 70 | 70 | 80 |
| Oat, Wild | 10 | 10 | 30 | 10 | 0 | 60 | 80 | 90 | 80 | 0 | 60 | 80 | 80 | 80 |
| Pigweed, Palmer | 40 | 90 | 70 | 90 | 90 | 90 | 90 | 40 | 90 | 90 | 80 | 60 | 80 | 50 |
| Ragweed | 40 | 80 | 80 | 70 | 70 | 80 | 70 | 30 | 50 | 40 | 80 | 60 | 80 | 70 |
| Ryegrass, Italian | 80 | 50 | 80 | 70 | 60 | 60 | 100 | 80 | 60 | 0 | 90 | 80 | 90 | 80 |
| Soybean | 100 | 100 | 100 | 100 | 100 | 100 | 100 | 100 | 100 | 100 | 100 | 100 | 100 | 100 |
| Wheat | 30 | 20 | 40 | 20 | 20 | 70 | 80 | 80 | 70 | 10 | 40 | 60 | 50 | 70 |
| | Compounds | | | | | | | | | | | | | |
| 125 g ai/ha | 279 | 280 | 282 | 283 | 284 | 285 | 288 | 289 | 290 | 291 | 292 | 294 | 299 | 30 0 |
| Postemergence | | | | | | | | | | | | | | |
| Barnyardgrass | 90 | 50 | — | — | — | — | 10 | 30 | 50 | 30 | 10 | 10 | 10 | 10 |
| Blackgrass | 80 | 80 | 0 | 10 | 0 | 0 | 0 | 30 | 10 | 0 | 0 | 30 | 20 | 0 |
| Corn | 90 | 90 | 0 | 20 | 0 | 0 | 0 | 20 | 30 | 20 | 10 | 30 | 10 | 0 |
| Foxtail, Giant | — | — | 0 | 20 | 10 | 0 | — | — | — | — | — | — | — | — |
| Foxtail, Green | 90 | 60 | — | — | — | — | 10 | 20 | 80 | 30 | 30 | 10 | 10 | 10 |
| Galium | 100 | 100 | 0 | 50 | 70 | 0 | 60 | 100 | 100 | 100 | 70 | 100 | 80 | 60 |
| Goosegrass | — | — | 0 | 10 | 10 | 0 | — | — | — | — | — | — | — | — |
| Horseweed | 0 | 30 | 0 | — | 0 | 0 | 0 | 0 | 40 | 60 | 20 | 30 | 10 | 30 |
| Kochia | 20 | 60 | 0 | 70 | 10 | 0 | 40 | 80 | 80 | 90 | 70 | 60 | 10 | 50 |
| Oat, Wild | 80 | 80 | 0 | 10 | 10 | 0 | 0 | 30 | 10 | 50 | 0 | 10 | 20 | 0 |
| Pigweed, Palmer | 70 | 70 | 0 | 60 | 40 | 0 | 10 | 90 | 100 | 100 | 80 | 40 | 40 | 30 |
| Ragweed | 50 | 70 | 0 | 10 | 0 | 0 | 30 | 60 | 70 | 70 | 30 | 50 | 0 | 0 |
| Ryegrass, Italian | 90 | 90 | 0 | 20 | 0 | 0 | 0 | 40 | 50 | 70 | 0 | 40 | 70 | 10 |
| Soybean | 100 | 100 | 0 | 90 | 20 | 0 | 60 | 100 | 100 | 90 | 100 | 90 | 60 | 70 |
| Wheat | 80 | 80 | 0 | 10 | 10 | 0 | 10 | 20 | 30 | 30 | 20 | 10 | 20 | 10 |
| | Compounds | | | | | | | | | | | | | |
| 125 g ai/ha | 301 | 302 | 303 | 305 | 306 | 307 | 308 | 310 | 311 | 312 | 313 | 314 | 315 | 316 |
| Postemergence | | | | | | | | | | | | | | |
| Barnyardgrass | 10 | 10 | 20 | 30 | 10 | 50 | 10 | 20 | 10 | 10 | 40 | 30 | 60 | 10 |
| Blackgrass | 90 | 0 | 90 | 40 | 0 | 70 | 10 | 50 | 0 | 0 | 70 | 80 | 70 | 50 |
| Corn | 10 | 10 | 20 | 10 | 0 | 10 | 10 | 10 | 10 | 10 | 80 | 80 | 30 | 20 |
| Foxtail, Giant | — | — | — | — | — | — | — | — | — | — | — | — | — | 10 |
| Foxtail, Green | 10 | 0 | 50 | 50 | 30 | 60 | 20 | 50 | 30 | 0 | 40 | 60 | 80 | — |
| Galium | 100 | — | 100 | 100 | 30 | 80 | 70 | 90 | 90 | 60 | 100 | 100 | 70 | 40 |
| Goosegrass | — | — | — | — | — | — | — | — | — | — | — | — | — | — |
| Horseweed | 20 | 60 | 0 | 30 | 30 | 20 | 20 | 0 | 50 | 0 | 90 | 100 | 0 | 0 |
| Kochia | 50 | 20 | 70 | 50 | 10 | 40 | 20 | 70 | 30 | 20 | 90 | 90 | 40 | 30 |
| Oat, Wild | 20 | 0 | 50 | 30 | 0 | 20 | 0 | 0 | 0 | 0 | 20 | 40 | 50 | 0 |
| Pigweed, Palmer | 10 | 10 | 40 | 30 | 0 | 20 | 10 | 70 | 20 | 10 | 80 | 60 | 80 | 30 |
| Ragweed | 30 | 20 | 50 | 70 | 30 | 0 | 0 | 30 | 0 | 0 | 60 | 80 | 70 | 0 |
| Ryegrass, Italian | 70 | 0 | 60 | 30 | 0 | 60 | 0 | 30 | 10 | 30 | 80 | 80 | 60 | 30 |
| Soybean | 100 | 80 | 100 | 100 | 30 | 100 | 90 | 90 | 70 | 80 | 100 | 100 | 70 | 100 |
| Wheat | 20 | 10 | 30 | 40 | 0 | 10 | 10 | 10 | 10 | 10 | 30 | 20 | 80 | 10 |
| | Compounds | | | | | | | | | | | | | |
| 125 g ai/ha | 317 | 318 | 319 | 320 | 321 | 322 | 323 | 324 | 325 | 326 | 327 | 328 | 329 | 330 |
| Postemergence | | | | | | | | | | | | | | |
| Barnyardgrass | 10 | 50 | 10 | 0 | 20 | 40 | 30 | 50 | 40 | 30 | 40 | 60 | 10 | 50 |
| Blackgrass | 0 | 60 | 10 | 0 | 60 | 10 | 50 | 90 | 10 | 80 | 30 | 60 | 50 | 40 |
| Corn | 10 | 70 | 20 | 10 | 50 | 10 | 10 | 70 | 30 | 10 | 70 | 20 | 30 | 70 |
| Foxtail, Giant | 10 | 90 | 30 | 20 | 70 | 50 | 20 | 80 | — | 70 | 60 | 90 | 50 | 50 |
| Foxtail, Green | — | — | — | — | — | — | — | — | 60 | — | — | — | — | — |
| Galium | 50 | 100 | 60 | 50 | 100 | 70 | 100 | 100 | 100 | 100 | 70 | 80 | 60 | 100 |
| Goosegrass | — | — | — | — | — | — | — | — | — | — | — | — | — | — |
| Horseweed | 0 | 0 | 0 | 10 | 0 | 0 | 30 | 60 | 0 | 0 | 0 | 0 | 0 | 0 |
| Kochia | 20 | 50 | 40 | 40 | 60 | 60 | 90 | 90 | 90 | 80 | 40 | 50 | 70 | 80 |
| Oat, Wild | 0 | 40 | 0 | 10 | 10 | 0 | 20 | 10 | 10 | 20 | 40 | 20 | 30 | 30 |
| Pigweed, Palmer | 10 | 20 | 30 | 0 | 10 | 50 | 80 | 90 | 90 | 20 | 30 | 50 | 20 | 70 |
| Ragweed | 0 | 0 | 0 | 0 | 30 | 40 | 60 | 60 | 50 | 10 | 30 | 40 | 40 | 30 |
| Ryegrass, Italian | 0 | 60 | 30 | 20 | 60 | 20 | 10 | 40 | 30 | 40 | 40 | 50 | 50 | 40 |
| Soybean | 70 | 100 | 100 | 50 | 90 | 60 | 100 | 100 | 100 | 100 | 100 | 100 | 100 | 100 |
| Wheat | 10 | 40 | 10 | 10 | 20 | 20 | 20 | 30 | 20 | 20 | 20 | 30 | 20 | 20 |
| | Compounds | | | | | | | | | | | | | |
| 125 g ai/ha | 331 | 332 | 333 | 334 | 335 | 336 | 337 | 338 | 339 | 340 | 341 | 342 | 343 | 344 |
| Postemergence | | | | | | | | | | | | | | |
| Barnyardgrass | 10 | 40 | 10 | 20 | 10 | 10 | 10 | 30 | 10 | 50 | 20 | 60 | 20 | 40 |
| Blackgrass | 0 | 30 | 0 | 40 | 0 | 30 | 40 | 40 | 0 | 20 | 10 | 60 | 40 | 80 |
| Corn | 10 | 30 | 10 | 40 | 10 | 20 | 10 | 80 | 0 | 60 | 0 | 70 | 90 | 30 |
| Foxtail, Giant | 30 | 40 | 20 | 70 | 20 | — | — | — | — | — | — | — | — | 70 |
| Foxtail, Green | — | — | — | — | — | 20 | 30 | 30 | 0 | 30 | 30 | 80 | 50 | — |
| Galium | 50 | 70 | 0 | 100 | 50 | 60 | 40 | 100 | 60 | 90 | 90 | 70 | 100 | 90 |
| Goosegrass | — | — | — | — | — | — | — | — | — | — | — | — | — | — |
| Horseweed | 0 | 0 | 0 | 0 | 0 | 0 | 0 | 10 | 0 | 30 | 60 | 10 | 30 | 30 |
| Kochia | 40 | 10 | 30 | 80 | 10 | 60 | 50 | 90 | 30 | 90 | 80 | 40 | 80 | 70 |
| Oat, Wild | 0 | 10 | 0 | 10 | 0 | 10 | 10 | 20 | 0 | 10 | 10 | 70 | 60 | 60 |
| Pigweed, Palmer | 20 | 70 | 0 | 30 | 10 | 20 | 20 | 90 | 50 | 80 | 80 | 90 | 50 | 20 |
| Ragweed | 0 | 0 | 0 | 0 | 0 | 20 | 20 | 30 | 0 | 70 | 60 | 50 | 60 | 70 |
| Ryegrass, Italian | 0 | 60 | 0 | 50 | 10 | 50 | 20 | 70 | 0 | 60 | 30 | 70 | 70 | 80 |
| Soybean | 90 | 90 | 50 | 100 | 90 | 80 | 90 | 100 | — | 100 | 100 | 100 | 100 | 80 |
| Wheat | 10 | 20 | 10 | 20 | 10 | 10 | 10 | 20 | 0 | 10 | 10 | 70 | 20 | 30 |
| | Compounds | | | | | | | | | | | | | |
| 125 g ai/ha | 345 | 346 | 347 | 348 | 349 | 350 | 352 | 353 | 354 | 355 | 356 | 357 | 358 | 359 |
| Postemergence | | | | | | | | | | | | | | |
| Barnyardgrass | 30 | 40 | 10 | 10 | 30 | 10 | 10 | 10 | 30 | 10 | 10 | 50 | 90 | 30 |
| Blackgrass | 50 | 80 | 10 | 70 | 90 | 10 | 0 | 0 | 0 | 0 | 0 | 90 | 90 | 60 |
| Corn | 30 | 10 | 10 | 10 | 80 | 10 | 0 | 10 | 20 | 10 | 10 | 40 | 90 | 40 |
| Foxtail, Giant | 60 | 90 | — | — | — | — | — | — | — | — | — | — | — | — |
| Foxtail, Green | — | — | 20 | 50 | 70 | 30 | 20 | 20 | 40 | 10 | 10 | 30 | 90 | 50 |
| Galium | 100 | 100 | 90 | 100 | 100 | 80 | 60 | 70 | 100 | — | 90 | 100 | 100 | 100 |
| Goosegrass | — | — | — | — | — | — | — | — | — | — | — | — | — | — |
| Horseweed | 0 | 0 | 0 | 0 | 10 | 40 | 0 | 20 | 30 | 0 | 30 | 60 | 70 | 70 |
| Kochia | 80 | 70 | 50 | 70 | 80 | 70 | 40 | 60 | 80 | — | 50 | 50 | 40 | 50 |
| Oat, Wild | 20 | 0 | 10 | 20 | 30 | 0 | 0 | 0 | 60 | 0 | 10 | 30 | 30 | 60 |
| Pigweed, Palmer | 60 | 40 | 80 | 90 | 50 | 100 | — | 90 | 80 | — | 40 | 50 | 50 | 60 |
| Ragweed | 40 | 30 | 20 | 30 | 30 | 30 | 20 | 50 | 50 | — | 20 | 20 | 50 | 60 |
| Ryegrass, Italian | 60 | 70 | 20 | 30 | 50 | 10 | 10 | 20 | 20 | 0 | 30 | 50 | 90 | 90 |
| Soybean | 100 | 90 | 100 | 100 | 100 | 100 | 80 | 80 | 100 | — | 100 | 100 | 100 | 100 |
| Wheat | 20 | 20 | 10 | 10 | 30 | 10 | 10 | 20 | 30 | 10 | 10 | 20 | 50 | 30 |
| | Compounds | | | | | | | | | | | | | |
| 125 g ai/ha | 360 | 361 | 362 | 363 | 364 | 377 | 378 | 379 | 380 | 382 | 386 | 387 | 388 | 391 |
| Postemergence | | | | | | | | | | | | | | |
| Barnyardgrass | 90 | 70 | 60 | 20 | 50 | 30 | 40 | 30 | 20 | 30 | 20 | 20 | 10 | 40 |
| Blackgrass | 80 | 80 | 70 | 30 | 90 | 0 | 30 | 30 | 20 | 10 | 0 | 60 | 0 | 60 |
| Corn | 90 | 20 | 30 | 10 | 30 | 20 | 20 | 30 | 10 | 20 | 10 | 10 | 10 | 60 |
| Foxtail, Giant | — | — | — | — | — | — | — | — | — | — | — | — | — | — |
| Foxtail, Green | 90 | 90 | 70 | 30 | 80 | 20 | 70 | 20 | 20 | 20 | 10 | 40 | 10 | 70 |
| Galium | 100 | 100 | 100 | 100 | 100 | 100 | 90 | 70 | 100 | 100 | 100 | 100 | 70 | 100 |
| Goosegrass | — | — | — | — | — | — | — | — | — | — | — | — | — | — |
| Horseweed | — | 0 | 0 | 0 | 0 | 50 | 10 | 0 | 60 | 50 | 30 | 50 | 0 | 0 |
| Kochia | 20 | 70 | 80 | 40 | 70 | 90 | 90 | 90 | 100 | 90 | 80 | 80 | 90 | 100 |
| Oat, Wild | 30 | 30 | 30 | 0 | 60 | 0 | 0 | 0 | 0 | 0 | 0 | 0 | 20 | 30 |
| Pigweed, Palmer | 60 | 20 | 30 | 70 | 70 | 90 | 90 | 80 | 90 | 100 | 80 | 90 | 100 | 90 |
| Ragweed | 50 | 60 | 50 | 0 | 70 | 80 | 70 | 30 | 90 | 90 | 50 | 70 | 60 | 60 |
| Ryegrass, Italian | 70 | 80 | 80 | 40 | 70 | 40 | 20 | 30 | 40 | 40 | 40 | 40 | 20 | 50 |
| Soybean | 100 | 100 | 100 | 100 | 100 | 100 | 100 | 100 | 100 | 100 | 100 | 100 | 100 | 100 |
| Wheat | 30 | 40 | 40 | 20 | 30 | 20 | 30 | 10 | 10 | 20 | 10 | 10 | 10 | 30 |
| | Compounds | | | | | | | | | | | | | |
| 125 g ai/ha | 392 | 393 | 394 | 395 | 396 | 397 | 398 | 399 | 40 0 | 401 | 402 | 403 | 408 | 410 |
| Postemergence | | | | | | | | | | | | | | |
| Barnyardgrass | 50 | 30 | 50 | 40 | 30 | 40 | 20 | 20 | 30 | 10 | 20 | 30 | 10 | 60 |
| Blackgrass | 80 | 0 | 50 | 40 | 30 | 80 | 70 | 0 | 40 | 10 | 20 | 20 | 0 | 80 |
| Corn | 30 | 20 | 20 | 70 | 50 | 70 | 10 | 0 | 30 | 0 | 10 | 20 | 10 | 70 |
| Foxtail, Giant | — | — | — | — | — | — | — | — | — | — | — | — | — | — |
| Foxtail, Green | 60 | 10 | 30 | 70 | 60 | 90 | 70 | 10 | 20 | 10 | 70 | 50 | 20 | 70 |
| Galium | 100 | 70 | 100 | 100 | 100 | — | — | — | — | — | — | 100 | 100 | 100 |
| Goosegrass | — | — | — | — | — | — | — | — | — | — | — | — | — | — |
| Horseweed | 60 | 0 | 0 | 50 | 0 | 40 | 60 | 50 | 40 | 60 | 50 | — | 0 | 70 |
| Kochia | 90 | 70 | 90 | 90 | 80 | 90 | 90 | 90 | 70 | 90 | 90 | 80 | 70 | 90 |
| Oat, Wild | 70 | 0 | 50 | 50 | 20 | 20 | 0 | 0 | 0 | 0 | 0 | 30 | 30 | 80 |
| Pigweed, Palmer | 100 | 60 | 80 | 90 | 90 | 100 | 90 | 100 | 100 | 100 | 90 | 90 | 80 | 80 |
| Ragweed | 90 | 60 | 70 | 70 | 70 | 100 | 70 | 70 | 60 | 60 | 90 | 70 | 50 | 90 |
| Ryegrass, Italian | 80 | 10 | 50 | 60 | 0 | 50 | 30 | 0 | 0 | 0 | 30 | 50 | 70 | 70 |
| Soybean | 100 | 70 | 100 | 100 | 100 | 50 | 20 | 0 | 20 | 0 | 40 | 100 | 100 | 100 |
| Wheat | 50 | 10 | 30 | 40 | 20 | 20 | 20 | 20 | 20 | 10 | 20 | 20 | 30 | 50 |

| | Compounds | | |
|---|---|---|---|
| 125 g ai/ha | 411 | 412 | 413 |
| Postemergence | | | |
| Barnyardgrass | 30 | 40 | 20 |
| Blackgrass | 10 | 60 | 20 |
| Corn | 0 | 20 | 10 |
| Foxtail, Giant | — | — | — |
| Foxtail, Green | 20 | 80 | 10 |
| Galium | 90 | 100 | 90 |
| Goosegrass | — | — | — |
| Horseweed | 0 | 20 | 10 |
| Kochia | 0 | 80 | 90 |
| Oat, Wild | 0 | 20 | 0 |
| Pigweed, Palmer | 90 | 80 | 100 |
| Ragweed | 70 | 90 | 70 |
| Ryegrass, Italian | 50 | 70 | 40 |
| Soybean | 100 | 100 | 100 |
| Wheat | 10 | 20 | 10 |
| | Compounds | | |
| 31 g ai/ha | 180 | 193 | 268 |
| Postemergence | | | |
| Barnyardgrass | 10 | 10 | 20 |
| Blackgrass | 0 | 0 | 30 |
| Corn | 0 | 0 | 20 |
| Foxtail, Green | 10 | 30 | 70 |
| Galium | 70 | 60 | 90 |
| Horseweed | 0 | 0 | — |
| Kochia | 30 | 20 | 40 |
| Oat, Wild | 0 | 0 | 20 |
| Pigweed, Palmer | 30 | 20 | 80 |
| Ragweed | 40 | 50 | 50 |
| Ryegrass, Italian | 0 | 20 | 50 |
| Soybean | 90 | 100 | 100 |
| Wheat | 10 | 10 | 30 |

### Test D

Plant species in the flooded paddy test selected from barnyardgrass (Echinochloa crus-galli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 to 14 days, after which time all species were compared to controls and visually evaluated. Plant response ratings, summarized in Table D, are based on a scale of 0 to 100 where 0 is no effect and 100 is complete control. A dash (-) response means no test result.

TABLE D

| | Compounds | | | | | | | | | | | | | |
|---|---|---|---|---|---|---|---|---|---|---|---|---|---|---|
| 250 g ai/ha | 150 | 151 | 152 | 153 | 154 | 155 | 156 | 157 | 158 | 165 | 167 | 168 | 169 | 170 |
| Flood | | | | | | | | | | | | | | |
| Barnyardgrass | 0 | 40 | 0 | 35 | 15 | 20 | 40 | 15 | 25 | 0 | 0 | 0 | 0 | 0 |
| Ducksalad | 0 | 50 | 0 | 70 | 10 | 60 | 65 | 0 | 65 | 0 | 0 | 0 | 90 | 55 |
| Rice | 0 | 45 | 0 | 15 | 10 | 15 | 25 | 20 | 0 | 0 | 0 | 0 | 0 | 15 |
| Sedge, Umbrella | 0 | 75 | 0 | 98 | 70 | 98 | 95 | 45 | 75 | 0 | 0 | 0 | 85 | 95 |
| | Compounds | | | | | | | | | | | | | |
| 250 g ai/ha | 171 | 172 | 173 | 174 | 175 | 177 | 178 | 179 | 180 | 191 | 192 | 194 | 195 | 196 |
| Flood | | | | | | | | | | | | | | |
| Barnyardgrass | 0 | 0 | 0 | 0 | 0 | 35 | 15 | 0 | 0 | 0 | 0 | 0 | 45 | 0 |
| Ducksalad | 60 | 0 | 0 | 0 | 0 | 50 | 95 | 0 | 65 | 0 | 0 | 0 | 70 | 0 |
| Rice | 0 | 0 | 0 | 0 | 0 | 0 | 15 | 0 | 0 | 0 | 0 | 0 | 0 | 0 |
| Sedge, Umbrella | 95 | 0 | 0 | 0 | 0 | 95 | 90 | 0 | 40 | 0 | 0 | 0 | 98 | 0 |
| | Compounds | | | | | | | | | | | | | |
| 250 g ai/ha | 203 | 207 | 210 | 211 | 212 | 213 | 214 | 215 | 216 | 217 | 218 | 219 | 220 | 221 |
| Flood | | | | | | | | | | | | | | |
| Barnyardgrass | 0 | 0 | 20 | 55 | 25 | 20 | 0 | 50 | 40 | 50 | 0 | 10 | 0 | 0 |
| Ducksalad | 0 | 0 | 85 | 100 | 95 | 90 | 70 | 100 | 100 | 85 | 60 | 50 | 0 | 0 |
| Rice | 0 | 0 | 15 | 50 | 40 | 0 | 0 | 55 | 10 | 20 | 0 | 0 | 0 | 0 |
| Sedge, Umbrella | 0 | 0 | 100 | 100 | 98 | 100 | 90 | 100 | 95 | 98 | 80 | 50 | 0 | 0 |
| | Compounds | | | | | | | | | | | | | |
| 250 g ai/ha | 223 | 224 | 225 | 227 | 228 | 229 | 230 | 231 | 232 | 233 | 234 | 235 | 236 | 240 |
| Flood | | | | | | | | | | | | | | |
| Barnyardgrass | 10 | 10 | 25 | 50 | 15 | 0 | 15 | 15 | 0 | 0 | 75 | 10 | 0 | 35 |
| Ducksalad | 80 | 95 | 100 | 80 | 80 | 0 | 70 | 85 | 0 | 0 | 100 | 90 | 75 | 80 |
| Rice | 10 | 15 | 15 | 10 | 25 | 0 | | 0 | 0 | 0 | 0 | 15 | 0 | 10 |
| Sedge, Umbrella | 95 | 98 | 100 | 98 | 95 | 0 | 98 | 100 | 0 | 0 | 100 | 90 | 95 | 100 |
| | Compounds | | | | | | | | | | | | | |
| 250 g ai/ha | 241 | 242 | 248 | 249 | 250 | 252 | 253 | 254 | 256 | 258 | 259 | 260 | 262 | 263 |
| Flood | | | | | | | | | | | | | | |
| Barnyardgrass | 50 | 55 | 0 | 0 | 0 | 35 | 0 | 25 | 45 | 35 | 0 | 0 | 0 | 20 |
| Ducksalad | 90 | 95 | 0 | 0 | 0 | 15 | 85 | 98 | 98 | 98 | 0 | 0 | 0 | 65 |
| Rice | 15 | 15 | 0 | 0 | 0 | 0 | 10 | 20 | 35 | 10 | 0 | 0 | 0 | 0 |
| Sedge, Umbrella | 100 | 98 | 0 | 0 | 0 | 15 | 70 | 98 | 98 | 98 | 0 | 0 | 0 | 85 |
| | Compounds | | | | | | | | | | | | | |
| 250 g ai/ha | 264 | 265 | 266 | 267 | 268 | 269 | 270 | 271 | 272 | 273 | 275 | 276 | 277 | 278 |
| Flood | | | | | | | | | | | | | | |
| Barnyardgrass | 35 | 30 | 0 | 35 | 40 | 45 | 15 | 0 | 0 | 0 | 35 | 35 | 55 | 45 |
| Ducksalad | 75 | 75 | 75 | 50 | 65 | 100 | 100 | 85 | 0 | 15 | 85 | 70 | 40 | 100 |
| Rice | 10 | 25 | 15 | 25 | 10 | 10 | 10 | 10 | 0 | 0 | 10 | 20 | 15 | 15 |
| Sedge, Umbrella | 80 | 90 | 95 | 98 | 100 | 98 | 95 | 100 | 0 | 40 | 95 | 80 | 98 | 100 |
| | Compounds | | | | | | | | | | | | | |
| 250 g ai/ha | 279 | 280 | 282 | 283 | 284 | 285 | 288 | 289 | 290 | 291 | 292 | 294 | 296 | 299 |
| Flood | | | | | | | | | | | | | | |
| Barnyardgrass | 60 | 25 | 0 | 0 | 0 | 0 | 0 | 50 | 10 | 40 | 0 | 0 | 0 | 0 |
| Ducksalad | 100 | 70 | 0 | 0 | 65 | 0 | 0 | 65 | 50 | 45 | 0 | 0 | 0 | 95 |
| Rice | 35 | 20 | 0 | 0 | 0 | 0 | 0 | 10 | 10 | 0 | 0 | 0 | 0 | 0 |
| Sedge, Umbrella | 100 | 95 | 0 | 0 | 0 | 0 | 0 | 100 | 85 | 95 | 0 | 0 | 0 | 25 |
| | Compounds | | | | | | | | | | | | | |
| 250 g ai/ha | 300 | 301 | 302 | 303 | 306 | 307 | 308 | 310 | 311 | 312 | 313 | 314 | 315 | 316 |
| Flood | | | | | | | | | | | | | | |
| Barnyardgrass | 0 | 20 | 0 | 0 | 0 | 0 | 0 | 10 | 0 | 0 | 35 | 15 | 15 | 0 |
| Ducksalad | 0 | 55 | 0 | 80 | 0 | 0 | 0 | 75 | 0 | 0 | 65 | 85 | 85 | 0 |
| Rice | 0 | 15 | 0 | 0 | 0 | 0 | 0 | 0 | 0 | 0 | 20 | 15 | 10 | 0 |
| Sedge, Umbrella | 0 | 85 | 0 | 90 | 0 | 0 | 0 | 80 | 0 | 0 | 98 | 100 | 60 | 0 |
| | Compounds | | | | | | | | | | | | | |
| 250 g ai/ha | 317 | 318 | 319 | 320 | 321 | 322 | 323 | 324 | 325 | 326 | 327 | 328 | 329 | 330 |
| Flood | | | | | | | | | | | | | | |
| Barnyardgrass | 0 | 45 | 0 | 0 | 0 | 0 | 30 | 40 | 0 | 15 | 15 | 15 | 0 | 0 |
| Ducksalad | 0 | 10 | 0 | 0 | 0 | 0 | 30 | 80 | 35 | 80 | 25 | 30 | 0 | 0 |
| Rice | 0 | 15 | 0 | 0 | 0 | 0 | 0 | 10 | 0 | 0 | 0 | 0 | 0 | 0 |
| Sedge, Umbrella | 0 | 45 | 0 | 0 | 0 | 0 | 70 | 98 | 75 | 95 | 70 | 85 | 0 | 0 |
| | Compounds | | | | | | | | | | | | | |
| 250 g ai/ha | 331 | 332 | 333 | 334 | 335 | 336 | 337 | 338 | 340 | 341 | 342 | 343 | 344 | 345 |
| Flood | | | | | | | | | | | | | | |
| Barnyardgrass | 0 | 0 | 0 | 10 | 0 | 0 | 0 | 10 | 0 | 25 | 0 | 35 | 25 | 40 |
| Ducksalad | 0 | 0 | 0 | 30 | 35 | 25 | 0 | 40 | 75 | 70 | 10 | 45 | 30 | 65 |
| Rice | 0 | 0 | 0 | 0 | 0 | 0 | 0 | 10 | 0 | 0 | 15 | 15 | 0 | 10 |
| Sedge, Umbrella | 0 | 0 | 0 | 70 | 35 | 0 | 0 | 45 | 60 | 90 | 15 | 65 | 70 | 60 |
| | Compounds | | | | | | | | | | | | | |
| 250 g ai/ha | 346 | 347 | 348 | 349 | 350 | 352 | 353 | 354 | 355 | 356 | 357 | 358 | 359 | 360 |
| Flood | | | | | | | | | | | | | | |
| Barnyardgrass | 0 | 0 | 30 | 0 | 0 | 0 | 0 | 0 | 0 | 0 | 10 | 30 | 15 | 45 |
| Ducksalad | 65 | 0 | 75 | 0 | 0 | 0 | 0 | 0 | 0 | 0 | 45 | 95 | 60 | 100 |
| Rice | 0 | 0 | 0 | 0 | 0 | 0 | 0 | 0 | 0 | 0 | 0 | 15 | 10 | 15 |
| Sedge, Umbrella | 85 | 0 | 45 | 95 | 0 | 0 | 0 | 0 | 0 | 0 | 90 | 90 | 65 | 90 |
| | Compounds | | | | | | | | | | | | | |
| 250 g ai/ha | 361 | 362 | 363 | 364 | 377 | 378 | 379 | 380 | 382 | 386 | 387 | 388 | 391 | 392 |
| Flood | | | | | | | | | | | | | | |
| Barnyardgrass | 30 | 35 | 0 | 10 | 0 | 0 | 35 | 55 | 50 | 0 | 25 | 0 | 10 | 40 |
| Ducksalad | 70 | 50 | 0 | 80 | 15 | 0 | 55 | 30 | 15 | 0 | 20 | 0 | 35 | 65 |
| Rice | 15 | 25 | 0 | 10 | 0 | 0 | 15 | 10 | 15 | 0 | 10 | 0 | 0 | 0 |
| Sedge, Umbrella | 95 | 95 | 0 | 95 | 55 | 0 | 65 | 40 | 25 | 0 | 70 | 0 | 45 | 65 |
| | Compounds | | | | | | | | | | | | | |
| 250 g ai/ha | 394 | 395 | 396 | 397 | 398 | 399 | 400 | 401 | 402 | 403 | 408 | 410 | 411 | 412 |
| Flood | | | | | | | | | | | | | | |
| Barnyardgrass | 15 | 0 | 0 | 50 | 25 | 0 | 15 | 0 | 25 | 0 | 10 | 30 | 0 | 25 |
| Ducksalad | 10 | 0 | 0 | 10 | 15 | 0 | 45 | 0 | 45 | 50 | 90 | 85 | 0 | 75 |
| Rice | 0 | 0 | 0 | 15 | 15 | 0 | 0 | 0 | 0 | 0 | 15 | 15 | 0 | 10 |
| Sedge, Umbrella | 0 | 0 | 0 | 45 | 45 | 0 | 40 | 0 | 25 | 45 | 98 | 95 | 0 | 90 |

| | Compound |
|---|---|
| 250 g ai/ha | 413 |
| Flood | |
| Barnyardgrass | 35 |
| Ducksalad | 15 |
| Rice | 10 |
| Sedge, Umbrella | 45 |

What is claimed is:

1. A compound of Formula 1, all stereoisomers, N-oxides, and salts thereof 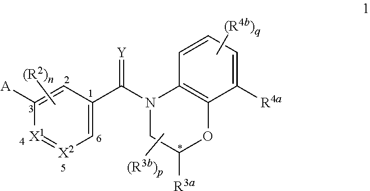
wherein
A is a 5- or 6-membered heterocyclic ring, containing ring members selected from carbon atoms and up to 4 heteroatoms independently selected from up to 2 O, up to 2 S and up to 4 N atoms, one or two carbon or sulfur ring members of the heterocycle can optionally be in the oxidized form of a carbonyl, sulfonyl, sulfinyl moiety, said ring bound to the remainder of Formula 1 through a carbon atom or a heteroatom, and optionally substituted with 1 to 4 R¹;
R¹ is independently R^{1a}, (R^{1b})ₘ or R^{1c} or any combination thereof;
R^{1a} is H, halogen, cyano, nitro, amino, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ alkyloxacycloalkyl, C₂-C₆ thiacycloalkyl, C₃-C₇ thiacycloalkylalkyl, C₃-C₇ alkylthiacycloalkyl, C₂-C₆ (O-thia)cycloalkyl, C₃-C₇ (O-thia)cycloalkylalkyl, C₃-C₇ alkyl(O-thia)cycloalkyl, C₂-C₆ (O₂thia)cycloalkyl, C₃-C₇ (O₂thia)cycloalkylalkyl, C₃-C₇ alkyl(O₂thia)cycloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl, C₁-C₇ haloalkoxy, C₁-C₇ alkylthio, C₂-C₇ alkylthioalkyl, C₁-C₅ alkylsulfinyl, C₁-C₅ alkylsulfonyl, C₁-C₄ alkylsulfonate, C₁-C₅ haloalkylthio, C₁-C₅ haloalkylsulfinyl, C₁-C₅ haloalkylsulfonyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₂-C₇ alkylthiocycloalkyl, C₄-C₇ alkylsulfinylcycloalkyl, C₄-C₇ alkylsulfonylcycloalkyl, C₄-C₇ haloalkylthiocycloalkyl, C₂-C₇ haloalkylsulfinylcycloalkyl, C₂-C₇ haloalkylsulfonylcycloalkyl, C₂-C₇ alkylsulfoximinoalkyl, C₂-C₅ cyanoalkyl, C₄-C₇ cyanocycloalkyl, C₁-C₄ nitroalkyl, C₁-C₇ alkylamino, C₂-C₇ dialkylamino, C₃-C₅ alkylcarbonyl(alkyl)amino, C₃-C₅ alkoxycarbonyl(alkyl)amino, C₂-C₄ alkoxysulfonyl(alkyl)amino, C₂-C₆ alkylcarbonyl, C₃-C₆ alkylcarbonylalkyl, C₂-C₆ alkoxycarbonyl, C₃-C₆ alkoxycarbonylalkyl, C₃-C₆ trialkylsilyl or C₅-C₈ trialkylsilylalkynyl; or
phenyl optionally substituted with up to 3 substituents independently selected from the group consisting of halogen, cyano, C₁-C₂ alkyl, C₁-C₂ haloalkyl, C₁-C₂ alkoxy and C₁-C₂ haloalkoxy;
R^{1b} is H, halogen, cyano, nitro, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy, C₁-C₄ alkylthio or C₂-C₄ alkoxycarbonyl;
m is 0, 1 or 2;
R^{1c} is H, C₁-C₇ alkyl, C₃-C₇ cycloalkyl or C₁-C₇ haloalkyl;
X¹ and X² are independently N or CR²;
n is 0, 1, 2 or 3;
each R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₃-C₅ cycloalkylsulfonate, C₁-C₄ haloalkylsulfonate, C₁-C₄ haloalkylthio, C₁-C₄ haloalkylsulfinyl, C₁-C₄ haloalkylsulfonyl, C₂-C₅ cyanoalkyl, C₄-C₆ cyanocycloalkyl or C₂-C₅ alkoxycarbonyl; or
two adjacent R² may be taken together to form a saturated or unsaturated 5- to 8-membered ring, containing carbon atoms and optionally 1 to 3 oxygen, sulfur or nitrogen atoms as ring members, one or two carbon or sulfur ring members of the heterocycle can optionally be in the oxidized form of a carbonyl, sulfonyl, sulfinyl moiety, said ring unsubstituted or substituted with at least one substituent independently selected from the group consisting of halogen, cyano, nitro, C₁-C₄ alkyl, C₃-C₆ cycloalkyl, C₁-C₄ haloalkyl, C₁-C₄ alkoxy and C₁-C₄ haloalkoxy;
Y is O or S;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ alkylsulfinylalkyl, C₂-C₇ alkylsulfonylalkyl, C₂-C₇ haloalkylthioalkyl, C₂-C₇ haloalkylsulfinylalkyl, C₂-C₇ haloalkylsulfonylalkyl, C₂-C₅ cyanoalkyl, C₄-C₆ cyanocycloalkyl, C₁-C₄ nitroalkyl, C₃-C₆ alkylcarbonylalkyl, C₂-C₆ oxacycloalkyl, C₂-C₆ oxacycloalkylalkyl, C₃-C₇ cycloalkoxy, C₄-C₇ cycloalkoxyalkyl or C₃-C₆ alkoxycarbonylalkyl;
each R^{3b} is independently H, halogen or C₁-C₃ alkyl; or;
R^{3a} and R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂; or
two R^{3b} are taken together with the carbon atom to which they are attached to form a 3- to 7-membered ring containing ring members selected from carbon atoms and up to 3 heteroatoms independently selected from 1 oxygen atom, 1 sulfur atom, and up to 3 nitrogen atoms, wherein up to 2 carbon atom ring members are independently selected from C(═O) and C(═S) and the sulfur atom ring member is selected from S, S(O) or S(O)₂;
p is 0, 1, 2 or 3;
R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl, C₅-C₈ trialkylsilylalkynyl, C₁-C₅ alkylthio, C₁-C₅ haloalkylthio or C₂-C₅ alkoxycarbonyl;
q is 0, 1 or 2;
each R^{4b} is independently H, halogen, cyano, nitro, C₁-C₄ alkyl, C₁-C₄ haloalkyl, C₁-C₄ alkoxy or C₁-C₄ alkylthio;
provided that the compounds of Formula 1 are other than:
[3-(5-cyclopropyl-1,2,4-oxadiazol-3-yl)phenyl](2-ethyl-2,3-dihydro-4H-1,4-benzoxazin-4-yl)-methanone (CAS Registry No. 1798020-19-5);
(8-chloro-2-ethyl-2,3-dihydro-4H-1,4-benzoxazin-4-yl)[3-(1H-tetrazol-1-yl)phenyl]methanone (CAS Registry No. 2093742-48-2);
(2-ethyl-2,3-dihydro-4H-1,4-benzoxazin-4-yl)[2-(1H-1,2,4-triazol-1-yl)-4-pyridinyl]-methanone (CAS Registry No. 1808378-56-4);
(8-chloro-2-ethyl-2,3-dihydro-4H-1,4-benzoxazin-4-yl)[2-(1H-1,2,4-triazol-1-yl)-4-pyridinyl]-methanone (CAS Registry No. 1808849-41-3);
(2,3-dihydro-2,7-dimethyl-4H-1,4-benzoxazin-4-yl)[2-(1H-1,2,4-triazol-1-yl)-4-pyridinyl]-methanone (CAS Registry No. 1436224-65-5);
(8-chloro-2,3-dihydro-2-methyl-4H-1,4-benzoxazin-4-yl)[2-(1H-pyrazol-1-yl)-4-pyridinyl]-methanone (CAS Registry No. 2224006-86-2);
(2,3-Dihydro-2-methyl-4H-1,4-benzoxazin-4-yl)[3-(1-pyrrolidinyl)phenyl]methanone (CAS Registry No. 2733463-68-6);
Methanone, (3,4-dihydrospiro[2H-1,4-benzoxazine-2,1′-cyclopropan]-4-yl)[3-(1-pyrrolidinyl)phenyl](CAS Registry No. 2733410-16-5);
(2,3-Dihydro-2-methyl-4H-1,4-benzoxazin-4-yl)[3-(1H-pyrrol-1-yl)phenyl]methanone (CAS Registry No. 2305402-15-5);
(3,4-Dihydrospiro[2H-1,4-benzoxazine-2,1′-cyclopropan]-4-yl)[3-(1H-pyrrol-1-yl)phenyl]methanone (CAS Registry No. 2305290-36-0);
(8-Chloro-2,3-dihydro-2-methyl-4H-1,4-benzoxazin-4-yl)[3-[5-(methoxymethyl)-1,3,4-oxadiazol-2-yl]phenyl]methanone (CAS Registry No. 2223792-20-7);
(7-Fluoro-2,3-dihydro-2-methyl-4H-1,4-benzoxazin-4-yl)[2-(4-methyl-1-piperazinyl)-4-pyridinyl]methanone (CAS Registry No. 2212440-53-2);
(7-Fluoro-2,3-dihydro-2-methyl-4H-1,4-benzoxazin-4-yl)[3-(1H-1,2,4-triazol-5-yl)phenyl]methanone (CAS Registry No. 2094921-82-9);
(2-Ethyl-2,3-dihydro-4H-1,4-benzoxazin-4-yl)[5-(4-morpholinyl)-3-pyridinyl]methanone (CAS Registry No. 1957585-10-2);
(8-Chloro-2,3-dihydro-2-methyl-4H-1,4-benzoxazin-4-yl)[5-(4-morpholinyl)-3-pyridinyl]methanone (CAS Registry No. 1957541-06-8);
(8-Chloro-2-ethyl-2,3-dihydro-4H-1,4-benzoxazin-4-yl)[5-(4-morpholinyl)-3-pyridinyl]methanone (CAS Registry No. 1956163-57-7);
(2,3-Dihydro-2,6-dimethyl-4H-1,4-benzoxazin-4-yl)[5-(4-morpholinyl)-3-pyridinyl]methanone (CAS Registry No. 1955383-94-4);
(2,3-Dihydro-2,2-dimethyl-4H-1,4-benzoxazin-4-yl)[5-(4-morpholinyl)-3-pyridinyl]methanone (CAS Registry No. 1955104-90-1);
(6-Chloro-2,3-dihydro-2-methyl-4H-1,4-benzoxazin-4-yl)[5-(4-morpholinyl)-3-pyridinyl]methanone (CAS Registry No. 1954398-26-5);
(2,3-Dihydro-6-methoxy-2-methyl-4H-1,4-benzoxazin-4-yl)[5-(4-morpholinyl)-3-pyridinyl]methanone (CAS Registry No. 1947266-43-4); and
(2,3-Dihydro-6-methoxy-2-methyl-4H-1,4-benzoxazin-4-yl)[3-(1H-imidazol-1-yl)phenyl]methanone (CAS Registry No. 1384688-76-9).

2. The compound of claim 1 wherein
A is selected from

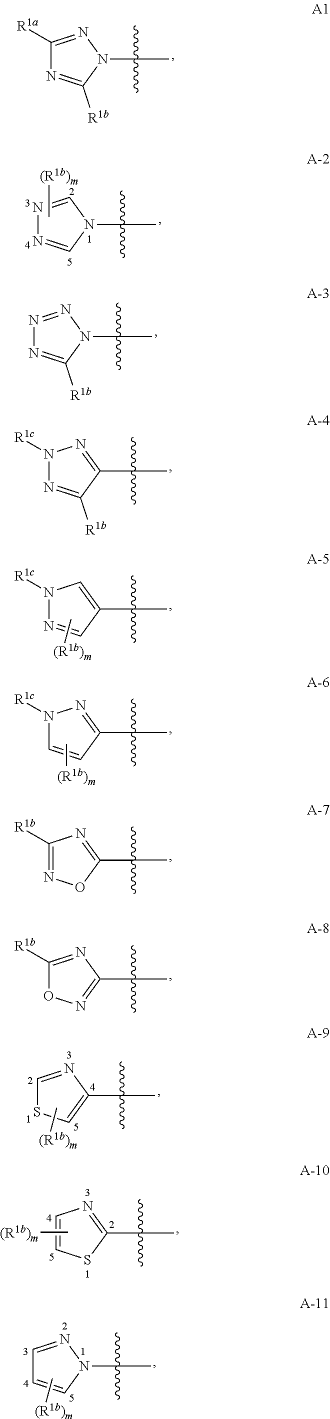

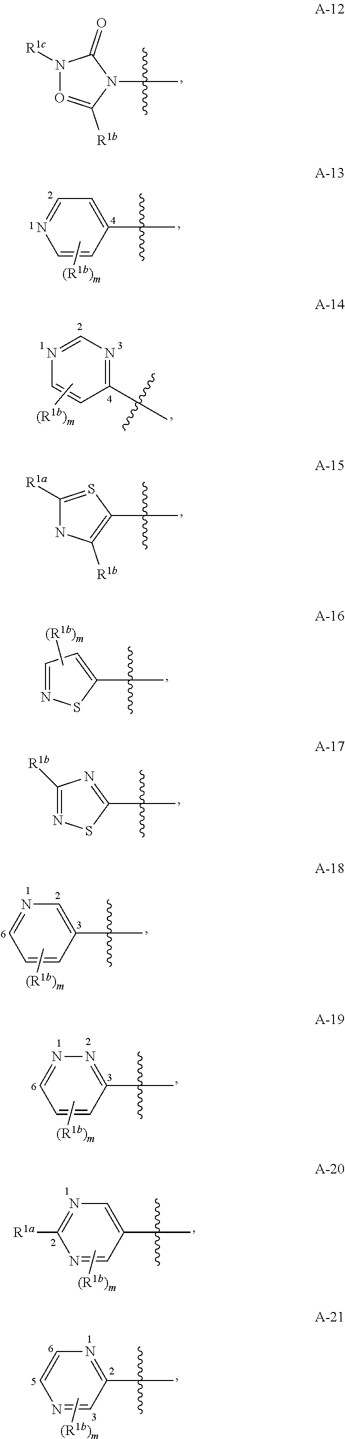

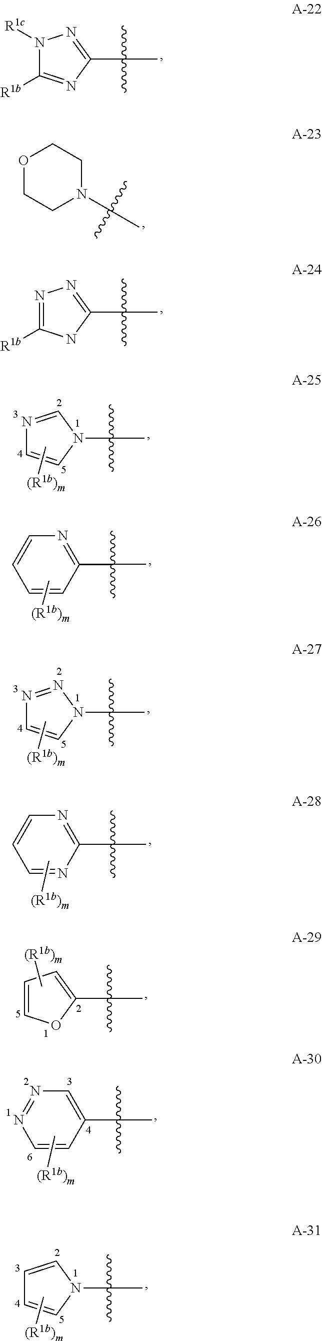

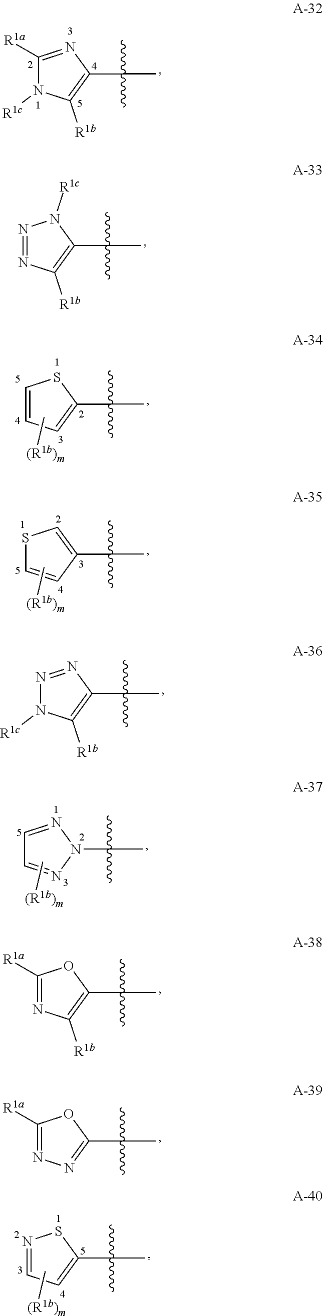

3. The compound of claim 1 wherein
A is A-1, A-4, A-5, A-12, A-14, A-15 or A-17.

4. The compound of claim 3 wherein
A is A-1.

5. The compound of claim 4 wherein
both X¹ and X² are CR²;
R^{1a} is H, halogen, cyano, C₁-C₇ alkyl, C₂-C₆ alkenyl, C₂-C₆ alkynyl, C₃-C₇ cycloalkyl, C₄-C₇ cycloalkylalkyl, C₄-C₇ alkylcycloalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₂-C₅ alkenyloxy, C₂-C₅ alkynyloxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₃-C₅ halocycloalkyl, C₂-C₄ alkoxyalkyl, C₁-C₄ alkoxy or C₂-C₄ alkoxycarbonyl;
R² is independently H, halogen, cyano, nitro, hydroxy, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl; or R² may be taken together to form a 5- or 6-membered ring, containing up to 2 oxygen atoms as the ring members.
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl, C₂-C₇ haloalkoxyalkyl, C₂-C₇ alkylthioalkyl, C₂-C₇ haloalkylthioalkyl, C₂-C₅ cyanoalkyl, C₁-C₄ nitroalkyl, C₃-C₆ alkylcarbonylalkyl, C₂-C₆ oxacycloalkyl, C₃-C₇ cycloalkoxy or C₃-C₆ alkoxycarbonylalkyl;
R^{3b} is H or halogen;
R^{4a} is H, halogen, cyano, nitro, C₁-C₅ alkyl, C₂-C₅ alkenyl, C₂-C₅ alkynyl, C₃-C₅ cycloalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy, C₃-C₆ trialkylsilyl or C₅-C₈ trialkylsilylalkynyl;
R^{4b} is H, halogen, cyano, nitro, C₁-C₄ alkyl, C₁-C₄ haloalkyl, C₁-C₄ alkoxy or C₁-C₄ alkylthio.

6. The compound of claim 5 wherein
R^{1a} is H, halogen, cyano, C₁-C₇ alkyl, C₃-C₇ cycloalkyl, C₄-C₇ cycloalkylalkyl, C₁-C₇ haloalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ oxacycloalkylalkyl, C₃-C₇ halocycloalkyl, C₄-C₇ haloalkylcycloalkyl, C₂-C₇ alkoxyalkyl, C₂-C₇ haloalkoxyalkyl, C₁-C₇ alkoxy, C₃-C₇ cycloalkoxy, C₄-C₇ cycloalkoxyalkyl or C₁-C₇ haloalkoxy;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₂-C₅ alkenyloxy, C₂-C₅ alkynyloxy, C₃-C₇ cycloalkoxy, C₄-C₇ cycloalkoxyalkyl, C₃-C₆ cycloalkyl, C₄-C₇ cycloalkylalkyl, C₁-C₅ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₅ haloalkoxy;
R^{3a} is halogen, C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₂-C₆ haloalkenyl, C₂-C₆ haloalkynyl, C₃-C₇ halocycloalkyl, C₄-C₇ haloalkylcycloalkyl, C₂-C₇ alkoxyalkyl or C₂-C₇ haloalkoxyalkyl;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H, halogen, cyano, C₁-C₄ alkyl, C₁-C₄ haloalkyl or C₁-C₄ alkoxy.

7. The compound of claim 6 wherein
R^{1a} is H, halogen, cyano, C₁-C₇ alkyl, C₃-C₇ cycloalkyl, C₄-C₇ cycloalkylalkyl, C₁-C₆ hydroxyalkyl, C₃-C₇ hydroxycycloalkyl, C₁-C₆ hydroxyhaloalkyl, C₄-C₈ alkoxycycloalkyl, C₂-C₇ alkoxyhaloalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl; and
R^{4b} is H or halogen.

8. The compound of claim 7 wherein
R^{1a} is H, Me, Et, i-Pro, i-Bu, Bu, t-Bu, Br, cyano, c-Bu, c-Pen, c-Hex, HOCH₂, HOC(Me)₂, CH₂OMe, CH₂O-i-Pro, CH₂CH₂OMe, CH₂-c-Hex or 3-oxetanyl;
R^{1b} is H, Me, i-Pro, CN, CF₃, F or Cl;
R² is independently H, OH, CN, OEt, propargyl, allyl, c-Pro, F, Cl, Br, CN, Me, Et, OMe, CF₃, OCF₃ or CH₂CF₃;
R^{3a} is Me, Et, Pro, i-Pro, CF₃, CH₂F or CH₂OMe; and
R^{4a} is H, CN, NO₂, F, Cl, Br, Me, Et, CF₃, CH₂F, OCF₃, OMe, CH₂OMe, CH═CH₂, C≡CSiMe₃, C≡CH or c-Pro.

9. The compound of claim 4 wherein
X¹ is N and X² is CR².
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

10. The compound of claim 9 wherein
R^{1a} is H;
R^{1b} is H;
R² is independently H, C₁-C₅ alkyl or C₁-C₅ alkoxy;
R^{3a} is C₁-C₇ alkyl, C₃-C₇ cycloalkyl, C₁-C₇ haloalkyl or C₂-C₇ alkoxyalkyl.
R^{3b} is H or halogen.
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl.
R^{4b} is H, halogen, cyano, C₁-C₄ alkyl, C₁-C₄ haloalkyl or C₁-C₄ alkoxy.

11. The compound of claim 4 wherein
X¹ is CR² and X² is N;
R^{1a} is H, halogen, cyano, C₁-C₇ alkyl, C₃-C₇ cycloalkyl, C₄-C₇ cycloalkylalkyl, C₂-C₆ oxacycloalkyl, C₃-C₇ halocycloalkyl, C₂-C₇ alkoxyalkyl, C₁-C₇ alkoxy, C₃-C₇ cycloalkoxy or C₄-C₇ cycloalkoxyalkyl;
R^{1b} is H, halogen, cyano, C₁-C₄ alkyl, C₃-C₅ cycloalkyl, C₁-C₄ haloalkyl, C₂-C₄ alkoxyalkyl or C₂-C₄ alkoxycarbonyl;
R² is independently H, halogen, cyano, OH, C₁-C₅ alkyl, C₂-C₅ alkenyl, C₂-C₅ alkynyl, C₃-C₆ cycloalkyl, C₁-C₅ haloalkyl, C₁-C₅ alkoxy or C₁-C₅ haloalkoxy;
R^{3a} is C₁-C₇ alkyl, C₂-C₆ alkenyl, C₂-C₆ alkynyl, C₃-C₇ cycloalkyl, C₁-C₇ alkoxy, C₄-C₇ cycloalkylalkyl, C₄-C₇ alkylcycloalkyl, C₁-C₇ haloalkyl, C₃-C₇ halocycloalkyl, C₂-C₇ alkoxyalkyl or C₂-C₇ haloalkoxyalkyl;
R^{3b} is H or halogen;
R^{4a} is H, halogen, cyano, NO₂, C₁-C₅ alkyl, C₂-C₅ alkenyl, C₂-C₅ alkynyl, C₃-C₅ cycloalkyl, C₁-C₅ alkoxy, C₁-C₅ haloalkoxy or C₅-C₈ trialkylsilylalkynyl; and
R^{4b} is H or halogen.

12. The compound of claim 4 wherein R² may be taken together to form a 5- or 6-membered ring, containing up to 2 oxygen atoms as the ring members.

13. The compound of claim 1 selected from the group consisting of
[(2S)-8-Chloro-2,3-dihydro-2-methyl-4H-1,4-benzoxazin-4-yl][2-methyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-[3-(1,1-dimethylethyl)-1H-1,2,4-triazol-1-yl]-2-methylphenyl]methanone;
[(2S)-8-Chloro-2,3-dihydro-2-methyl-4H-1,4-benzoxazin-4-yl][2-methoxy-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-ethyl-1H-1,2,4-triazol-1-yl)-2-methoxyphenyl]methanone;
[(2S)-8-Chloro-2,3-dihydro-2-methyl-4H-1,4-benzoxazin-4-yl][2-ethyl-5-[3-(1-methylethyl)-1H-1,2,4-triazol-1-yl]phenyl]methanone;
[(2S)-8-Chloro-2,3-dihydro-2-methyl-4H-1,4-benzoxazin-4-yl][5-(3-cyclobutyl-1H-1,2,4-triazol-1-yl)-2-methylphenyl]methanone;
[(2S)-8-Chloro-2,3-dihydro-2-methyl-4H-1,4-benzoxazin-4-yl][3-(3-cyclobutyl-1H-1,2,4-triazol-1-yl)phenyl]methanone;
[(2S)-2,3-Dihydro-2,8-dimethyl-4H-1,4-benzoxazin-4-yl][2-methoxy-5-(1H-1,2,4-triazol-1-yl)phenyl]methanone;

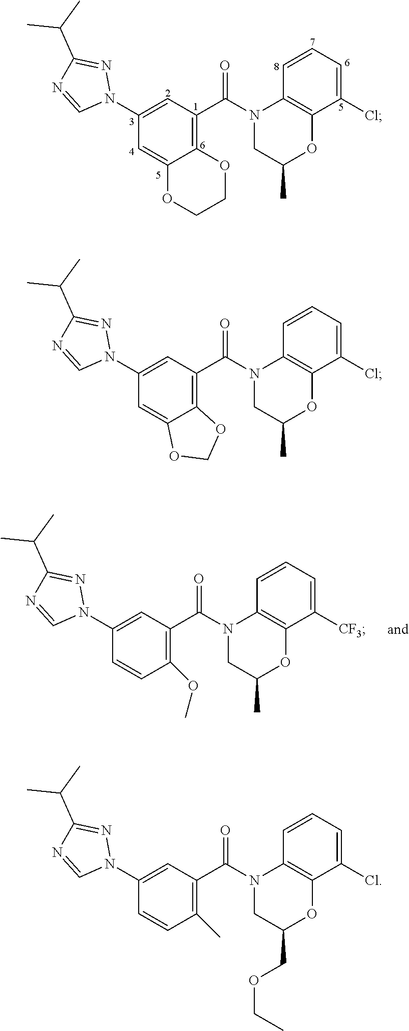 [SMILES: CC(C)c1ncn(-c2cc3c(c(C(=O)N4C[C@H](C)Oc5c(Cl)cccc54)c2)OCCO3)n1 ; CC(C)c1ncn(-c2cc3c(c(C(=O)N4C[C@H](C)Oc5c(Cl)cccc54)c2)OCO3)n1 ; COc1ccc(-n2cnc(C(C)C)n2)cc1C(=O)N1C[C@H](C)Oc2c1cccc2C(F)(F)F ; CCOC[C@H]1CN(C(=O)c2cc(-n3cnc(C(C)C)n3)ccc2C)c2cccc(Cl)c2O1]

14. A herbicidal composition comprising a compound of claim 1 and at least one component selected from the group consisting of surfactants, solid diluents and liquid diluents.

15. A herbicidal composition comprising a compound of claim 1, at least one additional active ingredient selected from the group consisting of other herbicides and herbicide safeners, and at least one component selected from the group consisting of surfactants, solid diluents and liquid diluents.

16. A herbicidal mixture comprising (a) a compound of claim 1, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b16) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners, and salts of compounds of (b1) through (b16).

17. A method for controlling the growth of undesired vegetation comprising contacting the vegetation or its environment with a herbicidally effective amount of a compound of claim 1.
